(19) United States
(12) Patent Application Publication
(10) Pub. No.: US 2024/0425499 A1
(43) Pub. Date: Dec. 26, 2024

(54) TYRO3 INHIBITORS

(71) Applicant: HALIA THERAPEUTICS, INC., Lehi, UT (US)

(72) Inventors: David James BEARSS, Lehi, UT (US); John Sai Keong KAUWE, III, Lehi, UT (US); Alexis Henri Abel MOLLARD, Lehi, UT (US)

(21) Appl. No.: 18/699,841

(22) PCT Filed: Oct. 10, 2022
(86) PCT No.: PCT/US2022/046183

§ 371 Date: Apr. 9, 2024

Related U.S. Application Data
(60) Provisional application No. 63/254,275, filed on Oct. 11, 2021.

(51) Int. Cl. C07D 471/04; A61K 31/437; A61K 31/444; A61K 31/4545; C07D 519/00

(52) U.S. Cl. CPC C07D 471/04; A61K 31/437; A61K 31/444; A61K 31/4545; C07D 519/00

(57) ABSTRACT

Compounds having activity as inhibitors of TYRO3 are provided. The compounds have Structure (I): (I) or a pharmaceutically acceptable salt, stereoisomer, tautomer, or prodrug thereof, wherein L, X, Y, Z, R¹, R^{2a}, R^{2c}, R³, p, and n are as defined herein. Methods associated with preparation and use of such compounds, pharmaceutical compositions comprising such compounds and methods to modulate the activity of TYRO3 are also provided.

### BACKGROUND

### Technical Field

Embodiments of the present disclosure are generally directed to compounds and methods for their preparation and use as therapeutic or prophylactic agents, for example for treatment of TYRO3 mediated diseases or disorders.

### Description of the Related Art

Tyrosine kinase receptors represent a superfamily of transmembrane proteins that relay signals from the extracellular environment to the cell. The signals are transmitted by ligands that bind to the extracellular domains of these kinases, thereby activating a signaling pathway. In this superfamily, the ligand is often a type of growth factor. A subfamily of kinases comprised in this superfamily are the TAM kinase subfamily, which comprises the Axl, TYRO3, and Mer tyrosine kinase receptors (Hafizi S and Dahlback B. Cytokine Growth Factor Rev 2006; 17: 295-304; Linger R M, et al. Adv Cancer Res 2008; 100: 35-83). This kinase subfamily has a single extracellular domain common to the N-end immunoglobulin domains and two type III fibronectin repeat structures. The structure of type III fibronectin repeats has a structural similarity to neural cell adhesion molecules (Neural Cell Adhesion Molecule, NCAM).

TAM receptor and ligand overexpression have been shown in a wide range of a disorders and diseases including, but not limited to cancer (including solid and hematological tumors), infectious diseases, and thrombosis, and correlate with poor prognosis in a variety of tumor types and their signals and promote survival, chemoresistance, motility, and invasion. In addition, their role in diminishing the innate immune response makes their inhibition a novel mechanism for reversing the immunosuppressive tumor microenvironment.

Accordingly, there is a need to develop inhibitors that will directly target TYRO3. Embodiments of the present disclosure fulfill this need and provide further related advantages.

### BRIEF SUMMARY

In brief, embodiments of the present disclosure provide compounds, including pharmaceutically acceptable salts, stereoisomers, tautomers, and prodrugs thereof, which are capable of modulating TYRO3 activity.

In one aspect, the disclosure provides compounds of Structure (I):

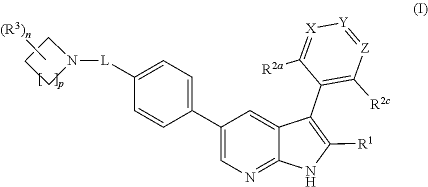

pharmaceutically acceptable salts, stereoisomers, tautomers, or prodrug thereof, wherein each of L, X, Y, Z, R¹, R^{2a}, R^{2c}, R³, p, and n are as defined herein.

In another aspect, pharmaceutical compositions comprising the disclosed compounds, and methods of use of the same for treatment of TYRO3 mediated diseases and disorders are also provided.

### DETAILED DESCRIPTION

In the following description, certain specific details are set forth in order to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is, as “including, but not limited to”.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, the terms “about” and “approximately” mean ±20%, ±10%, ±5% or ±1% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.
“Amino” refers to the —NH₂ radical.
“Carboxy” or “carboxyl” refers to the —CO₂H radical.
“Cyano” refers to the —CN radical.
“Hydroxy” or “hydroxyl” refers to the —OH radical.
“Oxo” refers to the ═O substituent.

“Alkyl” refers to a saturated, straight or branched hydrocarbon chain radical consisting solely of carbon and hydrogen atoms, having from one to twelve carbon atoms (C₁-C₁₂ alkyl), one to eight carbon atoms (C₁-C₈ alkyl) or one to six carbon atoms (C₁-C₆ alkyl), or any value within these ranges, such as C₄-C₆ alkyl and the like, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. The number of carbons referred to relates to the carbon backbone and carbon branching, but does not include carbon atoms belonging to any substituents. Unless stated otherwise specifically in the specification, an alkyl group is optionally substituted.

“Alkylene” or “alkylene chain” refers to a straight or branched divalent hydrocarbon chain linking the rest of the molecule to a radical group, consisting solely of carbon and hydrogen (unless substituted), containing no unsaturation, and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, alkylene is optionally substituted.

“Alkoxy” refers to a radical of the formula —ORₐ where Rₐ is an alkyl radical as defined above containing one to twelve carbon atoms (C₁-C₁₂ alkoxy), one to eight carbon atoms (C₁-C₈ alkoxy) or one to six carbon atoms (C₁-C₆ alkoxy), or any value within these ranges. Unless stated otherwise specifically in the specification, an alkoxy group is optionally substituted.

“Alkoxyalkyl” refers to a radical of the formula —RₐR_{b} where Rₐ is an alkyl radical as defined above and R_{b} is an alkoxy radical as defined above. An alkoxyalkyl may contain one to twelve carbon atoms (C₁-C₁₂ alkoxy), two to eight carbon atoms (C₂-C₈ alkoxy), or two to six carbon atoms (C₂-C₆ alkoxy), or any value within these ranges.

“Aminyl” refers to a radical of the formula —NRₐR_{b}, where Rₐ and R_{b} are each independently H or C₁-C₆ alkyl as defined above. When both of Rₐ and R_{b} are H, an “aminyl” group is the same as an “amino” group as defined above. The C₁-C₆ alkyl portion of an aminyl group is optionally substituted unless stated otherwise.

“Aromatic ring” refers to a cyclic planar molecule or portion of a molecule (i.e., a radical) with a ring of resonance bonds that exhibits increased stability relative to other connective arrangements with the same sets of atoms. Generally, aromatic rings contain a set of covalently bound co-planar atoms and comprises a number of π-electrons (for example, alternating double and single bonds) that is even but not a multiple of 4 (i.e., 4n+2π-electrons, where n=0, 1, 2, 3, etc.). Aromatic rings include, but are not limited to, phenyl, naphthenyl, imidazolyl, pyrrolyl, pyridinyl, pyrimidinyl, pyrazinyl, pyridonyl, pyridazinyl, pyrimidonyl. Unless stated otherwise specifically in the specification, an “aromatic ring” includes all radicals that are optionally substituted.

“Aryl” refers to a carbocyclic ring system radical comprising 6 to 18 carbon atoms, for example 6 to 10 carbon atoms (C₆-C₁₀ aryl) and at least one carbocyclic aromatic ring. For purposes of embodiments of this disclosure, the aryl radical is a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group is optionally substituted.

“Carbocyclic” or “carbocycle” refers to a ring system, wherein each of the ring atoms are carbon.

“Cycloalkyl” refers to a non-aromatic monocyclic or polycyclic carbocyclic radical consisting solely of carbon and hydrogen atoms, which may include fused or bridged ring systems, having from three to fifteen ring carbon atoms (C₃-C₁₅ cycloalkyl), from three to ten ring carbon atoms (C₃-C₁₀ cycloalkyl), or from three to eight ring carbon atoms (C₃-C₅ cycloalkyl), or any value within these ranges such as three to four carbon atoms (C₃-C₄ cycloalkyl), and which is saturated or partially unsaturated and attached to the rest of the molecule by a single bond.

Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group is optionally substituted.

“Cycloalkylalkyl” refers to an alkyl radical, as defined above that is substituted by a cycloalkyl radical. Unless stated otherwise specifically in the specification, a cycloalkylalkyl group is optionally substituted.

“Fused” refers to any ring structure described herein which is fused to another ring structure.

“Halo” refers to bromo, chloro, fluoro, or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group is optionally substituted.

“Hydroxylalkyl” refers to an alkyl radical, as defined above that is substituted by one or more hydroxyl radical. The hydroxyalkyl radical is joined at the main chain through the alkyl carbon atom. Unless stated otherwise specifically in the specification, a hydroxylalkyl group is optionally substituted.

“Aminylalkyl” refers to an alkyl radical, as defined above that is substituted by at least one aminyl substituent as defined above. Unless stated otherwise specifically in the specification, a hydroxylalkyl group is optionally substituted.

“Heterocyclyl” refers to a 3- to 18-membered, for example 3- to 10-membered or 3- to 8-membered, non-aromatic ring radical having one to ten ring carbon atoms (e.g., two to ten) and from one to six ring heteroatoms selected from the group consisting of nitrogen, oxygen and sulfur. Unless stated otherwise specifically in the specification, the heterocyclyl radical is partially or fully saturated and is a monocyclic, bicyclic, tricyclic or tetracyclic ring system, which may include fused, spirocyclic and/or bridged ring systems. Nitrogen, carbon and sulfur atoms in a heterocyclyl radical are optionally oxidized, and nitrogen atoms may be optionally quaternized. Examples of such heterocyclyl radicals include, but are not limited to, dioxolanyl, thienyl[1,3]dithianyl, decahydroisoquinolyl, furanonyl, imidazolinyl, imidazolidinyl, isothiazolidinyl, isoxazolidinyl, morpholinyl, octahydroindolyl, octahydroisoindolyl, hexahydro-1H-pyrrolizine, 2-oxopiperazinyl, 2-oxopiperidinyl, 2-oxopyrrolidinyl, oxazolidinyl, oxiranyl, piperidinyl, piperazinyl, 4-piperidonyl, azetidinyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group is optionally substituted.

“Heterocyclylalkyl” refers to a radical group of the formula —RₐR_{b} where Rₐ is an alkyl group and R_{b} is a heterocyclyl group as defined herein. Unless otherwise stated specifically in the specification, a heterocyclylalkyl group is optionally substituted.

“Heteroaryl” refers to a 5- to 18-membered, for example 5- to 6-membered, ring system radical comprising one to thirteen ring carbon atoms, one to six ring heteroatoms selected from the group consisting of nitrogen, oxygen and sulfur, and at least one aromatic ring. Heteroaryl radicals may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group is optionally substituted.

The term “substituted” as used herein means any of the above groups (e.g., alkyl, alkylene, alkylcarbonyl, alkoxy, alkoxyalkyl, aryl, cyanoalkyl, cycloalkyl, haloalkyl, heterocyclyl, heterocyclylalkyl, heteroaryl, heteroarylalkyl and/or hydroxylalkyl) wherein at least one hydrogen atom (e.g., 1, 2, 3, or all hydrogen atoms) is replaced by a bond to a non-hydrogen substituent. Examples of non-hydrogen substituents include, but are not limited to: amino, carboxyl, cyano, hydroxyl, halo, nitro, oxo, thiol, thioxo, alkyl, alkenyl, alkylcarbonyl, alkoxy, aryl, cyanoalkyl, cycloalkyl, haloalkyl, heterocyclyl, heterocyclylalkyl, heteroaryl, heteroarylalkyl and/or hydroxylalkyl substituents, each of which may also be optionally substituted with one or more of the above substituents.

The term “effective amount” or “therapeutically effective amount” refers to that amount of a compound described herein that is sufficient to effect the intended application including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refer to an approach for obtaining beneficial or desired results with respect to a disease, disorder or medical condition including but not limited to a therapeutic effect and/or a prophylactic effect. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. A prophylactic effect includes delaying or eliminating the appearance of a disease or condition, delaying or eliminating the onset of symptoms of a disease or condition, slowing, halting, or reversing the progression of a disease or condition, or any combination thereof. In certain embodiments, for prophylactic benefit, the compositions are administered to a subject at risk of developing a particular disease, or to a subject reporting one or more of the physiological symptoms of a disease, even though a diagnosis of this disease may not have been made.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

“Pharmaceutically acceptable salt” includes both acid and base addition salts.

“Pharmaceutically acceptable acid addition salt” refers to those salts which retain the biological effectiveness of the free bases, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable acid addition salts are those that are pharmacologically effective and suitable for contact with the tissues of patients without undue toxicity, irritation, or allergic response. Pharmaceutically acceptable acid addition salts which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness of the free acids, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable base addition salts are those that are pharmacologically effective and suitable for contact with the tissues of patients without undue toxicity, irritation, or allergic response. Pharmaceutically acceptable base addition salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some embodiments, pharmaceutically acceptable salts include quaternary ammonium salts such as quaternary amine alkyl halide salts (e.g., methyl bromide).

“Subject” refers to an animal, such as a mammal, for example a human. The methods described herein can be useful in both human therapeutics and veterinary applications. In some embodiments, the subject is a mammal, and in some embodiments, the subject is human.

“Mammal” includes humans and both domestic animals such as laboratory animals and household pets (e.g., cats, dogs, swine, cattle, sheep, goats, horses, rabbits), and non-domestic animals such as wildlife and the like.

“Prodrug” is meant to indicate a compound that may be converted under physiological conditions or by solvolysis to a biologically active compound described herein (e.g., compounds of Structure (I) or (II)). Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or thiol group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound and the like.

The term “in vivo” refers to an event that takes place in a subject's body.

Embodiments disclosed herein are also meant to encompass all pharmaceutically acceptable compounds of Structure (I) and (II).

Certain embodiments are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments include compounds produced by a process comprising administering a compound of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

Often crystallizations produce a solvate of the compounds disclosed herein. As used herein, the term “solvate” refers to an aggregate that comprises one or more compounds of the disclosure with one or more molecules of solvent. In some embodiments, the solvent is water, in which case the solvate is a hydrate. Alternatively, in other embodiments, the solvent is an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. In some aspects, the compounds of the disclosure are a true solvate, while in other cases, the compounds of the disclosure merely retain adventitious water or is a mixture of water plus some adventitious solvent.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

A “pharmaceutical composition” refers to formulations of compounds of the disclosure and a medium generally accepted in the art for the delivery of compounds of the disclosure to mammals, e.g., humans. Such a medium includes all pharmaceutically acceptable carriers, diluents or excipients therefor.

“Pharmaceutically acceptable carrier, diluent, or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

The compounds of the disclosure (i.e., compounds of Structure (I) or (II)) or their pharmaceutically acceptable salts may contain one or more center around which there exists geometric asymmetry and may thus give rise to stereoisomers. That is, some compounds of the disclosure may exist as enantiomers, diastereomers, and other stereoisomeric forms that are defined, in terms of absolute stereochemistry, as (R)- or (S)- or, as (D)- or (L)- for amino acids. Embodiments thus include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

Embodiments described herein include all manner of rotamers and conformationally restricted states of a compound of the disclosure. Atropisomers, which are stereoisomers arising because of hindered rotation about a single bond, where energy differences due to steric strain or other contributors create a barrier to rotation that is high enough to allow for isolation of individual conformers, are also included. As an example, certain compounds of the disclosure may exist as mixtures of atropisomers or purified or enriched for the presence of one atropisomer.

In some embodiments, the compounds of Structure (I) or (II) are a mixture of enantiomers or diastereomers. In other embodiments, the compounds of Structure (I) or (II) are substantially one enantiomer or diastereomer.

A “tautomer” refers to a proton shift from one atom of a molecule to another atom of the same molecule. Embodiments thus include tautomers of the disclosed compounds.

The chemical naming protocol and structure diagrams used herein are a modified form of the I.U.P.A.C. nomenclature system, using the ACD/Name Version 9.07 software program and/or ChemDraw Profesional Version 17.0.0.206 software naming program (CambridgeSoft). For complex chemical names employed herein, a substituent group is typically named before the group to which it attaches. For example, cyclopropylethyl comprises an ethyl backbone with a cyclopropyl substituent. Except as described below, all bonds are identified in the chemical structure diagrams herein, except for all bonds on some carbon atoms, which are assumed to be bonded to sufficient hydrogen atoms to complete the valency.

### Compounds

The disclosure provides compounds including pharmaceutically acceptable salts, stereoisomers, tautomers, and prodrugs thereof, which are capable of modulating TYRO3. Embodiments of the present disclosure provide a compound having the following Structure (I):

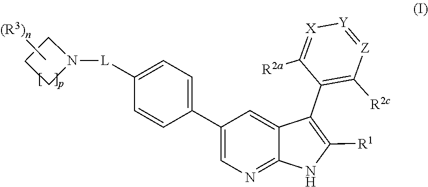

or a pharmaceutically acceptable salt, stereoisomer, tautomer, or prodrug thereof, wherein:
X is N or CR^{2d};
Y is N or CR^{2b};
Z is N or CR^{2e};
R¹ is hydrogen, C₁-C₆ alkyl, C₁-C₆ hydroxyalkyl, halo, C₁-C₆ haloalkyl, C₂-C₆ alkoxyalkyl, C₁-C₆ aminylalkyl, optionally substituted cycloalkylalkyl, or optionally substituted heteroaryl;
R^{2a}, R^{2b}, R^{2c}, R^{2d}, and R^{2e} are each, independently, hydrogen, C₁-C₆ alkyl, halo, cyano, C₁-C₆ haloalkyl, —R⁵OR⁶, or —R⁵N(R⁶)₂;
   or R^{2b} and R^{2e}, with the carbons to which they are attached, form a heterocyclyl optionally substituted with one or more alkyl groups;
   or R^{2b} and R^{2e}, with the adjacent carbons to which they are attached, form a heteroaryl that is optionally substituted with one or more alkyl groups;
R³ is, at each occurrence, independently alkyl, halo, —R⁵OR⁶, —R⁵OC(═O)R⁶,
   or —R⁵N(R⁶)₂;
   or two R³'s, when attached to different carbons, join to form —CH₂O—; or two R³'s, with the carbon to which they are both attached, form an optionally substituted heterocyclyl;
L is a direct bond or —C(R⁴)₂—;
R⁴ is, at each occurrence, independently hydrogen or alkyl;
R⁵ is, at each occurrence, independently a direct bond or an alkylene chain optionally substituted with one or more alkyl groups;
R⁶ is, at each occurrence, independently hydrogen, alkyl, or haloalkyl;
n is 0, 1, 2, or 3; and
p is 1, 2, or 3
   provided that:
when one or more occurrences of R³ are fluoro, then at least one of X and Y is N; and
when p is 2 and one occurrence of R³ is —OH, then at least one of X and Y is N.

In some embodiments, R¹ is hydrogen, C₁-C₃ alkyl, C₁-C₃ hydroxyalkyl, halo, C₁-C₂ haloalkyl, C₂-C₆ alkoxyalkyl, C₁-C₄ aminylalkyl, optionally substituted cyclopropylalkyl, or optionally substituted 5-membered heteroaryl. In certain embodiments, R¹ is methyl, ethyl, isopropyl, difluoromethyl, trifluoromethyl, methoxymethyl, ethoxymethyl, fluoro, chloro, bromo, or has one of the following structures:

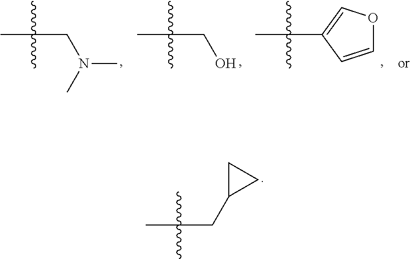

In certain embodiments, R¹ is hydrogen. In some specific embodiments, R¹ is methyl, ethyl, isopropyl, difluoromethyl, trifluoromethyl, methoxymethyl, fluoro, chloro, or bromo. In certain specific embodiments, R¹ is methyl, ethyl, difluoromethyl, methoxymethyl, ethoxymethyl, or chloro. In more specific embodiments, R¹ has one of the following structures:

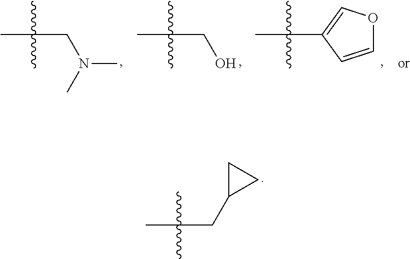

In some embodiments, X is N. In certain embodiments, Y is N. In some specific embodiments, Z is N. In certain specific embodiments, X is CR^{2d}. In some more specific embodiments, Y is CR^{2b}. In certain specific embodiments, Z is CR^{2c}.

In some more specific embodiments, the compound has one of the following structures (Ia), (Ib), (Ic), (Id), (Ie), (If), (Ig), (Ih), (Ii), or (Ij).

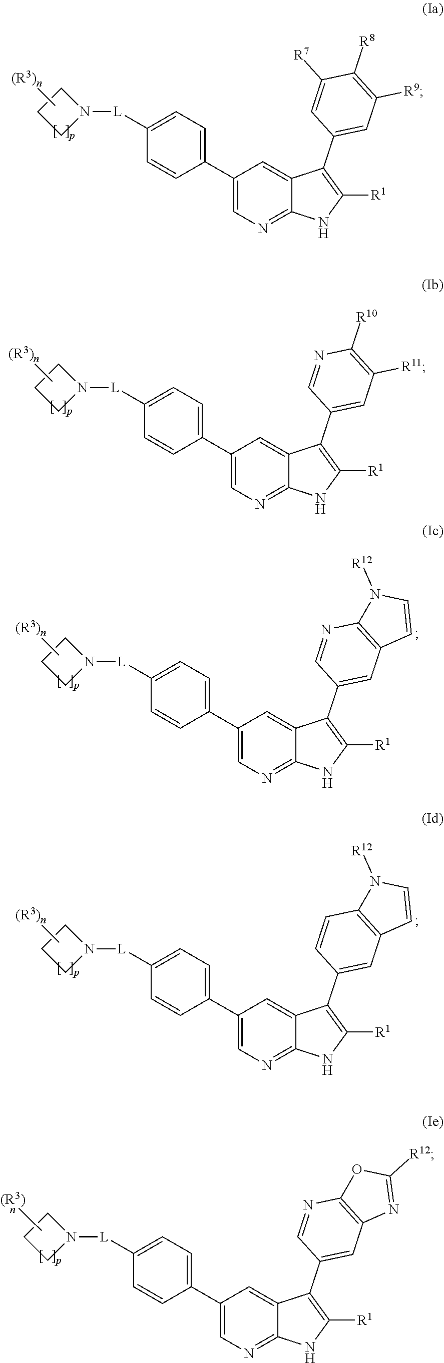

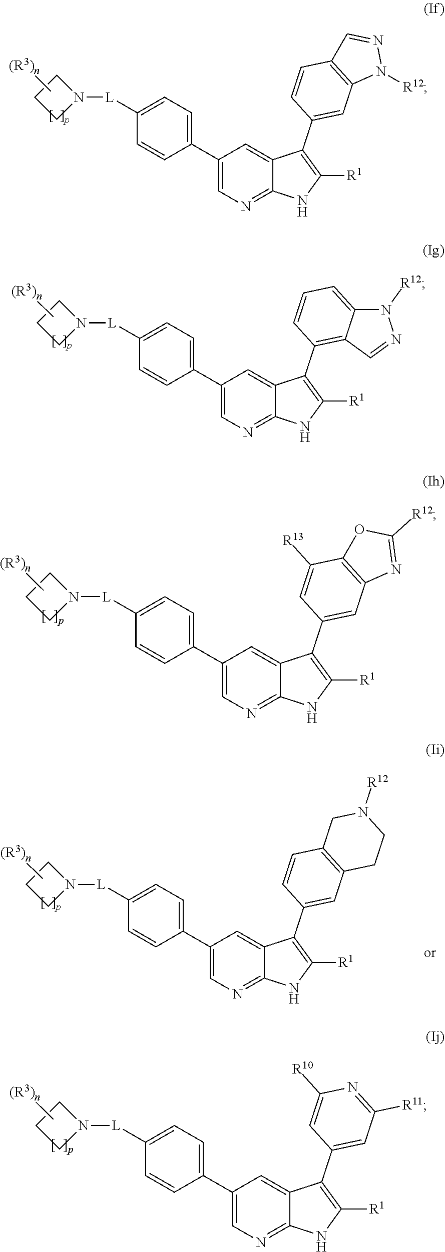

or a pharmaceutically acceptable salt, stereoisomer, tautomer, or prodrug thereof, wherein:
R⁷, R¹, R⁹, R¹⁰, and R¹¹ are each, independently, C₁-C₆ alkyl, halo, cyano, C₁-C₆ haloalkyl, —R⁵OR⁶, or —R⁵N(R⁶)₂;
R¹² is hydrogen or C₁-C₆ alkyl; and
R¹³ is hydrogen or halo.

In some embodiments, at least one of R^{2a}, R^{2b}, R^{2c}, R^{2d}, and R^{2e} is selected from the group consisting of C₁-C₃ alkyl, halo, cyano, C₁-C₂ haloalkyl, —R⁵OR⁶, or —R⁵N(R⁶)₂. In certain embodiments, at least one of R^{2a}, R^{2b}, R^{2c}, R^{2d}, and R^{2e} is selected from the group consisting of methyl, hydroxymethyl, hydroxyisopropyl, hydroxyl, cyano, fluoro, chloro, trifloromethyl, difluoromethyl, methoxy, isopropoxy, and dimethylaminomethyl.

In some more specific embodiments, L is a direct bond. In certain more specific embodiments, L is —C(R⁴)₂—. In some embodiments, L is —CH₂— or —CH(CH₃)—.

In certain embodiments, p is 1. In some other embodiments, p is 2. In still other embodiments, p is 3.

In some embodiments, n is 1, 2, or 3. In some embodiments, n is 0. In some embodiments, n is 1. In some other embodiments, n is 2. In still other embodiments, n is 2. In some embodiments, n is 3.

In some embodiments, at least one occurrence of R³ is methyl, hydroxyl, methoxy, ethoxy, methylamino, dimethylamino, fluoro, methoxymethyl, hydroxymethyl, or —OC(═O)CH₃. In some embodiments, each occurrence of R³ is methyl, hydroxyl, methoxy, ethoxy, methylamino, dimethylamino, fluoro, methoxymethyl, hydroxymethyl, or —OC(═O)CH₃.

In certain more specific embodiments,

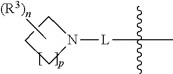

has one of the following structures:

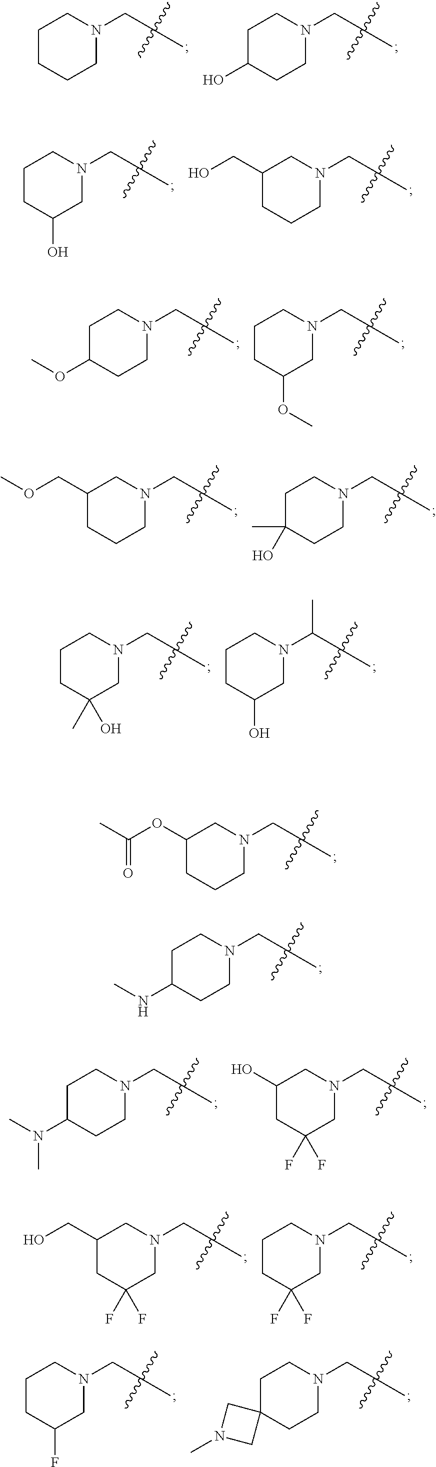

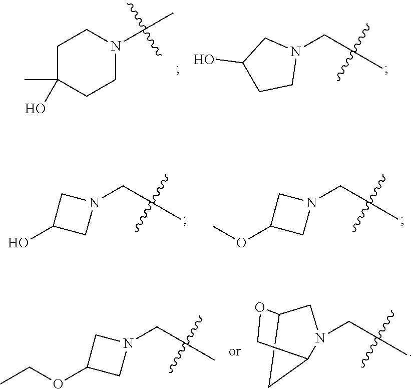

In some specific embodiments,

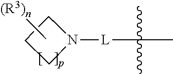

has one of the following structures:

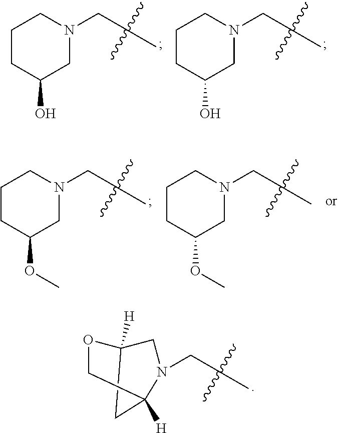

In some embodiments, the compound is an inhibitor of TYRO3.

In various different embodiments, the compound has one of the structures set forth in Table 1 below, or a pharmaceutically acceptable salt, stereoisomer, tautomer, or prodrug thereof. Compounds in Table 1 were prepared as described in the Examples or methods known in the art and analyzed by mass spectrometry and/or ¹H NMR.

TABLE 1
Representative Compounds of Structure (I)

| No. | Structure | Name |
|---|---|---|
| 1 | 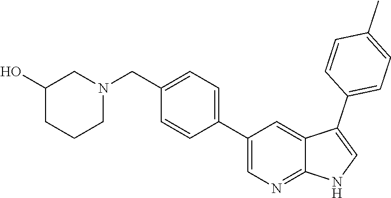 [SMILES: Cc1ccc(-c2c[nH]c3ncc(-c4ccc(CN5CCCC(O)C5)cc4)cc23)cc1] | 1-(4-(3-(p-tolyl)-1H- pyrrolo[2,3-b]pyridin-5- yl)benzyl)piperidin-3-ol |
| 2 | 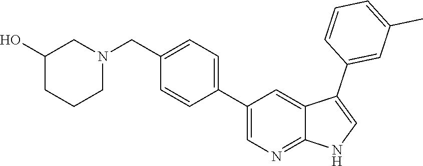 [SMILES: Cc1cccc(-c2c[nH]c3ncc(-c4ccc(CN5CCCC(O)C5)cc4)cc23)c1] | 1-(4-(3-(m-tolyl)-1H- pyrrolo[2,3-b]pyridin-5- yl)benzyl)piperidin-3-ol |
| 3 | 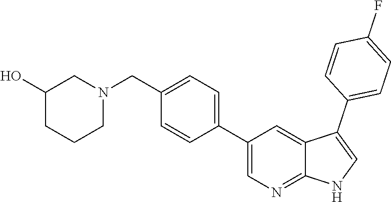 [SMILES: OC1CCCN(Cc2ccc(-c3cnc4[nH]cc(-c5ccc(F)cc5)c4c3)cc2)C1] | 1-(4-(3-(4-fluorophenyl)-1H- pyrrolo[2,3-b]pyridin-5- yl)benzyl)piperidin-3-ol |
| 4 | 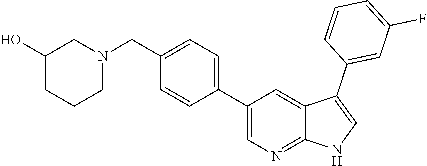 [SMILES: OC1CCCN(Cc2ccc(-c3cnc4[nH]cc(-c5cccc(F)c5)c4c3)cc2)C1] | 1-(4-(3-(3-fluorophenyl)-1H- pyrrolo[2,3-b]pyridin-5- yl)benzyl)piperidin-3-ol |
| 5 | 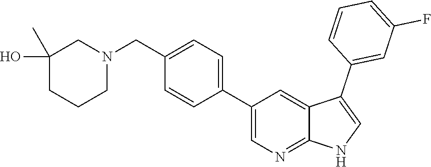 [SMILES: CC1(O)CCCN(Cc2ccc(-c3cnc4[nH]cc(-c5cccc(F)c5)c4c3)cc2)C1] | 1-(4-(3-(3-fluorophenyl)-1H- pyrrolo[2,3-b]pyridin-5- yl)benzyl)-3-methylpiperidin-3- ol |
| 6 | 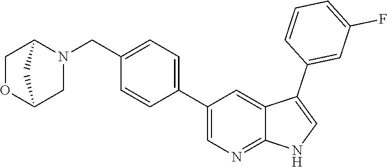 [SMILES: Fc1cccc(-c2c[nH]c3ncc(-c4ccc(CN5C[C@@H]6C[C@H]5CO6)cc4)cc23)c1] | (1S,4S)-5-(4-(3-(3- fluorophenyl)-1H-pyrrolo[2,3- b]pyridin-5-yl)benzyl)-2-oxa-5- azabicyclo[2.2.1]heptane |
| 7 | 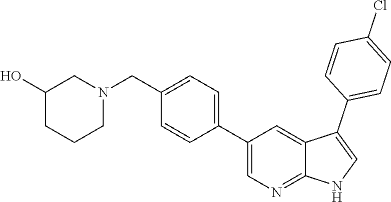 [SMILES: OC1CCCN(Cc2ccc(-c3cnc4[nH]cc(-c5ccc(Cl)cc5)c4c3)cc2)C1] | 1-(4-(3-(4-chlorophenyl)-1H- pyrrolo[2,3-b]pyridin-5- yl)benzyl)piperidin-3-ol |
| 8 | 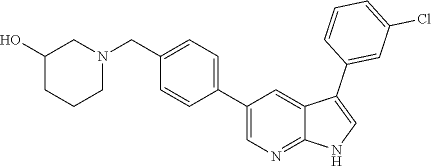 [SMILES: OC1CCCN(Cc2ccc(-c3cnc4[nH]cc(-c5cccc(Cl)c5)c4c3)cc2)C1] | 1-(4-(3-(3-chlorophenyl)-1H- pyrrolo[2,3-b]pyridin-5- yl)benzyl)piperidin-3-ol |
| 9 | 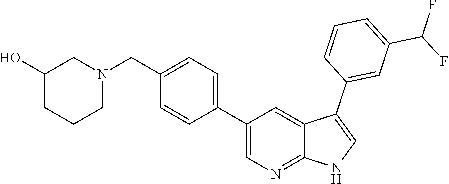 [SMILES: OC1CCCN(Cc2ccc(-c3cnc4[nH]cc(-c5cccc(C(F)F)c5)c4c3)cc2)C1] | 1-(4-(3-(3- (difluoromethyl)phenyl)-1H- pyrrolo[2,3-b]pyridin-5- yl)benzyl)piperidin-3-ol |
| 10 | 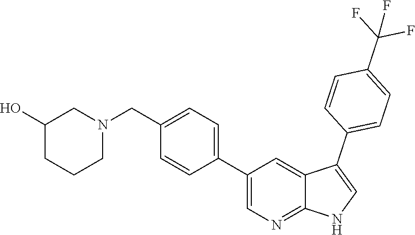 [SMILES: OC1CCCN(Cc2ccc(-c3cnc4[nH]cc(-c5ccc(C(F)(F)F)cc5)c4c3)cc2)C1] | 1-(4-(3-(4- (trifluoromethyl)phenyl)-1H- pyrrolo[2,3-b]pyridin-5- yl)benzyl)piperidin-3-ol |
| 11 | 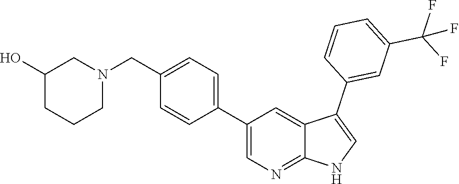 [SMILES: OC1CCCN(Cc2ccc(-c3cnc4[nH]cc(-c5cccc(C(F)(F)F)c5)c4c3)cc2)C1] | 1-(4-(3-(3- (trifluoromethyl)phenyl)-1H- pyrrolo[2,3-b]pyridin-5- yl)benzyl)piperidin-3-ol |
| 12 | 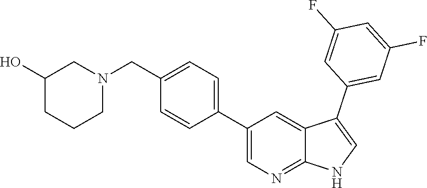 [SMILES: OC1CCCN(Cc2ccc(-c3cnc4[nH]cc(-c5cc(F)cc(F)c5)c4c3)cc2)C1] | 1-(4-(3-(3,5-difluorophenyl)- 1H-pyrrolo[2,3-b]pyridin-5- yl)benzyl)piperidin-3-ol |
| 13 | 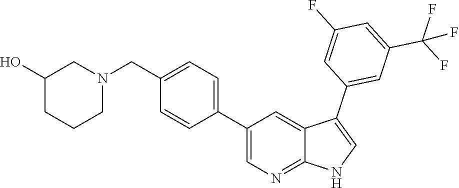 [SMILES: OC1CCCN(Cc2ccc(-c3cnc4[nH]cc(-c5cc(F)cc(C(F)(F)F)c5)c4c3)cc2)C1] | 1-(4-(3-(3-fluoro-5- (trifluoromethyl)phenyl)-1H- pyrrolo[2,3-b]pyridin-5- yl)benzyl)piperidin-3-ol |
| 14 | 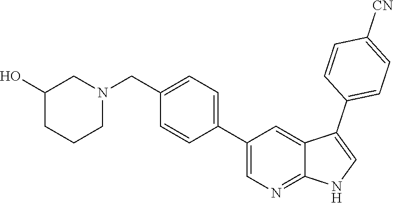 [SMILES: N#Cc1ccc(-c2c[nH]c3ncc(-c4ccc(CN5CCCC(O)C5)cc4)cc23)cc1] | 4-(5-(4-((3-hydroxypiperidin-1- yl)methyl)phenyl)-1H- pyrrolo[2,3-b]pyridin-3- yl)benzonitrile |
| 15 | 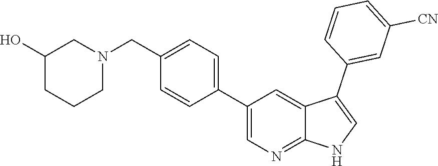 [SMILES: N#Cc1cccc(-c2c[nH]c3ncc(-c4ccc(CN5CCCC(O)C5)cc4)cc23)c1] | 3-(5-(4-((3-hydroxypiperidin-1- yl)methyl)phenyl)-1H- pyrrolo[2,3-b]pyridin-3- yl)benzonitrile |
| 16 | 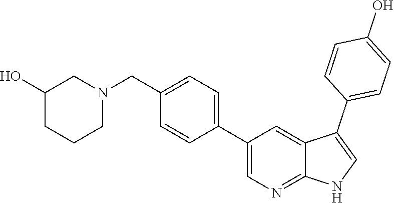 [SMILES: Oc1ccc(-c2c[nH]c3ncc(-c4ccc(CN5CCCC(O)C5)cc4)cc23)cc1] | 1-(4-(3-(4-hydroxyphenyl)-1H- pyrrolo[2,3-b]pyridin-5- yl)benzyl)piperidin-3-ol |
| 17 | 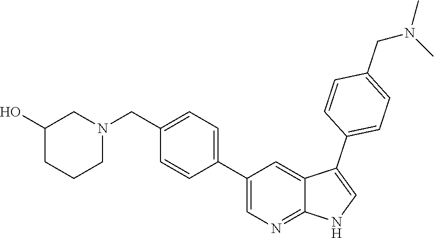 [SMILES: CN(C)Cc1ccc(-c2c[nH]c3ncc(-c4ccc(CN5CCCC(O)C5)cc4)cc23)cc1] | 1-(4-(3-(4- ((dimethylamino)methyl)phenyl)- 1H-pyrrolo[2,3-b]pyridin-5- yl)benzyl)piperidin-3-ol |
| 18 | 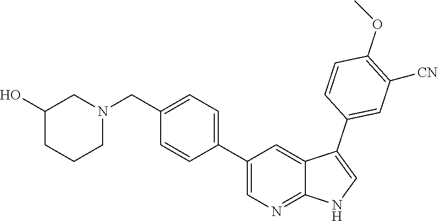 [SMILES: COc1ccc(-c2c[nH]c3ncc(-c4ccc(CN5CCCC(O)C5)cc4)cc23)cc1C#N] | 5-(5-(4-((3-hydroxypiperidin-1- yl)methyl)phenyl)-1H- pyrrolo[2,3-b]pyridin-3-yl)-2- methoxybenzonitrile |
| 19 | 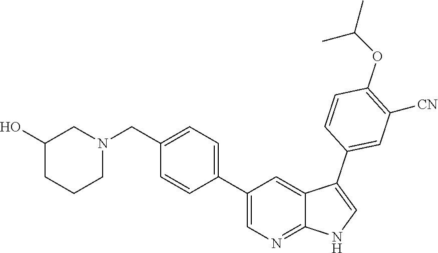 [SMILES: CC(C)Oc1ccc(-c2c[nH]c3ncc(-c4ccc(CN5CCCC(O)C5)cc4)cc23)cc1C#N] | 5-(5-(4-((3-hydroxypiperidin-1- yl)methyl)phenyl)-1H- pyrrolo[2,3-b]pyridin-3-yl)-2- isopropoxybenzonitrile |
| 20 | 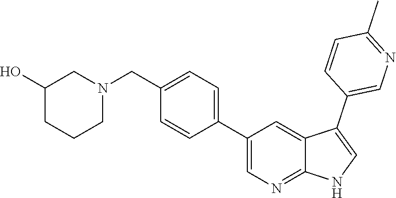 [SMILES: Cc1ccc(-c2c[nH]c3ncc(-c4ccc(CN5CCCC(O)C5)cc4)cc23)cn1] | 1-(4-(3-(6-methylpyridin-3-yl)- 1H-pyrrolo[2,3-b]pyridin-5- yl)benzyl)piperidin-3-ol |
| 21 | 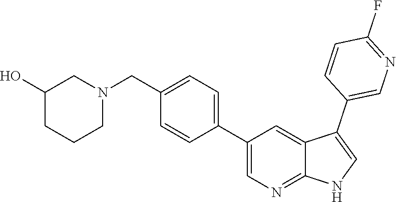 [SMILES: OC1CCCN(Cc2ccc(-c3cnc4[nH]cc(-c5ccc(F)nc5)c4c3)cc2)C1] | 1-(4-(3-(6-fluoropyridin-3-yl)- 1H-pyrrolo[2,3-b]pyridin-5- yl)benzyl)piperidin-3-ol |
| 22 | 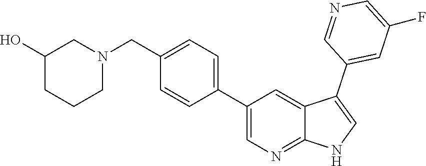 [SMILES: OC1CCCN(Cc2ccc(-c3cnc4[nH]cc(-c5cncc(F)c5)c4c3)cc2)C1] | 1-(4-(3-(5-fluoropyridin-3-yl)- 1H-pyrrolo[2,3-b]pyridin-5- yl)benzyl)piperidin-3-ol |
| 23 | 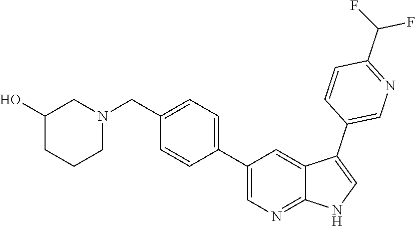 [SMILES: OC1CCCN(Cc2ccc(-c3cnc4[nH]cc(-c5ccc(C(F)F)nc5)c4c3)cc2)C1] | 1-(4-(3-(6- (difluoromethyl)pyridin-3-yl)- 1H-pyrrolo[2,3-b]pyridin-5- yl)benzyl)piperidin-3-ol |
| 24 | 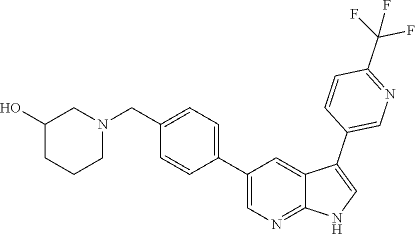 [SMILES: OC1CCCN(Cc2ccc(-c3cnc4[nH]cc(-c5ccc(C(F)(F)F)nc5)c4c3)cc2)C1] | 1-(4-(3-(6- (trifluoromethyl)pyridin-3-yl)- 1H-pyrrolo[2,3-b]pyridin-5- yl)benzyl)piperidin-3-ol |
| 25 | 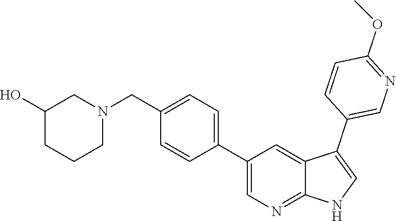 [SMILES: COc1ccc(-c2c[nH]c3ncc(-c4ccc(CN5CCCC(O)C5)cc4)cc23)cn1] | 1-(4-(3-(6-methoxypyridin-3- yl)-1H-pyrrolo[2,3-b]pyridin-5- yl)benzyl)piperidin-3-ol Racemate |
| 26 | 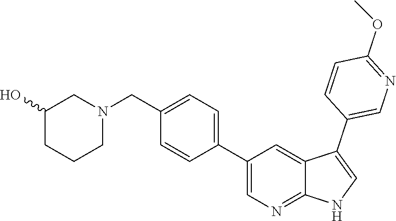 [SMILES: COc1ccc(-c2c[nH]c3ncc(-c4ccc(CN5CCCC(O)C5)cc4)cc23)cn1] | 1-(4-(3-(6-methoxypyridin-3- yl)-1H-pyrrolo[2,3-b]pyridin-5- yl)benzyl)piperidin-3-ol Enantiomer 1 |
| 27 | 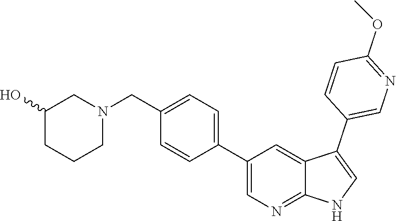 [SMILES: COc1ccc(-c2c[nH]c3ncc(-c4ccc(CN5CCCC(O)C5)cc4)cc23)cn1] | 1-(4-(3-(6-methoxypyridin-3- yl)-1H-pyrrolo[2,3-b]pyridin-5- yl)benzyl)piperidin-3-ol Enantiomer 2 |
| 28 | 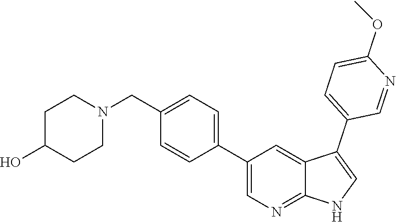 [SMILES: COc1ccc(-c2c[nH]c3ncc(-c4ccc(CN5CCC(O)CC5)cc4)cc23)cn1] | 1-(4-(3-(6-methoxypyridin-3- yl)-1H-pyrrolo[2,3-b]pyridin-5- yl)benzyl)piperidin-4-ol |
| 29 | 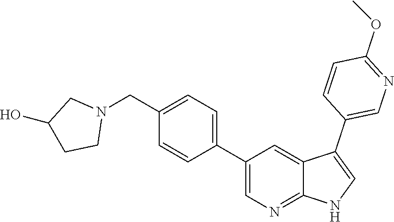 [SMILES: COc1ccc(-c2c[nH]c3ncc(-c4ccc(CN5CCC(O)C5)cc4)cc23)cn1] | 1-(4-(3-(6-methoxypyridin-3- yl)-1H-pyrrolo[2,3-b]pyridin-5- yl)benzyl)pyrrolidin-3-ol |
| 30 | 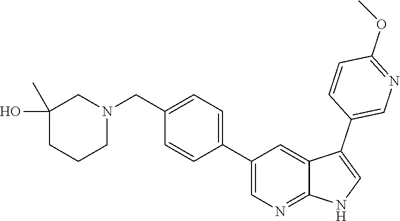 [SMILES: COc1ccc(-c2c[nH]c3ncc(-c4ccc(CN5CCCC(C)(O)C5)cc4)cc23)cn1] | 1-(4-(3-(6-methoxypyridin-3- yl)-1H-pyrrolo[2,3-b]pyridin-5- yl)benzyl)-3-methylpiperidin-3- ol |
| 31 | 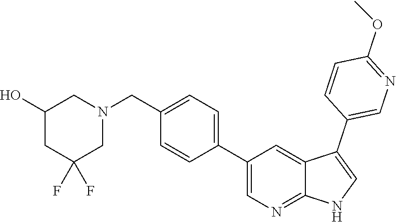 [SMILES: COc1ccc(-c2c[nH]c3ncc(-c4ccc(CN5CC(O)CC(F)(F)C5)cc4)cc23)cn1] | 5,5-difluoro-1-(4-(3-(6- methoxypyridin-3-yl)-1H- pyrrolo[2,3-b]pyridin-5- yl)benzyl)piperidin-3-ol |
| 32 | 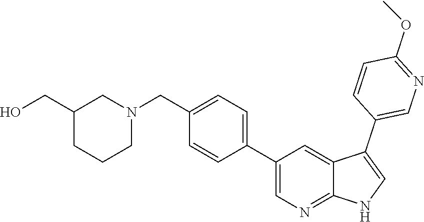 [SMILES: COc1ccc(-c2c[nH]c3ncc(-c4ccc(CN5CCCC(CO)C5)cc4)cc23)cn1] | (1-(4-(3-(6-methoxypyridin-3- yl)-1H-pyrrolo[2,3-b]pyridin-5- yl)benzyl)piperidin-3- yl)methanol |
| 33 | 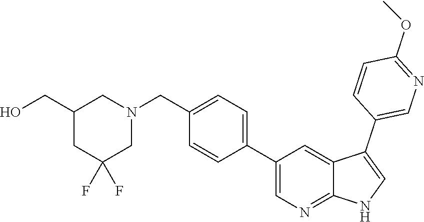 [SMILES: COc1ccc(-c2c[nH]c3ncc(-c4ccc(CN5CC(CO)CC(F)(F)C5)cc4)cc23)cn1] | (5,5-difluoro-1-(4-(3-(6- methoxypyridin-3-yl)-1H- pyrrolo[2,3-b]pyridin-5- yl)benzyl)piperidin-3- yl)methanol |
| 34 | 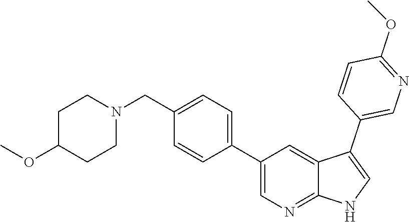 [SMILES: COc1ccc(-c2c[nH]c3ncc(-c4ccc(CN5CCC(OC)CC5)cc4)cc23)cn1] | 5-(4-((4-methoxypiperidin-1- yl)methyl)phenyl)-3-(6- methoxypyridin-3-yl)-1H- pyrrolo[2,3-b]pyridine |
| 35 | 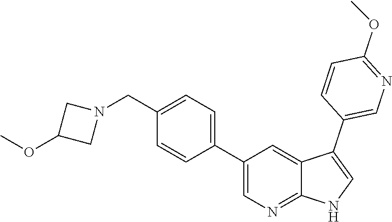 [SMILES: COc1ccc(-c2c[nH]c3ncc(-c4ccc(CN5CC(OC)C5)cc4)cc23)cn1] | 5-(4-((3-methoxyazetidin-1- yl)methyl)phenyl)-3-(6- methoxypyridin-3-yl)-1H- pyrrolo[2,3-b]pyridine |
| 36 | 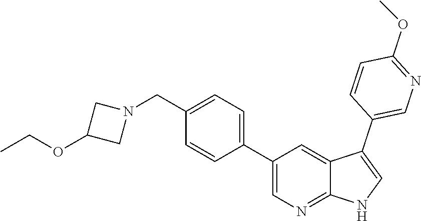 [SMILES: CCOC1CN(Cc2ccc(-c3cnc4[nH]cc(-c5ccc(OC)nc5)c4c3)cc2)C1] | 5-(4-((3-ethoxyazetidin-1- yl)methyl)phenyl)-3-(6- methoxypyridin-3-yl)-1H- pyrrolo[2,3-b]pyridine |
| 37 | 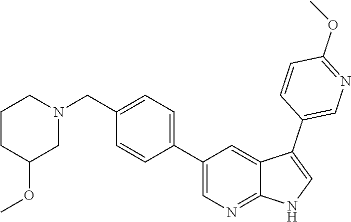 [SMILES: COc1ccc(-c2c[nH]c3ncc(-c4ccc(CN5CCCC(OC)C5)cc4)cc23)cn1] | 5-(4-((3-methoxypiperidin-1- yl)methyl)phenyl)-3-(6- methoxypyridin-3-yl)-1H- pyrrolo[2,3-b]pyridine Racemate |
| 38 | 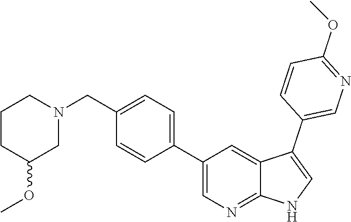 [SMILES: COc1ccc(-c2c[nH]c3ncc(-c4ccc(CN5CCCC(OC)C5)cc4)cc23)cn1] | 5-(4-((3-methoxypiperidin-1- yl)methyl)phenyl)-3-(6- methoxypyridin-3-yl)-1H- pyrrolo[2,3-b]pyridine Enantiomer 1 |
| 39 | 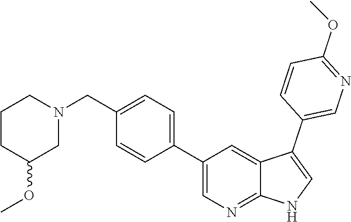 [SMILES: COc1ccc(-c2c[nH]c3ncc(-c4ccc(CN5CCCC(OC)C5)cc4)cc23)cn1] | 5-(4-((3-methoxypiperidin-1- yl)methyl)phenyl)-3-(6- methoxypyridin-3-yl)-1H- pyrrolo[2,3-b]pyridine Enantiomer 2 |
| 40 | 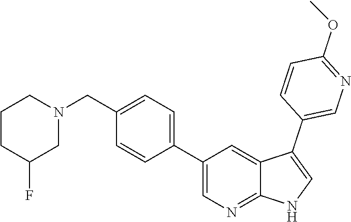 [SMILES: COc1ccc(-c2c[nH]c3ncc(-c4ccc(CN5CCCC(F)C5)cc4)cc23)cn1] | 5-(4-((3-fluoropiperidin-1- yl)methyl)phenyl)-3-(6- methoxypyridin-3-yl)-1H- pyrrolo[2,3-b]pyridine |
| 41 | 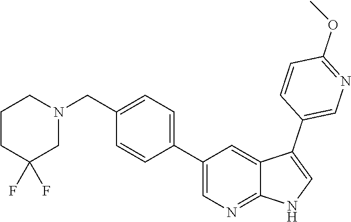 [SMILES: COc1ccc(-c2c[nH]c3ncc(-c4ccc(CN5CCCC(F)(F)C5)cc4)cc23)cn1] | 5-(4-((3,3-difluoropiperidin-1- yl)methyl)phenyl)-3-(6- methoxypyridin-3-yl)-1H- pyrrolo[2,3-b]pyridine |
| 42 | 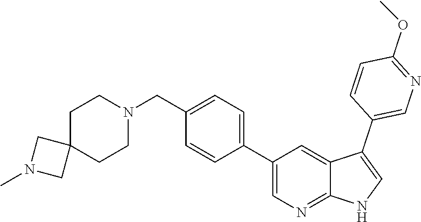 [SMILES: COc1ccc(-c2c[nH]c3ncc(-c4ccc(CN5CCC6(CC5)CN(C)C6)cc4)cc23)cn1] | 7-(4-(3-(6-methoxypyridin-3- yl)-1H-pyrrolo[2,3-b]pyridin-5- yl)benzyl)-2-methyl-2,7- diazaspiro[3.5]nonane |
| 43 | 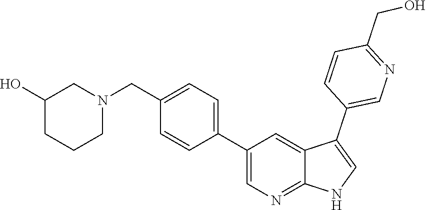 [SMILES: OCc1ccc(-c2c[nH]c3ncc(-c4ccc(CN5CCCC(O)C5)cc4)cc23)cn1] | 1-(4-(3-(6- (hydroxymethyl)pyridin-3-yl)- 1H-pyrrolo[2,3-b]pyridin-5- yl)benzyl)piperidin-3-ol |
| 44 | 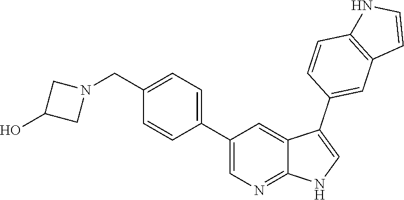 [SMILES: OC1CN(Cc2ccc(-c3cnc4[nH]cc(-c5ccc6[nH]ccc6c5)c4c3)cc2)C1] | 1-(4-(3-(1H-indol-5-yl)-1H- pyrrolo[2,3-b]pyridin-5- yl)benzyl)azetidin-3-ol |
| 45 | 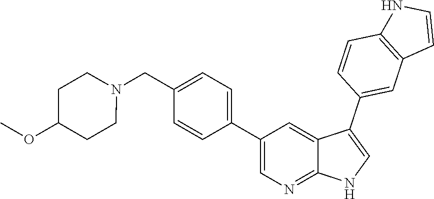 [SMILES: COC1CCN(Cc2ccc(-c3cnc4[nH]cc(-c5ccc6[nH]ccc6c5)c4c3)cc2)CC1] | 3-(1H-indol-5-yl)-5-(4-((4- methoxypiperidin-1- yl)methyl)phenyl)-1H- pyrrolo[2,3-b]pyridine |
| 46 | 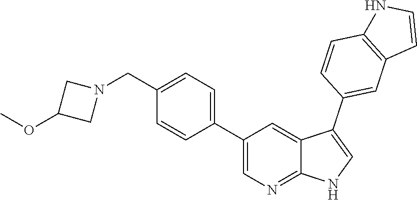 [SMILES: COC1CN(Cc2ccc(-c3cnc4[nH]cc(-c5ccc6[nH]ccc6c5)c4c3)cc2)C1] | 3-(1H-indol-5-yl)-5-(4-((3- methoxyazetidin-1- yl)methyl)phenyl)-1H- pyrrolo[2,3-b]pyridine |
| 47 | 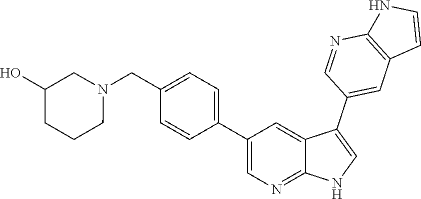 [SMILES: OC1CCCN(Cc2ccc(-c3cnc4[nH]cc(-c5cnc6[nH]ccc6c5)c4c3)cc2)C1] | 1-(4-(1H,1′H-[3,5′- bipyrrolo[2,3-b]pyridin]-5- yl)benzyl)piperidin-3-ol |
| 48 | 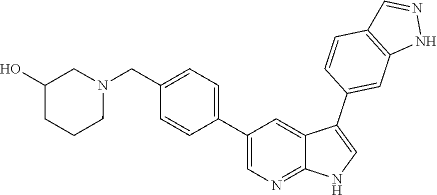 [SMILES: OC1CCCN(Cc2ccc(-c3cnc4[nH]cc(-c5ccc6cn[nH]c6c5)c4c3)cc2)C1] | 1-(4-(3-(1H-indazol-6-yl)-1H- pyrrolo[2,3-b]pyridin-5- yl)benzyl)piperidin-3-ol |
| 49 | 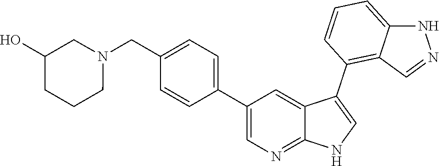 [SMILES: OC1CCCN(Cc2ccc(-c3cnc4[nH]cc(-c5cccc6[nH]ncc56)c4c3)cc2)C1] | 1-(4-(3-(1H-indazol-4-yl)-1H- pyrrolo[2,3-b]pyridin-5- yl)benzyl)piperidin-3-ol |
| 50 | 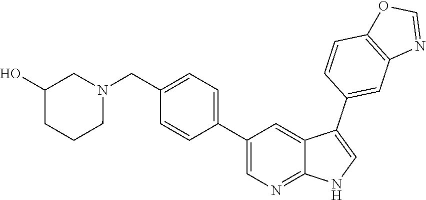 [SMILES: OC1CCCN(Cc2ccc(-c3cnc4[nH]cc(-c5ccc6ocnc6c5)c4c3)cc2)C1] | 1-(4-(3-(benzo[d]oxazol-5-yl)- 1H-pyrrolo[2,3-b]pyridin-5- yl)benzyl)piperidin-3-ol |
| 51 | 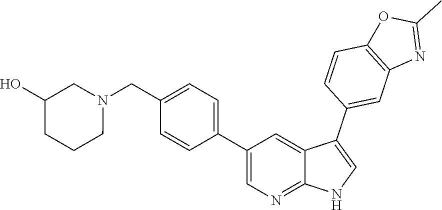 [SMILES: Cc1nc2cc(-c3c[nH]c4ncc(-c5ccc(CN6CCCC(O)C6)cc5)cc34)ccc2o1] | 1-(4-(3-(2- methylbenzo[d]oxazol-5-yl)- 1H-pyrrolo[2,3-b]pyridin-5- yl)benzyl)piperidin-3-ol |
| 52 | 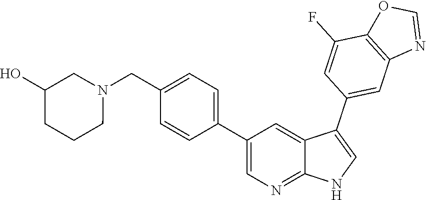 [SMILES: OC1CCCN(Cc2ccc(-c3cnc4[nH]cc(-c5cc(F)c6ocnc6c5)c4c3)cc2)C1] | 1-(4-(3-(7- fluorobenzo[d]oxazol-5-yl)-1H- pyrrolo[2,3-b]pyridin-5- yl)benzyl)piperidin-3-ol |
| 53 | 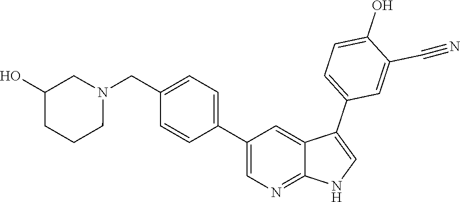 [SMILES: N#Cc1cc(-c2c[nH]c3ncc(-c4ccc(CN5CCCC(O)C5)cc4)cc23)ccc1O] | 2-hydroxy-5-(5-(4-((3- hydroxypiperidin-1- yl)methyl)phenyl)-1H- pyrrolo[2,3-b]pyridin-3- yl)benzonitrile |
| 54 | 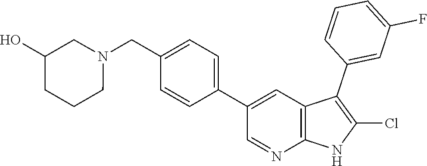 [SMILES: OC1CCCN(Cc2ccc(-c3cnc4[nH]c(Cl)c(-c5cccc(F)c5)c4c3)cc2)C1] | 1-(4-(2-chloro-3-(3- fluorophenyl)-1H-pyrrolo[2,3- b]pyridin-5-yl)benzyl)piperidin- 3-ol |
| 55 | 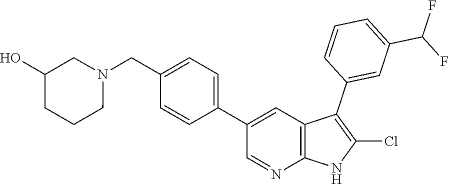 [SMILES: OC1CCCN(Cc2ccc(-c3cnc4[nH]c(Cl)c(-c5cccc(C(F)F)c5)c4c3)cc2)C1] | 1-(4-(2-chloro-3-(3- (difluoromethyl)phenyl)-1H- pyrrolo[2,3-b]pyridin-5- yl)benzyl)piperidin-3-ol |
| 56 | 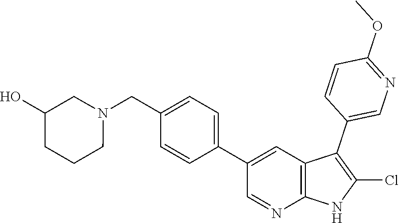 [SMILES: COc1ccc(-c2c(Cl)[nH]c3ncc(-c4ccc(CN5CCCC(O)C5)cc4)cc23)cn1] | 1-(4-(2-chloro-3-(6- methoxypyridin-3-yl)-1H- pyrrolo[2,3-b]pyridin-5- yl)benzyl)piperidin-3-ol |
| 57 | 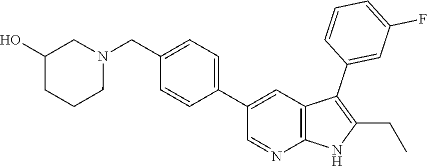 [SMILES: CCc1[nH]c2ncc(-c3ccc(CN4CCCC(O)C4)cc3)cc2c1-c1cccc(F)c1] | 1-(4-(2-ethyl-3-(3- fluorophenyl)-1H-pyrrolo[2,3- b]pyridin-5-yl)benzyl)piperidin- 3-ol |
| 58 | 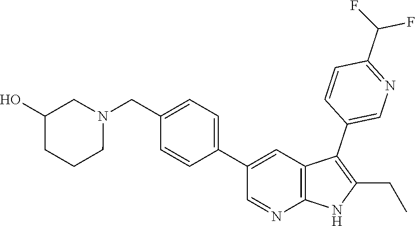 [SMILES: CCc1[nH]c2ncc(-c3ccc(CN4CCCC(O)C4)cc3)cc2c1-c1ccc(C(F)F)nc1] | 1-(4-(3-(6- (difluoromethyl)pyridin-3-yl)-2- ethyl-1H-pyrrolo[2,3-b]pyridin- 5-yl)benzyl)piperidin-3-ol |
| 59 | 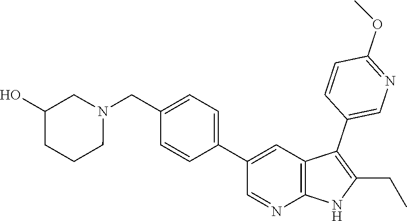 [SMILES: CCc1[nH]c2ncc(-c3ccc(CN4CCCC(O)C4)cc3)cc2c1-c1ccc(OC)nc1] | 1-(4-(2-ethyl-3-(6- methoxypyridin-3-yl)-1H- pyrrolo[2,3-b]pyridin-5- yl)benzyl)piperidin-3-ol |
| 60 | 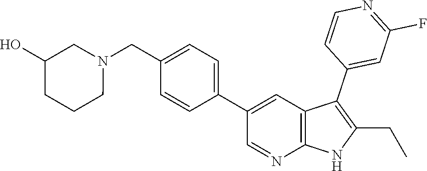 [SMILES: CCc1[nH]c2ncc(-c3ccc(CN4CCCC(O)C4)cc3)cc2c1-c1ccnc(F)c1] | 1-(4-(2-ethyl-3-(2- fluoropyridin-4-yl)-1H- pyrrolo[2,3-b]pyridin-5- yl)benzyl)piperidin-3-ol |
| 61 | 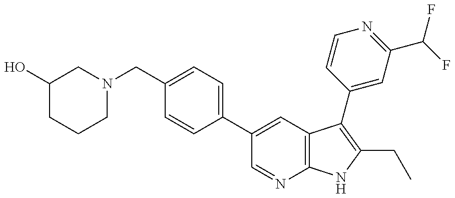 [SMILES: CCc1[nH]c2ncc(-c3ccc(CN4CCCC(O)C4)cc3)cc2c1-c1ccnc(C(F)F)c1] | 1-(4-(3-(2- (difluoromethyl)pyridin-4-yl)-2- ethyl-1H-pyrrolo[2,3-b]pyridin- 5-yl)benzyl)piperidin-3-ol |
| 62 | 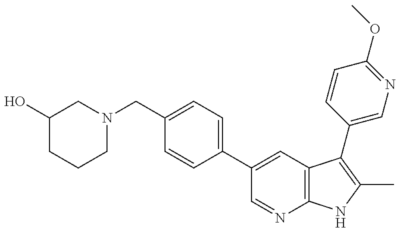 [SMILES: COc1ccc(-c2c(C)[nH]c3ncc(-c4ccc(CN5CCCC(O)C5)cc4)cc23)cn1] | 1-(4-(3-(6-methoxypyridin-3- yl)-2-methyl-1H-pyrrolo[2,3- b]pyridin-5-yl)benzyl)piperidin- 3-ol |
| 63 | 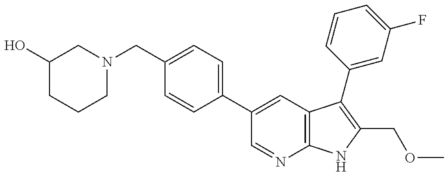 [SMILES: COCc1[nH]c2ncc(-c3ccc(CN4CCCC(O)C4)cc3)cc2c1-c1cccc(F)c1] | 1-(4-(3-(3-fluorophenyl)-2- (methoxymethyl)-1H- pyrrolo[2,3-b]pyridin-5- yl)benzyl)piperidin-3-ol |
| 64 | 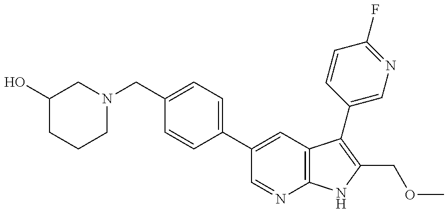 [SMILES: COCc1[nH]c2ncc(-c3ccc(CN4CCCC(O)C4)cc3)cc2c1-c1ccc(F)nc1] | 1-(4-(3-(6-fluoropyridin-3-yl)-2- (methoxymethyl)-1H- pyrrolo[2,3-b]pyridin-5- yl)benzyl)piperidin-3-ol |
| 65 | 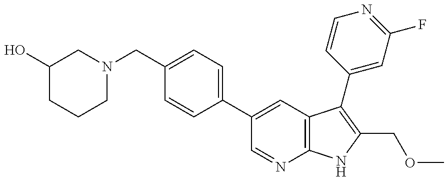 [SMILES: COCc1[nH]c2ncc(-c3ccc(CN4CCCC(O)C4)cc3)cc2c1-c1ccnc(F)c1] | 1-(4-(3-(2-fluoropyridin-4-yl)-2- (methoxymethyl)-1H- pyrrolo[2,3-b]pyridin-5- yl)benzyl)piperidin-3-ol |
| 66 | 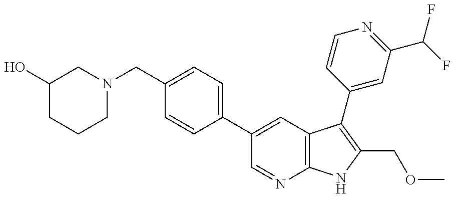 [SMILES: COCc1[nH]c2ncc(-c3ccc(CN4CCCC(O)C4)cc3)cc2c1-c1ccnc(C(F)F)c1] | 1-(4-(3-(2- (difluoromethyl)pyridin-4-yl)-2- (methoxymethyl)-1H- pyrrolo[2,3-b]pyridin-5- yl)benzyl)piperidin-3-ol |
| 67 | 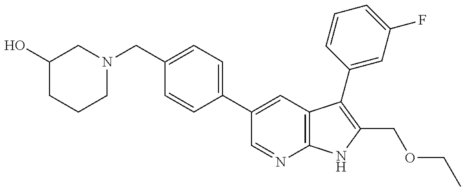 [SMILES: CCOCc1[nH]c2ncc(-c3ccc(CN4CCCC(O)C4)cc3)cc2c1-c1cccc(F)c1] | 1-(4-(2-(ethoxymethyl)-3-(3- fluorophenyl)-1H-pyrrolo[2,3- b]pyridin-5-yl)benzyl)piperidin- 3-ol |
| 68 | 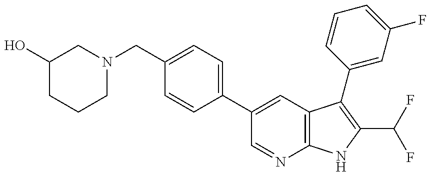 [SMILES: OC1CCCN(Cc2ccc(-c3cnc4[nH]c(C(F)F)c(-c5cccc(F)c5)c4c3)cc2)C1] | 1-(4-(2-(difluoromethyl)-3-(3- fluorophenyl)-1H-pyrrolo[2,3- b]pyridin-5-yl)benzyl)piperidin- 3-ol |
| 69 | 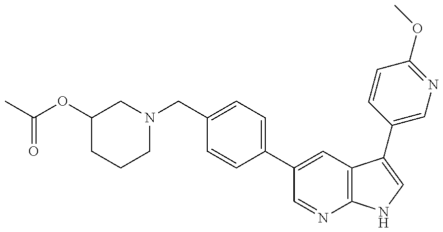 [SMILES: COc1ccc(-c2c[nH]c3ncc(-c4ccc(CN5CCCC(OC(C)=O)C5)cc4)cc23)cn1] | 1-(4-(3-(6-methoxypyridin-3- yl)-1H-pyrrolo[2,3-b]pyridin-5- yl)benzyl)piperidin-3-yl acetate |
| 70 | 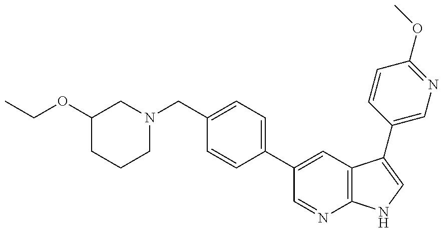 [SMILES: CCOC1CCCN(Cc2ccc(-c3cnc4[nH]cc(-c5ccc(OC)nc5)c4c3)cc2)C1] | 5-(4-((3- (methoxymethyl)piperidin-1- yl)methyl)phenyl)-3-(6- methoxypyridin-3-yl)-1H- pyrrolo[2,3-b]pyridine |
| 71 | 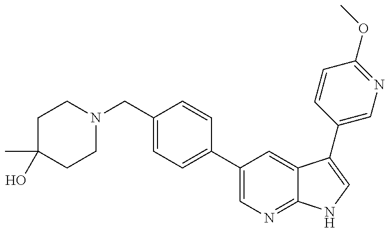 [SMILES: COc1ccc(-c2c[nH]c3ncc(-c4ccc(CN5CCC(C)(O)CC5)cc4)cc23)cn1] | 1-(4-(3-(6-methoxypyridin-3- yl)-1H-pyrrolo[2,3-b]pyridin-5- yl)benzyl)-4-methylpiperidin-4- ol |
| 72 | 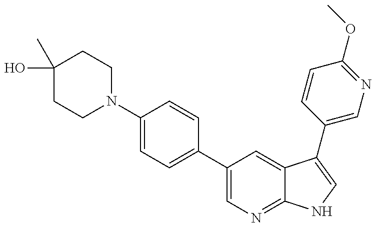 [SMILES: COc1ccc(-c2c[nH]c3ncc(-c4ccc(N5CCC(C)(O)CC5)cc4)cc23)cn1] | 1-(4-(3-(6-methoxypyridin-3- yl)-1H-pyrrolo[2,3-b]pyridin-5- yl)phenyl)-4-methylpiperidin-4- ol |
| 73 | 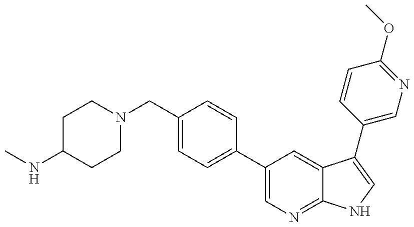 [SMILES: CNC1CCN(Cc2ccc(-c3cnc4[nH]cc(-c5ccc(OC)nc5)c4c3)cc2)CC1] | 1-(4-(3-(6-methoxypyridin-3- yl)-1H-pyrrolo[2,3-b]pyridin-5- yl)phenyl)-N-methylpiperidin-4- amine |
| 74 | 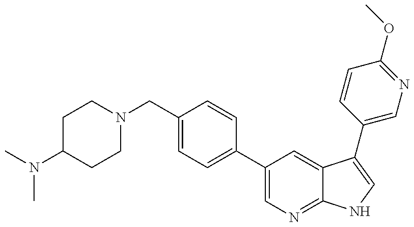 [SMILES: COc1ccc(-c2c[nH]c3ncc(-c4ccc(CN5CCC(N(C)C)CC5)cc4)cc23)cn1] | 1-(4-(3-(6-methoxypyridin-3- yl)-1H-pyrrolo[2,3-b]pyridin-5- yl)benzyl)-N,N- dimethylpiperidin-4-amine |
| 75 | 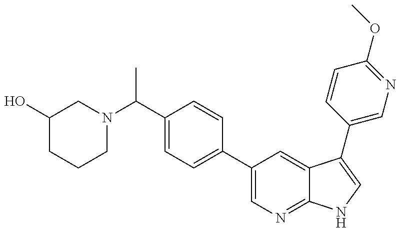 [SMILES: COc1ccc(-c2c[nH]c3ncc(-c4ccc(C(C)N5CCCC(O)C5)cc4)cc23)cn1] | 1-(1-(4-(3-(6-methoxypyridin-3- yl)-1H-pyrrolo[2,3-b]pyridin-5- yl)phenyl)ethyl)piperidin-3-ol |
| 76 | 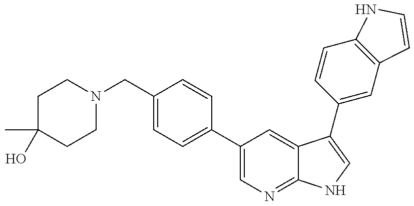 [SMILES: CC1(O)CCN(Cc2ccc(-c3cnc4[nH]cc(-c5ccc6[nH]ccc6c5)c4c3)cc2)CC1] | 1-(4-(3-(1H-indol-5-yl)-1H- pyrrolo[2,3-b]pyridin-5- yl)benzyl)-4-methylpiperidin-4- ol |
| 77 | 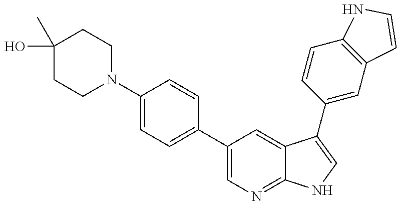 [SMILES: CC1(O)CCN(c2ccc(-c3cnc4[nH]cc(-c5ccc6[nH]ccc6c5)c4c3)cc2)CC1] | 1-(4-(3-(1H-indol-5-yl)-1H- pyrrolo[2,3-b]pyridin-5- yl)phenyl)-4-methylpiperidin-4- ol |
| 78 | 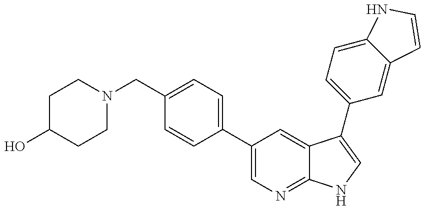 [SMILES: OC1CCN(Cc2ccc(-c3cnc4[nH]cc(-c5ccc6[nH]ccc6c5)c4c3)cc2)CC1] | 1-(4-(3-(1H-indol-5-yl)-1H- pyrrolo[2,3-b]pyridin-5- yl)benzyl)piperidin-4-ol |
| 79 | 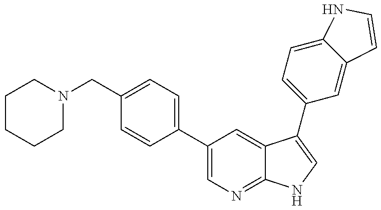 [SMILES: c1cc2cc(-c3c[nH]c4ncc(-c5ccc(CN6CCCCC6)cc5)cc34)ccc2[nH]1] | 3-(1H-indol-5-yl)-5-(4- (piperidin-1-ylmethyl)phenyl)- 1H-pyrrolo[2,3-b]pyridine |
| 80 | 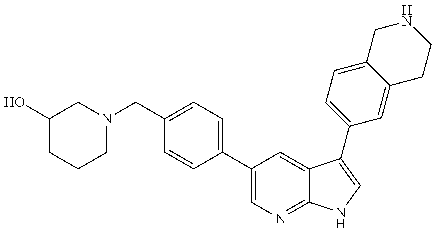 [SMILES: OC1CCCN(Cc2ccc(-c3cnc4[nH]cc(-c5ccc6c(c5)CCNC6)c4c3)cc2)C1] | 1-(4-(3-(1,2,3,4- tetrahydroisoquinolin-6-yl)-1H- pyrrolo[2,3-b]pyridin-5- yl)benzyl)piperidin-3-ol |
| 81 | 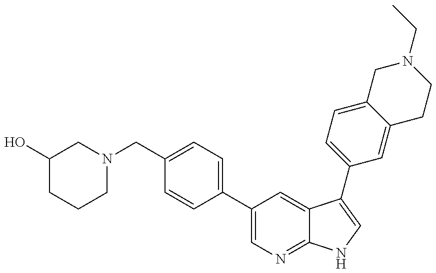 [SMILES: CCN1CCc2cc(-c3c[nH]c4ncc(-c5ccc(CN6CCCC(O)C6)cc5)cc34)ccc2C1] | 1-(4-(3-(2-ethyl-1,2,3,4- tetrahydroisoquinolin-6-yl)-1H- pyrrolo[2,3-b]pyridin-5- yl)benzyl)piperidin-3-ol |
| 82 | 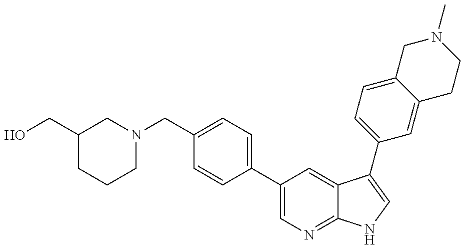 [SMILES: CN1CCc2cc(-c3c[nH]c4ncc(-c5ccc(CN6CCCC(CO)C6)cc5)cc34)ccc2C1] | (1-(4-(3-(2-methyl-1,2,3,4- tetrahydroisoquinolin-6-yl)-1H- pyrrolo[2,3-b]pyridin-5- yl)benzyl)piperidin-3- yl)methanol |
| 83 | 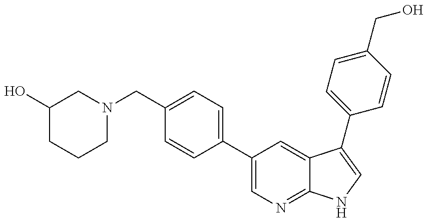 [SMILES: OCc1ccc(-c2c[nH]c3ncc(-c4ccc(CN5CCCC(O)C5)cc4)cc23)cc1] | 1-(4-(3-(4- (hydroxymethyl)phenyl)-1H- pyrrolo[2,3-b]pyridin-5- yl)benzyl)piperidin-3-ol |
| 84 | 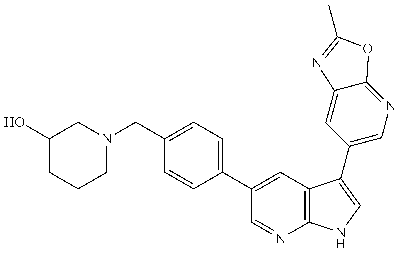 [SMILES: Cc1nc2cc(-c3c[nH]c4ncc(-c5ccc(CN6CCCC(O)C6)cc5)cc34)cnc2o1] | 1-(4-(3-(2-methyloxazolo[5,4- b]pyridin-6-yl)-1H-pyrrolo[2,3- b]pyridin-5-yl)benzyl)piperidin- 3-ol |
| 85 | 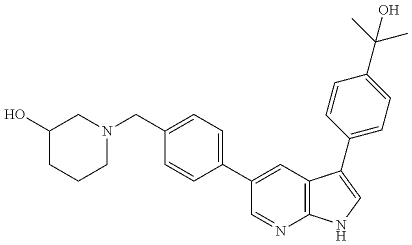 [SMILES: CC(C)(O)c1ccc(-c2c[nH]c3ncc(-c4ccc(CN5CCCC(O)C5)cc4)cc23)cc1] | 1-(4-(3-(4-(2-hydroxypropan-2- yl)phenyl)-1H-pyrrolo[2,3- b]pyridin-5-yl)benzyl)piperidin- 3-ol |
| 86 | 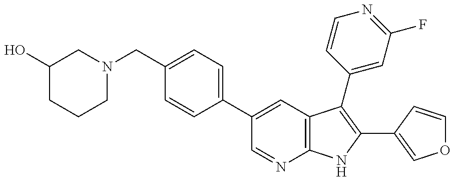 [SMILES: OC1CCCN(Cc2ccc(-c3cnc4[nH]c(-c5ccoc5)c(-c5ccnc(F)c5)c4c3)cc2)C1] | 1-(4-(3-(2-fluoropyridin-4-yl)-2- (furan-3-yl)-1H-pyrrolo[2,3- b]pyridin-5-yl)benzyl)piperidin- 3-ol |
| 87 | 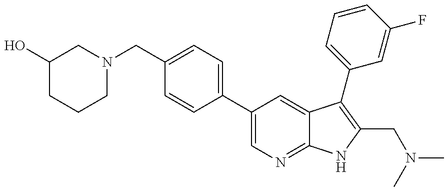 [SMILES: CN(C)Cc1[nH]c2ncc(-c3ccc(CN4CCCC(O)C4)cc3)cc2c1-c1cccc(F)c1] | 1-(4-(2- ((dimethylamino)methyl)-3-(3- fluorophenyl)-1H-pyrrolo[2,3- b]pyridin-5-yl)benzyl)piperidin- 3-ol |
| 88 | 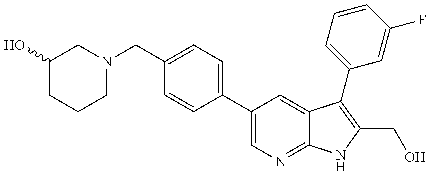 [SMILES: OCc1[nH]c2ncc(-c3ccc(CN4CCCC(O)C4)cc3)cc2c1-c1cccc(F)c1] | 1-(4-(3-(3-fluorophenyl)-2- (hydroxymethyl)-1H- pyrrolo[2,3-b]pyridin-5- yl)benzyl)piperidin-3-ol Enantiomer 1 |
| 89 | 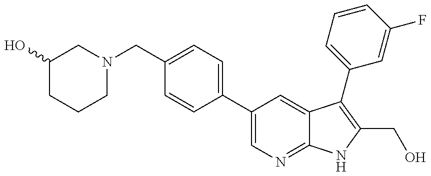 [SMILES: OCc1[nH]c2ncc(-c3ccc(CN4CCCC(O)C4)cc3)cc2c1-c1cccc(F)c1] | 1-(4-(3-(3-fluorophenyl)-2- (hydroxymethyl)-1H- pyrrolo[2,3-b]pyridin-5- yl)benzyl)piperidin-3-ol Enantiomer 2 |
| 90 | 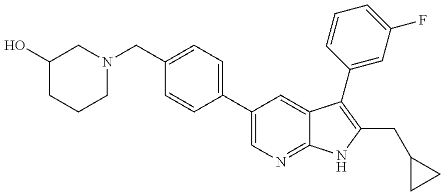 [SMILES: OC1CCCN(Cc2ccc(-c3cnc4[nH]c(CC5CC5)c(-c5cccc(F)c5)c4c3)cc2)C1] | 1-(4-(2-(cyclopropylmethyl)-3- (3-fluorophenyl)-1H- pyrrolo[2,3-b]pyridin-5- yl)benzyl)piperidin-3-ol |

It is understood that in the present description, combinations of substituents and/or variables of the depicted formulae are permissible only if such contributions result in stable compounds.

In an additional embodiment, various compounds of the disclosure which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

Methods for producing the compounds described herein is provided below. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described herein.

The following General Reaction Schemes illustrate examples of the preparation of compounds of Structure (I):

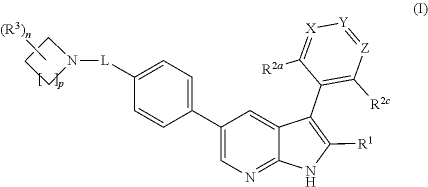

or pharmaceutically acceptable salts, stereoisomers, tautomers, or prodrug thereof, wherein each of L, X, Y, Z, R¹, R^{2a}, R^{2c}, R³, p, and n are as defined herein. Additionally, as it will be appreciated by those skilled in the art, the methods described herein can be used to synthesize compounds of Structure (II).

### GENERAL REACTION SCHEME 1

The following General Reaction Scheme, wherein RP is a protecting group (e.g., Tosylate, Boc, Fmoc, etc.) and X, Y, Z, R¹, R^{2a}, R^{2b}, R³, L, and n have the meanings described herein, illustrates examples of methods of making a subset of compounds of Structure (I).

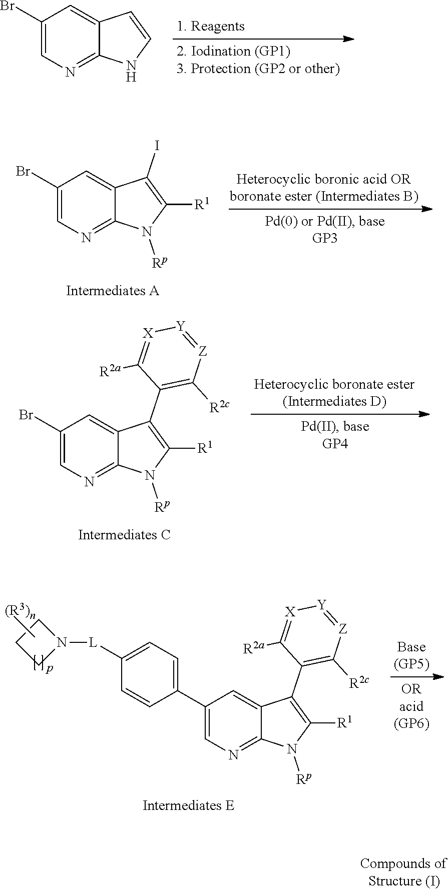

As shown in General Reaction Scheme 1, optional functionalization at the 2-position of 5-bromo-7-azaindole followed by iodination at the 3-position and protection of the free NH affords Intermediates A which can be subjected to palladium-catalyzed (hetero)arylation at the 3-position with boronic acid or boronate ester Intermediates B to form Intermediates C. Subsequent palladium-catalyzed (hetero)arylation at the 5-position with boronate ester Intermediates D produces Intermediates E which can be converted to compounds of Structure (I) after removal of the protecting group (e.g., using General Procedure GP5 or General Procedure GP6).

GP1 in Reaction Scheme 1 refers to General Procedure GP1 detailed herein below. GP2 refers to General Procedure GP2, and so on. Additionally, Intermediates A-E are described in more detail herein below as well.

Any of the above reaction scheme can be modified at any step to add and/or modify a substituent may be added or modified as appropriate during any stage of the overall synthesis of desired compounds.

It will also be appreciated by those skilled in the art that in the processes for preparing the compounds described herein the functional groups of intermediate compounds may need to be protected by suitable protecting groups. Such functional groups include, but are not limited to, hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl, or arylalkyl esters. Protecting groups are optionally added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin, or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs.” Prodrugs of compounds of this disclosure are included within the scope of embodiments of the disclosure.

### Pharmaceutical Compositions

Other embodiments are directed to pharmaceutical compositions. The pharmaceutical composition comprises any one (or more) of the foregoing compounds and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound as disclosed herein and an additional therapeutic agent (e.g., anticancer agent). Non-limiting examples of such therapeutic agents are described herein below.

Suitable routes of administration include, but are not limited to, oral, intravenous, rectal, aerosol, parenteral, ophthalmic, pulmonary, transmucosal, transdermal, vaginal, optic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the compound is delivered in a targeted drug delivery system, for example, in a liposome coated with and organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In treatment methods according to embodiments of the disclosure, an effective amount of at least one compound of Structure (I) or (II) is administered to a subject suffering from or diagnosed as having such a disease, disorder, or medical condition. Effective amounts or doses may be ascertained by methods such as modeling, dose escalation studies or clinical trials, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

The compounds according to the disclosure are effective over a wide dosage range. For example, in the treatment of adult humans, dosages from 10 to 5000 mg, from 100 to 5000 mg, from 1000 mg to 4000 mg per day, and from 1000 to 3000 mg per day are examples of dosages that are used in some embodiments. The exact dosage will depend upon the route of administration, the form in which the compound is administered, the subject to be treated, the body weight of the subject to be treated, and the preference and experience of the attending physician.

In some embodiments, compounds of the disclosure are administered in a single dose. Typically, such administration will be by injection, e.g., intravenous injection, in order to introduce the agent quickly. However, other routes are used as appropriate. A single dose of a compound of the disclosure may also be used for treatment of an acute condition.

In some embodiments, compounds of the disclosure are administered in multiple doses. In some embodiments, dosing is about once, twice, three times, four times, five times, six times, or more than six times per day. In other embodiments, dosing is about once a month, once every two weeks, once a week, or once every other day. In another embodiment compounds of the disclosure and another agent (e.g., anti-cancer agent) are administered together about once per day to about 6 times per day. In another embodiment the administration of compounds of the disclosure and an agent continues for less than about 7 days. In yet another embodiment the administration continues for more than about 6, 10, 14, 28 days, two months, six months, or one year. In some cases, continuous dosing is achieved and maintained as long as necessary.

Administration of compounds of the disclosure may continue as long as necessary. In some embodiments, compounds of the disclosure are administered for more than 1, 2, 3, 4, 5, 6, 7, 14, or 28 days. In some embodiments, compounds of the disclosure are administered for less than 28, 14, 7, 6, 5, 4, 3, 2, or 1 day. In some embodiments, compounds of the disclosure are administered chronically on an ongoing basis, e.g., for the treatment of chronic effects.

In some embodiments, the compounds of the disclosure are administered in individual dosage forms. It is known in the art that due to intersubject variability in compound pharmacokinetics, individualization of dosing regimen is necessary for optimal therapy.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the disclosed compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising one or more compounds of Structure (I) or (II), and a pharmaceutically acceptable carrier.

Provided herein are pharmaceutical compositions comprising one or more compounds selected from compounds of Structure (I) or (II) and pharmaceutically acceptable diluent(s), excipient(s), and carrier(s). In certain embodiments, the compounds described are administered as pharmaceutical compositions in which one or more compounds selected from compounds of Structure (I) or (II) are mixed with other active ingredients, as in combination therapy. Encompassed herein are all combinations of actives set forth in the combination therapies section below and throughout this disclosure. In specific embodiments, the pharmaceutical compositions include one or more compounds of Structure (I) or (II).

A pharmaceutical composition, as used herein, refers to a mixture of one or more compounds selected from compounds of Structure (I) or (II) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, therapeutically effective amounts of one or more compounds selected from compounds of Structure (I) or (II) provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more compounds selected from compounds of Structure (I) or (II) are formulated in aqueous solutions. In specific embodiments, the aqueous solution is selected from, by way of example only, a physiologically compatible buffer, such as Hank's solution, Ringer's solution, or physiological saline buffer. In other embodiments, one or more compounds selected from compounds of Structure (I) or (II) are formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of one or more compounds selected from compounds of Structure (I) or (II) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

Pharmaceutical compositions include at least one pharmaceutically acceptable carrier, diluent or excipient, and one or more compounds selected from compounds of Structure (I) or (II), described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical compositions comprising one or more compounds selected from compounds of Structure (I) or (II) illustratively takes the form of a liquid where the agents are present in solution, in suspension or both. Typically when the composition is administered as a suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, aqueous suspensions contain one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of one or more compounds selected from compounds of Structure (I) or (II). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Compositions may include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In some embodiments, the concentration of one or more compounds selected from compounds of Structure (I) or (II) provided in the pharmaceutical compositions of the present disclosure is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds selected from compounds of Structure (I) or (II) provided in the pharmaceutical compositions of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the amount the one or more compounds selected from compounds of Structure (I) or (II) provided in the pharmaceutical compositions of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the one or more compounds selected from compounds of Structure (I) or (II) provided in the pharmaceutical compositions of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

Packaging materials for use in packaging pharmaceutical compositions described herein include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

### Methods

Embodiments of the present disclosure are useful as modulators of TYRO3 in a host species. Therefore, the compounds of Structure (I) and (II) are also useful in the treatment of conditions mediated TYRO3.

Accordingly, one embodiment provides a method of treating a disease or disorder, comprising administering a therapeutically effective amount of a compound of any one of claims x or a pharmaceutically acceptable salt, stereoisomer, tautomer, or prodrug thereof, or the pharmaceutical composition of claim x, to a subject in need thereof.

In more specific embodiments, the disease or disorder is a TYRO3-mediated disease or disorder. In some specific embodiments, the disease or disorder is cancer. In more specific embodiments, the disease or disorder comprises a solid tumor. In some embodiments, the disease or disorder is leukemia, lymphoma, brain cancer, genitourinary tract cancer, endocrine system cancer, gastrointestinal tract cancer, colon cancer, rectal cancer, breast cancer, kidney cancer, lymphatic system cancer, stomach cancer, lung cancer, pancreatic cancer, skin cancer, or combinations thereof. In more specific embodiments, the disease or disorder is bladder tumor, diffuse large B-Cell lymphoma, adenoid cystic carcinoma of salivary gland, Burkitt lymphoma, multiple myeloma, pancreatic ductal adenocarcinoma, hairy cell leukemia, metastatic prostate cancer, melanoma, colorectal cancer, or a combination thereof.

In some specific embodiments, the cancer is glioma. In certain embodiments, the glioma is glioblastoma multiforme, an ependymoma, astrocytoma, oligodendroglioma, oligoastrocytoma, a juvenile pilocytic astrocytoma, subependymal giant cell astrocytoma, ganglioglioma, subependymoma, xanthoastrocytoma, anaplastic, brainstem glioma, cerebellar astrocytoma, childhood desmoplastic astrocytoma, subependymal giant cell astrocytoma, diffuse astrocytoma, mixed glioma, optic glioma, cerebral gliomatosis, paraganglioma, ganglioglioma cells, or a combination thereof.

One embodiment provides a method for inhibiting a TYRO3-mediated disease or disorder, the method comprising administering a therapeutically effective amount of a compound having the following Structure (II):

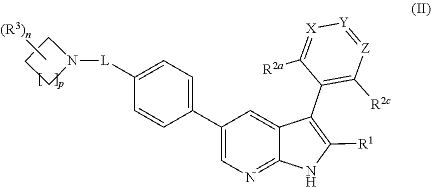

or a pharmaceutically acceptable salt, stereoisomer, tautomer, or prodrug thereof, wherein:
X is N or CR^{2d};
Y is N or CR^{2b};
Z is N or CR^{2e};
R¹ is hydrogen, C₁-C₆ alkyl, C₁-C₆ hydroxyalkyl, halo, C₁-C₆ haloalkyl, C₂-C₆ alkoxyalkyl, C₁-C₆ aminylalkyl, optionally substituted cycloalkylalkyl, or optionally substituted heteroaryl;
R^{2a}, R^{2b}, R^{2c}, R^{2d}, and R^{2e} are each, independently, hydrogen, C₁-C₆ alkyl, halo, cyano, C₁-C₆ haloalkyl, —R⁵OR⁶, or —R⁵N(R⁶)₂;
   or R^{2b} and R^{2e}, with the carbons to which they are attached, form a heterocyclyl optionally substituted with one or more alkyl groups;
   or R^{2b} and R^{2e}, with the adjacent carbons to which they are attached, form a heteroaryl that is optionally substituted with one or more alkyl groups;
R³ is, at each occurrence, independently alkyl, halo, —R⁵OR⁶, —R⁵OC(═O)R⁶,
   or —R⁵N(R⁶)₂;
   or two R³'s, when attached to different carbons, join to form —CH₂O—; or two R³'s, with the carbon to which they are both attached, form an optionally substituted heterocyclyl;
L is a direct bond or —C(R⁴)₂—;
R⁴ is, at each occurrence, independently hydrogen or alkyl;
R⁵ is, at each occurrence, independently a direct bond or an alkylene chain optionally substituted with one or more alkyl groups;
R⁶ is, at each occurrence, independently hydrogen, alkyl, or haloalkyl;
n is 0, 1, 2, or 3; and
p is 1, 2, or 3.

In more specific embodiments, the disease or disorder is cancer. In some more specific embodiments, the disease or disorder comprises a solid tumor. In some more specific embodiments, the disease or disorder is leukemia, lymphoma, brain cancer, genitourinary tract cancer, endocrine system cancer, gastrointestinal tract cancer, colon cancer, rectal cancer, breast cancer, kidney cancer, lymphatic system cancer, stomach cancer, lung cancer, pancreatic cancer, skin cancer, or combinations thereof. In certain more specific embodiments, the disease or disorder is bladder tumor, diffuse large B-Cell lymphoma, adenoid cystic carcinoma of salivary gland, Burkitt lymphoma, multiple myeloma, pancreatic ductal adenocarcinoma, hairy cell leukemia, metastatic prostate cancer, melanoma, colorectal cancer, or a combination thereof.

In some embodiments, the cancer is glioma. In some embodiments, the glioma is glioblastoma multiforme, an ependymoma, astrocytoma, oligodendroglioma, oligoastrocytoma, a juvenile pilocytic astrocytoma, subependymal giant cell astrocytoma, ganglioglioma, subependymoma, xanthoastrocytoma, anaplastic, brainstem glioma, cerebellar astrocytoma, childhood desmoplastic astrocytoma, subependymal giant cell astrocytoma, diffuse astrocytoma, mixed glioma, optic glioma, cerebral gliomatosis, paraganglioma, ganglioglioma cells, or a combination thereof.

The host or patient can belong to any mammalian species, for example a primate species, particularly humans; rodents, including mice, rats and hamsters; rabbits; horses, cows, dogs, cats, etc. Animal models are of interest for experimental investigations, providing a model for treatment of human disease.

Also included herein are methods of treatment in which at least one compound of Structure (I) or (II) is administered in combination with an anti-inflammatory or a therapeutic agent. Anti-inflammatory agents include but are not limited to NSAIDs, non-specific and COX-2 specific cyclooxygenase enzyme inhibitors, gold compounds, corticosteroids, methotrexate, tumor necrosis factor (TNF) antagonists, immunosuppressants, and methotrexate. Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine.

Examples of NSAIDs also include COX-2 specific inhibitors such as celecoxib, valdecoxib, lumiracoxib dnd/or etoricoxib.

In some embodiments, the anti-inflammatory agent is a salicylate. Salicylates include by are not limited to acetylsalicylic acid or aspirin, sodium salicylate, and choline and magnesium salicylates.

The anti-inflammatory agent may also be a corticosteroid. For example, the corticosteroid may be cortisone, dexamethasone, methylprednisolone, prednisolone, prednisolone sodium phosphate, or prednisone.

In additional embodiments the anti-inflammatory agent is a gold compound such as gold sodium thiomalate or auranofin.

The disclosure also includes embodiments in which the anti-inflammatory agent is a metabolic inhibitor such as a dihydrofolate reductase inhibitor, such as methotrexate or a dihydroorotate dehydrogenase inhibitor, such as leflunomide.

Therapeutic agents can also include agents for pain and inflammation such as histamine and histamine antagonists, bradykinin and bradykinin antagonists, 5-hydroxytryptamine (serotonin), lipid substances that are generated by biotransformation of the products of the selective hydrolysis of membrane phospholipids, eicosanoids, prostaglandins, thromboxanes, leukotrienes, aspirin, nonsteroidal anti-inflammatory agents, analgesic-antipyretic agents, agents that inhibit the synthesis of prostaglandins and thromboxanes, selective inhibitors of the inducible cyclooxygenase, selective inhibitors of the inducible cyclooxygenase-2, autacoids, paracrine hormones, somatostatin, gastrin, cytokines that mediate interactions involved in humoral and cellular immune responses, lipid-derived autacoids, eicosanoids, β-adrenergic agonists, ipratropium, glucocorticoids, methylxanthines, sodium channel blockers, opioid receptor agonists, calcium channel blockers, membrane stabilizers and leukotriene inhibitors.

Other embodiments of the disclosure pertain to combinations in which at least one anti-inflammatory compound is an anti-monoclonal antibody (such as eculizumab or pexelizumab), a TNF antagonist, such as entanercept, or infliximab, which is an anti-TNF alpha monoclonal antibody.

Therapeutic agents used in combination with the compounds of Structure (I) can also include small molecule compounds that inhibit the activation of NLRP3 inflammasomes, such as MCC950, sulforaphane, isoliquiritigenin, β-hydroxybutyrate, flufenamic acid, mefenamic acid, 3,4-methylenedioxy-β-nitrostyrene (MNS), and parthenolide.

Still other embodiments of the disclosure pertain to combinations in which at least one active agent is an immunosuppressant compound such as an immunosuppressant compound chosen from methotrexate, leflunomide, cyclosporine, tacrolimus, azathioprine, and mycophenolate mofetil. The disclosed compounds of Structure (I) or (II) can be administered in combination with other known therapeutic agents, including anticancer agents. As used here, the term “anticancer agent” relates to any agent which is administered to a patient with cancer for the purposes of treating the cancer.

In some embodiments the anti-cancer agents belong to the following categories:

Alkylating agents, such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone, apaziquone, fotemustine, glufosfamide, palifosfamide, pipobroman, trofosfamide, uramustine, TH-3024, or VAL-0834;

Platinum Compounds, such as carboplatin, cisplatin, eptaplatin, miriplatine hydrate, oxaliplatin, lobaplatin, nedaplatin, picoplatin, satraplatin, lobaplatin, nedaplatin, picoplatin, or satraplatin;

DNA altering agents, such as amrubicin, bisantrene, decitabine, mitoxantrone, procarbazine, trabectedin, clofarabine; amsacrine, brostallicin, pixantrone, or laromustine;

Topoisomerase Inhibitors: such as etoposide, irinotecan, razoxane, sobuzoxane, teniposide, topotecan; amonafide, belotecan, elliptinium acetate, or voreloxin;

Microtubule modifiers: such as cabazitaxel, docetaxel, eribulin, ixabepilone, paclitaxel, vinblastine, vincristine, vinorelbine, vindesine, vinflunine; fosbretabulin, or tesetaxel;

Antimetabolites, such as asparaginase, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur; doxifluridine, elacytarabine, raltitrexed, sapacitabine, tegafur, or trimetrexate;

Anticancer antibiotics, such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin, aclarubicin, peplomycin, or pirarubicin;

Hormones/Antagonists, such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol; acolbifene, danazol, deslorelin, epitiostanol, orteronel, or enzalutamide;

Aromatase inhibitors, such as aminoglutethimide, anastrozole, exemestane, fadrozole, letrozole, testolactone, or formestane;

Small molecule kinase inhibitors, such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib; afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifamib, tivantinib, tivozanib, trametinib, pimasertib, brivanib alaninate, or cediranib.

In some embodiments, medicaments which are administered in conjunction with the compounds described herein include any suitable drugs usefully delivered by inhalation for example, analgesics, e.g. codeine, dihydromorphine, ergotamine, fentanyl or morphine; anginal preparations, e.g. diltiazem; antiallergics, e.g. cromoglycate, ketotifen or nedocromil; anti-infectives, e.g. cephalosporins, penicillins, streptomycin, sulphonamides, tetracyclines or pentamidine; antihistamines, e.g. methapyrilene; anti-inflammatories, e.g. beclomethasone, flunisolide, budesonide, tipredane, triamcinolone acetonide or fluticasone; antitussives, e.g. noscapine; bronchodilators, e.g. ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoetharine, tulobuterol, orciprenaline or (−)-4-amino-3,5-dichloro-α-[[[6-[2-(2-pyridinyl)ethoxy]hexyl]-amino]methyl]benzenemethanol; diuretics, e.g., amiloride; anticholinergics, e.g., ipratropium, atropine or oxitropium; hormones, e.g., cortisone, hydrocortisone or prednisolone; xanthines, e.g., aminophylline, choline theophyllinate, lysine theophyllinate or theophylline; and therapeutic proteins and peptides, e.g., insulin or glucagon. It will be clear to a person skilled in the art that, where appropriate, the medicaments are used in the form of salts (e.g., as alkali metal or amine salts or as acid addition salts) or as esters (e.g., lower alkyl esters) or as solvates (e.g., hydrates) to optimize the activity and/or stability of the medicament.

The agents disclosed herein or other suitable agents are administered depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the present disclosure can be administered just followed by and any of the agents described above, or vice versa. In some embodiments of the separate administration protocol, a compound of the disclosure and any of the agents described above are administered a few minutes apart, or a few hours apart, or a few days apart.

In some embodiments, the compounds of Structure (I) or (II) are administered as a mono-therapy.

Embodiments of the methods of the disclosure can be performed either in vitro or in vivo. The susceptibility of a particular cell to treatment with the compounds of Structure (I) or (II) can be particularly determined by in vitro tests, whether in the course of research or clinical application.

The examples and preparations provided below further illustrate and exemplify the compounds of the present disclosure and methods of preparing and testing such compounds. It is to be understood that the scope of the present disclosure is not limited in any way by the scope of the following examples and preparations. In the following examples, and throughout the specification and claims, molecules with a single stereocenter, unless otherwise noted, exist as a racemic mixture. Those molecules with two or more stereocenters, unless otherwise noted, exist as a racemic mixture of diastereomers. Single enantiomers/diastereomers may be obtained by methods known to those skilled in the art.

### EXAMPLES

The following examples are provided for exemplary purposes.

### General Procedures

All proton NMR experiments were recorded on a Bruker AVANCE NEO Spectrometer equipped with a BBFO probe at 400 MHz. Deuterated solvents contained less than 0.05% v/v tetramethylsilane which was used as the reference signal (set at 0.00 ppm). When deuterated solvents did not contain tetramethylsilane, the residual nondeuterated solvent peaks were used as a reference signal, as per published guidelines (J. Org. Chem. 1997, 62(21), 7512-7515). Chemical shifts are expressed in parts per million (ppm, 6 units).

Coupling constants are in hertz (Hz). Splitting patterns describe apparent multiplicities and are designated as s (singlet), d (doublet), dd (doublet of doublets), t (triplet), q (quartet), m (multiplet), qt (quintuplet) or bs (broad singlet).

LC/MS analyses were performed on an Agilent Technologies UHPLC 1290 Infinity II with a G6125 MS detector and Waters system (Empower 3 software) with SQD (Single Quadrupole Detector).

Microwave reactions were conducted with a CEM Discover SP Module using standard protocols.

General Procedure GP1—Iodination of 7-azaindoles

NIS (1.5 eq.) was added to a solution of 7-azaindole precursor (1.0 eq) in acetone and the resulting mixture was stirred at 25° C. for 30 min to 1 h. The resulting precipitate was collected by filtration, washed with acetone, and dried under vacuum to afford the desired 3-iodo-7-azaindole compound.

General Procedure GP2—Tosylation of 7-azaindoles

NaH (60% in mineral oil, 1.2 eq.) was added to a solution of 7-azaindole precursor (1.0 eq) in DMF at 0° C. and the resulting suspension was stirred for 30 min. Tosyl chloride (1.2 eq) was added portionwise and the resulting mixture was allowed to warm to 25° C. and was stirred for 4 h. The reaction mixture was then quenched with water, the resulting precipitate was collected by filtration, washed with water, and dried under reduced pressure to afford the desired 1-tosyl-7-azaindole compound.

### General Procedure GP3—Suzuki Coupling at the 3-Position of 7-Azaindoles

A mixture of 3-iodo-7-azaindole Intermediate A (1.0 eq.), (hetero)aryl boronic acid Intermediate B (1.1 eq.), and K₂CO₃ (2.0 eq.) in toluene and ethanol (1:1) or 1,4-dioxane and water (1:1) was purged with argon for 15 min. Pd(PPh₃)₄ (0.05 eq) or Pd(dppf)Cl₂·CH₂Cl₂ (0.05 eq) was then added and the reaction mixture was stirred at 90° C. for 7 h. Following completion of the reaction (as indicated by TLC), the reaction mixture was diluted with ice water and extracted three times with EtOAc. The combined organic phases were washed with brine, dried over anhydrous Na₂SO₄, filtered, and concentrated under reduce pressure to give crude material which was purified by flash chromatography (eluting with 10 to 30% EtOAc in hexane), giving the desired compound.

General Procedure GP4—Suzuki Coupling at the 5-Position of 7-azaindoles

A mixture of halo Intermediate E or I (1.0 eq.), boronate ester Intermediate D (1.1 eq.), and Cs₂CO₃ (2.0 eq.) in 1,4-dioxane or DMF and water (4:1) was purged with argon for 15 min. Pd(dppf)Cl₂·CH₂Cl₂ (0.05 eq) was then added and the reaction mixture was stirred at 110° C. for 10 h. Following completion of the reaction (as indicated by TLC), the mixture was diluted with ice water and extracted three times with EtOAc. The combined organic phases were washed with brine, dried over anhydrous Na₂SO₄, filtered, and concentrated under reduce pressure to give crude material which was purified by flash chromatography (eluting with EtOAc in hexanes or MeOH in DCM), giving the desired compound.

### General Procedure GP5—Tosyl Removal

NaOH (5M in water, 5 eq.) or TBAF (1M in THF, 5 eq.) was added to a solution of tosylated precursor (1 eq.) in MeOH and acetone (1:1) or THE and the resulting mixture was stirred at 25° C. for 2 h. After completion of the reaction, the mixture was concentrated under reduced pressure, giving a residue which was diluted with water and extracted three times with DCM. The combined organic phases were washed with brine, dried over anhydrous Na₂SO₄, filtered, and concentrated under reduced pressure to give crude material which was purified by flash chromatography or preparative HPLC, giving the desired compound.

### General Procedure GP6-Boc Removal

TFA (5 eq.) or HCl (4M in 1,4-dioxane, 5 eq.) was added to a solution of Boc-protected precursor (1 eq.) in DCM or 1,4-dioxane and the resulting mixture was stirred at 25° C. for 2 h. Following completion of the reaction (as indicated by TLC), the mixture was concentrated under reduced pressure, diluted with saturated NaHCO₃, and extracted three times with DCM. The combined organic phases were washed with brine, dried over anhydrous Na₂SO₄, filtered, and concentrated under reduced pressure. The resulting crude material was triturated with Et₂O, giving the desired free amine.

### General Procedure GP7—Preparation of Boronate Esters

Bis(pinacolato)diboron (1.2 to 1.3 eq.) and potassium acetate (2 to 3 eq.) were added to a solution of halo precursor (1 eq.) in 1,4-dioxane and the resulting mixture was degassed with argon for 15 min. Pd(dppf)Cl₂—CH₂Cl₂ (0.05 eq) was then added and the reaction mixture was stirred at 100° C. for 5 to 12 hours. Following completion of the reaction, the mixture was diluted with water and extracted three times with EtOAc. The combined organic phases were washed with brine, dried over anhydrous Na₂SO₄, filtered, and concentrated under reduce pressure to give crude material which was purified by flash chromatography (eluting with EtOAc in hexane), giving the desired boronate ester.

General Procedure GP8—Amination of 4-(bromomethyl)phenylboronate esters

K₂CO₃ (3 eq.) and the desired amine (1.2 eq.) were added to a solution of 2-[4-(bromomethyl)phenyl]-4,4,5,5-tetramethyl-1,3,2-dioxaborolane (1.0 eq) in acetone and the resulting mixture was stirred at 25° C. for 16 h. Following completion of the reaction, the reaction mixture was filtered through a pad of diatomaceous earth (i.e., Celite®) which was then rinsed with EtOAc. The combined filtrates were concentrated under reduced pressure to give a residue which was taken in EtOAc, washed with water and brine, dried over anhydrous Na₂SO₄, filtered, and concentrated under reduced pressure to give the desired compound which was used without further purification.

### General Procedure GP9—Reductive Amination

Na(CH₃COO)₃BH (2.0 eq.) or NaBH₃CN (2.E780 eq) and a catalytic amount of acetic acid were or molecular sieves were added to a solution of the appropriate aldehyde (1.0 eq.) and amine Intermediate F (1.5 eq.) in DCM or MeOH and the resulting mixture was stirred at 45 to 60° C. for 2 to 4 h. Following completion of the reaction, the mixture was concentrated under reduced pressure, the resulting crude material was diluted with water and extracted twice with DCM. The combined organic phases were washed with brine, dried over anhydrous Na₂SO₄, filtered, and concentrated under reduced pressure to afford the desired compound.

### General Procedure GP10—Mesylation

Methanesulfonylchloride (1.5 eq.) was added to a solution of alcohol precursor (1.0 eq) and triethylamine (3 eq.) in DCM at 0° C. and the resulting mixture was allowed to warm to 25° C. and was stirred for 2 h. Following completion of the reaction, the reaction mixture was quenched with water and extracted twice with DCM. The combined organic phases were dried over anhydrous Na₂SO₄, filtered, and concentrated under reduced pressure to afford the desired compound.

### General Procedure GP11—Grignard Reaction

Methylmagnesium bromide (3M in Et₂O, 1.5 to 2.0 eq.) was added dropwise to a solution of the appropriate ketone (1.0 eq) in THE (5 mL) at 0° C. The resulting mixture was allowed to warm to 25° C. and was stirred for 2 h. Following completion of the reaction, the mixture was quenched with a saturated NH₄Cl solution (5 mL) and extracted with three times with EtOAc. The combined organic phases were washed with brine, dried over anhydrous Na₂SO₄, filtered, and concentrated under reduced pressure to give crude material which was purified by flash chromatography (eluting with EtOAc in n-hexane), affording the desired compound.

### Abbreviations

° C. (degree Celsius); ¹H NMR (proton Nuclear Magnetic Resonance); DCM (dichloromethane); DMAP (4-dimethylaminopyridine); DMF (N,N-dimethylformamide); DMSO-d₆ (deuterated dimethylsulfoxide); eq (equivalent); Et₂O (diethyl ether); EtOAc (ethyl acetate); g (gram); h (hour); HPLC (High Performance Liquid Chromatography); LC/MS (Liquid Chromatography Mass Spectrometry); LDA (lithium diisopropylamide); M (molar); MeOH (methanol); mg (milligram); min (minute); mL (milliliter); mmol (millimole); NIS (N-iodosuccinimide); Pd(PPh₃)₄ (tetrakis(triphenylphosphine)palladium(0)); Pd(dppf)Cl₂·CH₂Cl₂ (1,1′-bis(diphenylphosphino)ferrocene-palladium(II)dichloride dichloromethane complex); pTsOH (p-toluenesulfonic acid monohydrate); TBAF (tetra-n-butylammonium fluoride); tBu (tert-butyl); tBuOH (tert-butanol); TFA (trifluoroacetic acid); THE (tetrahydrofuran); TLC (Thin Layer Chromatography).

### PREPARATION OF KEY SYNTHETIC INTERMEDIATES

### Intermediates A

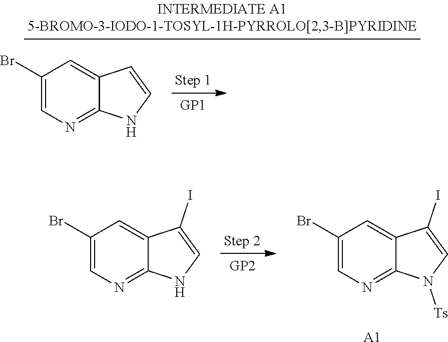 [SMILES: Brc1cnc2[nH]ccc2c1 ; Brc1cnc2[nH]cc(I)c2c1 ; Cc1ccc(S(=O)(=O)n2cc(I)c3cc(Br)cnc32)cc1 ; "A1"]

### Step 1: Synthesis of 5-bromo-3-iodo-1H-pyrrolo[2,3-b]pyridine

The title compound was synthesized by following General Procedure GP1, starting from 5-bromo-1H-pyrrolo[2,3-b]pyridine (10.0 g, 50.76 mmol), and was obtained as an off-white solid (10.5 g, 62% yield). LC/MS: 324.9 [M+H].

### Step 2: Synthesis of 5-bromo-3-iodo-1-tosyl-1H-pyrrolo[2,3-b]pyridine

The title compound was synthesized by following General Procedure GP2, starting from 5-bromo-3-iodo-1H-pyrrolo[2,3-b]pyridine (5.00 g, 15.5 mmol), and was obtained as an off-white solid (5.5 g, 74% yield). LC/MS: 476.6 [M+H].

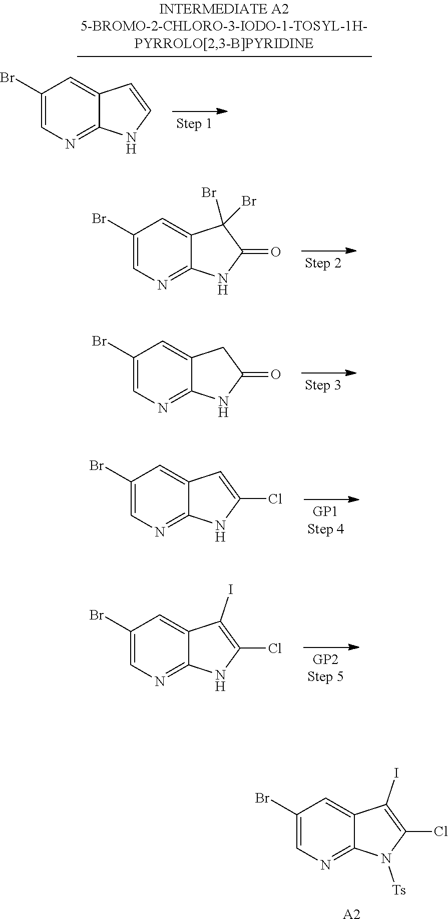

### Step 1: Synthesis of 3,3,5-tribromo-1,3-dihydro-2H-pyrrolo[2,3-b]pyridin-2-one

Bromine (3.91 mL, 76.1 mmol) was added dropwise to a solution of 5-bromo-1H-pyrrolo[2,3-b]pyridine (5.00 g, 25.4 mmol) in tBuOH (30 mL) and water (20 mL) and the resulting mixture was stirred at 25° C. for 1 h. Following completion of the reaction, the mixture was diluted with water (20 mL) and extracted with DCM (2×50 mL). The combined organic phases were washed with brine (25 mL), dried over anhydrous Na₂SO₄, filtered, and concentrated under reduced pressure to afford the title compound as a brown gum (7.0 g, crude) which was used without further purification. LC/MS: 372.8 [M+H].

### Step 2: Synthesis of 5-bromo-1,3-dihydro-2H-pyrrolo[2,3-b]pyridin-2-one

NH₄Cl (17.3 g, 324 mmol) in water (50 mL) and zinc (24.0 g, 367 mmol) were added to a solution of 3,3,5-tribromo-1,3-dihydro-2H-pyrrolo[2,3-b]pyridin-2-one (8.0 g, 21.6 mmol) in THE (150 mL) at 0° C. and the resulting mixture was stirred at 25° C. for 30 min. Following completion of the reaction, the reaction mixture was filtered through a pad of diatomaceous earth (i.e., Celite®). The organic phase was separated, the aqueous layer was extracted with THE (50 mL), the combined organic layers were washed with brine (50 mL), dried over anhydrous Na₂SO₄, filtered, and concentrated under reduced pressure to give crude material which was triturated with MeOH (50 mL). The resulting solid was collected by filtration and dried under reduced pressure to afford the title compound as an off-white solid (3.00 g, 65% yield). LC/MS: 213.0 [M+H].

### Step 3: Synthesis of 5-bromo-2-chloro-1H-pyrrolo[2,3-b]pyridine

A stirred solution of 5-bromo-1,3-dihydro-2H-pyrrolo[2,3-b]pyridin-2-one (1.50 g, 7.04 mmol) in POCl₃ (10 mL) was heated to 110° C. for 5 h. Following completion of the reaction, the mixture was concentrated under reduced pressure, diluted with ice water, and neutralized with NaOH (1M solution in water). The resulted solid was collected by filtration, washed with n-hexane, and dried under reduced pressure to afford the title compound as a yellow solid (1.00 g, 92% yield). ¹H NMR (400 MHz, DMSO-d₆) δ=12.74 (s, 1H), 8.25 (s, 1H), 8.12 (s, 1H), 6.47 (s, 1H). LC/MS: 231.9 [M+H].

### Step 4: Synthesis of 5-bromo-2-chloro-3-iodo-1H-pyrrolo[2,3-b]pyridine

The title compound was synthesized by following General Procedure GP1, starting from 5-bromo-2-chloro-1H-pyrrolo[2,3-b]pyridine (0.80 g, 3.46 mmol), and was obtained as a brown solid (0.90 g, 73% yield). ¹H NMR (400 MHz, DMSO-d₆) δ=13.26 (s, 1H), 8.31 (s, 1H), 7.82 (s, 1H). LC/MS: 356.8 [M+H].

### Step 5: Synthesis of 5-bromo-2-chloro-3-iodo-1-tosyl-1H-pyrrolo[2,3-b]pyridine

The title compound was synthesized by following General Procedure GP2, starting from 5-bromo-2-chloro-3-iodo-1H-pyrrolo[2,3-b]pyridine (0.40 g, 1.12 mmol), and was obtained as a brown solid (0.30 g, 52% yield). ¹H NMR (400 MHz, DMSO-d₆) δ=8.54 (s, 1H), 7.96-7.90 (m, 3H), 7.44-7.42 (m, 2H), 2.34 (s, 3H). LC/MS: 511.9 [M+H].

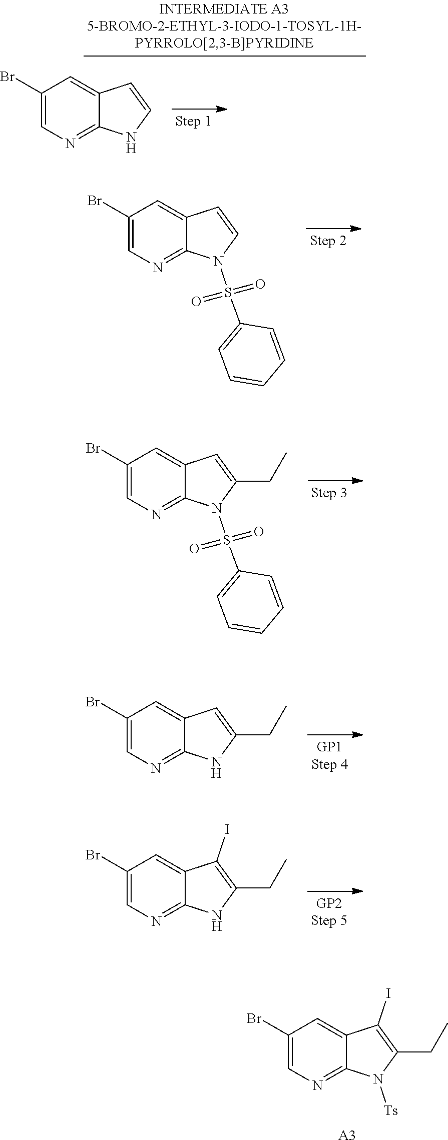

### Step 1: Synthesis of 5-bromo-1-(phenylsulfonyl)-1H-pyrrolo[2,3-b]pyridine

NaH (60% in mineral oil, 1.22 g, 30.5 mmol) was added to a solution of 5-bromo-1H-pyrrolo[2,3-b]pyridine (5.00 g, 25.4 mmol) in DMF (50 mL) at 0° C. and the resulting suspension was stirred for 30 min. Benzenesulfonyl chloride (3.89 mL, 30.5 mmol) was then added and the resulting mixture was allowed to warm to 25° C. and was stirred for 1 h. Following completion of the reaction, the mixture was quenched with ice-cold water (100 mL), the resulting precipitate was collected by filtration, washed with water (20 mL) then n-pentane (2×20 mL), and dried under reduced pressure to afford the tiled compound as an off-white solid (7.5 g, 87% yield). LC/MS: 337.0 [M+H].

### Step 2: Synthesis of 5-bromo-2-ethyl-1-(phenylsulfonyl)-1H-pyrrolo[2,3-b]pyridine

LDA (1M in THF, 11.1 mL, 22.2 mmol) was added to a solution of 5-bromo-1-(phenylsulfonyl)-1H-pyrrolo[2,3-b]pyridine (5.00 g, 14.8 mmol) in THE (50 mL) at −78° C. and the resulting solution was stirred at −78° C. for 30 min. Iodoethane (3.58 mL, 44.5 mmol) in THE (5 mL) was then added and the reaction mixture was allowed to warm to 25° C. and was stirred for 3 h. Following completion of the reaction, the mixture was quenched with saturated NH₄Cl (20 mL) and extracted with EtOAc (50 mL). The organic phase was washed with brine (5 mL), dried over anhydrous Na₂SO₄, filtered, and concentrated under reduced pressure to give crude material which was purified by flash chromatography (eluting with EtOAc in n-hexane), affording the title compound as an off-white solid (0.5 g, 9% yield). LC/MS: 364.7 [M+H].

### Step 3: Synthesis of 5-bromo-2-ethyl-1H-pyrrolo[2,3-b]pyridine

NaOH (2M in water, 3.42 mL, 6.84 mmol) was added to a solution of 5-bromo-2-ethyl-1-(phenylsulfonyl)-1H-pyrrolo[2,3-b]pyridine (0.5 g, 1.37 mmol) in MeOH (7 mL) and acetone (3 mL) and the resulting solution was stirred at 60° C. for 1 h. Following completion of the reaction, the solvent were removed under reduced pressure and the residue was dissolved in water (10 mL) then extracted with DCM (20 mL). The organic phase was washed with brine (5 mL), dried over anhydrous Na₂SO₄, filtered, and concentrated under reduced pressure to afford the title compound as a brown solid (0.3 g, crude) which was used without further purification. LC/MS: 225.0 [M+H].

### Step 4: Synthesis of 5-bromo-2-ethyl-3-iodo-1H-pyrrolo[2,3-b]pyridine

The title compound was synthesized by following General Procedure GP1, starting from 5-bromo-2-ethyl-1H-pyrrolo[2,3-b]pyridine (0.3 g, 1.33 mmol), and was obtained as a brown solid (0.4 g, 85% yield). ¹H NMR (400 MHz, DMSO-d₆) δ=12.29 (s, 1H), 8.21-8.20 (m, 1H), 7.71-7.70 (m, 1H), 2.76-2.71 (m, 2H), 1.21 (t, J=8.0 Hz, 3H). LC/MS: 350.7 [M+H].

### Step 5: Synthesis of 5-bromo-2-ethyl-3-iodo-1-tosyl-1H-pyrrolo[2,3-b]pyridine

The title compound was synthesized by following General Procedure GP2, starting from 5-bromo-2-ethyl-3-iodo-1H-pyrrolo[2,3-b]pyridine (0.4 g, 1.17 mmol), and was obtained as an off-white solid (0.5 g, 84% yield). ¹H NMR (400 MHz, DMSO-d₆) δ=8.41 (s, 1H), 7.95-7.93 (m, 2H), 7.88-7.87 (m, 1H), 7.40-7.38 (m, 2H), 3.22-3.17 (m, 2H), 2.32 (s, 3H), 1.27 (t, J=7.2 Hz, 3H). LC/MS: 504.6 [M+H].

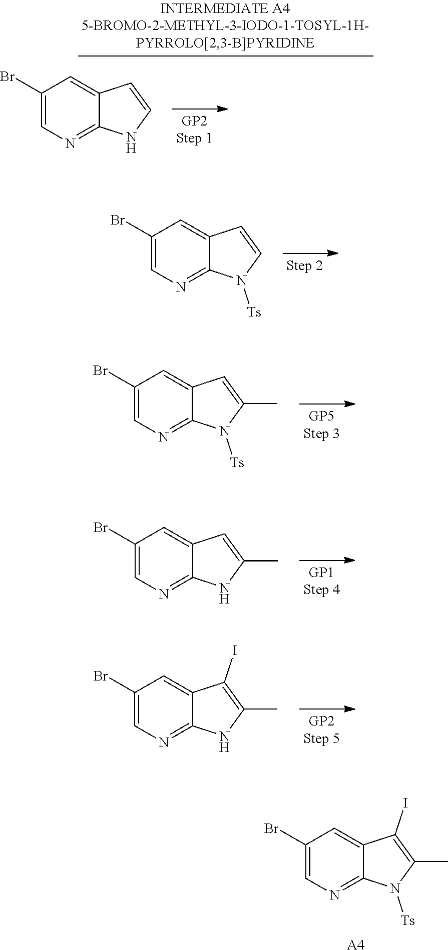

### Step 1: Synthesis of 5-bromo-1-tosyl-1H-pyrrolo[2,3-b]pyridine

The title compound was synthesized by following General Procedure GP2, starting from 5-bromo-1H-pyrrolo[2,3-b]pyridine (5.0 g, 15.5 mmol), and was obtained as an off-white solid (5.5 g, 74% yield). LC/MS: 350.7 [M+H].

### Step 2: Synthesis of 5-bromo-2-methyl-1-tosyl-1H-pyrrolo[2,3-b]pyridine

n-Butyllithium (2M in cyclohexane, 12.7 mL, 25.4 mmol) was added dropwise to a stirred solution of diisopropylamine (3.78 mL, 26.9 mmol) in THE (30 mL) at −78° C. and the resulting solution was stirred at −78° C. for 30 min. 5-bromo-1-tosyl-1H-pyrrolo[2,3-b]pyridine (3.50 g, 9.97 mmol) in THE (10 mL) was then added and the resulting mixture was stirred at −78° C. for 40 min. Methyl iodide (1.86 mL, 29.9 mmol) in THE (10 mL) was then added dropwise and the resulting mixture was allowed to warm to 25° C. and was stirred for 4 h. Following completion of the reaction, the mixture was cooled to 0° C., quenched with saturated NH₄Cl (50 mL), and extracted with EtOAc (2×100 mL). The combined organic phases were washed with brine (50 mL), dried over anhydrous Na₂SO₄, filtered, and concentrated under reduced pressure to give crude material which was purified by flash chromatography (eluting with EtOAc in n-hexane), affording the title compound (2.50 g, 68% yield) as an off-white solid. ¹H NMR (400 MHz, DMSO-d₆) δ=8.41-8.40 (m, 1H), 8.22-8.20 (m, 1H), 8.05-8.00 (m, 2H), 7.50-7.44 (m, 2H), 6.59-6.58 (m, 1H), 2.75 (s, 3H), 2.39 (s, 3H). LC/MS: 366.7 [M+H].

### Step 3: Synthesis of 5-bromo-2-methyl-1H-pyrrolo[2,3-b]pyridine

The title compound was synthesized by following General Procedure GP5, starting from 5-bromo-2-methyl-1-tosyl-1H-pyrrolo[2,3-b]pyridine (2.50 g, 6.84 mmol), and was obtained as an off-white solid (1.00 g, 69% yield). ¹H NMR (400 MHz, DMSO-d₆) δ=11.68 (s, 1H), 8.10-8.09 (m, 1H), 7.98-7.97 (m, 1H), 6.11 (s, 1H), 2.37 (s, 3H). LC/MS: 211.0 [M+H].

### Step 4: Synthesis of 5-bromo-3-iodo-2-methyl-1H-pyrrolo[2,3-b]pyridine

The title compound was synthesized by following General Procedure GP1, starting from 5-bromo-2-methyl-1H-pyrrolo[2,3-b]pyridine (1.00 g, 4.74 mmol), and was obtained as an off-white solid (1.50 g, 93% yield). ¹H NMR (400 MHz, DMSO-d₆) δ=12.28 (s, 1H), 8.22-8.21 (m, 1H), 7.71-7.70 (m, 1H), 2.41 (s, 3H). LC/MS: 338.9 [M+H].

### Step 5: Synthesis of 5-bromo-3-iodo-2-methyl-1-tosyl-1H-pyrrolo[2,3-b]pyridine

The title compound was synthesized by following General Procedure GP2, starting from 5-bromo-3-iodo-2-methyl-1H-pyrrolo[2,3-b]pyridine (1.50 g, 4.45 mmol), and was obtained as an off-white solid (1.60 g, 73% yield). LC/MS: 490.9 [M+H].

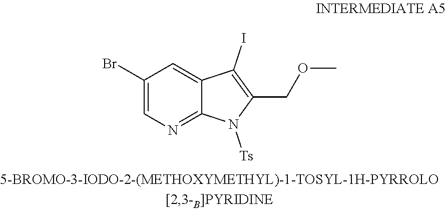 [SMILES: COCc1c(I)c2cc(Br)cnc2n1S(=O)(=O)c1ccc(C)cc1]

The title compound was synthesized in a manner similar to Intermediate A4, using iodo(methoxy)methane instead of methyl iodide in step 2. LC/MS: 366.9 [M+H].

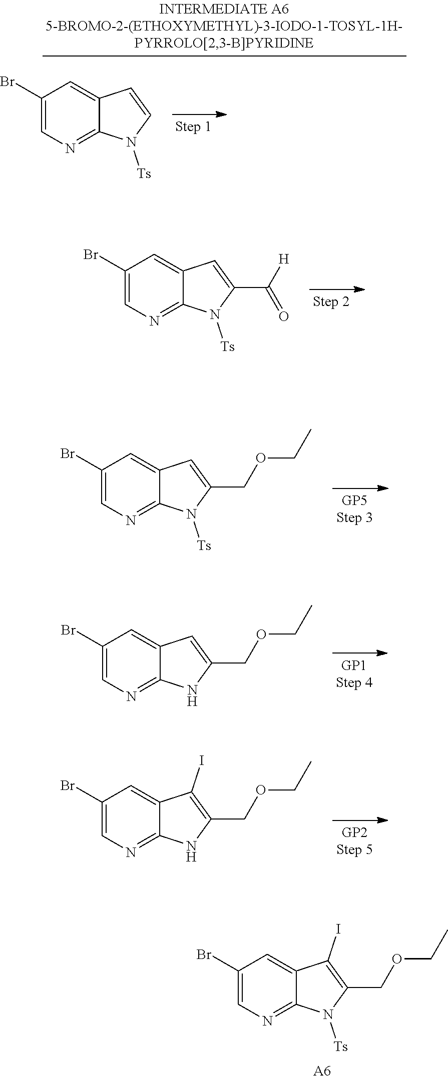

### Step 1: Synthesis of 5-bromo-1-tosyl-1H-pyrrolo[2,3-b]pyridine-2-carbaldehyde

LDA (2M in THF/heptane, 10.7 mL, 21.4 mmol) was added dropwise over 15 min to a solution of 5-bromo-1-tosyl-1H-pyrrolo[2,3-b]pyridine (step 1 of Intermediate A4, 5.00 g, 14.2 mmol) in THE (100 mL) at −78° C. and the resulting solution was stirred at −78° C. for 1 h. DMF (4.43 mL, 56.9 mmol) was then added dropwise over 5 min and the resulting solution was stirred at −78° C. for 20 min. Following completion of the reaction, the mixture was quenched with saturated NH₄Cl (20 mL) at −78° C. and the resulting precipitate was removed by filtration. The filtrate was extracted with EtOAc (2×100 mL), the combined organic phases were washed with brine (40 mL), dried over anhydrous Na₂SO₄, filtered, and concentrated under reduced pressure to give crude material which was purified by flash chromatography (eluting with EtOAc in n-hexane), affording the title compound (3.00 g, 55% yield) as an off-white solid. ¹H NMR (400 MHz, DMSO-d₆) δ=10.42 (s, 1H), 8.67 (d, J=2.0 Hz, 1H), 8.48 (d, J=2.0 Hz, 1H), 8.02 (d, J=8.4 Hz, 2H), 7.46-7.41 (m, 3H), 2.32 (s, 3H). LC/MS: 378.7 [M+H].

### Step 2: Synthesis of 5-bromo-2-(ethoxymethyl)-1-tosyl-1H-pyrrolo[2,3-b]pyridine

Ethanol (0.770 mL, 13.2 mmol), triethylsilane (0.927 mL, 5.80 mmol), and trifluoromethanesulfonic acid (0.046 mL, 0.527 mmol) were added to a solution of 5-bromo-1-tosyl-1H-pyrrolo[2,3-b]pyridine-2-carbaldehyde (2.00 g, 5.270 mmol) in nitromethane (10 mL) and the resulting mixture was stirred at 25° C. for 2 h. Following completion of the reaction, the mixture was concentrated under reduced pressure to give a residue which was diluted with water (20 mL) and extracted with EtOAc (30 mL). The organic phase was washed with brine (15 mL), dried over anhydrous Na₂SO₄, filtered, and concentrated under reduced pressure. The resulting crude material was purified by flash chromatography (eluting with EtOAc in n-hexane) to afford the title compound (0.5 g, 23% yield) as a white solid. ¹H NMR (400 MHz, DMSO-d₆) δ=8.43-8.42 (m, 1H), 8.13-8.11 (m, 2H), 7.90-7.88 (m, 1H), 7.29-7.27 (m, 2H), 6.57 (s, 1H), 4.98 (s, 2H), 3.73-3.68 (m, 2H), 2.41 (s, 3H), 1.30 (t, J=9.2 Hz, 3H). LC/MS: 411.3 [M+H].

### Step 3: Synthesis of 5-bromo-2-(ethoxymethyl)-1H-pyrrolo[2,3-b]pyridine

The title compound was synthesized by following General Procedure GP5, starting from 5-bromo-2-(ethoxymethyl)-1-tosyl-1H-pyrrolo[2,3-b]pyridine (0.5 g, 1.22 mmol), and was obtained as a white solid (0.25 g, crude) which was used without further purification. LC/MS: 256.3 [M+H].

### Step 4: Synthesis of 5-bromo-2-(ethoxymethyl)-3-iodo-1H-pyrrolo[2,3-b]pyridine

The title compound was synthesized by following General Procedure GP1, starting from 5-bromo-2-(ethoxymethyl)-1H-pyrrolo[2,3-b]pyridine (0.25 g, 0.98 mmol), and was obtained as a white solid (0.22 g, 59% yield). LC/MS: 380.9 [M+H].

### Step 5: Synthesis of 5-bromo-2-(ethoxymethyl)-3-iodo-1-tosyl-1H-pyrrolo[2,3-b]pyridine

The title compound was synthesized by following General Procedure GP2, starting from 5-bromo-2-(ethoxymethyl)-3-iodo-1H-pyrrolo[2,3-b]pyridine (0.22 g, 0.577 mmol), and was obtained as a white solid (0.25 g, 80% yield). LC/MS: 535.3 [M+H].

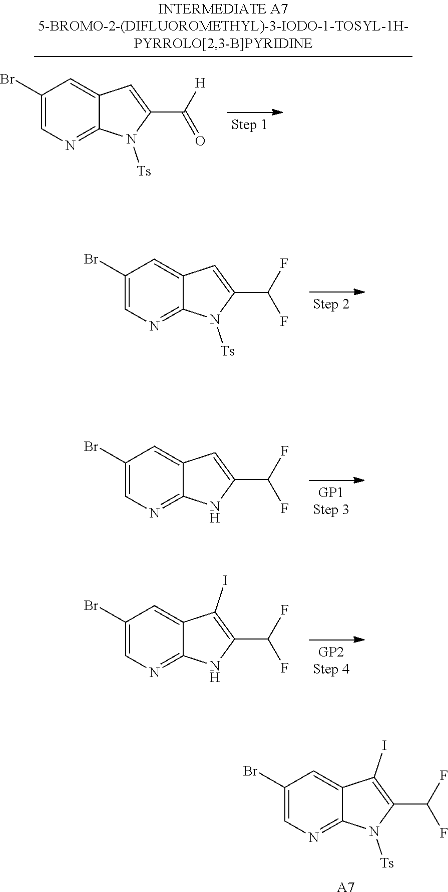 [SMILES: Cc1ccc(S(=O)(=O)n2c(C=O)cc3cc(Br)cnc32)cc1 ; Cc1ccc(S(=O)(=O)n2c(C(F)F)cc3cc(Br)cnc32)cc1 ; FC(F)c1cc2cc(Br)cnc2[nH]1 ; FC(F)c1[nH]c2ncc(Br)cc2c1I ; Cc1ccc(S(=O)(=O)n2c(C(F)F)c(I)c3cc(Br)cnc32)cc1 ; "A7"]

### Step 1: Synthesis of 5-bromo-2-(difluoromethyl)-1-tosyl-1H-pyrrolo[2,3-b]pyridine

(Diethylamino)sulfur trifluoride (1.88 mL, 14.2 mmol) was added dropwise to a solution of 5-bromo-1-tosyl-1H-pyrrolo[2,3-b]pyridine-2-carbaldehyde (step 1 of Intermediate A6, 1.80 g, 4.75 mmol) in DCM (30 mL) at 0° C. and the resulting mixture was stirred at 25° C. for 3 h. Following completion of the reaction, the mixture was quenched with saturated NaHCO₃ (30 mL) and extracted with DCM (2×50 mL). The combined organic phases were washed with brine (50 mL), dried over anhydrous Na₂SO₄, filtered, and concentrated under reduced pressure. The resulting crude material was triturated with methanol (20 mL), collected by filtration, and dried under reduced pressure to afford the title compound (1.50 g, 78% yield) as an off-white solid. ¹H NMR (400 MHz, DMSO-d₆) δ=8.56 (s, 1H), 8.39 (s, 1H), 8.02 (d, J=7.6 Hz, 2H), 7.62 (t, J=5.4 Hz, 1H), 7.41 (d, J=7.6 Hz, 2H), 7.18 (s, 1H), 2.33 (s, 3H). LC/MS: 400.7 [M+H].

### Step 2: Synthesis of 5-bromo-2-(difluoromethyl)-1-tosyl-1H-pyrrolo[2,3-b]pyridine

TBAF (1M in THF, 7.48 mL, 7.48 mmol) was added to a solution of 5-bromo-2-(difluoromethyl)-1-tosyl-1H-pyrrolo[2,3-b]pyridine (1.50 g, 3.74 mmol) in THE (15 mL) and the resulting mixture was stirred at 25° C. for 1 h. Following completion of the reaction, the mixture was diluted with water (20 mL) and extracted with EtOAc (60 mL). The organic phase was washed with saturated NaHCO₃ (10 mL) and brine (15 mL), dried over anhydrous Na₂SO₄, filtered, and concentrated under reduced pressure. Ther resulting crude material was triturated with MeOH (20 mL), collected by filtration, and dried under reduced pressure to afford the title compound (0.7 g, 75% yield) as an off-white solid. LC/MS: 249.0 [M+H].

### Step 3: Synthesis of 5-bromo-2-(difluoromethyl)-3-iodo-1H-pyrrolo[2,3-b]pyridine

The title compound was synthesized by following General Procedure GP1, starting from 5-bromo-2-(difluoromethyl)-1-tosyl-1H-pyrrolo[2,3-b]pyridine (0.5 g, 2.02 mmol), and was obtained as a pale yellow solid (0.6 g, 79% yield). ¹H NMR (400 MHz, DMSO-d₆) δ=13.15 (s, 1H), 8.45-8.44 (m, 1H), 8.01-8.00 (m, 1H), 7.17 (t, J=5.4 Hz, 1H). LC/MS: 372.9 [M+H].

### Step 4: Synthesis of 5-bromo-2-(difluoromethyl)-3-iodo-1-tosyl-1H-pyrrolo[2,3-b]pyridine

The title compound was synthesized by following General Procedure GP2, starting from 5-bromo-2-(difluoromethyl)-3-iodo-1H-pyrrolo[2,3-b]pyridine (0.6 g, 1.61 mmol), and was obtained as a yellow solid (0.6 g, 70% yield). LC/MS: 526.6 [M+H].

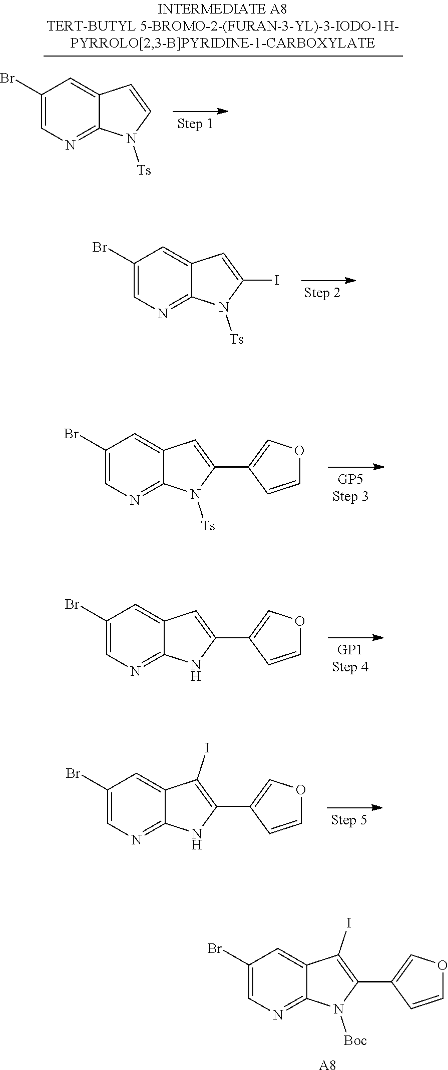

### Step 1: Synthesis of 5-bromo-2-iodo-1-tosyl-1H-pyrrolo[2,3-b]pyridine

LDA (2M in THF, 8.54 mL, 17.1 mmol) was added dropwise to as solution of 5-bromo-1-tosyl-1H-pyrrolo[2,3-b]pyridine (step 1 of Intermediate A4, 5.00 g, 14.2 mmol) in THE (50 mL) at −78° C. and the resulting mixture was stirred at −78° C. for 1 h. Iodine (7.95 g, 31.3 mmol) in THE (5 mL) was then added and the resulting mixture was allowed to warm to 25° C. and was stirred for 16 h. Following completion of the reaction, the mixture was quenched with saturated NH₄Cl (10 mL) and extracted with EtOAc (50 mL). The organic phase was washed with brine (25 mL), dried over anhydrous Na₂SO₄, filtered, and concentrated under reduced pressure to give crude material which was purified by flash chromatography (eluting with EtOAc in n-hexane), affording the title compound (4.50 g, 66% yield) as an off-white solid. ¹H NMR (400 MHz, DMSO-d₆) δ=8.41 (d, J=2.4 Hz, 1H), 8.18 (d, J=2.0 Hz, 1H), 7.95-7.70 (m, 2H), 7.44-7.41 (m, 2H), 7.17 (s, 1H), 2.34 (s, 3H). LC/MS: 476.9 [M+H].

### Step 2: Synthesis of 5-bromo-2-(furan-3-yl)-1-tosyl-1H-pyrrolo[2,3-b]pyridine

A solution of 5-bromo-2-iodo-1-tosyl-1H-pyrrolo[2,3-b]pyridine (2.00 g, 3.14 mmol), furan-3-ylboronic acid (0.352 g, 3.14 mmol), and NaHCO₃ (0.733 g, 6.92 mmol) in 1,4-dioxane (40 mL) and water (4 mL) was purged with nitrogen for 10 min. Pd(PPh₃)₄ (0.182 g 0.157 mmol) was then added and the resulting mixture was stirred 100° C. for 16 h in a sealed tube. Following completion of the reaction, the mixture was filtered through a pad of diatomaceous earth (i.e., Celite®) and the resulting filtrate was concentrated under vacuum. The resulting crude material was diluted with water (20 mL) and extracted with EtOAc (50 mL). The organic phase was washed with brine (15 mL), dried over anhydrous Na₂SO₄, filtered, and concentrated under reduced pressure to give crude material which was purified by flash chromatography (eluting with EtOAc in n-hexane), affording the title compound (1.00 g, 76% yield) as a yellow gum. LC/MS: 417.3 [M+H].

### Step 3: Synthesis of 5-bromo-2-(furan-3-yl)-1H-pyrrolo[2,3-b]pyridine

The title compound was synthesized by following General Procedure GP5, starting from 5-bromo-2-(furan-3-yl)-1-tosyl-1H-pyrrolo[2,3-b]pyridine (1.00 g, 2.40 mmol), and was obtained as an off-white solid (0.5 g, 79% yield). LC/MS: 262.8 [M+H].

### Step 4: Synthesis of 5-bromo-2-(furan-3-yl)-3-iodo-1H-pyrrolo[2,3-b]pyridine

The title compound was synthesized by following General Procedure GP1, starting from 5-bromo-2-(furan-3-yl)-1H-pyrrolo[2,3-b]pyridine (0.5 g, 1.90 mmol), and was obtained as a white solid (0.5 g, 67% yield). LC/MS: 262.8 [M+H].

### Step 5: Synthesis of tert-butyl 5-bromo-2-(furan-3-yl)-3-iodo-1H-pyrrolo[2,3-b]pyridine-1-carboxylate

Triethylamine (0.535 mL, 3.86 mmol), DMAP (0.015 g, 0.129 mmol), and di-tert-butyl dicarbonate (0.344 mL, 1.54 mmol) were added to a solution of 5-bromo-2-(furan-3-yl)-3-iodo-1H-pyrrolo[2,3-b]pyridine (0.500 g, 1.29 mmol) in THE (10 mL) and the resulting mixture stirred at 25° C. for 16 h. Following completion of the reaction, the mixture was diluted with water (20 mL) and extracted with EtOAc (50 mL). The organic phase was washed with brine (15 mL), dried over anhydrous Na₂SO₄, filtered, and concentrated under reduced pressure to give crude material which was purified by flash chromatography (eluting with EtOAc in n-hexane), affording the title compound (0.5 g, 79% yield) as an off-white solid. LC/MS: 489.3 [M+H].

INTERMEDIATES B

| Intermediate | B1 | B2 | B3 | B4 | B5 |
|---|---|---|---|---|---|
| Structure | 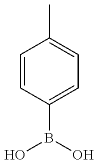 | 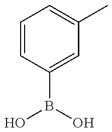 | 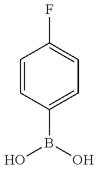 | 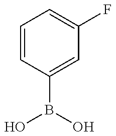 | 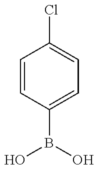 |
| Intermediate | B6 | B7 | B8 | B9 | B10 |
| Structure | 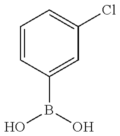 | 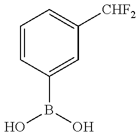 | 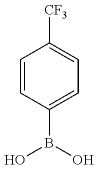 | 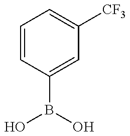 | 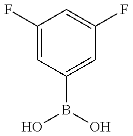 |
| Intermediate | B11 | B12 | B13 | B14 | B15 |
| Structure | 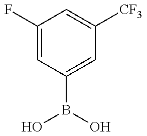 | 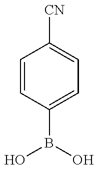 | 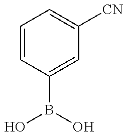 | 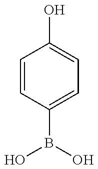 | 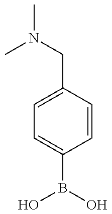 |
| Intermediate | B16 | B17 | B18 | B19 | B20 |
| Structure | 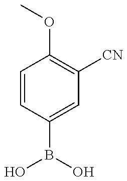 | 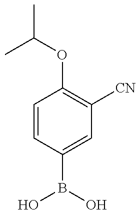 | 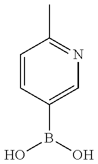 | 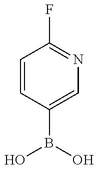 | 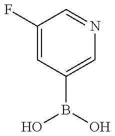 |
| Intermediate | B21 | B22 | B23 | B24 | B25 |
| Structure | 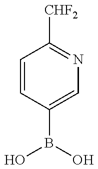 | 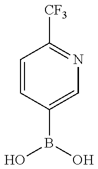 | 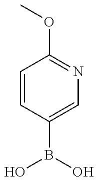 | 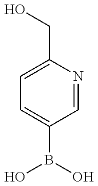 | 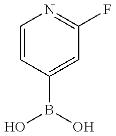 |
| Intermediate | B26 | B27 | B28 | B29 | B30 |
| Structure | 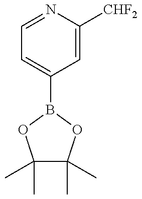 | 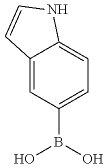 | 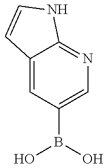 | 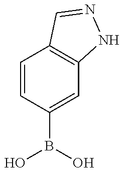 | 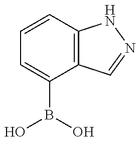 |
| Intermediate | B31 | B32 | B33 | B34 | B35 |
| Structure | 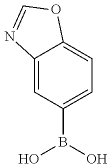 | 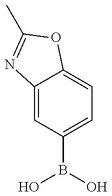 | 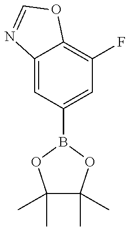 | 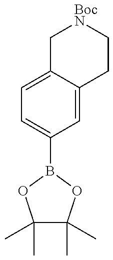 | 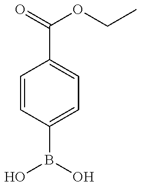 |
| Intermediate | B36 | B37 | B38 | | |
| Structure | 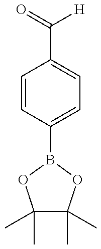 | 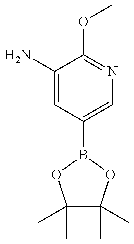 | 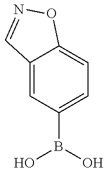 | | |

All Intermediates B are commercially available except for:

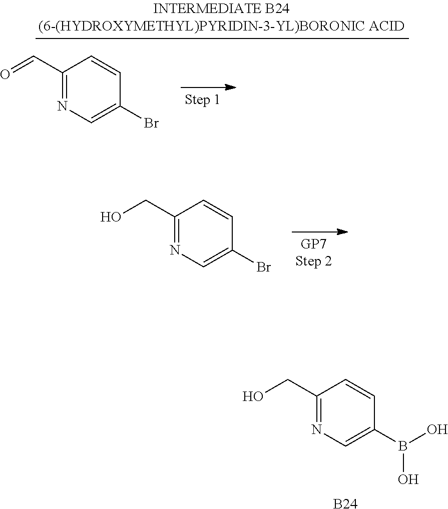

### Step 1: Synthesis of (5-bromopyridin-2-yl)methanol

NaBH₄ (0.1 g, 2.96 mmol) was added to a solution of 5-bromopicolinaldehyde (0.5 g, 2.69 mmol) in THE (10 mL) and EtOH (5 mL) at 0° C. and the resulting mixture was stirred at 25° C. for 1 h. Following completion of the reaction, the mixture was quenched with ice, extracted with EtOAc (20 mL), washed with brine (20 mL), dried over anhydrous Na₂SO₄, filtered, and concentrated under reduced pressure to give crude material which was purified by flash chromatography (eluting with MeOH in DCM) to afford the title compound as a white solid (0.5 g, quantitative). LC/MS: 190.0 [M+H]

### Step 2: Synthesis of (6-(hydroxymethyl)pyridin-3-yl)boronic acid

The title compound was synthesized by following General Procedure GP7, starting from (5-bromopyridin-2-yl)methanol (0.500 g, 2.66 mmol). The expected boronate ester underwent hydrolysis during the purification and the corresponding boronic acid was obtained as a brown solid (0.5 g, crude) which was used without further purification. LC/MS: 154.1 [M+H].

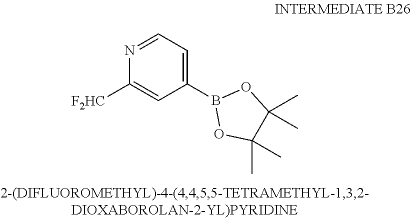 [SMILES: CC1(C)OB(c2ccnc(C(F)F)c2)OC1(C)C]

The title compound was synthesized by following General Procedure GP7, starting from 4-bromo-2-(difluoromethyl)pyridine (0.5 g, 2.40 mmol), and was obtained as a colorless oil (0.5 g, 81% yield). LC/MS: 256.2 [M+H].

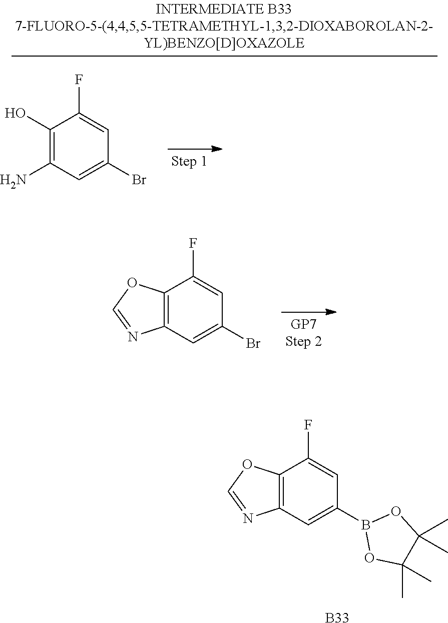

### Step 1: Synthesis of 5-bromo-7-fluorobenzo[d]oxazole

pTsOH (0.138 g, 0.728 mmol) was added to a solution of 2-amino-4-bromo-6-fluorophenol (1.500 g, 7.28 mmol) in trimethyl orthoformate (15 mL) and the resulting mixture was stirred at 85° C. for 2 h. Following completion of the reaction, the mixture was concentrated under reduced pressure, giving crude material which was purified by flash chromatography (eluting with EtOAc in n-hexane) to afford the title compound (0.7 g, 44% yield) as a brown oil. ¹H NMR (400 MHz, DMSO-d₆) δ=8.90 (s, 1H), 7.94-7.93 (m, 1H), 7.73-7.70 (m, 1H). LC/MS: 215.9 [M+H].

### Step 2: Synthesis of 7-fluoro-5-(4,4,5,5-tetramethyl-1,3,2-dioxaborolan-2-yl)benzo[d]oxazole

The title compound was synthesized by following General Procedure GP7 starting from 5-bromo-7-fluorobenzo[d]oxazole (0.6 g, 2.78 mmol), and was obtained as a brown solid (0.4 g, 54% yield). ¹H NMR (400 MHz, DMSO-d₆): δ 8.10 (s, 1H), 8.02 (s, 1H), 7.58-7.55 (m, 1H), 1.25 (s, 12H). LC/MS: 264.3 [M+H].

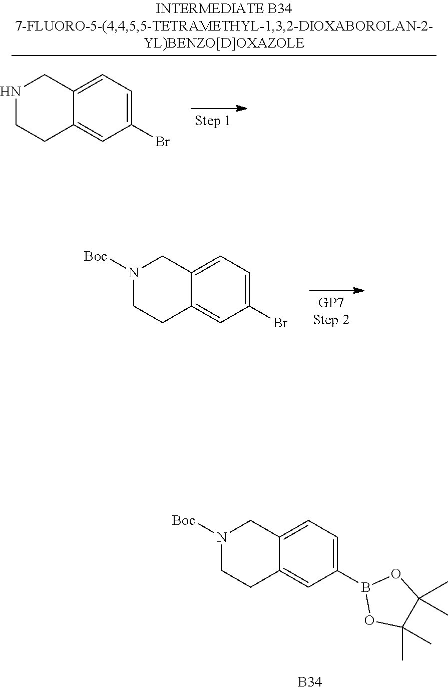 [SMILES: Brc1ccc2c(c1)CCNC2 ; CC(C)(C)OC(=O)N1CCc2cc(Br)ccc2C1 ; "B34" ; CC(C)(C)OC(=O)N1CCc2cc(B3OC(C)(C)C(C)(C)O3)ccc2C1]

Step 1: Synthesis of tert-butyl 6-bromo-3,4-dihydroisoquinoline-2(1H)-carboxylate

Di-tert-butyl dicarbonate (1.62 mL, 7.07 mmol) was added to a solution of 6-bromo-1,2,3,4-tetrahydroisoquinoline (1.00 g, 4.71 mmol) in DCM (20 mL) and the resulting mixture was stirred at 25° C. for 1 h. Following completion of the reaction, the mixture was concentrated under reduced pressure, giving crude material which was purified by flash chromatography (eluting with EtOAc in n-hexane) to afford the title compound (1.30 g, 88% yield) as a colorless oil. ¹H NMR (400 MHz, DMSO-d₆) δ=7.30-7.26 (m, 2H), 6.97 (d, J=7.2 Hz, 1H), 4.50 (s, 2H), 3.61 (s, 2H), 2.80 (s, 2H), 1.48 (s, 9H). LC/MS: 255.0 [M+H-tBu].

### Step 2: Synthesis of 7-fluoro-5-(4,4,5,5-tetramethyl-1,3,2-dioxaborolan-2-yl)benzo[d]oxazole

The title compound was synthesized by following General Procedure GP7, starting from tert-butyl 6-bromo-3,4-dihydroisoquinoline-2(1H)-carboxylate (1.30 g, 4.16 mmol), and was obtained as a colorless oil (1.10 g, 73% yield). LC/MS: 304.2 [M+H].

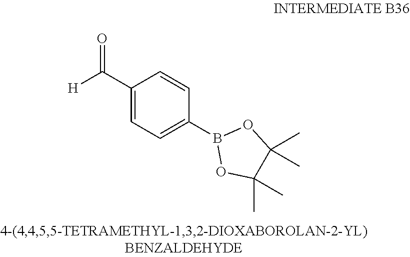 [SMILES: CC1(C)OB(c2ccc(C=O)cc2)OC1(C)C]

The title compound was synthesized by following General Procedure GP7, starting from 4-bromobenzaldehyde (1.0 g, 5.4 mmol), and was obtained as an off-white solid (1.2 g, 96% yield). LC/MS: 256.2 [M+H].

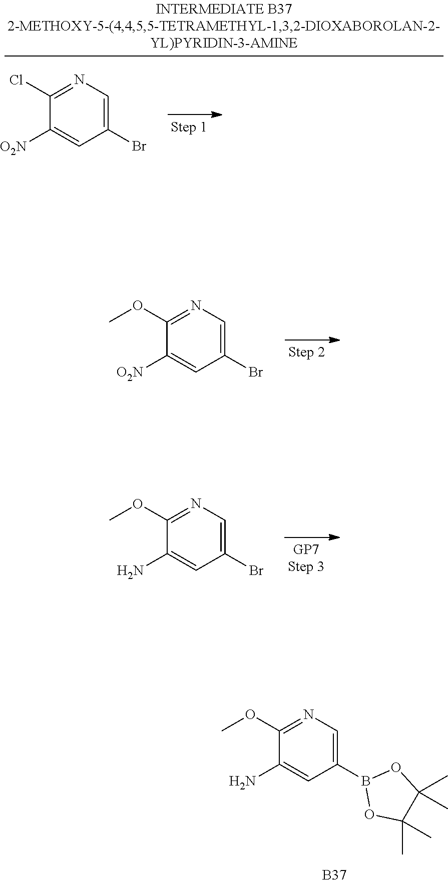

### Step 1: Synthesis of 5-bromo-2-methoxy-3-nitropyridine

Sodium methoxide (2.28 g, 42.1 mmol) was added to a solution of 5-bromo-2-chloro-3-nitropyridine (2.00 g, 8.42 mmol) in MeOH (60 mL) and the resulting mixture was stirred at reflux temperature for 12 h. Following completion of the reaction, the mixture was concentrated under reduced pressure. The residue was diluted with water (50 mL) and extracted with EtOAc (2×100 mL), the combined organic phases were washed with brine (50 mL), dried over anhydrous Na₂SO₄, filtered, and concentrated under reduced pressure to give the title compound (1.80 g, 92% yield) as a brown solid. ¹H NMR (400 MHz, CDCl₃) δ=8.45 (s, 1H), 8.39 (s, 1H), 4.11 (s, 3H).

### Step 2: Synthesis of 5-bromo-2-methoxypyridin-3-amine

A suspension of zinc dust (1.40 g, 21.5 mmol) and NH₄Cl (1.15 g, 21.5 mmol) in water (1 mL) was added to a solution of 5-bromo-2-methoxy-3-nitropyridine (1.00 g, 4.29 mmol) in methanol (20 mL) and the resulting mixture was stirred at 65° C. for 2 h. Following completion of the reaction, the mixture was filtered through a pad of diatomaceous earth (i.e., Celite®), and the filtrate was concentrated under reduced pressure. The residue was diluted with a saturated NaHCO₃ solution (15 mL) and extracted with EtOAc (2×50 mL). The combined organic phases were washed with brine (10 mL), dried over anhydrous Na₂SO₄, filtered, and concentrated under reduced pressure to give the title compound (0.6 g, crude) as a brown solid which was used without further purification. LC/MS: 205.9 [M+H].

### Step 3: Synthesis of 2-methoxy-5-(4,4,5,5-tetramethyl-1,3,2-dioxaborolan-2-yl)pyridin-3-amine

The title compound was synthesized by following General Procedure GP7, starting from 5-bromo-2-methoxypyridin-3-amine (0.4 g, 1.97 mmol), and was obtained as a brown solid (0.4 g, 81% yield). LC/MS: 251.2 [M+H].

### Intermediates C

The following Intermediates C were synthesized by reacting the appropriate 3-iodo-pyrrolo[2,3-b]pyridine Intermediate A and boronate ester Intermediate B as described in General Procedure GP3:

| | | | Starting | |
|---|---|---|---|---|
| Int. | Structure | Name | materials | LC/MS |
| C1 | 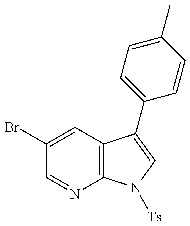 [SMILES: Cc1ccc(-c2cn(S(=O)(=O)c3ccc(C)cc3)c3ncc(Br)cc23)cc1] | 5-bromo-3-(p-tolyl)-1- tosyl-1H-pyrrolo[2,3- b]pyridine | A1, B1 | 441.1 [M + H] |
| C2 | 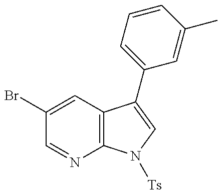 [SMILES: Cc1ccc(S(=O)(=O)n2cc(-c3cccc(C)c3)c3cc(Br)cnc32)cc1] | 5-bromo-3-(p-tolyl)-1- tosyl-1H-pyrrolo[2,3- b]pyridine | A1, B2 | 441.1 [M + H] |
| C3 | 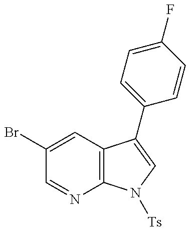 [SMILES: Cc1ccc(S(=O)(=O)n2cc(-c3ccc(F)cc3)c3cc(Br)cnc32)cc1] | 5-bromo-3-(4- fluorophenyl)-1-tosyl-1H- pyrrolo[2,3-b]pyridine | A1, B3 | 445.1 [M + H] |
| C4 | 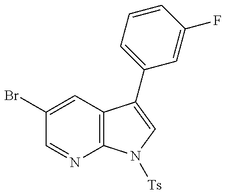 [SMILES: Cc1ccc(S(=O)(=O)n2cc(-c3cccc(F)c3)c3cc(Br)cnc32)cc1] | 5-bromo-3-(3- fluorophenyl)-1-tosyl-1H- pyrrolo[2,3-b]pyridine | A1, B4 | 445.1 [M + H] |
| C5 | 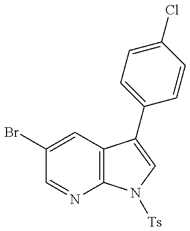 [SMILES: Cc1ccc(S(=O)(=O)n2cc(-c3ccc(Cl)cc3)c3cc(Br)cnc32)cc1] | 5-bromo-3-(4- chlorophenyl)-1-tosyl-1H- pyrrolo[2,3-b]pyridine | A1, B5 | 460.1 [M + H] |
| C6 | 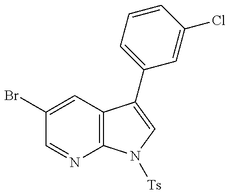 [SMILES: Cc1ccc(S(=O)(=O)n2cc(-c3cccc(Cl)c3)c3cc(Br)cnc32)cc1] | 5-bromo-3-(3- chlorophenyl)-1-tosyl-1H- pyrrolo[2,3-b]pyridine | A1, B6 | 460.1 [M + H] |
| C7 | 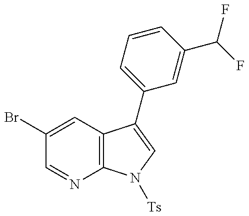 [SMILES: Cc1ccc(S(=O)(=O)n2cc(-c3cccc(C(F)F)c3)c3cc(Br)cnc32)cc1] | 5-bromo-3-(3- (difluoromethyl)phenyl)- 1-tosyl-1H-pyrrolo[2,3- b]pyridine | A1, B7 | 477.2 [M + H] |
| C8 | 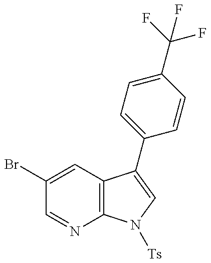 [SMILES: Cc1ccc(S(=O)(=O)n2cc(-c3ccc(C(F)(F)F)cc3)c3cc(Br)cnc32)cc1] | 5-bromo-1-tosyl-3-(4- (trifluoromethyl)phenyl)- 1H-pyrrolo[2,3-b]pyridine | A1, B8 | 495.1 [M + H] |
| C9 | 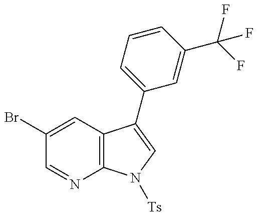 [SMILES: Cc1ccc(S(=O)(=O)n2cc(-c3cccc(C(F)(F)F)c3)c3cc(Br)cnc32)cc1] | 5-bromo-1-tosyl-3-(3- (trifluoromethyl)phenyl)- 1H-pyrrolo[2,3-b]pyridine | A1, B9 | 495.2 [M + H] |
| C10 | 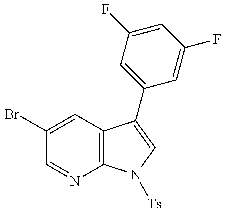 [SMILES: Cc1ccc(S(=O)(=O)n2cc(-c3cc(F)cc(F)c3)c3cc(Br)cnc32)cc1] | 5-bromo-3-(3,5- difluorophenyl)-1-tosyl- 1H-pyrrolo[2,3-b]pyridine | A1, B10 | 463.1 [M + H] |
| C11 | 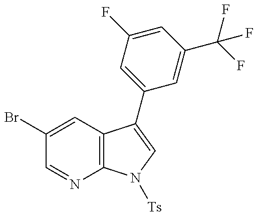 [SMILES: Cc1ccc(S(=O)(=O)n2cc(-c3cc(F)cc(C(F)(F)F)c3)c3cc(Br)cnc32)cc1] | 5-bromo-3-(3-fluoro-5- (trifluoromethyl)phenyl)- 1-tosyl-1H-pyrrolo[2,3- b]pyridine | A1, B11 | 513.2 [M + H] |
| C12 | 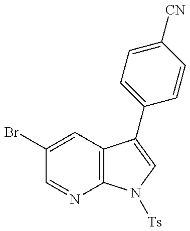 [SMILES: Cc1ccc(S(=O)(=O)n2cc(-c3ccc(C#N)cc3)c3cc(Br)cnc32)cc1] | 4-(5-bromo-1-tosyl-1H- pyrrolo[2,3-b]pyridin-3- yl)benzonitrile | A1, B12 | 452.1 [M + H] |
| C13 | 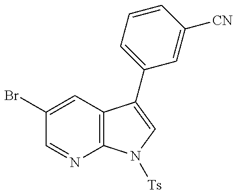 [SMILES: Cc1ccc(S(=O)(=O)n2cc(-c3cccc(C#N)c3)c3cc(Br)cnc32)cc1] | 3-(5-bromo-1-tosyl-1H- pyrrolo[2,3-b]pyridin-3- yl)benzonitrile | A1, B13 | 452.1 [M + H] |
| C14 | 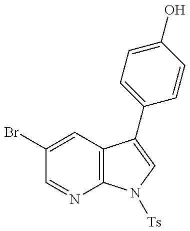 [SMILES: Cc1ccc(S(=O)(=O)n2cc(-c3ccc(O)cc3)c3cc(Br)cnc32)cc1] | 4-(5-bromo-1-tosyl-1H- pyrrolo[2,3-b]pyridin-3- yl)phenol | A1, B14 | 443.1 [M + H] |
| C15 | 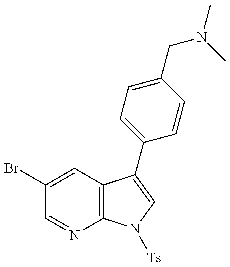 [SMILES: Cc1ccc(S(=O)(=O)n2cc(-c3ccc(CN(C)C)cc3)c3cc(Br)cnc32)cc1] | 1-(4-(5-bromo-1-tosyl-1H- pyrrolo[2,3-b]pyridin-3- yl)phenyl)-N,N- dimethylmethanamine | A1, B15 | 484.0 [M + H] |
| C16 | 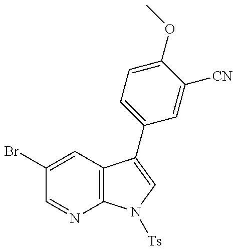 [SMILES: COc1ccc(-c2cn(S(=O)(=O)c3ccc(C)cc3)c3ncc(Br)cc23)cc1C#N] | 5-(5-bromo-1-tosyl-1H- pyrrolo[2,3-b]pyridin-3- yl)-2-methoxybenzonitrile | A1, B16 | 482.2 [M + H] |
| C17 | 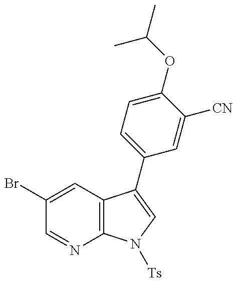 [SMILES: Cc1ccc(S(=O)(=O)n2cc(-c3ccc(OC(C)C)c(C#N)c3)c3cc(Br)cnc32)cc1] | 5-(5-bromo-1-tosyl-1H- pyrrolo[2,3-b]pyridin-3- yl)-2- isopropoxybenzonitrile | A1, B17 | 510.1 [M + H] |
| C18 | 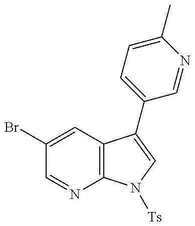 [SMILES: Cc1ccc(S(=O)(=O)n2cc(-c3ccc(C)nc3)c3cc(Br)cnc32)cc1] | 5-bromo-3-(6- methylpyridin-3-yl)-1- tosyl-1H-pyrrolo[2,3- b]pyridine | A1, B18 | 442.0 [M + H] |
| C19 | 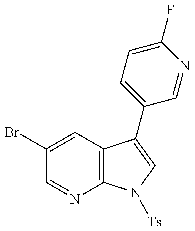 [SMILES: Cc1ccc(S(=O)(=O)n2cc(-c3ccc(F)nc3)c3cc(Br)cnc32)cc1] | 5-bromo-3-(6- fluoropyridin-3-yl)-1- tosyl-1H-pyrrolo[2,3- b]pyridine | A1, B19 | 446.2 [M + H] |
| C20 | 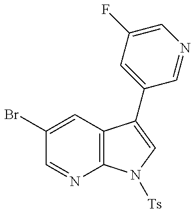 [SMILES: Cc1ccc(S(=O)(=O)n2cc(-c3cncc(F)c3)c3cc(Br)cnc32)cc1] | 5-bromo-3-(5- fluoropyridin-3-yl)-1- tosyl-1H-pyrrolo[2,3- b]pyridine | A1, B20 | 446.0 [M + H] |
| C21 | 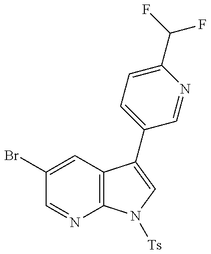 [SMILES: Cc1ccc(S(=O)(=O)n2cc(-c3ccc(C(F)F)nc3)c3cc(Br)cnc32)cc1] | 5-bromo-3-(6- (difluoromethyl)pyridin-3- yl)-1-tosyl-1H- pyrrolo[2,3-b]pyridine | A1, B21 | 478.2 [M + H] |
| C22 | 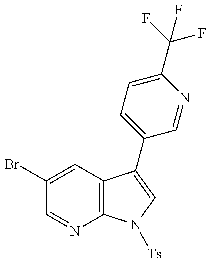 [SMILES: Cc1ccc(S(=O)(=O)n2cc(-c3ccc(C(F)(F)F)nc3)c3cc(Br)cnc32)cc1] | 5-bromo-3-(6- (trifluoromethyl)pyridin- 3-yl)-1-tosyl-1H- pyrrolo[2,3-b]pyridine | A1, B22 | 496.1 [M + H] |
| C23 | 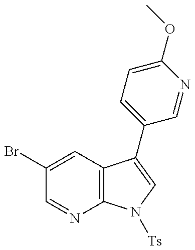 [SMILES: COc1ccc(-c2cn(S(=O)(=O)c3ccc(C)cc3)c3ncc(Br)cc23)cn1] | 5-bromo-3-(6- methoxypyridin-3-yl)-1- tosyl-1H-pyrrolo[2,3- b]pyridine | A1, B23 | 458.2 [M + H] |
| C24 | 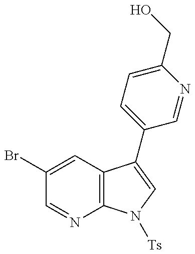 [SMILES: Cc1ccc(S(=O)(=O)n2cc(-c3ccc(CO)nc3)c3cc(Br)cnc32)cc1] | (5-(5-bromo-1-tosyl-1H- pyrrolo[2,3-b]pyridin-3- yl)pyridin-2-yl)methanol | A1, B24 | 458.1 [M + H] |
| C25 | 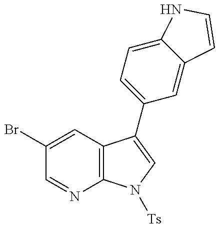 [SMILES: Cc1ccc(S(=O)(=O)n2cc(-c3ccc4[nH]ccc4c3)c3cc(Br)cnc32)cc1] | 5-bromo-3-(1H-indol-5- yl)-1-tosyl-1H- pyrrolo[2,3-b]pyridine | A1, B27 | 466.2 [M + H] |
| C26 | 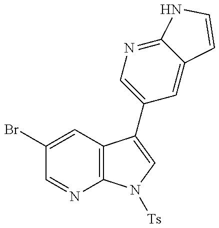 [SMILES: Cc1ccc(S(=O)(=O)n2cc(-c3cnc4[nH]ccc4c3)c3cc(Br)cnc32)cc1] | 5-bromo-1-tosyl-1H,1′H- 3,5'-bipyrrolo[2,3- b]pyridine | A1, B28 | 467.2 [M + H] |
| C27 | 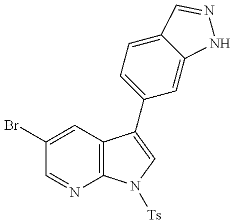 [SMILES: Cc1ccc(S(=O)(=O)n2cc(-c3ccc4cn[nH]c4c3)c3cc(Br)cnc32)cc1] | 6-(5-bromo-1-tosyl-1H- pyrrolo[2,3-b]pyridin-3- yl)-1H-indazole | A1, B29 | 467.2 [M + H] |
| C28 | 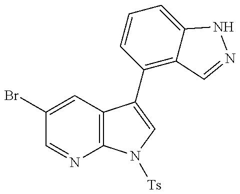 [SMILES: Cc1ccc(S(=O)(=O)n2cc(-c3cccc4[nH]ncc34)c3cc(Br)cnc32)cc1] | 4-(5-bromo-1-tosyl-1H- pyrrolo[2,3-b]pyridin-3- yl)-1H-indazole | A1, B30 | 467.1 [M + H] |
| C29 | 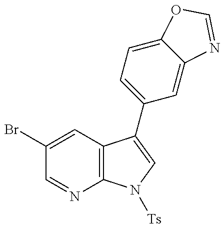 [SMILES: Cc1ccc(S(=O)(=O)n2cc(-c3ccc4ocnc4c3)c3cc(Br)cnc32)cc1] | 5-(5-bromo-1-tosyl-1H- pyrrolo[2,3-b]pyridin-3- yl)benzo[d]oxazole | A1, B31 | 468.2 [M + H] |
| C30 | 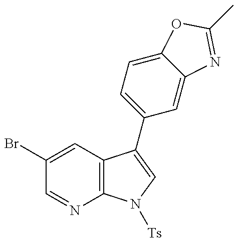 [SMILES: Cc1ccc(S(=O)(=O)n2cc(-c3ccc4oc(C)nc4c3)c3cc(Br)cnc32)cc1] | 5-(5-bromo-1-tosyl-1H- pyrrolo[2,3-b]pyridin-3- yl)-2- methylbenzo[d]oxazole | A1, B32 | 482.0 [M + H] |
| C31 | 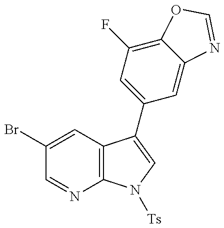 [SMILES: Cc1ccc(S(=O)(=O)n2cc(-c3cc(F)c4ocnc4c3)c3cc(Br)cnc32)cc1] | 5-(5-bromo-1-tosyl-1H- pyrrolo[2,3-b]pyridin-3- yl)-7- fluorobenzo[d]oxazole | A1, B33 | 486.1 [M + H] |
| C32 | 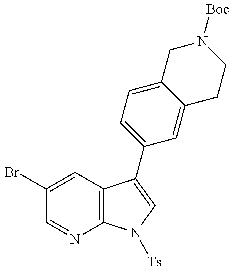 [SMILES: Cc1ccc(S(=O)(=O)n2cc(-c3ccc4c(c3)CCN(C(=O)OC(C)(C)C)C4)c3cc(Br)cnc32)cc1] | tert-butyl 6-(5-bromo-1- tosyl-1H-pyrrolo[2,3- b]pyridin-3-yl)-3,4- dihydroisoquinoline- 2(1H)-carboxylate | A1, B34 | 582.1 [M + H] |
| C33 | 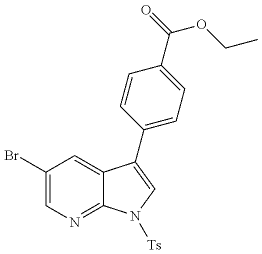 [SMILES: CCOC(=O)c1ccc(-c2cn(S(=O)(=O)c3ccc(C)cc3)c3ncc(Br)cc23)cc1] | ethyl 4-(5-bromo-1-tosyl- 1H-pyrrolo[2,3-b]pyridin- 3-yl)benzoate | A1, B35 | 499.1 [M + H] |
| C34 | 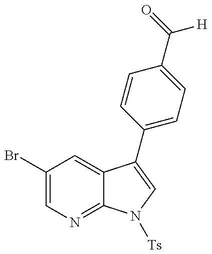 [SMILES: Cc1ccc(S(=O)(=O)n2cc(-c3ccc(C=O)cc3)c3cc(Br)cnc32)cc1] | 4-(5-bromo-1-tosyl-1H- pyrrolo[2,3-b]pyridin-3- yl)benzaldehyde | A1, B36 | 455.0 [M + H] |
| C35 | 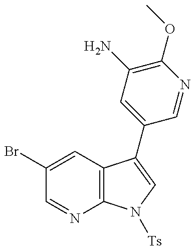 [SMILES: COc1ncc(-c2cn(S(=O)(=O)c3ccc(C)cc3)c3ncc(Br)cc23)cc1N] | 5-(5-bromo-1-tosyl-1H- pyrrolo[2,3-b]pyridin-3- yl)-2-methoxypyridin-3- amine | A1, B37 | 475.2 [M + H] |
| C36 | 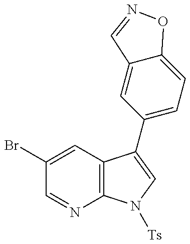 [SMILES: Cc1ccc(S(=O)(=O)n2cc(-c3ccc4oncc4c3)c3cc(Br)cnc32)cc1] | 5-(5-bromo-1-tosyl-1H- pyrrolo[2,3-b]pyridin-3- yl)benzo[d]isoxazole | A1, B38 | 468.1 [M + H] |
| C37 | 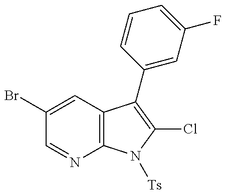 [SMILES: Cc1ccc(S(=O)(=O)n2c(Cl)c(-c3cccc(F)c3)c3cc(Br)cnc32)cc1] | 5-bromo-2-chloro-3-(3- fluorophenyl)-1-tosyl- 1H-pyrrolo[2,3- b]pyridine | A2, B4 | 479.2 [M + H] |
| C38 | 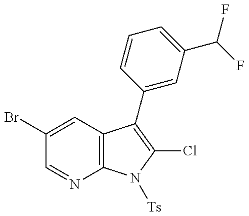 [SMILES: Cc1ccc(S(=O)(=O)n2c(Cl)c(-c3cccc(C(F)F)c3)c3cc(Br)cnc32)cc1] | 5-bromo-2-chloro-3-(3- (difluoromethyl)phenyl)- 1-tosyl-1H-pyrrolo[2,3- b]pyridine | A2, B7 | 511.3 [M + H] |
| C39 | 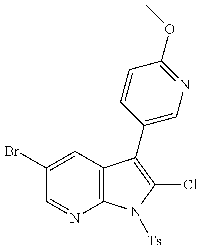 [SMILES: COc1ccc(-c2c(Cl)n(S(=O)(=O)c3ccc(C)cc3)c3ncc(Br)cc23)cn1] | 5-bromo-2-chloro-3-(6- methoxypyridin-3-yl)-1- tosyl-1H-pyrrolo[2,3- b]pyridine | A2, B23 | 492.1 [M + H] |
| C40 | 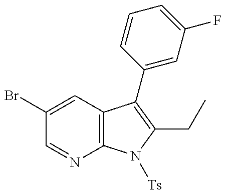 [SMILES: CCc1c(-c2cccc(F)c2)c2cc(Br)cnc2n1S(=O)(=O)c1ccc(C)cc1] | 5-bromo-2-ethyl-3-(3- fluorophenyl)-1-tosyl- 1H-pyrrolo[2,3- b]pyridine | A3, B4 | 473.2 [M + H] |
| C41 | 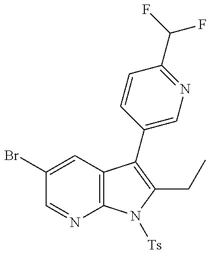 [SMILES: CCc1c(-c2ccc(C(F)F)nc2)c2cc(Br)cnc2n1S(=O)(=O)c1ccc(C)cc1] | 5-bromo-3-(6- (difluoromethyl)pyridin- 3-yl)-2-ethyl-1-tosyl-1H- pyrrolo[2,3-b]pyridine | A3, B21 | 506.1 [M + H] |
| C42 | 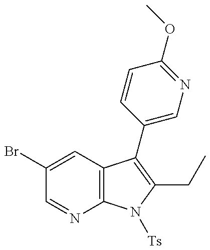 [SMILES: CCc1c(-c2ccc(OC)nc2)c2cc(Br)cnc2n1S(=O)(=O)c1ccc(C)cc1] | 5-bromo-2-ethyl-3-(6- methoxypyridin-3-yl)-1- tosyl-1H-pyrrolo[2,3- b]pyridine | A3, B23 | 486.2 [M + H] |
| C43 | 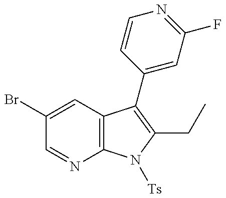 [SMILES: CCc1c(-c2ccnc(F)c2)c2cc(Br)cnc2n1S(=O)(=O)c1ccc(C)cc1] | 5-bromo-2-ethyl-3-(2- fluoropyridin-4-yl)-1- tosyl-1H-pyrrolo[2,3- b]pyridine | A3, B25 | 474.0 [M + H] |
| C44 | 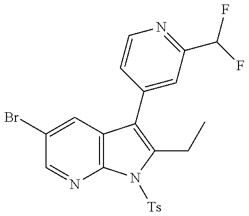 [SMILES: CCc1c(-c2ccnc(C(F)F)c2)c2cc(Br)cnc2n1S(=O)(=O)c1ccc(C)cc1] | 5-bromo-3-(2- (difluoromethyl)pyridin- 4-yl)-2-ethyl-1-tosyl-1H- pyrrolo[2,3-b]pyridine | A3, B26 | 506.1 [M + H] |
| C45 | 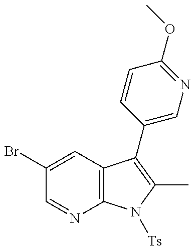 [SMILES: COc1ccc(-c2c(C)n(S(=O)(=O)c3ccc(C)cc3)c3ncc(Br)cc23)cn1] | 5-bromo-3-(6- methoxypyridin-3-yl)-2- methyl-1-tosyl-1H- pyrrolo[2,3-b]pyridine | A4, B23 | 471.0 [M + H] |
| C46 | 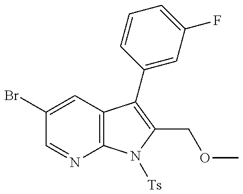 [SMILES: COCc1c(-c2cccc(F)c2)c2cc(Br)cnc2n1S(=O)(=O)c1ccc(C)cc1] | 5-bromo-3-(3- fluorophenyl)-2- (methoxymethyl)-1- tosyl-1H-pyrrolo[2,3- b]pyridine | A5, B4 | 489.1 [M + H] |
| C47 | 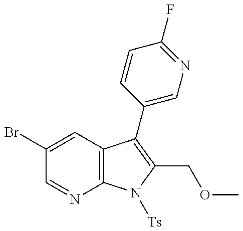 [SMILES: COCc1c(-c2ccc(F)nc2)c2cc(Br)cnc2n1S(=O)(=O)c1ccc(C)cc1] | 5-bromo-3-(6- fluoropyridin-3-yl)-2- (methoxymethyl)-1- tosyl-1H-pyrrolo[2,3- b]pyridine | A5, B19 | 490.0 [M + H] |
| C48 | 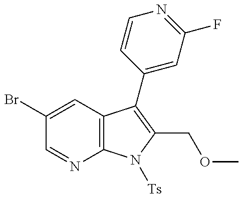 [SMILES: COCc1c(-c2ccnc(F)c2)c2cc(Br)cnc2n1S(=O)(=O)c1ccc(C)cc1] | 5-bromo-3-(2- fluoropyridin-4-yl)-2- (methoxymethyl)-1- tosyl-1H-pyrrolo[2,3- b]pyridine | A5, B25 | 490.0 [M + H] |
| C49 | 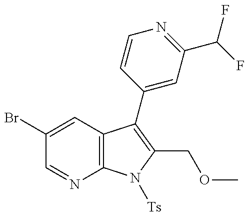 [SMILES: COCc1c(-c2ccnc(C(F)F)c2)c2cc(Br)cnc2n1S(=O)(=O)c1ccc(C)cc1] | 5-bromo-3-(2- (difluoromethyl)pyridin- 4-yl)-2- (methoxymethyl)-1- tosyl-1H-pyrrolo[2,3- b]pyridine | A5, B26 | 522.2 [M + H] |
| C50 | 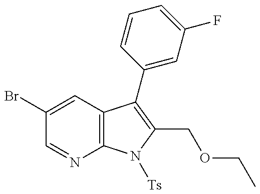 [SMILES: CCOCc1c(-c2cccc(F)c2)c2cc(Br)cnc2n1S(=O)(=O)c1ccc(C)cc1] | 5-bromo-2- (ethoxymethyl)-3-(3- fluorophenyl)-1-tosyl- 1H-pyrrolo[2,3- b]pyridine | A6, B4 | 503.3 [M + H] |
| C51 | 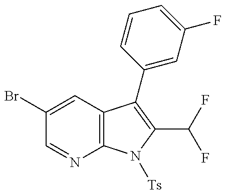 [SMILES: Cc1ccc(S(=O)(=O)n2c(C(F)F)c(-c3cccc(F)c3)c3cc(Br)cnc32)cc1] | 5-bromo-2- (difluoromethyl)-3-(3- fluorophenyl)-1-tosyl- 1H-pyrrolo[2,3- b]pyridine | A7, B4 | 495.0 [M + H] |
| C52 | 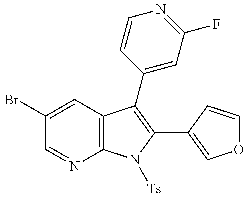 [SMILES: Cc1ccc(S(=O)(=O)n2c(-c3ccoc3)c(-c3ccnc(F)c3)c3cc(Br)cnc32)cc1] | tert-butyl 5-bromo-3-(2- fluoropyridin-4-yl)-2- (furan-3-yl)-1H- pyrrolo[2,3-b]pyridine-1- carboxylate | A8, B25 | 459.3 [M + H] |

### Intermediates D

The following Intermediates D were synthesized by reacting 2-[4-(bromomethyl) phenyl]-4,4,5,5-tetramethyl-1,3,2-dioxaborolane with the appropriate amine starting material as described in General Procedure GP8:

| | | | Amine | |
|---|---|---|---|---|
| | | | starting | |
| Int. | Structure | Name | material | LC/MS |
| D1 | 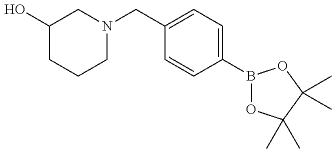 [SMILES: CC1(C)OB(c2ccc(CN3CCCC(O)C3)cc2)OC1(C)C] | 1-(4-(4,4,5,5-tetramethyl- 1,3,2-dioxaborolan-2- yl)benzyl)piperidin-3-ol | 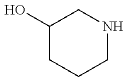 | 318.3 [M + H] |
| D2 | 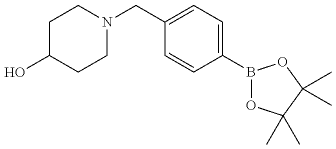 [SMILES: CC1(C)OB(c2ccc(CN3CCC(O)CC3)cc2)OC1(C)C] | 1-(4-(4,4,5,5-tetramethyl- 1,3,2-dioxaborolan-2- yl)benzyl)piperidin-4-ol | 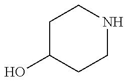 | 318.3 [M + H] |
| D3 | 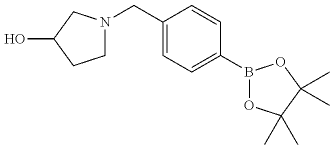 [SMILES: CC1(C)OB(c2ccc(CN3CCC(O)C3)cc2)OC1(C)C] | 1-(4-(4,4,5,5-tetramethyl- 1,3,2-dioxaborolan-2- yl)benzyl)pyrrolidin-3-ol | 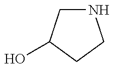 | 304.1 [M + H] |
| D4 | 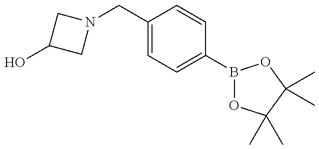 [SMILES: CC1(C)OB(c2ccc(CN3CC(O)C3)cc2)OC1(C)C] | 1-(4-(4,4,5,5-tetramethyl- 1,3,2-dioxaborolan-2- yl)benzyl)azetidin-3-ol | 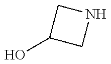 | 290.1 [M + H] |
| D5 | 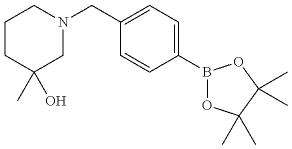 [SMILES: CC1(O)CCCN(Cc2ccc(B3OC(C)(C)C(C)(C)O3)cc2)C1] | 3-methyl-1-(4-(4,4,5,5- tetramethyl-1,3,2- dioxaborolan-2- yl)benzyl)piperidin-3-ol | 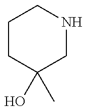 | 332.3 [M + H] |
| D6 | 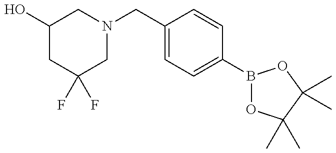 [SMILES: CC1(C)OB(c2ccc(CN3CC(O)CC(F)(F)C3)cc2)OC1(C)C] | 5,5-difluoro-1-(4- (4,4,5,5-tetramethyl- 1,3,2-dioxaborolan-2- yl)benzyl)piperidin-3-ol | 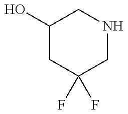 | 354.2 [M + H] |
| D7 | 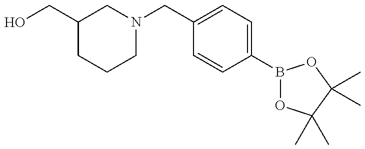 [SMILES: CC1(C)OB(c2ccc(CN3CCCC(CO)C3)cc2)OC1(C)C] | (1-(4-(4,4,5,5- tetramethyl-1,3,2- dioxaborolan-2- yl)benzyl)piperidin-3- yl)methanol | 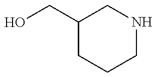 | 332.3 [M + H] |
| D8 | 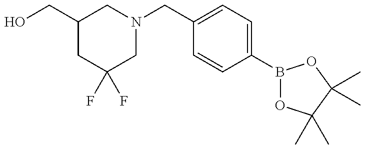 [SMILES: CC1(C)OB(c2ccc(CN3CC(CO)CC(F)(F)C3)cc2)OC1(C)C] | (5,5-difluoro-1-(4- (4,4,5,5-tetramethyl- 1,3,2-dioxaborolan-2- yl)benzyl)piperidin-3- yl)methanol | 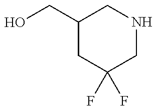 | 368.0 [M + H] |
| D9 | 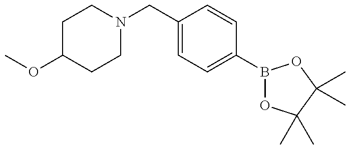 | 4-methoxy-1-(4-(4,4,5,5- tetramethyl-1,3,2- dioxaborolan-2- yl)benzyl)piperidine | 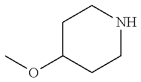 | 332.3 [M + H] |
| D10 | 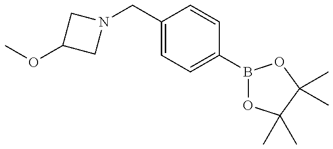 | 3-methoxy-1-(4-(4,4,5,5- tetramethyl-1,3,2- dioxaborolan-2- yl)benzyl)azetidine | 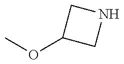 | 304.3 [M + H] |
| D11 | 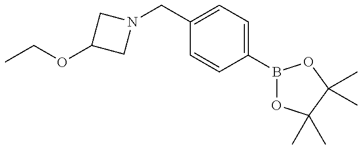 [SMILES: CCOC1CN(Cc2ccc(B3OC(C)(C)C(C)(C)O3)cc2)C1] | 3-ethoxy-1-(4-(4,4,5,5- tetramethyl-1,3,2- dioxaborolan-2- yl)benzyl)azetidine | 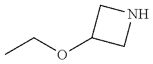 | 318.3 [M + H] |
| D12 | 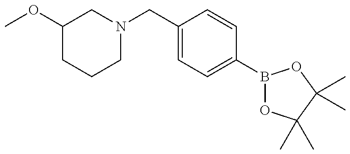 | 3-methoxy-1-(4-(4,4,5,5- tetramethyl-1,3,2- dioxaborolan-2- yl)benzyl)piperidine | 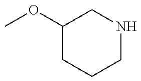 | 332.3 [M + H] |
| D13 | 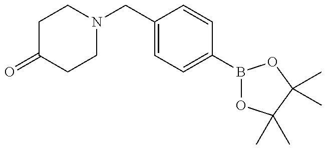 [SMILES: CC1(C)OB(c2ccc(CN3CCC(=O)CC3)cc2)OC1(C)C] | 1-(4-(4,4,5,5-tetramethyl- 1,3,2-dioxaborolan-2- yl)benzyl)piperidin-4-one | 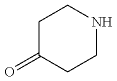 | 315.2 [M + H] |
| D14 | 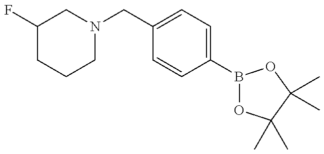 [SMILES: CC1(C)OB(c2ccc(CN3CCCC(F)C3)cc2)OC1(C)C] | 3-fluoro-1-(4-(4,4,5,5- tetramethyl-1,3,2- dioxaborolan-2- yl)benzyl)piperidine | 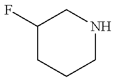 | 320.3 [M + H] |
| D15 | 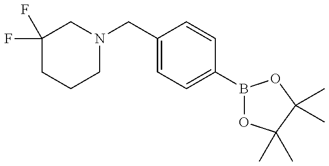 [SMILES: CC1(C)OB(c2ccc(CN3CCCC(F)(F)C3)cc2)OC1(C)C] | 3,3-difluoro-1-(4- (4,4,5,5-tetramethyl- 1,3,2-dioxaborolan-2- yl)benzyl)piperidine | 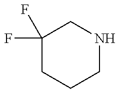 | 338.3 [M + H] |
| D16 | 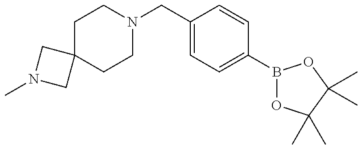 | 2-methyl-7-(4-(4,4,5,5- tetramethyl-1,3,2- dioxaborolan-2- yl)benzyl)-2,7- diazaspiro[3.5]nonane | 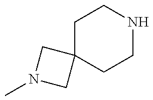 | 357.3 [M + H] |
| D17 | 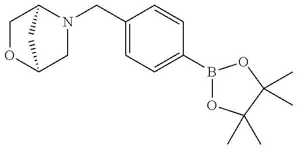 [SMILES: CC1(C)OB(c2ccc(CN3C[C@@H]4C[C@H]3CO4)cc2)OC1(C)C] | (1S,4S)-5-(4-(4,4,5,5- tetramethyl-1,3,2- dioxaborolan-2- yl)benzyl)-2-oxa-5- azabicyclo[2.2.1]heptane | 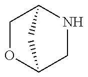 | 316.2 [M + H] |

All amine starting materials are commercially available except for 3-methylpiperidin-3-ol (precursor to Intermediate D35) which was prepared as follows:

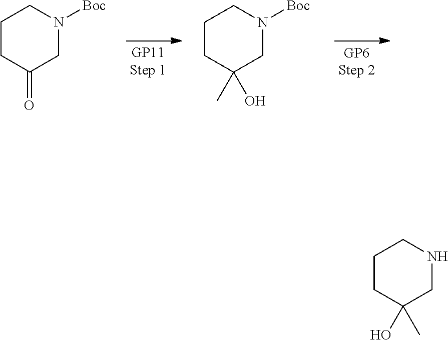

### Step 1: Synthesis of tert-butyl 3-hydroxy-3-methylpiperidine-1-carboxylate

The title compound was synthesized by following General Procedure GP 11, starting from commercially available tert-butyl 3-oxopiperidine-1-carboxylate (1.000 g, 5.02 mmol) in ethoxyethane (10 mL), and was obtained as a yellow gum (0.88 g, 81% yield) which was used without further purification. ¹H NMR (400 MHz, CDCl₃) δ=3.73-3.70 (m, 3H), 2.99 (s, 2H), 1.75-1.61 (m, 6H), 1.45 (m, 18H), 1.21 (s, 3H). LC/MS: 215.3 [M+H].

### Step 2: Synthesis of 3-methylpiperidin-3-ol

The title compound was synthesized by following General Procedure G6, starting from tert-butyl 3-hydroxy-3-methylpiperidine-1-carboxylate (0.88 g, 4.09 mmol), and was obtained as a yellow gum (0.75 g, TFA salt) which was used without further purification. LC/MC: 116.2 [M+H].

The following Intermediates D were synthesized by following General Procedure GP7:

| | | | Starting | |
|---|---|---|---|---|
| Int. | Structure | Name | material | LC/MS |
| D18 | 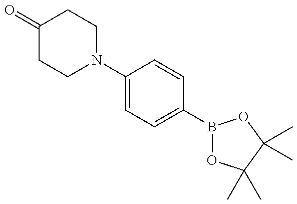 [SMILES: CC1(C)OB(c2ccc(N3CCC(=O)CC3)cc2)OC1(C)C] | 1-(4-(4,4,5,5-tetramethyl- 1,3,2-dioxaborolan-2- yl)phenyl)piperidin-4-one | | 302.4 [M + H] |
| D19 | 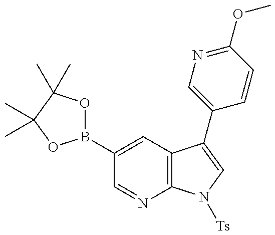 | 3-(6-methoxypyridin-3-yl)-5- (4,4,5,5-tetramethyl-1,3,2- dioxaborolan-2-yl)-1-tosyl- 1H-pyrrolo[2,3-b]pyridine | C23 | 506.3 [M + H] |

### Intermediates E

Intermediates E were synthesized by following General Procedure GP4:

| | | | Starting | |
|---|---|---|---|---|
| Int. | Structure | Name | materials | LC/MS |
| E1 | 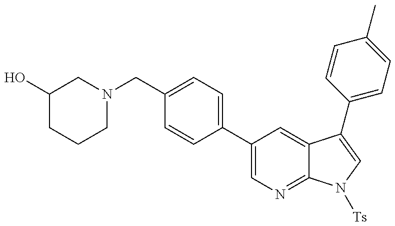 [SMILES: Cc1ccc(-c2cn(S(=O)(=O)c3ccc(C)cc3)c3ncc(-c4ccc(CN5CCCC(O)C5)cc4)cc23)cc1] | 1-(4-(3-(p-tolyl)-1-tosyl- 1H-pyrrolo[2,3-b]pyridin- 5-yl)benzyl)piperidin-3-ol | C1, D1 | 552.1 [M + H] |
| E2 | 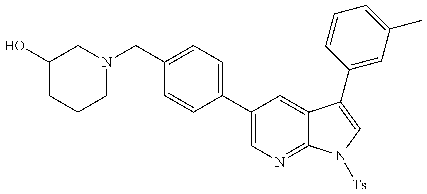 [SMILES: Cc1ccc(S(=O)(=O)n2cc(-c3cccc(C)c3)c3cc(-c4ccc(CN5CCCC(O)C5)cc4)cnc32)cc1] | 1-(4-(3-(m-tolyl)-1-tosyl- 1H-pyrrolo[2,3-b]pyridin- 5-yl)benzyl)piperidin-3-ol | C2, D1 | 552.2 [M + H] |
| E3 | 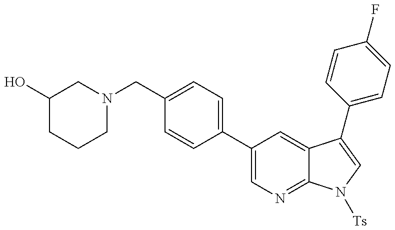 [SMILES: Cc1ccc(S(=O)(=O)n2cc(-c3ccc(F)cc3)c3cc(-c4ccc(CN5CCCC(O)C5)cc4)cnc32)cc1] | 1-(4-(3-(4-fluorophenyl)-1- tosyl-1H-pyrrolo[2,3- b]pyridin-5- yl)benzyl)piperidin-3-ol | C3, D1 | 556.1 [M + H] |
| E4 | 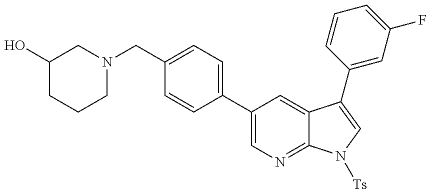 [SMILES: Cc1ccc(S(=O)(=O)n2cc(-c3cccc(F)c3)c3cc(-c4ccc(CN5CCCC(O)C5)cc4)cnc32)cc1] | 1-(4-(3-(3-fluorophenyl)-1- tosyl-1H-pyrrolo[2,3- b]pyridin-5- yl)benzyl)piperidin-3-ol | C4, D1 | 556.1 [M + H] |
| E5 | 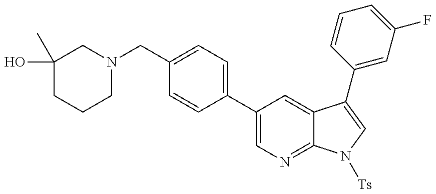 [SMILES: Cc1ccc(S(=O)(=O)n2cc(-c3cccc(F)c3)c3cc(-c4ccc(CN5CCCC(C)(O)C5)cc4)cnc32)cc1] | 1-(4-(3-(3-fluorophenyl)-1- tosyl-1H-pyrrolo[2,3- b]pyridin-5-yl)benzyl)-3- methylpiperidin-3-ol | C4, D5 | 570.0 [M + H] |
| E6 | 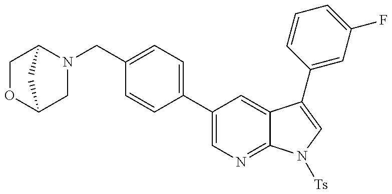 [SMILES: Cc1ccc(S(=O)(=O)n2cc(-c3cccc(F)c3)c3cc(-c4ccc(CN5C[C@@H]6C[C@H]5CO6)cc4)cnc32)cc1] | (1S,4S)-5-(4-(3-(3- fluorophenyl)-1-tosyl-1H- pyrrolo[2,3-b]pyridin-5- yl)benzyl)-2-oxa-5- azabicyclo[2.2.1]heptane | C4, D17 | 554.3 [M + H] |
| E7 | 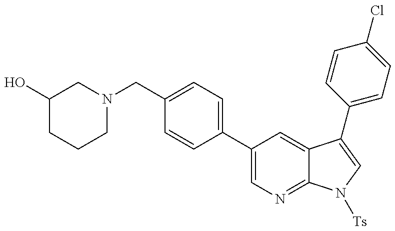 [SMILES: Cc1ccc(S(=O)(=O)n2cc(-c3ccc(Cl)cc3)c3cc(-c4ccc(CN5CCCC(O)C5)cc4)cnc32)cc1] | 1-(4-(3-(4-chlorophenyl)-1- tosyl-1H-pyrrolo[2,3- b]pyridin-5- yl)benzyl)piperidin-3-ol | C5, D1 | 572.3 [M + H] |
| E8 | 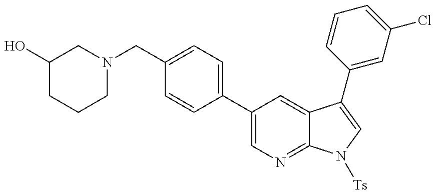 [SMILES: Cc1ccc(S(=O)(=O)n2cc(-c3cccc(Cl)c3)c3cc(-c4ccc(CN5CCCC(O)C5)cc4)cnc32)cc1] | 1-(4-(3-(3-chlorophenyl)-1- tosyl-1H-pyrrolo[2,3- b]pyridin-5- yl)benzyl)piperidin-3-ol | C6, D1 | 572.3 [M + H] |
| E9 | 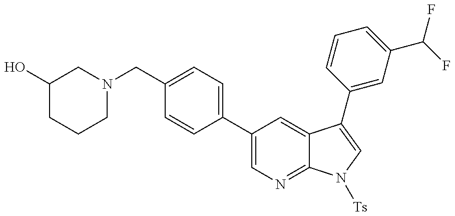 [SMILES: Cc1ccc(S(=O)(=O)n2cc(-c3cccc(C(F)F)c3)c3cc(-c4ccc(CN5CCCC(O)C5)cc4)cnc32)cc1] | 1-(4-(3-(3- (difluoromethyl)phenyl)-1- tosyl-1H-pyrrolo[2,3- b]pyridin-5- yl)benzyl)piperidin-3-ol | C7, D1 | 588.2 [M + H] |
| E10 | 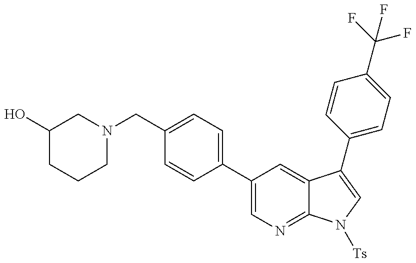 [SMILES: Cc1ccc(S(=O)(=O)n2cc(-c3ccc(C(F)(F)F)cc3)c3cc(-c4ccc(CN5CCCC(O)C5)cc4)cnc32)cc1] | 1-(4-(1-tosyl-3-(4- (trifluoromethyl)phenyl)- 1H-pyrrolo[2,3-b]pyridin- 5-yl)benzyl)piperidin-3-ol | C8, D1 | 606.3 [M + H] |
| E11 | 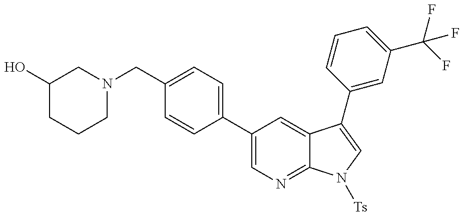 [SMILES: Cc1ccc(S(=O)(=O)n2cc(-c3cccc(C(F)(F)F)c3)c3cc(-c4ccc(CN5CCCC(O)C5)cc4)cnc32)cc1] | 1-(4-(1-tosyl-3-(3- (trifluoromethyl)phenyl)- 1H-pyrrolo[2,3-b]pyridin- 5-yl)benzyl)piperidin-3-ol | C9, D1 | 606.2 [M + H] |
| E12 | 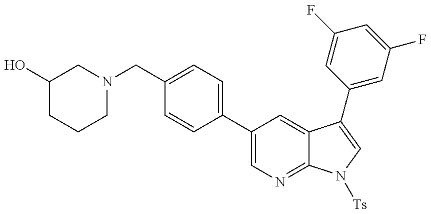 [SMILES: Cc1ccc(S(=O)(=O)n2cc(-c3cc(F)cc(F)c3)c3cc(-c4ccc(CN5CCCC(O)C5)cc4)cnc32)cc1] | 1-(4-(3-(3,5- difluorophenyl)-1-tosyl- 1H-pyrrolo[2,3-b]pyridin- 5-yl)benzyl)piperidin-3-ol | C10, D1 | 574.3 [M + H] |
| E13 | 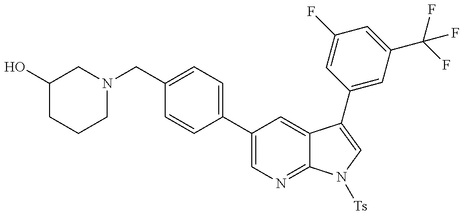 [SMILES: Cc1ccc(S(=O)(=O)n2cc(-c3cc(F)cc(C(F)(F)F)c3)c3cc(-c4ccc(CN5CCCC(O)C5)cc4)cnc32)cc1] | 1-(4-(3-(3-fluoro-5- (trifluoromethyl)phenyl)-1- tosyl-1H-pyrrolo[2,3- b]pyridin-5- yl)benzyl)piperidin-3-ol | C11, D1 | 624.2 [M + H] |
| E14 | 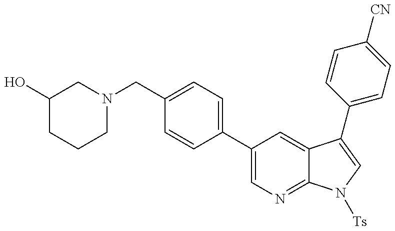 [SMILES: Cc1ccc(S(=O)(=O)n2cc(-c3ccc(C#N)cc3)c3cc(-c4ccc(CN5CCCC(O)C5)cc4)cnc32)cc1] | 4-(5-(4-((3- hydroxypiperidin-1- yl)methyl)phenyl)-1-tosyl- 1H-pyrrolo[2,3-b]pyridin- 3-yl)benzonitrile | C12, D1 | 563.2 [M + H] |
| E15 | 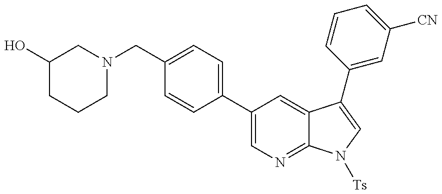 [SMILES: Cc1ccc(S(=O)(=O)n2cc(-c3cccc(C#N)c3)c3cc(-c4ccc(CN5CCCC(O)C5)cc4)cnc32)cc1] | 3-(5-(4-((3- hydroxypiperidin-1- yl)methyl)phenyl)-1-tosyl- 1H-pyrrolo[2,3-b]pyridin- 3-yl)benzonitrile | C13, D1 | 563.3 [M + H] |
| E16 | 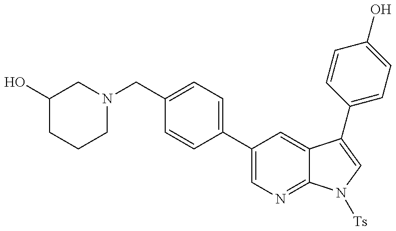 [SMILES: Cc1ccc(S(=O)(=O)n2cc(-c3ccc(O)cc3)c3cc(-c4ccc(CN5CCCC(O)C5)cc4)cnc32)cc1] | 1-(4-(3-(4-hydroxyphenyl)- 1-tosyl-1H-pyrrolo[2,3- b]pyridin-5- yl)benzyl)piperidin-3-ol | C14, D1 | 554.2 [M + H] |
| E17 | 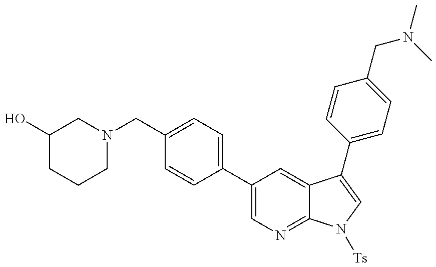 [SMILES: Cc1ccc(S(=O)(=O)n2cc(-c3ccc(CN(C)C)cc3)c3cc(-c4ccc(CN5CCCC(O)C5)cc4)cnc32)cc1] | 1-(4-(3-(4- ((dimethylamino)methyl) phenyl)-1-tosyl-1H- pyrrolo[2,3-b]pyridin-5- yl)benzyl)piperidin-3-ol | C15, D1 | 594.3 [M + H] |
| E18 | 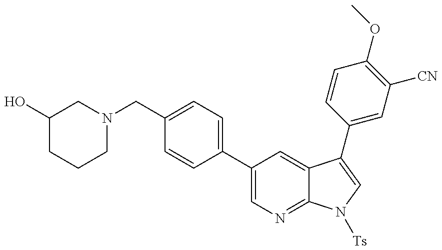 [SMILES: COc1ccc(-c2cn(S(=O)(=O)c3ccc(C)cc3)c3ncc(-c4ccc(CN5CCCC(O)C5)cc4)cc23)cc1C#N] | 5-(5-(4-((3- hydroxypiperidin-1- yl)methyl)phenyl)-1-tosyl- 1H-pyrrolo[2,3-b]pyridin- 3-yl)-2- methoxybenzonitrile | C16, D1 | 593.0 [M + H] |
| E19 | 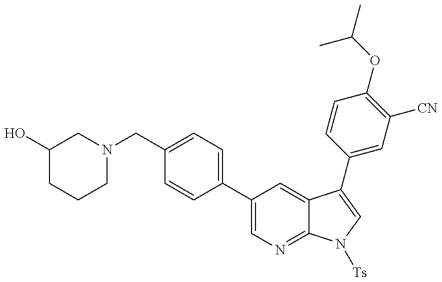 [SMILES: Cc1ccc(S(=O)(=O)n2cc(-c3ccc(OC(C)C)c(C#N)c3)c3cc(-c4ccc(CN5CCCC(O)C5)cc4)cnc32)cc1] | 5-(5-(4-((3- hydroxypiperidin-1- yl)methyl)phenyl)-1-tosyl- 1H-pyrrolo[2,3-b]pyridin- 3-yl)-2- isopropoxybenzonitrile | C17, D1 | 621.2 [M + H] |
| E20 | 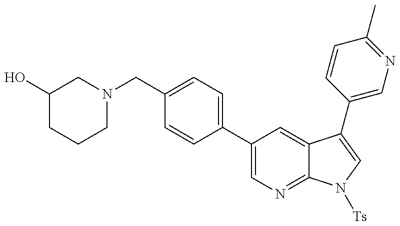 [SMILES: Cc1ccc(S(=O)(=O)n2cc(-c3ccc(C)nc3)c3cc(-c4ccc(CN5CCCC(O)C5)cc4)cnc32)cc1] | 1-(4-(3-(6-methylpyridin- 3-yl)-1-tosyl-1H- pyrrolo[2,3-b]pyridin-5- yl)benzyl)piperidin-3-ol | C18, D1 | 553.1 [M + H] |
| E21 | 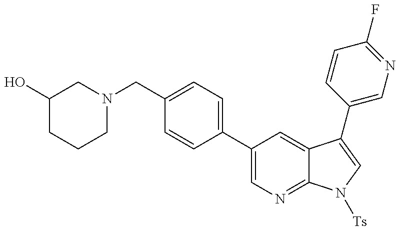 [SMILES: Cc1ccc(S(=O)(=O)n2cc(-c3ccc(F)nc3)c3cc(-c4ccc(CN5CCCC(O)C5)cc4)cnc32)cc1] | 1-(4-(3-(6-fluoropyridin-3- yl)-1-tosyl-1H-pyrrolo[2,3- b]pyridin-5- yl)benzyl)piperidin-3-ol | C19, D1 | 557.2 [M + H] |
| E22 | 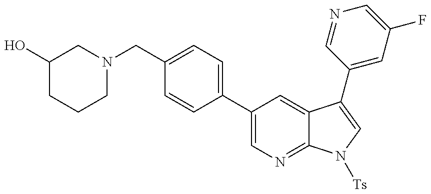 [SMILES: Cc1ccc(S(=O)(=O)n2cc(-c3cncc(F)c3)c3cc(-c4ccc(CN5CCCC(O)C5)cc4)cnc32)cc1] | 1-(4-(3-(5-fluoropyridin-3- yl)-1-tosyl-1H-pyrrolo[2,3- b]pyridin-5- yl)benzyl)piperidin-3-ol | C20, D1 | 557.3 [M + H] |
| E23 | 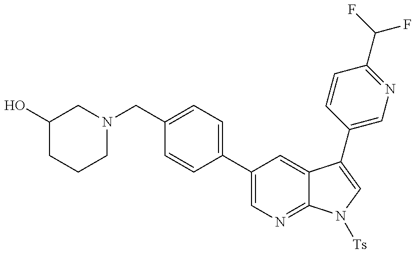 [SMILES: Cc1ccc(S(=O)(=O)n2cc(-c3ccc(C(F)F)nc3)c3cc(-c4ccc(CN5CCCC(O)C5)cc4)cnc32)cc1] | 1-(4-(3-(6- (difluoromethyl)pyridin-3- yl)-1-tosyl-1H-pyrrolo[2,3- b]pyridin-5- yl)benzyl)piperidin-3-ol | C21, D1 | 559.2 [M + H] |
| E24 | 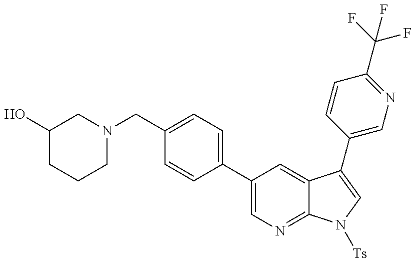 [SMILES: Cc1ccc(S(=O)(=O)n2cc(-c3ccc(C(F)(F)F)nc3)c3cc(-c4ccc(CN5CCCC(O)C5)cc4)cnc32)cc1] | 1-(4-(1-tosyl-3-(6- (trifluoromethyl)pyridin-3- yl)-1H-pyrrolo[2,3- b]pyridin-5- yl)benzyl)piperidin-3-ol | C22, D1 | 607.0 [M + H] |
| E25 | 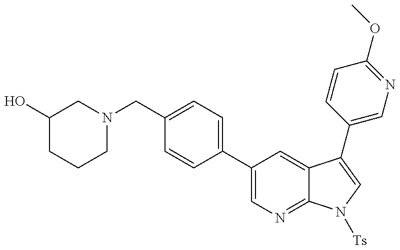 [SMILES: COc1ccc(-c2cn(S(=O)(=O)c3ccc(C)cc3)c3ncc(-c4ccc(CN5CCCC(O)C5)cc4)cc23)cn1] | 1-(4-(3-(6-methoxypyridin- 3-yl)-1-tosyl-1H- pyrrolo[2,3-b]pyridin-5- yl)benzyl)piperidin-3-ol | C23, D1 | 569.3 [M + H] |
| E26 | 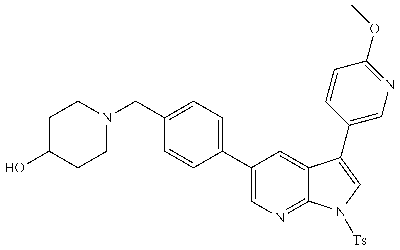 [SMILES: COc1ccc(-c2cn(S(=O)(=O)c3ccc(C)cc3)c3ncc(-c4ccc(CN5CCC(O)CC5)cc4)cc23)cn1] | 1-(4-(3-(6-methoxypyridin- 3-yl)-1-tosyl-1H- pyrrolo[2,3-b]pyridin-5- yl)benzyl)piperidin-4-ol | C23, D2 | 569.3 [M + H] |
| E27 | 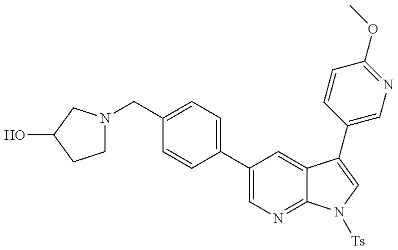 [SMILES: COc1ccc(-c2cn(S(=O)(=O)c3ccc(C)cc3)c3ncc(-c4ccc(CN5CCC(O)C5)cc4)cc23)cn1] | 1-(4-(3-(6-methoxypyridin- 3-yl)-1-tosyl-1H- pyrrolo[2,3-b]pyridin-5- yl)benzyl)pyrrolidin-3-ol | C23, D3 | 555.3 [M + H] |
| E28 | 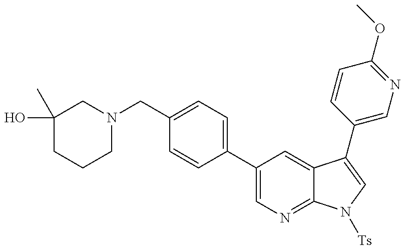 [SMILES: COc1ccc(-c2cn(S(=O)(=O)c3ccc(C)cc3)c3ncc(-c4ccc(CN5CCCC(C)(O)C5)cc4)cc23)cn1] | 1-(4-(3-(6-methoxypyridin- 3-yl)-1-tosyl-1H- pyrrolo[2,3-b]pyridin-5- yl)benzyl)-3- methylpiperidin-3-ol | C23, D5 | 583.3 [M + H] |
| E29 | 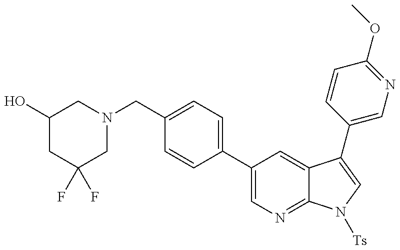 [SMILES: COc1ccc(-c2cn(S(=O)(=O)c3ccc(C)cc3)c3ncc(-c4ccc(CN5CC(O)CC(F)(F)C5)cc4)cc23)cn1] | 5,5-difluoro-1-(4-(3-(6- methoxypyridin-3-yl)-1- tosyl-1H-pyrrolo[2,3- b]pyridin-5- yl)benzyl)piperidin-3-ol | C23, D6 | 605.3 [M + H] |
| E30 | 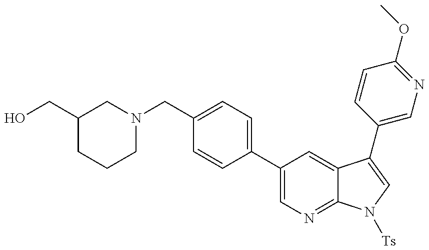 [SMILES: COc1ccc(-c2cn(S(=O)(=O)c3ccc(C)cc3)c3ncc(-c4ccc(CN5CCCC(CO)C5)cc4)cc23)cn1] | (1-(4-(3-(6- methoxypyridin-3-yl)-1- tosyl-1H-pyrrolo[2,3- b]pyridin-5- yl)benzyl)piperidin-3- yl)methanol | C23, D7 | 583.3 [M + H] |
| E31 | 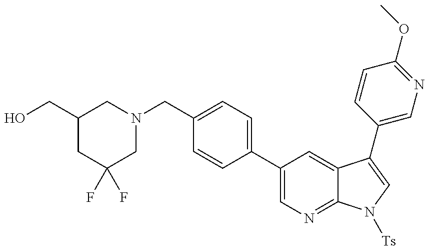 [SMILES: COc1ccc(-c2cn(S(=O)(=O)c3ccc(C)cc3)c3ncc(-c4ccc(CN5CC(CO)CC(F)(F)C5)cc4)cc23)cn1] | (5,5-difluoro-1-(4-(3-(6- methoxypyridin-3-yl)-1- tosyl-1H-pyrrolo[2,3- b]pyridin-5- yl)benzyl)piperidin-3- yl)methanol | C23, D8 | 619.2 [M + H] |
| E32 | 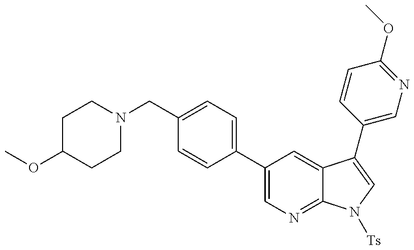 [SMILES: COc1ccc(-c2cn(S(=O)(=O)c3ccc(C)cc3)c3ncc(-c4ccc(CN5CCC(OC)CC5)cc4)cc23)cn1] | 5-(4-((4-methoxypiperidin- 1-yl)methyl)phenyl)-3-(6- methoxypyridin-3-yl)-1- tosyl-1H-pyrrolo[2,3- b]pyridine | C23, D9 | 583.3 [M + H] |
| E33 | 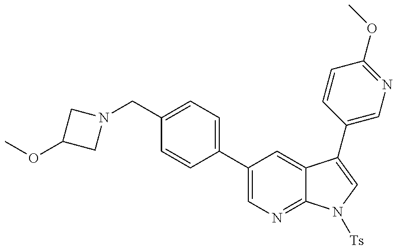 [SMILES: COc1ccc(-c2cn(S(=O)(=O)c3ccc(C)cc3)c3ncc(-c4ccc(CN5CC(OC)C5)cc4)cc23)cn1] | 5-(4-((3-methoxyazetidin- 1-yl)methyl)phenyl)-3-(6- methoxypyridin-3-yl)-1- tosyl-1H-pyrrolo[2,3- b]pyridine | C23, D10 | 555.3 [M + H] |
| E34 | 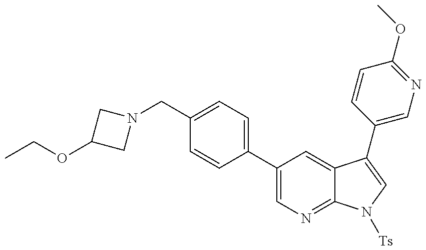 [SMILES: CCOC1CN(Cc2ccc(-c3cnc4c(c3)c(-c3ccc(OC)nc3)cn4S(=O)(=O)c3ccc(C)cc3)cc2)C1] | 5-(4-((3-ethoxyazetidin-1- yl)methyl)phenyl)-3-(6- methoxypyridin-3-yl)-1- tosyl-1H-pyrrolo[2,3- b]pyridine | C23, D11 | 569.2 [M + H] |
| E35 | 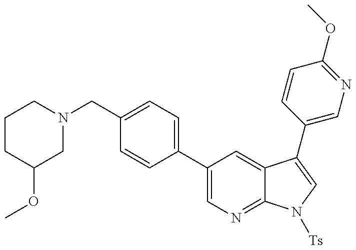 [SMILES: COc1ccc(-c2cn(S(=O)(=O)c3ccc(C)cc3)c3ncc(-c4ccc(CN5CCCC(OC)C5)cc4)cc23)cn1] | 5-(4-((3-methoxypiperidin- 1-yl)methyl)phenyl)-3-(6- methoxypyridin-3-yl)-1- tosyl-1H-pyrrolo[2,3- b]pyridine Racemate | C23, D12 | 583.2 [M + H] |
| E36 | 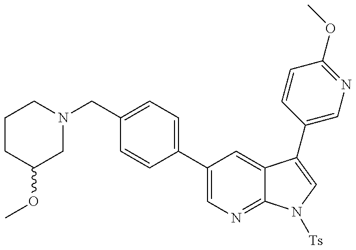 [SMILES: COc1ccc(-c2cn(S(=O)(=O)c3ccc(C)cc3)c3ncc(-c4ccc(CN5CCCC(OC)C5)cc4)cc23)cn1] | 5-(4-((3-methoxypiperidin- 1-yl)methyl)phenyl)-3-(6- methoxypyridin-3-yl)-1- tosyl-1H-pyrrolo[2,3- b]pyridine Enantiomer 1 | C23, D12 | 583.2 [M + H] |
| E37 | 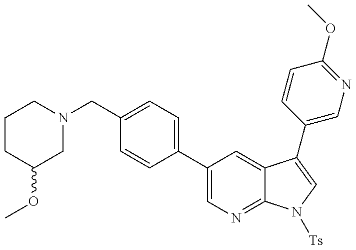 [SMILES: COc1ccc(-c2cn(S(=O)(=O)c3ccc(C)cc3)c3ncc(-c4ccc(CN5CCCC(OC)C5)cc4)cc23)cn1] | 5-(4-((3-methoxypiperidin- 1-yl)methyl)phenyl)-3-(6- methoxypyridin-3-yl)-1- tosyl-1H-pyrrolo[2,3- b]pyridine Enantiomer 2 | C23, D12 | 583.3 [M + H] |
| E38 | 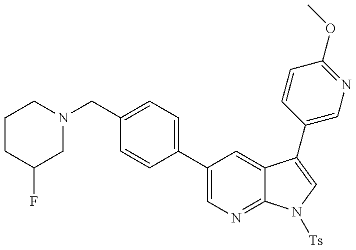 [SMILES: COc1ccc(-c2cn(S(=O)(=O)c3ccc(C)cc3)c3ncc(-c4ccc(CN5CCCC(F)C5)cc4)cc23)cn1] | 5-(4-((3-fluoropiperidin-1- yl)methyl)phenyl)-3-(6- methoxypyridin-3-yl)-1- tosyl-1H-pyrrolo[2,3- b]pyridine | C23, D14 | 571.2 [M + H] |
| E39 | 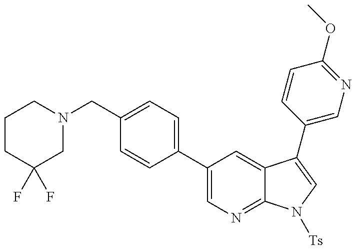 [SMILES: COc1ccc(-c2cn(S(=O)(=O)c3ccc(C)cc3)c3ncc(-c4ccc(CN5CCCC(F)(F)C5)cc4)cc23)cn1] | 5-(4-((3,3- difluoropiperidin-1- yl)methyl)phenyl)-3-(6- methoxypyridin-3-yl)-1- tosyl-1H-pyrrolo[2,3- b]pyridine | C23, D15 | 589.2 [M + H] |
| E40 | 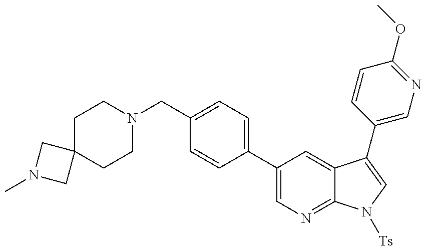 [SMILES: COc1ccc(-c2cn(S(=O)(=O)c3ccc(C)cc3)c3ncc(-c4ccc(CN5CCC6(CC5)CN(C)C6)cc4)cc23)cn1] | 7-(4-(3-(6- methoxypyridin-3-yl)-1- tosyl-1H-pyrrolo[2,3- b]pyridin-5-yl)benzyl)-2- methyl-2,7- diazaspiro[3.5]nonane | C23, D16 | 608.2 [M + H] |
| E41 | 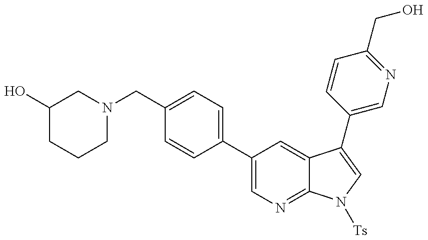 [SMILES: Cc1ccc(S(=O)(=O)n2cc(-c3ccc(CO)nc3)c3cc(-c4ccc(CN5CCCC(O)C5)cc4)cnc32)cc1] | 1-(4-(3-(6- (hydroxymethyl)pyridin- 3-yl)-1-tosyl-1H- pyrrolo[2,3-b]pyridin-5- yl)benzyl)piperidin-3-ol | C24, D1 | 569.1 [M + H] |
| E42 | 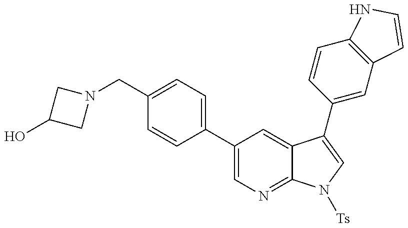 [SMILES: Cc1ccc(S(=O)(=O)n2cc(-c3ccc4[nH]ccc4c3)c3cc(-c4ccc(CN5CC(O)C5)cc4)cnc32)cc1] | 1-(4-(3-(1H-indol-5-yl)-1- tosyl-1H-pyrrolo[2,3- b]pyridin-5- yl)benzyl)azetidin-3-ol | C25, D4 | 549.2 [M + H] |
| E43 | 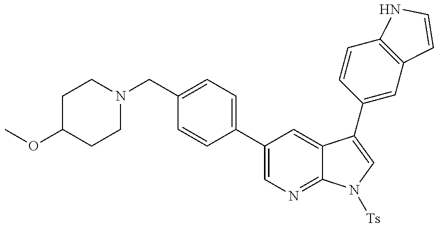 [SMILES: COC1CCN(Cc2ccc(-c3cnc4c(c3)c(-c3ccc5[nH]ccc5c3)cn4S(=O)(=O)c3ccc(C)cc3)cc2)CC1] | 3-(1H-indol-5-yl)-5-(4- ((4-methoxypiperidin-1- yl)methyl)phenyl)-1-tosyl- 1H-pyrrolo[2,3-b]pyridine | C25, D9 | 591.2 [M + H] |
| E44 | 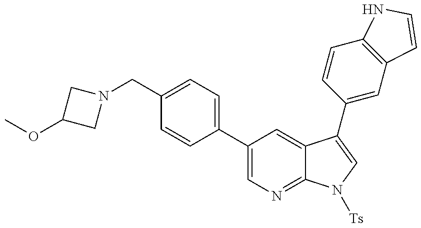 [SMILES: COC1CN(Cc2ccc(-c3cnc4c(c3)c(-c3ccc5[nH]ccc5c3)cn4S(=O)(=O)c3ccc(C)cc3)cc2)C1] | 3-(1H-indol-5-yl)-5-(4- ((3-methoxyazetidin-1- yl)methyl)phenyl)-1-tosyl- 1H-pyrrolo[2,3-b]pyridine | C25, D10 | 563.3 [M + H] |
| E45 | 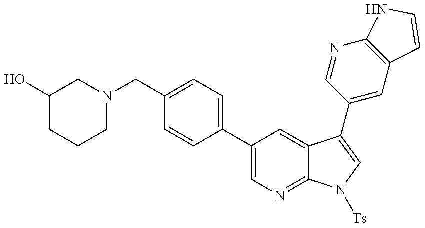 [SMILES: Cc1ccc(S(=O)(=O)n2cc(-c3cnc4[nH]ccc4c3)c3cc(-c4ccc(CN5CCCC(O)C5)cc4)cnc32)cc1] | 1-(4-(1-tosyl-1H,1′H- [3,5'-bipyrrolo[2,3- b]pyridin]-5- yl)benzyl)piperidin-3-ol | C26, D1 | 578.2 [M + H] |
| E46 | 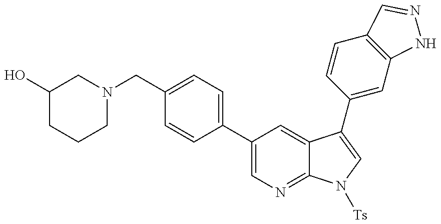 [SMILES: Cc1ccc(S(=O)(=O)n2cc(-c3ccc4cn[nH]c4c3)c3cc(-c4ccc(CN5CCCC(O)C5)cc4)cnc32)cc1] | 1-(4-(3-(1H-indazol-6-yl)- 1-tosyl-1H-pyrrolo[2,3- b]pyridin-5- yl)benzyl)piperidin-3-ol | C27, D1 | 578.2 [M + H] |
| E47 | 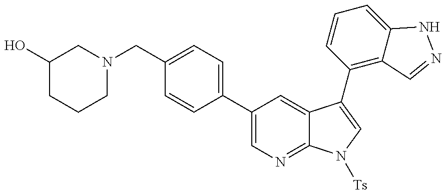 [SMILES: Cc1ccc(S(=O)(=O)n2cc(-c3cccc4[nH]ncc34)c3cc(-c4ccc(CN5CCCC(O)C5)cc4)cnc32)cc1] | 1-(4-(3-(1H-indazol-4-yl)- 1-tosyl-1H-pyrrolo[2,3- b]pyridin-5- yl)benzyl)piperidin-3-ol | C28, D1 | 578.3 [M + H] |
| E48 | 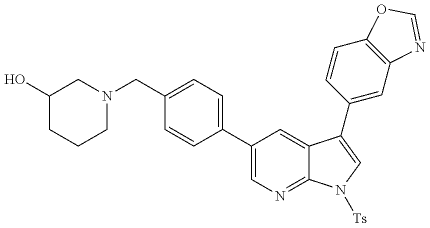 [SMILES: Cc1ccc(S(=O)(=O)n2cc(-c3ccc4ocnc4c3)c3cc(-c4ccc(CN5CCCC(O)C5)cc4)cnc32)cc1] | 1-(4-(3-(benzo[d]oxazol- 5-yl)-1-tosyl-1H- pyrrolo[2,3-b]pyridin-5- yl)benzyl)piperidin-3-ol | C29, D1 | 579.2 [M + H] |
| E49 | 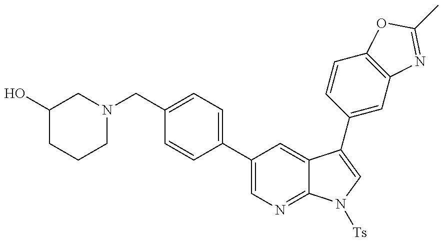 [SMILES: Cc1ccc(S(=O)(=O)n2cc(-c3ccc4oc(C)nc4c3)c3cc(-c4ccc(CN5CCCC(O)C5)cc4)cnc32)cc1] | 1-(4-(3-(2- methylbenzo[d]oxazol-5- yl)-1-tosyl-1H- pyrrolo[2,3-b]pyridin-5- yl)benzyl)piperidin-3-ol | C30, D1 | 593.3 [M + H] |
| E50 | 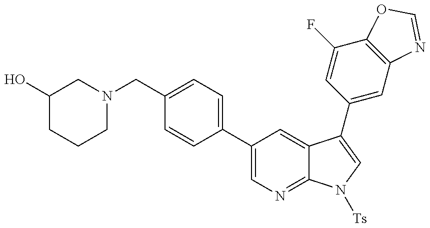 [SMILES: Cc1ccc(S(=O)(=O)n2cc(-c3cc(F)c4ocnc4c3)c3cc(-c4ccc(CN5CCCC(O)C5)cc4)cnc32)cc1] | 1-(4-(3-(7- fluorobenzo[d]oxazol-5- yl)-1-tosyl-1H- pyrrolo[2,3-b]pyridin-5- yl)benzyl)piperidin-3-ol | C31, D1 | 597.1 [M + H] |
| E51 | 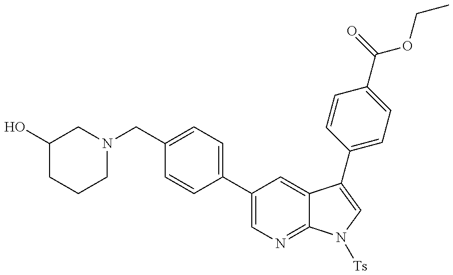 [SMILES: CCOC(=O)c1ccc(-c2cn(S(=O)(=O)c3ccc(C)cc3)c3ncc(-c4ccc(CN5CCCC(O)C5)cc4)cc23)cc1] | ethyl 4-(5-(4-((3- hydroxypiperidin-1- yl)methyl)phenyl)-1-tosyl- 1H-pyrrolo[2,3-b]pyridin- 3-yl)benzoate | C33, D1 | 610.3 [M + H] |
| E52 | 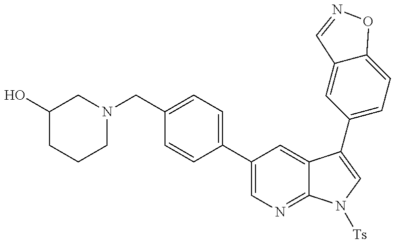 [SMILES: Cc1ccc(S(=O)(=O)n2cc(-c3ccc4oncc4c3)c3cc(-c4ccc(CN5CCCC(O)C5)cc4)cnc32)cc1] | 1-(4-(3-(benzo[d]isoxazol- 5-yl)-1-tosyl-1H- pyrrolo[2,3-b]pyridin-5- yl)benzyl)piperidin-3-ol | C36, D1 | 579.4 [M + H] |
| E53 | 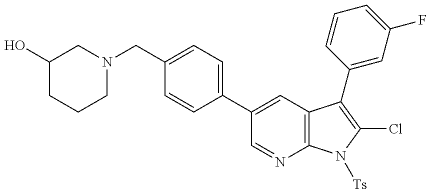 [SMILES: Cc1ccc(S(=O)(=O)n2c(Cl)c(-c3cccc(F)c3)c3cc(-c4ccc(CN5CCCC(O)C5)cc4)cnc32)cc1] | 1-(4-(2-chloro-3-(3- fluorophenyl)-1-tosyl-1H- pyrrolo[2,3-b]pyridin-5- yl)benzyl)piperidin-3-ol | C37, D1 | 590.2 [M + H] |
| E54 | 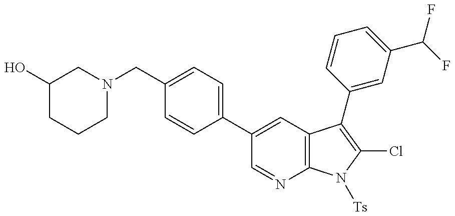 [SMILES: Cc1ccc(S(=O)(=O)n2c(Cl)c(-c3cccc(C(F)F)c3)c3cc(-c4ccc(CN5CCCC(O)C5)cc4)cnc32)cc1] | 1-(4-(2-chloro-3-(3- (difluoromethyl)phenyl)- 1-tosyl-1H-pyrrolo[2,3- b]pyridin-5- yl)benzyl)piperidin-3-ol | C38, D1 | 622.3 [M + H] |
| E55 | 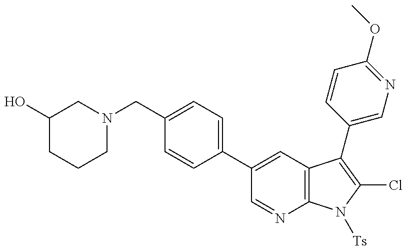 [SMILES: COc1ccc(-c2c(Cl)n(S(=O)(=O)c3ccc(C)cc3)c3ncc(-c4ccc(CN5CCCC(O)C5)cc4)cc23)cn1] | 1-(4-(2-chloro-3-(6- methoxypyridin-3-yl)-1- tosyl-1H-pyrrolo[2,3- b]pyridin-5- yl)benzyl)piperidin-3-ol | C39, D1 | 603.3 [M + H] |
| E56 | 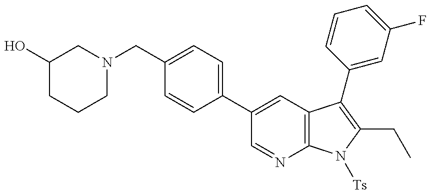 [SMILES: CCc1c(-c2cccc(F)c2)c2cc(-c3ccc(CN4CCCC(O)C4)cc3)cnc2n1S(=O)(=O)c1ccc(C)cc1] | 1-(4-(2-ethyl-3-(3- fluorophenyl)-1-tosyl-1H- pyrrolo[2,3-b]pyridin-5- yl)benzyl)piperidin-3-ol | C40, D1 | 584.4 [M + H] |
| E57 | 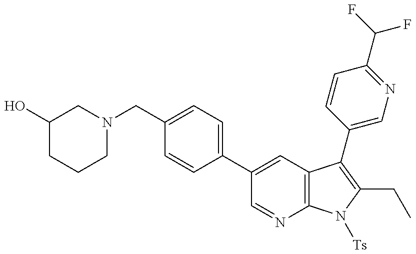 [SMILES: CCc1c(-c2ccc(C(F)F)nc2)c2cc(-c3ccc(CN4CCCC(O)C4)cc3)cnc2n1S(=O)(=O)c1ccc(C)cc1] | 1-(4-(3-(6- (difluoromethyl)pyridin-3- yl)-2-ethyl-1-tosyl-1H- pyrrolo[2,3-b]pyridin-5- yl)benzyl)piperidin-3-ol | C41, D1 | 617.2 [M + H] |
| E58 | 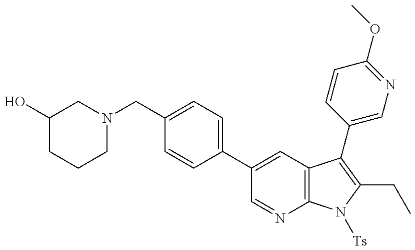 [SMILES: CCc1c(-c2ccc(OC)nc2)c2cc(-c3ccc(CN4CCCC(O)C4)cc3)cnc2n1S(=O)(=O)c1ccc(C)cc1] | 1-(4-(2-ethyl-3-(6- methoxypyridin-3-yl)-1- tosyl-1H-pyrrolo[2,3- b]pyridin-5- yl)benzyl)piperidin-3-ol | C42, D1 | 597.3 [M + H] |
| E59 | 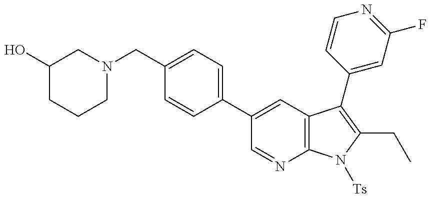 [SMILES: CCc1c(-c2ccnc(F)c2)c2cc(-c3ccc(CN4CCCC(O)C4)cc3)cnc2n1S(=O)(=O)c1ccc(C)cc1] | 1-(4-(2-ethyl-3-(2- fluoropyridin-4-yl)-1- tosyl-1H-pyrrolo[2,3- b]pyridin-5- yl)benzyl)piperidin-3-ol | C43, D1 | 585.2 [M + H] |
| E60 | 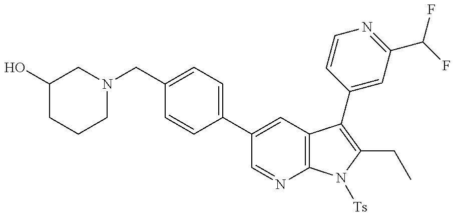 [SMILES: CCc1c(-c2ccnc(C(F)F)c2)c2cc(-c3ccc(CN4CCCC(O)C4)cc3)cnc2n1S(=O)(=O)c1ccc(C)cc1] | 1-(4-(3-(2- (difluoromethyl)pyridin-4- yl)-2-ethyl-1-tosyl-1H- pyrrolo[2,3-b]pyridin-5- yl)benzyl)piperidin-3-ol | C44, D1 | 617.1 [M + H] |
| E61 | 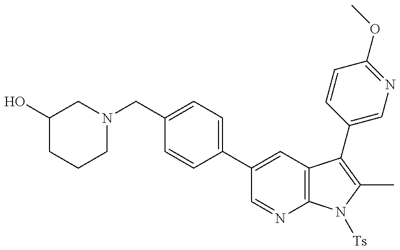 [SMILES: COc1ccc(-c2c(C)n(S(=O)(=O)c3ccc(C)cc3)c3ncc(-c4ccc(CN5CCCC(O)C5)cc4)cc23)cn1] | 1-(4-(2-methyl-3-(6- methoxypyridin-3-yl)-1- tosyl-1H-pyrrolo[2,3- b]pyridin-5- yl)benzyl)piperidin-3-ol | C45, D1 | 583.3 [M + H] |
| E62 | 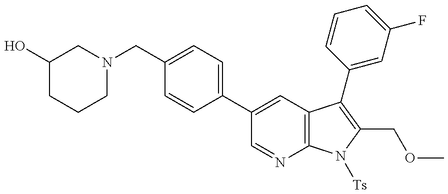 [SMILES: COCc1c(-c2cccc(F)c2)c2cc(-c3ccc(CN4CCCC(O)C4)cc3)cnc2n1S(=O)(=O)c1ccc(C)cc1] | 1-(4-(3-(3-fluorophenyl)- 2-(methoxymethyl)-1- tosyl-1H-pyrrolo[2,3- b]pyridin-5- yl)benzyl)piperidin-3-ol | C46, D1 | 600.3 [M + H] |
| E63 | 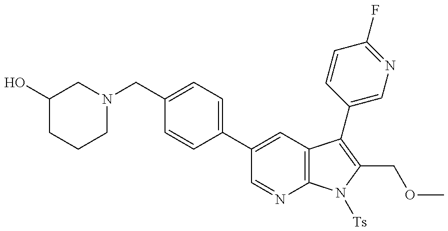 [SMILES: COCc1c(-c2ccc(F)nc2)c2cc(-c3ccc(CN4CCCC(O)C4)cc3)cnc2n1S(=O)(=O)c1ccc(C)cc1] | 1-(4-(3-(6-fluoropyridin- 3-yl)-2-(methoxymethyl)- 1-tosyl-1H-pyrrolo[2,3- b]pyridin-5- yl)benzyl)piperidin-3-ol | C47, D1 | 601.2 [M + H] |
| E64 | 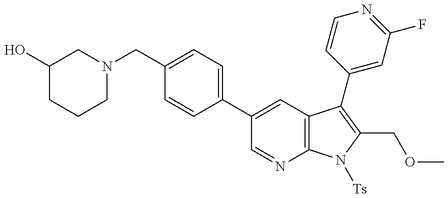 [SMILES: COCc1c(-c2ccnc(F)c2)c2cc(-c3ccc(CN4CCCC(O)C4)cc3)cnc2n1S(=O)(=O)c1ccc(C)cc1] | 1-(4-(3-(2-fluoropyridin- 4-yl)-2-(methoxymethyl)- 1-tosyl-1H-pyrrolo[2,3- b]pyridin-5- yl)benzyl)piperidin-3-ol | C48, D1 | 601.2 [M + H] |
| E65 | 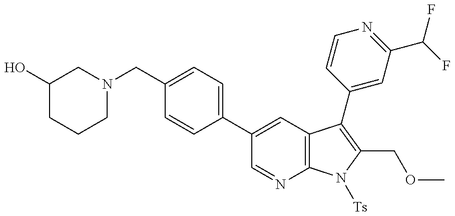 [SMILES: COCc1c(-c2ccnc(C(F)F)c2)c2cc(-c3ccc(CN4CCCC(O)C4)cc3)cnc2n1S(=O)(=O)c1ccc(C)cc1] | 1-(4-(3-(2- (difluoromethyl)pyridin-4- yl)-2-(methoxymethyl)-1- tosyl-1H-pyrrolo[2,3- b]pyridin-5- yl)benzyl)piperidin-3-ol | C49, D1 | 633.3 [M + H] |
| E66 | 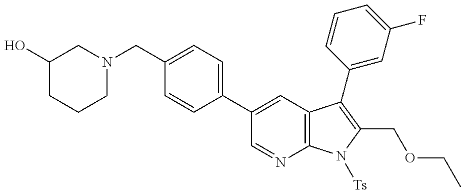 [SMILES: CCOCc1c(-c2cccc(F)c2)c2cc(-c3ccc(CN4CCCC(O)C4)cc3)cnc2n1S(=O)(=O)c1ccc(C)cc1] | 1-(4-(2-(ethoxymethyl)-3- (3-fluorophenyl)-1-tosyl- 1H-pyrrolo[2,3-b]pyridin- 5-yl)benzyl)piperidin-3-ol | C50, D1 | 614.3 [M + H] |
| E67 | 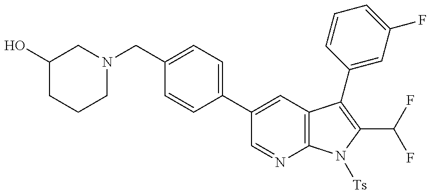 [SMILES: Cc1ccc(S(=O)(=O)n2c(C(F)F)c(-c3cccc(F)c3)c3cc(-c4ccc(CN5CCCC(O)C5)cc4)cnc32)cc1] | 1-(4-(2-(difluoromethyl)- 3-(3-fluorophenyl)-1- tosyl-1H-pyrrolo[2,3- b]pyridin-5- yl)benzyl)piperidin-3-ol | C51, D1 | 606.2 [M + H] |
| E68 | 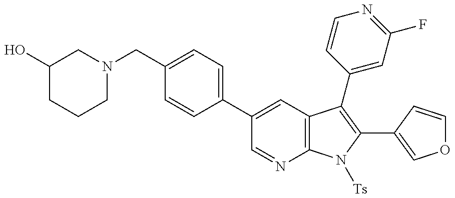 [SMILES: Cc1ccc(S(=O)(=O)n2c(-c3ccoc3)c(-c3ccnc(F)c3)c3cc(-c4ccc(CN5CCCC(O)C5)cc4)cnc32)cc1] | 1-(4-(3-(2-fluoropyridin- 4-yl)-2-(furan-3-yl)-1- tosyl-1H-pyrrolo[2,3- b]pyridin-5- yl)benzyl)piperidin-3-ol | C52, D1 | 569.3 [M + H] |

Intermediates E36 and E37 were obtained by subjecting Intermediate E35 to chiral preparative HPLC.

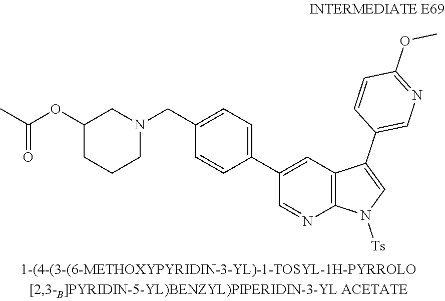 [SMILES: COc1ccc(-c2cn(S(=O)(=O)c3ccc(C)cc3)c3ncc(-c4ccc(CN5CCCC(OC(C)=O)C5)cc4)cc23)cn1]

Acetic anhydride (0.133 mL, 1.41 mmol) was added to a solution of 1-(4-(3-(6-methoxypyridin-3-yl)-1-tosyl-1H-pyrrolo[2,3-b]pyridin-5-yl)benzyl)piperidin-3-ol (E25, 0.4 g, 0.703 mmol) in pyridine (5 mL) and the resulting mixture was stirred at 25° C. for 16 h. Following completion of the reaction, the mixture was diluted with EtOAc (20 mL) and washed with 2N HCl solution (10 mL). The organic phase was washed with brine (5 mL), dried over anhydrous Na₂SO₄, filtered, and concentrated under reduced pressure to give crude material which was purified by flash chromatography (eluting with MeOH in DCM), affording the title compound (0.35 g, 81% yield) as an off-white solid. ¹H NMR (400 MHz, DMSO-d₆) δ=8.70 (s, 1H), 8.65 (s, 1H), 8.38 (s, 1H), 8.20-8.18 (m, 1H), 8.06-8.04 (m, 2H), 7.73-7.71 (m, 2H), 7.43-7.37 (m, 4H), 6.94-6.92 (m, 1H), 4.66 (s, 1H), 3.90 (s, 3H), 3.56-3.48 (m, 2H), 2.77-2.75 (m, 1H), 2.57-2.55 (m, 1H), 2.33 (s, 3H), 2.07-2.05 (m, 2H), 1.96 (s, 3H), 1.80-1.77 (m, 1H), 1.65-1.63 (m, 1H), 1.49-1.47 (m, 1H), 1.32-1.30 (m, 1H). LC/MS: 611.3 [M+H].

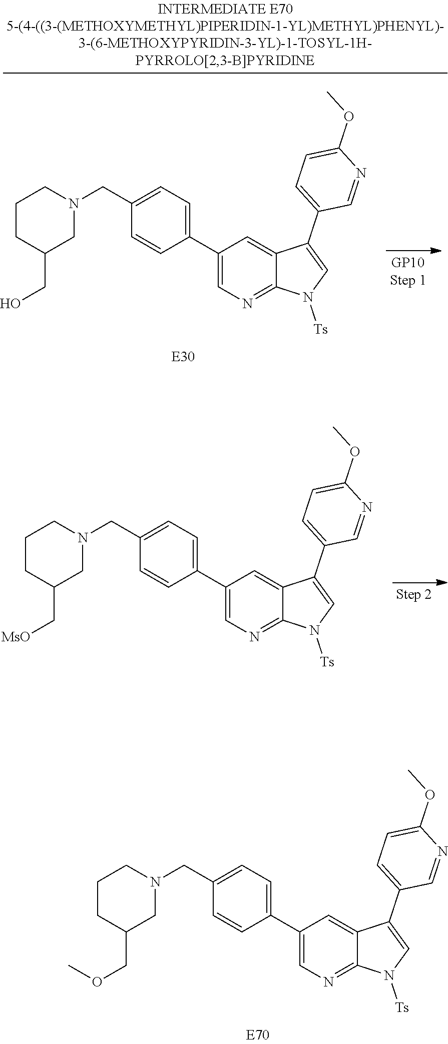 [SMILES: COc1ccc(-c2cn(S(=O)(=O)c3ccc(C)cc3)c3ncc(-c4ccc(CN5CCCC(CO)C5)cc4)cc23)cn1 ; COc1ccc(-c2cn(S(=O)(=O)c3ccc(C)cc3)c3ncc(-c4ccc(CN5CCCC(COS(C)(=O)=O)C5)cc4)cc23)cn1 ; COCC1CCCN(Cc2ccc(-c3cnc4c(c3)c(-c3ccc(OC)nc3)cn4S(=O)(=O)c3ccc(C)cc3)cc2)C1]

### Step 1: Synthesis of (1-(4-(3-(6-methoxypyridin-3-yl)-1-tosyl-1H-pyrrolo[2,3-b]pyridin-5-yl)benzyl)piperidin-3-yl)methyl methanesulfonate

The title compound was synthesized by following General Procedure GP10, starting from (1-(4-(3-(6-methoxypyridin-3-yl)-1-tosyl-1H-pyrrolo[2,3-b]pyridin-5-yl)benzyl)piperidin-3-yl)methanol (E30, 0.25 g, 0.429 mmol), and was obtained as an off-white solid (0.2 g, crude) which was used without further purification. ¹H NMR (400 MHz, DMSO-d₆) δ=8.70-8.65 (m, 2H), 8.38 (s, 1H), 8.27 (s, 1H), 8.19 (d, J=8.0 Hz, 1H), 8.06-8.04 (m, 2H), 7.73-7.71 (m, 2H), 7.43-7.37 (m, 4H), 6.94-6.92 (m, 1H), 4.08 (s, 2H), 3.90 (s, 3H), 3.49 (s, 2H), 3.12 (s, 3H), 2.74-2.63 (m, 2H), 2.33 (s, 3H), 2.00-1.98 (m, 3H), 1.63-1.61 (m, 2H). LC/MS: 661.2 [M+H].

### Step 2: Synthesis of 5-(4-((3-(methoxymethyl)piperidin-1-yl)methyl)phenyl)-3-(6-methoxypyridin-3-yl)-1H-pyrrolo[2,3-b]pyridine

Sodium pieces (0.1 g, 4.348 mmol) were added to a solution of (1-(4-(3-(6-methoxypyridin-3-yl)-1-tosyl-1H-pyrrolo[2,3-b]pyridin-5-yl)benzyl)piperidin-3-yl)methyl methanesulfonate (0.2 g, 0.303 mmol) in MeOH (5 mL) and toluene (5.00 mL) and the resulting mixture was heated to 100° C. for 8 h in a sealed tube. Following completion of the reaction, the mixture was concentrated under reduced pressure, diluted with water (10 mL), and extracted with EtOAc (20 mL). The organic phase was washed with brine (5 mL), dried over anhydrous Na₂SO₄, filtered, and concentrated under reduced pressure to give crude material which was purified by flash chromatography (eluting with MeOH in DCM), affording the title compound (0.03 g, 22% yield) as an off-white solid. ¹H NMR (400 MHz, DMSO-d₆) δ=11.97 (s, 1H), 8.56 (d, J=10.0 Hz, 2H), 8.36 (s, 1H), 8.10-8.08 (m, 1H), 7.86 (s, 1H), 7.70 (d, J=8.0 Hz, 2H), 7.36 (d, J=8.0 Hz, 2H), 6.89-6.87 (s, 1H), 3.90 (s, 3H), 3.48-3.45 (m, 4H), 3.15 (s, 3H), 3.10-3.08 (m, 1H), 2.84-2.80 (m, 2H), 1.93-1.91 (m, 1H), 1.79-1.75 (m, 2H), 1.68-1.65 (m, 2H), 1.48-1.45 (m, 1H). LC/MS: 443.3 [M+H].

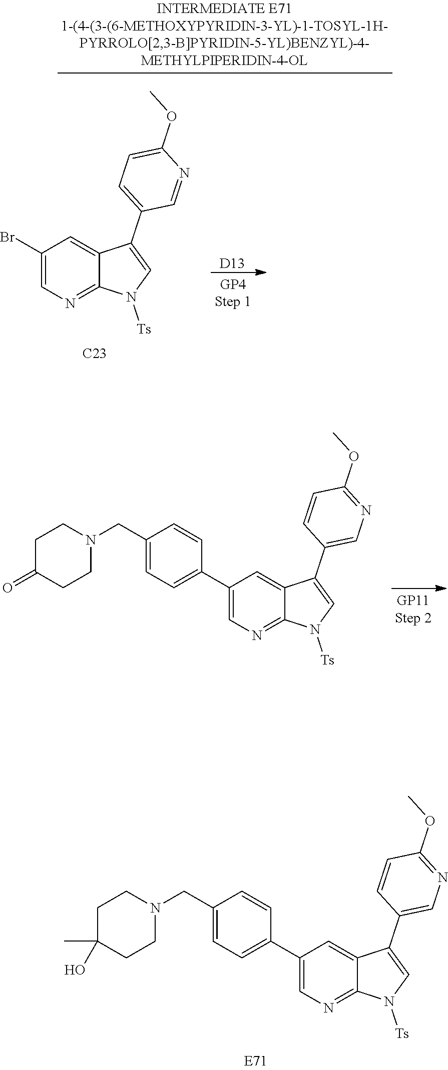 [SMILES: COc1ccc(-c2cn(S(=O)(=O)c3ccc(C)cc3)c3ncc(Br)cc23)cn1 ; "D13" ; "C23" ; COc1ccc(-c2cn(S(=O)(=O)c3ccc(C)cc3)c3ncc(-c4ccc(CN5CCC(=O)CC5)cc4)cc23)cn1 ; COc1ccc(-c2cn(S(=O)(=O)c3ccc(C)cc3)c3ncc(-c4ccc(CN5CCC(C)(O)CC5)cc4)cc23)cn1]

### Step 1: Synthesis of 1-(4-(3-(6-methoxypyridin-3-yl)-1-tosyl-1H-pyrrolo[2,3-b]pyridin-5-yl)benzyl)piperidin-4-one

The title compound was synthesized by following General Procedure GP4, starting from 5-bromo-3-(6-methoxypyridin-3-yl)-1-tosyl-1H-pyrrolo[2,3-b]pyridine (C23, 0.500 g 1.09 mmol) and 1-(4-(4,4,5,5-tetramethyl-1,3,2-dioxaborolan-2-yl)benzyl)piperidin-4-one (D13, 0.344 g 1.09 mmol), and was obtained as a pale brown liquid (0.4 g 65% yield). LC/MS: 567.1 [M+H].

### Step 2: Synthesis of 1-(4-(3-(6-methoxypyridin-3-yl)-1-tosyl-1H-pyrrolo[2,3-b]pyridin-5-yl)benzyl)-4-methylpiperidin-4-ol

The title compound was synthesized by following General Procedure GP 11, starting from 1-(4-(3-(6-methoxypyridin-3-yl)-1-tosyl-1H-pyrrolo[2,3-b]pyridin-5-yl)benzyl)piperidin-4-one (0.2 g, 0.353 mmol), and was obtained as an off-white solid (0.12 g 58% yield). LC/MS: 583.4 [M+H].

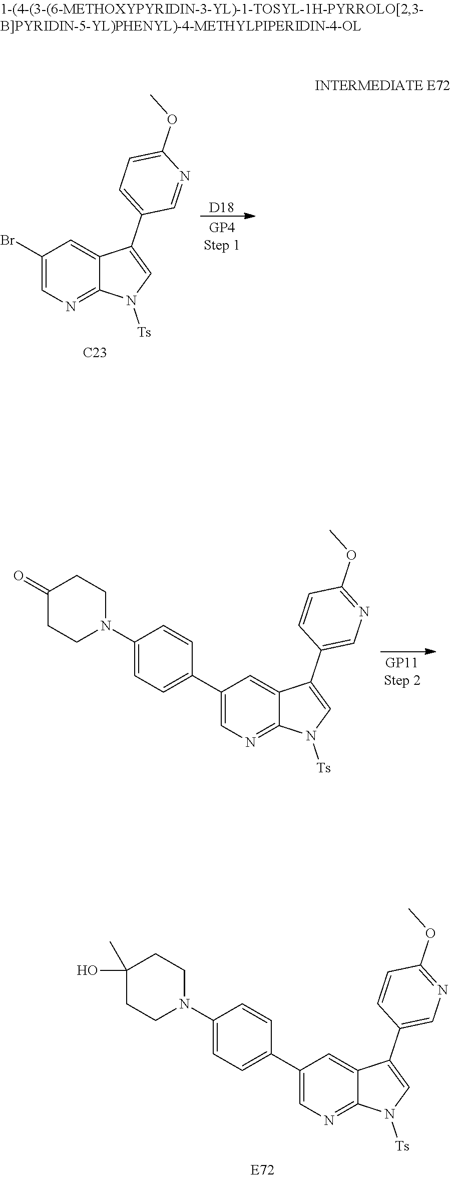

### Step 1: Synthesis of 1-(4-(3-(6-methoxypyridin-3-yl)-1-tosyl-1H-pyrrolo[2,3-b]pyridin-5-yl)phenyl)piperidin-4-one

The title compound was synthesized by following General Procedure GP4, starting from 5-bromo-3-(6-methoxypyridin-3-yl)-1-tosyl-1H-pyrrolo[2,3-b]pyridine (C23, 0.50 g, 1.09 mmol) and 1-(4-(4,4,5,5-tetramethyl-1,3,2-dioxaborolan-2-yl)phenyl)piperidin-4-one (D18, 0.32 g, 1.09 mmol), and was obtained as a brown solid (0.25 g, 41% yield). LC/MS: 553.2 [M+H].

### Step 2: Synthesis of 1-(4-(3-(6-methoxypyridin-3-yl)-1-tosyl-1H-pyrrolo[2,3-b]pyridin-5-yl)phenyl)-4-methylpiperidin-4-ol

The title compound was synthesized by following General Procedure GP 11, starting from 1-(4-(3-(6-methoxypyridin-3-yl)-1-tosyl-1H-pyrrolo[2,3-b]pyridin-5-yl)phenyl)piperidin-4-one (0.25 g, 0.452 mmol), and was obtained as a brown liquid (0.1 g, 39% yield). LC/MS: 569.5 [M+H].

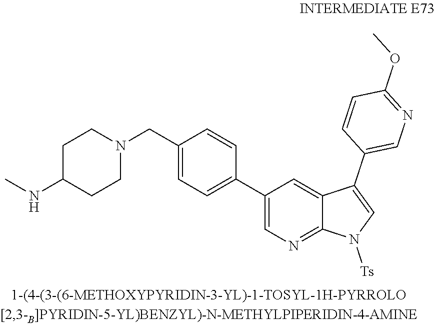 [SMILES: CNC1CCN(Cc2ccc(-c3cnc4c(c3)c(-c3ccc(OC)nc3)cn4S(=O)(=O)c3ccc(C)cc3)cc2)CC1]

The title compound was synthesized by following General Procedure GP9, starting from 1-(4-(3-(6-methoxypyridin-3-yl)-1-tosyl-1H-pyrrolo[2,3-b]pyridin-5-yl)benzyl)piperidin-4-one (step 1 of Intermediate E71, 0.200 g, 0.353 mmol) and methanamine (0.047 g, 0.706 mmol), and was obtained as a brown gum (0.12 g, 60% yield). LC/MS: 582.3 [M+H].

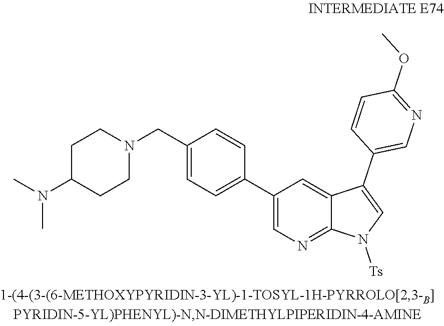 [SMILES: COc1ccc(-c2cn(S(=O)(=O)c3ccc(C)cc3)c3ncc(-c4ccc(CN5CCC(N(C)C)CC5)cc4)cc23)cn1]

The title compound was synthesized by following General Procedure GP9, starting from 1-(4-(3-(6-methoxypyridin-3-yl)-1-tosyl-1H-pyrrolo[2,3-b]pyridin-5-yl)benzyl)piperidin-4-one (step 1 of Intermediate E71, 0.20 g, 0.35 mmol) and dimethylamine (1M in THF, 0.37 mL, 0.37 mmol), and was obtained as an off-white solid (0.1 g, 47% yield). LC/MS: 596.3 [M+H].

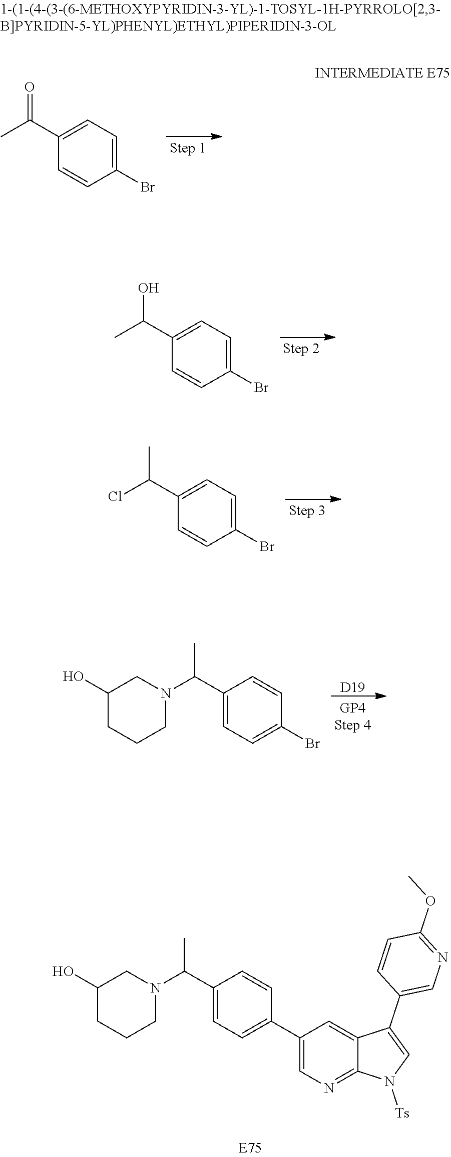

### Step 1: Synthesis of 1-(4-bromophenyl)ethan-1-ol

NaBH₄ (1.80 g, 50.2 mmol) was added to a solution of 1-(4-bromophenyl)ethan-1-one (1.00 g, 5.02 mmol) in MeOH (15 mL) at 0° C. and the resulting mixture was allowed to warm to 25° C. and was stirred for 1 h. Following completion of the reaction (as indicated by LC/MS and TLC), the mixture was concentrated under reduced pressure to give crude material which was purified by flash chromatography (eluting with EtOAc in n-hexane), affording the title compound (0.9 g, 89% yield) as a colorless liquid. ¹H NMR (400 MHz, DMSO-d₆) δ=7.47 (d, J=8.0 Hz, 2H), 7.28 (d, J=8.0 Hz, 2H), 5.23 (s, 1H), 4.67 (d, J=12.0 Hz, 1H), 1.27 (d, J=8.0 Hz, 3H). LC/MS: 201.1 [M+H].

### Step 2: Synthesis of 1-bromo-4-(1-chloroethyl)benzene

Thionyl chloride (0.39 mL, 5.37 mmol) was added to a solution of 1-(4-bromophenyl)ethan-1-ol (0.90 g, 4.48 mmol) in DCM (10 mL) at 0° C. and the resulting mixture was allowed to warm to 25° C. and was stirred for 1 h. Following completion of the reaction (as indicated by TLC), the mixture was concentrated under reduced pressure to give crude material which was triturated with Et₂O, affording the title compound (0.9 g, 69% yield) as a colorless liquid which was used without further purification. ¹H NMR (400 MHz, CDCl₃) δ=7.49-7.47 (m, 2H), 7.30-7.28 (m, 2H), 5.06-5.01 (m, 1H), 1.83-1.81 (d, J=8.0 Hz, 3H). LC/MS: 219.5 [M+H].

### Step 3: Synthesis of 1-(1-(4-bromophenyl)ethyl)piperidin-3-ol

K₂CO₃ (1.70 g, 12.3 mmol) was added to a solution of 1-bromo-4-(1-chloroethyl)benzene (0.900 g, 4.10 mmol) and piperidin-3-ol (0.415 g, 4.10 mmol) in DMF (10 mL) and the resulting mixture was stirred at 25° C. for 16 h. Following completion of the reaction (as indicated by LC/MS and TLC), the mixture was filtered through a pad of diatomaceous earth (i.e., Celite®) which was then rinsed with EtOAc (2×25 mL). The combined filtrates were washed with cold water (2×10 mL) and brine (10 mL), dried over anhydrous Na₂SO₄, filtered, and concentrated under reduced pressure to give crude material which was purified by flash chromatography (eluting with 0 to 10% MeOH in DCM), affording the title compound (0.8 g, 69% yield) as a colorless liquid. LC/MS: 286.1 [M+H].

### Step 4: Synthesis of 1-(1-(4-(3-(6-methoxypyridin-3-yl)-1-tosyl-1H-pyrrolo[2,3-b]pyridin-5-yl)phenyl)ethyl)piperidin-3-ol

The title compound was synthesized by following General Procedure GP4, starting from 1-(1-(4-bromophenyl)ethyl)piperidin-3-ol (0.225 g, 0.791 mmol) and 3-(6-methoxypyridin-3-yl)-5-(4,4,5,5-tetramethyl-1,3,2-dioxaborolan-2-yl)-1-tosyl-1H-pyrrolo[2,3-b]pyridine (D19, 0.400 g, 0.791 mmol), and was obtained as an off-white solid (0.2 g, 43% yield). LC/MS: 583.3 [M+H].

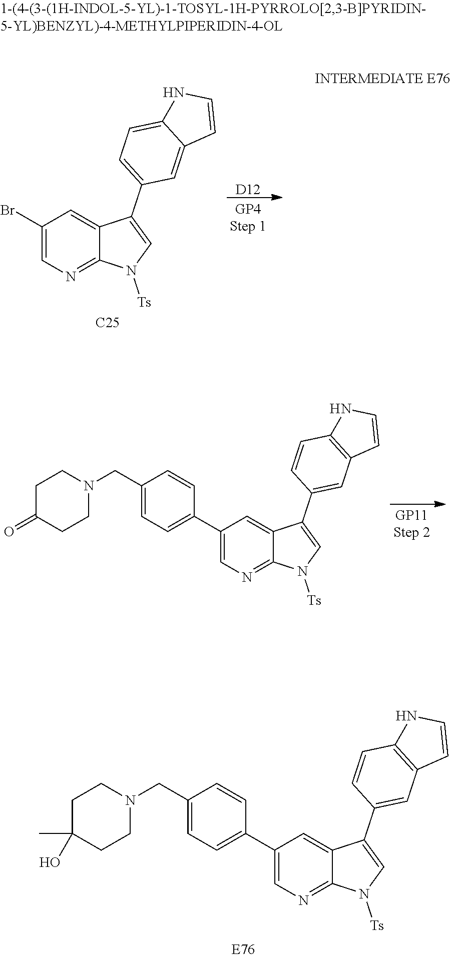 [SMILES: Cc1ccc(S(=O)(=O)n2cc(-c3ccc4[nH]ccc4c3)c3cc(Br)cnc32)cc1 ; "D12" ; "C25" ; Cc1ccc(S(=O)(=O)n2cc(-c3ccc4[nH]ccc4c3)c3cc(-c4ccc(CN5CCC(=O)CC5)cc4)cnc32)cc1 ; Cc1ccc(S(=O)(=O)n2cc(-c3ccc4[nH]ccc4c3)c3cc(-c4ccc(CN5CCC(C)(O)CC5)cc4)cnc32)cc1]

The title compound was synthesized as described for Intermediate E71, using 5-bromo-3-(1H-indol-5-yl)-1-tosyl-1H-pyrrolo[2,3-b]pyridine (C25, 0.5 g, 1.07 mmol) in step 1, and was obtained as an off-white solid (0.1 g, 16% yield over 2 steps). LC/MS: 591.2 [M+H].

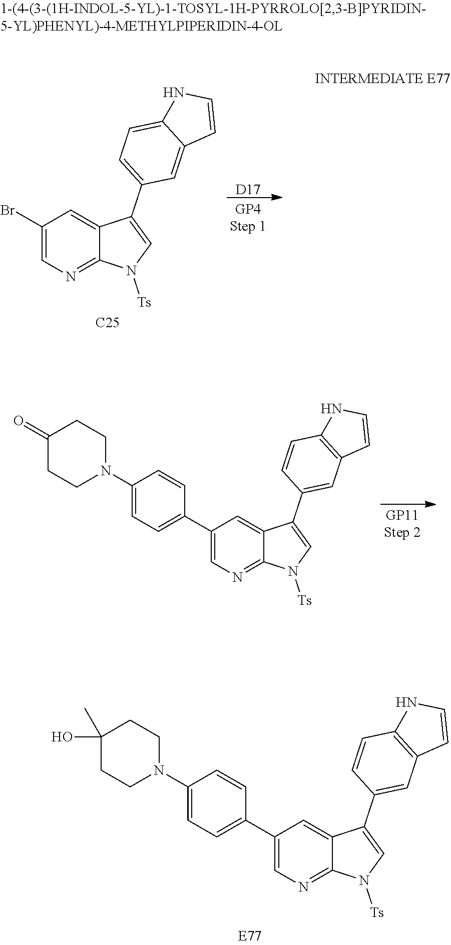 [SMILES: Cc1ccc(S(=O)(=O)n2cc(-c3ccc4[nH]ccc4c3)c3cc(Br)cnc32)cc1 ; "D17" ; "C25" ; Cc1ccc(S(=O)(=O)n2cc(-c3ccc4[nH]ccc4c3)c3cc(-c4ccc(N5CCC(=O)CC5)cc4)cnc32)cc1 ; Cc1ccc(S(=O)(=O)n2cc(-c3ccc4[nH]ccc4c3)c3cc(-c4ccc(N5CCC(C)(O)CC5)cc4)cnc32)cc1]

The title compound was synthesized as described for Intermediate E72, using 5-bromo-3-(1H-indol-5-yl)-1-tosyl-1H-pyrrolo[2,3-b]pyridine (C25, 0.3 g, 0.643 mmol) in step 1, and was obtained as a brown liquid (0.09 g, 24% yield over 2 steps). LC/MS: 577.5 [M+H].

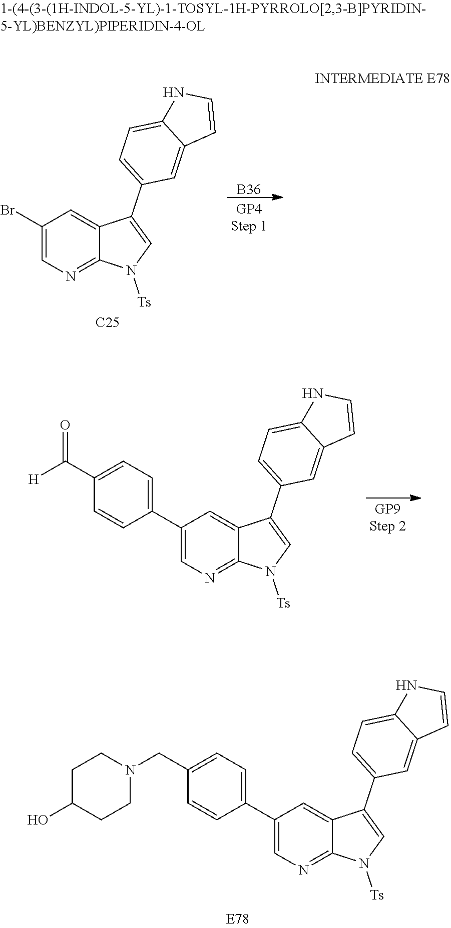 [SMILES: Cc1ccc(S(=O)(=O)n2cc(-c3ccc4[nH]ccc4c3)c3cc(Br)cnc32)cc1 ; "B36" ; "C25" ; Cc1ccc(S(=O)(=O)n2cc(-c3ccc4[nH]ccc4c3)c3cc(-c4ccc(C=O)cc4)cnc32)cc1 ; Cc1ccc(S(=O)(=O)n2cc(-c3ccc4[nH]ccc4c3)c3cc(-c4ccc(CN5CCC(O)CC5)cc4)cnc32)cc1]

### Step 1: Synthesis of 4-(3-(1H-indol-5-yl)-1-tosyl-1H-pyrrolo[2,3-b]pyridin-5-yl)benzaldehyde

The title compound was synthesized by following General Procedure GP4, starting from 5-bromo-3-(1H-indol-5-yl)-1-tosyl-1H-pyrrolo[2,3-b]pyridine (C25, 0.500 g 1.07 mmol) and 4-(4,4,5,5-tetramethyl-1,3,2-dioxaborolan-2-yl)benzaldehyde (B36, 0.274 g 1.18 mmol), and was obtained as a brown solid (0.26 g, 49% yield). ¹H NMR (400 MHz, DMSO-d₆) δ=11.19 (s, 1H), 10.05 (s, 1H), 8.79 (s, 1H), 8.51 (s, 1H), 8.15-8.11 (m, 3H), 8.04-8.04 (m, 5H), 7.62-7.60 (m, 2H), 7.51-7.49 (m, 3H), 6.50 (s, 1H), 2.33 (s, 3H). LC/MS: 492.3 [M+H].

### Step 2: Synthesis of 1-(4-(3-(1H-indol-5-yl)-1-tosyl-1H-pyrrolo[2,3-b]pyridin-5-yl)benzyl)piperidin-4-ol

The title compound was synthesized by following General Procedure GP9, starting from 4-(3-(1H-indol-5-yl)-1-tosyl-1H-pyrrolo[2,3-b]pyridin-5-yl)benzaldehyde (0.25 g, 0.508 mmol) and piperidin-4-ol (61.6 mg, 0.609 mmol), and was obtained as an off-white solid (0.18 g, 62% yield). ¹H NMR (400 MHz, DMSO-d₆) δ=11.19 (s, 1H), 8.68 (s, 1H), 8.38 (s, 1H), 8.07-8.05 (m, 3H), 7.96 (s, 1H), 7.68-7.66 (m, 2H), 7.60-7.58 (m, 2H), 7.43-7.38 (m, 5H), 6.50 (s, 1H), 4.52 (s, 1H), 4.35-3.43 (m, 3H), 2.65-2.63 (m, 2H), 2.33 (s, 3H), 2.06-2.02 (m, 2H), 1.67-1.65 (m, 2H), 1.37-1.35 (m, 2H). LC/MS: 577.3 [M+H].

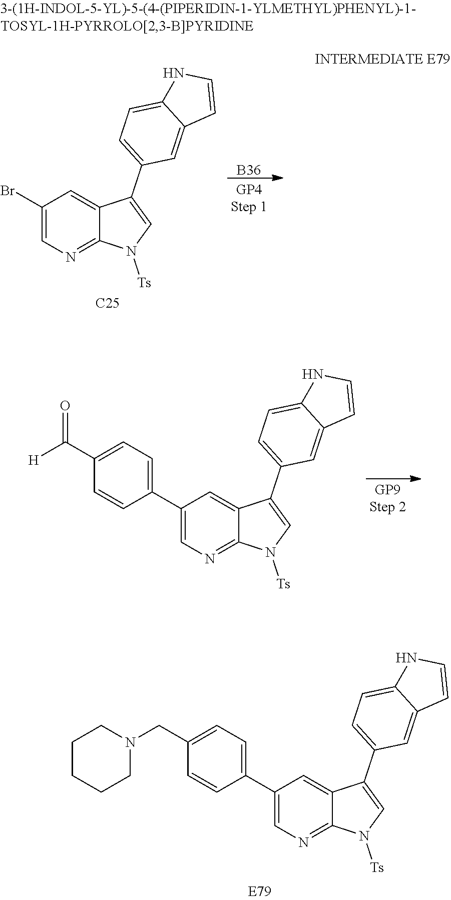 [SMILES: Cc1ccc(S(=O)(=O)n2cc(-c3ccc4[nH]ccc4c3)c3cc(Br)cnc32)cc1 ; "B36" ; "C25" ; Cc1ccc(S(=O)(=O)n2cc(-c3ccc4[nH]ccc4c3)c3cc(-c4ccc(C=O)cc4)cnc32)cc1 ; Cc1ccc(S(=O)(=O)n2cc(-c3ccc4[nH]ccc4c3)c3cc(-c4ccc(CN5CCCCC5)cc4)cnc32)cc1]

The title compound was synthesized as described for Intermediate E78, using 4-(3-(1H-indol-5-yl)-1-tosyl-1H-pyrrolo[2,3-b]pyridin-5-yl)benzaldehyde (0.15 g, 0.30 mmol) and piperidine (0.044 mL, 0.45 mmol) in step 2, and was obtained as a pale-yellow gum (14% yield over 2 steps). LC/MS: 561.4 [M+H].

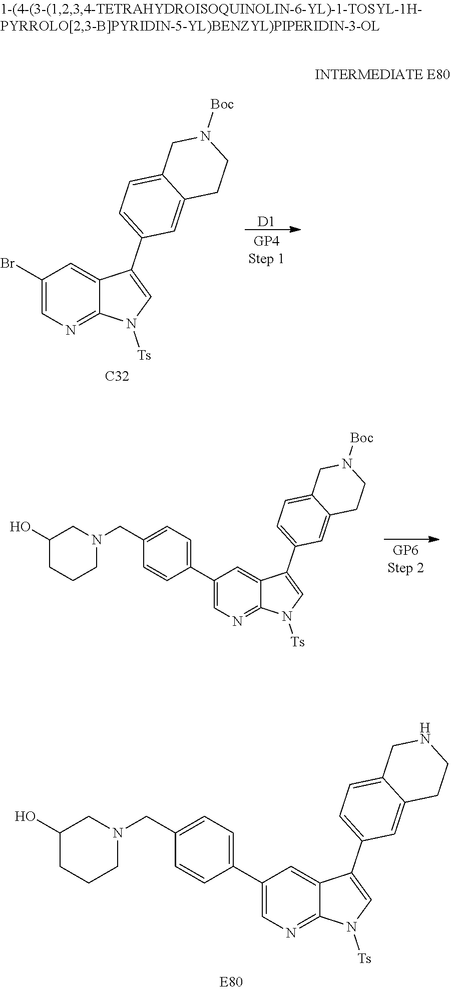 [SMILES: Cc1ccc(S(=O)(=O)n2cc(-c3ccc4c(c3)CCN(C(=O)OC(C)(C)C)C4)c3cc(Br)cnc32)cc1 ; "D1" ; "C32" ; Cc1ccc(S(=O)(=O)n2cc(-c3ccc4c(c3)CCN(C(=O)OC(C)(C)C)C4)c3cc(-c4ccc(CN5CCCC(O)C5)cc4)cnc32)cc1 ; Cc1ccc(S(=O)(=O)n2cc(-c3ccc4c(c3)CCNC4)c3cc(-c4ccc(CN5CCCC(O)C5)cc4)cnc32)cc1]

### Step 1: Synthesis of tert-butyl 6-(5-(4-((3-hydroxypiperidin-1-yl)methyl)phenyl)-1-tosyl-1H-pyrrolo[2,3-b]pyridin-3-yl)-3,4-dihydroisoquinoline-2(1H)-carboxylate

The title compound was synthesized by following General Procedure GP4, starting from tert-butyl 6-(5-bromo-1-tosyl-1H-pyrrolo[2,3-b]pyridin-3-yl)-3,4-dihydroisoquinoline-2(1H)-carboxylate (C32, 0.5 g, 0.858 mmol) and 1-(4-(4,4,5,5-tetramethyl-1,3,2-dioxaborolan-2-yl)benzyl)piperidin-3-ol (D1, 0.3 g, 0.944 mmol) and was obtained as an off-white solid (0.4 g, 67% yield). LC/MS: 693.3 [M+H].

### Step 2: Synthesis of 1-(4-(3-(1,2,3,4-tetrahydroisoquinolin-6-yl)-1-tosyl-1H-pyrrolo[2,3-b]pyridin-5-yl)benzyl)piperidin-3-ol

The title compound was synthesized by following General Procedure GP6, starting from tert-butyl 6-(5-(4-((3-hydroxypiperidin-1-yl)methyl)phenyl)-1-tosyl-1H-pyrrolo[2,3-b]pyridin-3-yl)-3,4-dihydroisoquinoline-2(1H)-carboxylate (0.4 g, 0.577 mmol), and was obtained as an off-white solid (0.280 g, 81% yield). LC/MS: 593.1 [M+H].

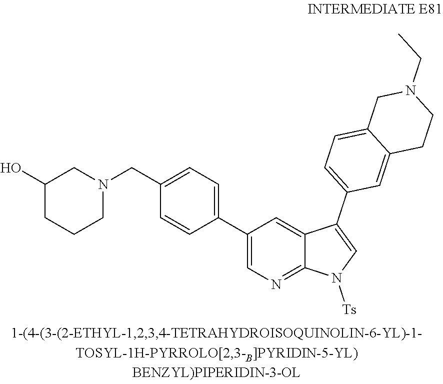 [SMILES: CCN1CCc2cc(-c3cn(S(=O)(=O)c4ccc(C)cc4)c4ncc(-c5ccc(CN6CCCC(O)C6)cc5)cc34)ccc2C1]

Acetaldehyde (0.026 g, 0.607 mmol) and acetic acid (0.3 mL) were added to a solution of 1-(4-(3-(1,2,3,4-tetrahydroisoquinolin-6-yl)-1-tosyl-1H-pyrrolo[2,3-b]pyridin-5-yl)benzyl)piperidin-3-ol (E80, 0.180 g, 0.304 mmol) in methanol (5 mL) and the resulting solution was stirred at 25° C. for 1 h. NaBH₃CN (0.038 g, 0.607 mmol) was then added and stirring was continued for another 4 h. Following completion of the reaction, the mixture was concentrated under reduced pressure, diluted with water (10 mL), and extracted with DCM (30 mL). The organic phase was washed with brine (10 mL), dried over anhydrous Na₂SO₄, filtered, and concentrated under reduced pressure to give crude material which was purified by flash chromatography (eluting with MeOH in DCM), affording the title compound (0.15 g, 79% yield) as a brown solid. LC/MS: 621.3 [M+H].

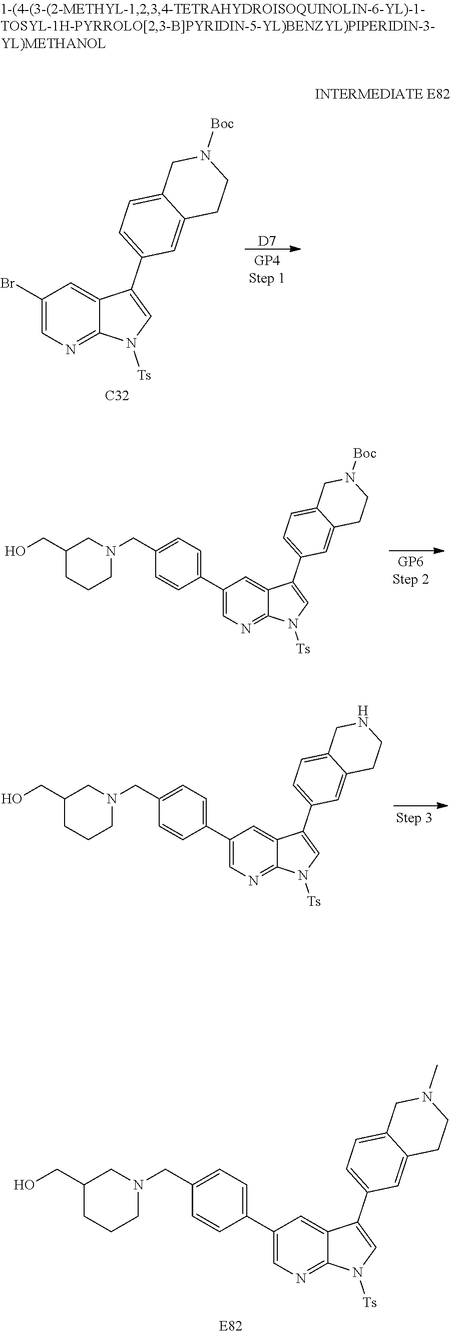 [SMILES: Cc1ccc(S(=O)(=O)n2cc(-c3ccc4c(c3)CCN(C(=O)OC(C)(C)C)C4)c3cc(Br)cnc32)cc1 ; "D7" ; "C32" ; Cc1ccc(S(=O)(=O)n2cc(-c3ccc4c(c3)CCN(C(=O)OC(C)(C)C)C4)c3cc(-c4ccc(CN5CCCC(CO)C5)cc4)cnc32)cc1 ; Cc1ccc(S(=O)(=O)n2cc(-c3ccc4c(c3)CCNC4)c3cc(-c4ccc(CN5CCCC(CO)C5)cc4)cnc32)cc1 ; Cc1ccc(S(=O)(=O)n2cc(-c3ccc4c(c3)CCN(C)C4)c3cc(-c4ccc(CN5CCCC(CO)C5)cc4)cnc32)cc1]

### Step 1: Synthesis of tert-butyl 6-(5-(4-((3-(hydroxymethyl)piperidin-1-yl)methyl)phenyl)-1-tosyl-1H-pyrrolo[2,3-b]pyridin-3-yl)-3,4-dihydroisoquinoline-2(1H)-carboxylate

The title compound was synthesized by following General Procedure GP4, starting from tert-butyl 6-(5-bromo-1-tosyl-1H-pyrrolo[2,3-b]pyridin-3-yl)-3,4-dihydroisoquinoline-2(1H)-carboxylate (C32, 0.25 g, 0.429 mmol) and (1-(4-(4,4,5,5-tetramethyl-1,3,2-dioxaborolan-2-yl)benzyl)piperidin-3-yl)methanol (D7, 0.142 g, 0.429 mmol), and was obtained as an off-white solid (0.25 g, 82% yield). LC/MS: 707.3 [M+H].

### Step 2: Synthesis of (1-(4-(3-(1,2,3,4-tetrahydroisoquinolin-6-yl)-1-tosyl-1H-pyrrolo[2,3-b]pyridin-5-yl)benzyl)piperidin-3-yl)methanol

The title compound was synthesized by following General Procedure GP6, starting from tert-butyl 6-(5-(4-((3-(hydroxymethyl)piperidin-1-yl)methyl)phenyl)-1-tosyl-1H-pyrrolo[2,3-b]pyridin-3-yl)-3,4-dihydroisoquinoline-2(1H)-carboxylate (0.2 g, 0.240 mmol), and was obtained as an off-white solid (0.140 mg, 95% yield). LC/MS: 607.3 [M+H].

### Step 3: Synthesis of (1-(4-(3-(2-methyl-1,2,3,4-tetrahydroisoquinolin-6-yl)-1-tosyl-1H-pyrrolo[2,3-b]pyridin-5-yl)benzyl)piperidin-3-yl)methanol

The title compound was synthesized as described for Intermediate E81, starting from (1-(4-(3-(1,2,3,4-tetrahydroisoquinolin-6-yl)-1-tosyl-1H-pyrrolo[2,3-b]pyridin-5-yl)benzyl)piperidin-3-yl)methanol (0.140 g, 0.231 mmol) and paraformaldehyde (0.020 g, 0.692 mmol), and was obtained as an off-white solid (0.130 g, 90% yield). LC/MS: 621.3 [M+H].

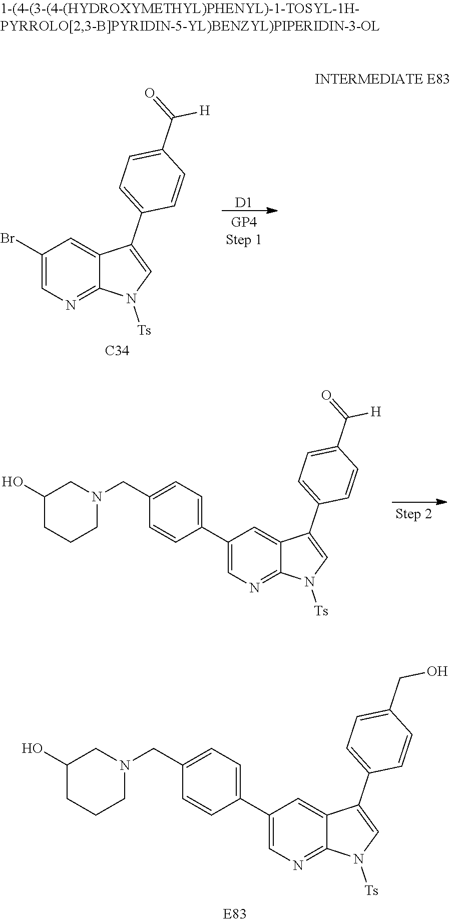 [SMILES: Cc1ccc(S(=O)(=O)n2cc(-c3ccc(C=O)cc3)c3cc(Br)cnc32)cc1 ; "D1" ; "C34" ; Cc1ccc(S(=O)(=O)n2cc(-c3ccc(C=O)cc3)c3cc(-c4ccc(CN5CCCC(O)C5)cc4)cnc32)cc1 ; Cc1ccc(S(=O)(=O)n2cc(-c3ccc(CO)cc3)c3cc(-c4ccc(CN5CCCC(O)C5)cc4)cnc32)cc1]

### Step 1: Synthesis of 4-(5-(4-((3-hydroxypiperidin-1-yl)methyl)phenyl)-1-tosyl-1H-pyrrolo[2,3-b]pyridin-3-yl)benzaldehyde

The title compound was synthesized by following General Procedure GP4, starting from 4-(5-bromo-1-tosyl-1H-pyrrolo[2,3-b]pyridin-3-yl)benzaldehyde (C34, 0.300 g, 0.659 mmol) and 1-(4-(4,4,5,5-tetramethyl-1,3,2-dioxaborolan-2-yl)benzyl)piperidin-3-ol (D1, 0.230 g, 0.725 mmol), and was obtained as a gum (0.23 g, 62% yield). LC/MS: 566.2 [M+H].

### Step 2: Synthesis of 1-(4-(3-(4-(hydroxymethyl)phenyl)-1-tosyl-1H-pyrrolo[2,3-b]pyridin-5-yl)benzyl)piperidin-3-ol

NaBH₄ (0.021 g, 0.610 mmol) was added to a solution of 4-(5-(4-((3-hydroxypiperidin-1-yl)methyl)phenyl)-1-tosyl-1H-pyrrolo[2,3-b]pyridin-3-yl)benzaldehyde (0.230 g, 0.407 mmol) in MeOH (10 mL) at 0° C. and the resulting solution was allowed to warm to 25° C. and was stirred for 2 h. Following completion of reaction, the mixture was filtered through a pad of diatomaceous earth (i.e., Celite®) which was then rinsed with DCM (2×20 mL). The combined filtrates were washed with water (10 mL) and brine (10 mL), dried over anhydrous Na₂SO₄, filtered, and concentrated under the reduced pressure to give the title compound (0.15 g, crude) as yellow solid which was used without further purification. LC/MS: 568.2 [M+H]

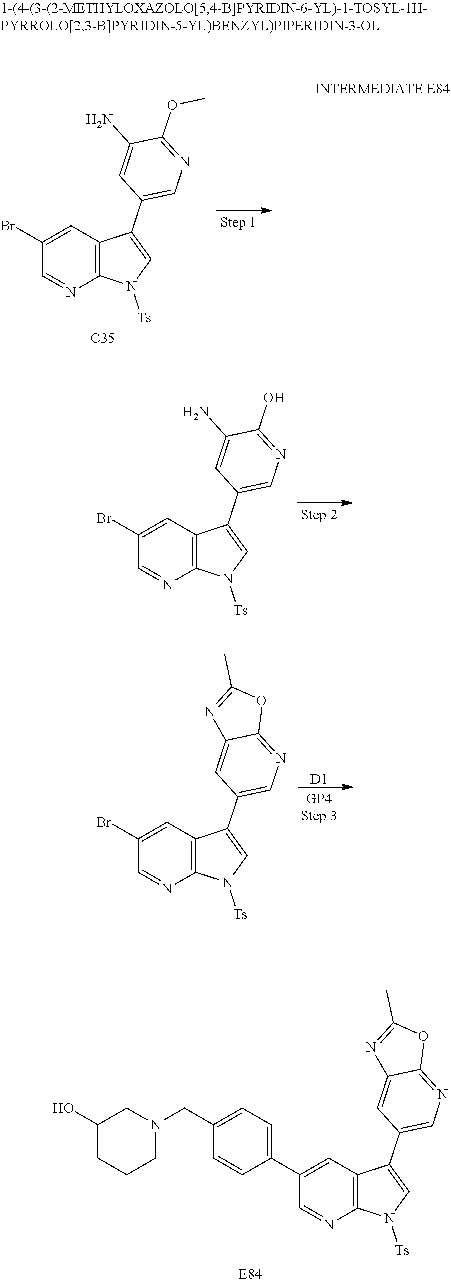

### Step 1: Synthesis of 3-amino-5-(5-bromo-1-tosyl-1H-pyrrolo[2,3-b]pyridin-3-yl)pyridin-2-ol

HCl (2M in 1,4-dioxane, 20 mL, 40.0 mmol) was added to a solution of 5-(5-bromo-1-tosyl-1H-pyrrolo[2,3-b]pyridin-3-yl)-2-methoxypyridin-3-amine (C35, 0.5 g, 1.06 mmol) in 1,4-dioxane (10 mL) at 0° C. and the resulting mixture was heated to 90° C. for 3 h. Following completion of the reaction, the mixture was concentrated under reduced pressure, giving a residue which was dissolved in water (15 mL), basified with solid NaHCO₃ to pH 8, and extracted with EtOAc (2×50 mL). The combined organic phases were washed with brine (50 mL), dried over anhydrous Na₂SO₄, filtered, and concentrated under reduced pressure to give the title compound (0.35 g, crude) as a brown gum which was used without further purification. LC/MS: 459.0 [M+H].

### Step 2: Synthesis of 6-(5-bromo-1-tosyl-1H-pyrrolo[2,3-b]pyridin-3-yl)-2-methyloxazolo[5,4-b]pyridine

A mixture of 1,1,1-trimethoxyethane (0.45 g, 3.810 mmol) and 3-amino-5-(5-bromo-1-tosyl-1H-pyrrolo[2,3-b]pyridin-3-yl)pyridin-2-ol (0.35 g, 0.762 mmol) was heated to 120° C. for 16 h in a sealed tube. Following completion of the reaction, the mixture was diluted with water (50 mL) and extracted with EtOAc (2×100 mL). The combined organic phases were washed with brine (50 mL), dried over anhydrous Na₂SO₄, filtered, and concentrated under reduced pressure to give crude material which was purified by flash chromatography (eluting with 10 to 50% EtOAc in n-hexane), affording the title compound (0.1 g, 27% yield) as an off-white solid. ¹H NMR (400 MHz, CDCl₃) δ=8.54-8.48 (m, 2H), 8.16-8.09 (m, 4H), 7.94 (s, 1H), 7.32 (d, J=8.0 Hz, 2H), 2.73 (s, 3H), 2.41 (s, 3H). LC/MS: 483.0 [M+H].

### Step 3: Synthesis of 1-(4-(3-(2-methyloxazolo[5,4-b]pyridin-6-yl)-1-tosyl-1H-pyrrolo[2,3-b]pyridin-5-yl)benzyl)piperidin-3-ol

The title compound was synthesized by following General Procedure GP4, starting from 6-(5-bromo-1-tosyl-1H-pyrrolo[2,3-b]pyridin-3-yl)-2-methyloxazolo[5,4-b]pyridine (0.180 g, 0.372 mmol) and 1-(4-(4,4,5,5-tetramethyl-1,3,2-dioxaborolan-2-yl)benzyl)piperidin-3-ol (D1, 0.118 g, 0.372 mmol), and was obtained as a pale brown solid (0.14 g, 6300). LC/MS: 593.2 [M+H].

### PREPARATION OF EXAMPLES

The following examples were synthesized by following General Procedure GP5:

| | Structure | Start. | | ¹H NMR |
|---|---|---|---|---|
| Ex. | Name | Mat. | LC/MS | 400 MHz, DMSO-d₆, δ = |
| 1 | 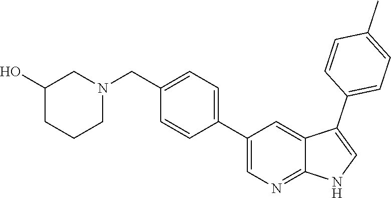 [SMILES: Cc1ccc(-c2c[nH]c3ncc(-c4ccc(CN5CCCC(O)C5)cc4)cc23)cc1] | E1 | 398.4 [M + H] | 11.91 (s, 1H), 8.53 (s, 1H), 8.37 (s, 1H), 7.83 (s, 1H), 7.67 (t, J = 6.2 Hz, 4H), 7.38 (d, J = 6.8 Hz, 2H), 7.26 (d, J = 7.2 Hz, 2H), 4.52 (s, 1H), 3.56-3.53 (m, 1H), 3.44-3.31 (m, 2H), 2.81 (d, J = 9.2 Hz, 1H), 2.67 (d, J = 10.4 Hz, 1H), 2.33 (s, 3H), 1.86-1.85 (m, 2H), 1.81-1.78 (m, 1H), 1.70-1.68 (m, 1H), 1.59-1.57 (m, 1H), 1.09-1.03 (m, 1H) |
| | 1-(4-(3-(p-tolyl)-1H- | | | |
| | pyrrolo[2,3-b]pyridin-5- | | | |
| | yl)benzyl)piperidin-3-ol | | | |
| 2 | 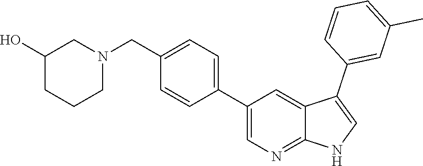 [SMILES: Cc1cccc(-c2c[nH]c3ncc(-c4ccc(CN5CCCC(O)C5)cc4)cc23)c1] | E2 | 398.4 [M + H] | 11.95 (s, 1H), 8.54 (s, 1H), 8.39 (s, 1H), 7.86 (s, 1H), 7.69 (d, J = 7.2 Hz, 2H), 7.56 (s, 2H), 7.38 (d, J = 7.2 Hz, 2H), 7.33 (t, J = 7.2 Hz, 1H), 7.07 (d, J = 7.6Hz, 1H), 4.57 (d, J = 3.2 Hz, 1H), 3.56-3.53 (m, 1H), 3.45-3.42 (m, 2H), 2.83-2.80 (m, 1H), 2.69- 2.66 (m, 1H), 2.38 (s, 3H), 1.90- 1.59 (m, 4H), 1.44-1.40 (m, 1H), 1.07-1.04 (m, 1H) |
| | 1-(4-(3-(m-tolyl)-1H- | | | |
| | pyrrolo[2,3-b]pyridin-5- | | | |
| | yl)benzyl)piperidin-3-ol | | | |
| 3 | 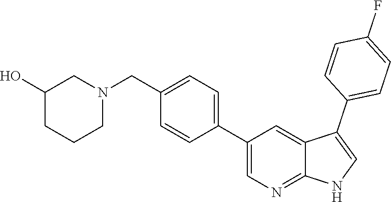 [SMILES: OC1CCCN(Cc2ccc(-c3cnc4[nH]cc(-c5ccc(F)cc5)c4c3)cc2)C1] | E3 | 402.0 [M + H] | 11.97 (bs, 1H), 8.53 (s, 1H), 8.37 (s, 1H), 7.87 (s, 1H), 7.80 (t, J = 5.6 Hz, 2H), 7.68 (d, J = 8Hz, 2H), 7.37 (d, J = 8.0 Hz, 2H), 7.24 (t, J = 8.8 Hz, 2H), 4.56 (d, J = 4.8 Hz, 1H), 3.55- 3.43 (m, 4H), 2.81-2.78 (m, 2H), 1.88-1.58 (m, 3H), 1.42-1.39 (m, 1H), 1.05-1.03 (m, 1H) |
| | 1-(4-(3-(4-fluorophenyl)-1H- | | | |
| | pyrrolo[2,3-b]pyridin-5- | | | |
| | yl)benzyl)piperidin-3-ol | | | |
| 4 | 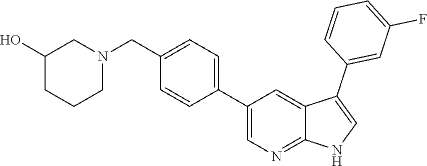 [SMILES: OC1CCCN(Cc2ccc(-c3cnc4[nH]cc(-c5cccc(F)c5)c4c3)cc2)C1] | E4 | 402.1 [M + H] | 12.07 (bs, 1H), 8.54 (s, 1H), 8.42 (s, 1H), 8.00 (s, 1H), 7.69- 7.43 (m, 6H), 7.37 (d, J = 7.6 Hz, 1H), 7.05 (t, J = 6.8 Hz, 1H), 4.55 (d, J = 4.4 Hz, 1H), 3.55-3.52 (m, 1H), 3.44-3.40 (m, 2H), 2.81-2.79 (m, 1H), 2.67- 2.64 (m, 1H), 1.88-1.56 (m, 4H), 1.05-1.03 (m, 2H) |
| | 1-(4-(3-(3-fluorophenyl)-1H- | | | |
| | pyrrolo[2,3-b]pyridin-5- | | | |
| | yl)benzyl)piperidin-3-ol | | | |
| 5 | 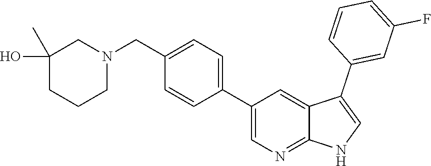 [SMILES: CC1(O)CCCN(Cc2ccc(-c3cnc4[nH]cc(-c5cccc(F)c5)c4c3)cc2)C1] 1-(4-(3-(3-fluorophenyl)-1H- pyrrolo[2,3-b]pyridin-5- yl)benzyl)-3-methylpiperidin-3-ol | E5 | 416.2 [M + H] | 12.08 (s, 1H), 8.58 (d, J = 2.1 Hz, 1H), 8.46 (d, J = 2.2 Hz, 1H), 8.02 (d, J = 2.7 Hz, 1H), 7.74 (d, J = 7.8 Hz, 2H), 7.71- 7.65 (m, 1H), 7.61 (d, J = 10.7, Hz, 1H), 7.49 (dd, J = 8.0, 6.4 Hz, 1H), 7.43 (d, J = 7.8 Hz, 2H), 7.08 (dd, J = 8.6, 2.6 Hz, 1H), 4.15 (s, 1H), 3.55 (d, J = 13.4 Hz, 1H), 3.48 (d, J = 13.4 Hz, 1H), 2.56 (d, J = 1.9 Hz, 1H), 2.46 (d, J = 1.9 Hz, 1H), 2.26 (d, J = 10.6 Hz, 2H), 2.11 (d, J = 10.9 Hz, 1H), 1.65 (s, 1H), 1.51-1.33 (m, 2H), 1.14 (s, 3H) |
| 6 | 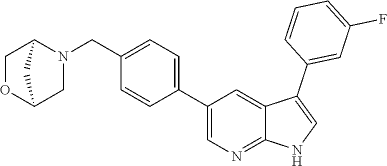 [SMILES: Fc1cccc(-c2c[nH]c3ncc(-c4ccc(CN5C[C@@H]6C[C@H]5CO6)cc4)cc23)c1] | E6 | 398.2 [M − H] | 12.08 (s, 1H), 8.57 (s, 1H), 8.44 (s, 1H), 8.02 (s, 1H), 7.73-7.66 (m, 3H), 7.61 (d, J = 10.4 Hz, 1H), 7.51-7.45 (m, 3H), 7.10- 7.05 (m, 1H), 4.37 (s, 1H), 3.96 (d, J = 7.6 Hz, 1H), 3.81-3.73 (m, 2H), 3.55 (d, J = 7.2 Hz, 1H), 3.48 (s, 1H), 2.77 (d, J = 9.6 Hz, 1H), 2.47 (s, 1H), 1.83 (d, J = 9.2 Hz, 1H), 1.61 (d, J = 9.2 Hz, 1H) |
| | (15,4S)-5-(4-(3-(3- | | | |
| | fluorophenyl)-1H-pyrrolo[2,3- | | | |
| | b]pyridin-5-yl)benzyl)-2-oxa-5- | | | |
| | azabicyclo[2.2.1 ]heptane | | | |
| 7 | 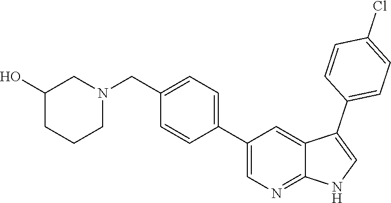 [SMILES: OC1CCCN(Cc2ccc(-c3cnc4[nH]cc(-c5ccc(Cl)cc5)c4c3)cc2)C1] | E7 | 417.9 [M + H] | 12.02 (s, 1H), 8.54 (s, 1H), 8.40 (s, 1H), 7.94 (s, 1H), 7.80 (d, J = 12 Hz, 2H), 7.69 (d, J = 8 Hz, 2H), 7.46 (d, J = 8 Hz, 2H), 7.37 (d, J = 8 Hz, 2H), 4.52 (d, J = 4 Hz, 1H), 3.55-3.52 (m, 1H), 3.44-3.41 (m, 2H), 2.80 (d, J = 8 Hz, 1H), 2.30-1.86 (m, 1H), 1.86-1.79 (m, 1H), 1.79-1.70 (m, 1H), 1.70-1.62 (m, 2H), 1.62- 1.58 (m, 2H) |
| | 1-(4-(3-(4-chlorophenyl)-1H- | | | |
| | pyrrolo[2,3-b]pyridin-5- | | | |
| | yl)benzyl)piperidin-3-ol | | | |
| 8 | 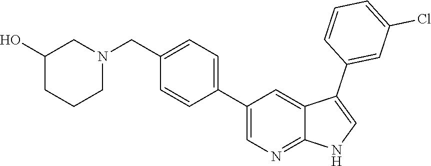 [SMILES: OC1CCCN(Cc2ccc(-c3cnc4[nH]cc(-c5cccc(Cl)c5)c4c3)cc2)C1] | E8 | 418.1 [M + H] | 12.11 (s, 1H), 8.53 (s, 1H), 8.38 (s, 1H), 7.96 (s, 1H), 7.75 (s, 2H), 7.68-7.66 (m, 2H), 7.46 (t, J = 8 Hz, 1H), 7.38 (d, 2H), 7.28 (m, 1H), 3.51-3.41 (m, 5H), 2.79 (d, J = 8 Hz, 1H), 2.65 (s, 1H), 1.88-1.69 (m, 3H), 1.67-1.57 (m, 1H), 1.41-1.39 (m, 1H) |
| | 1-(4-(3-(3-chlorophenyl)-1H- | | | |
| | pyrrolo[2,3-b]pyridin-5- | | | |
| | yl)benzyl)piperidin-3-ol | | | |
| 9 | 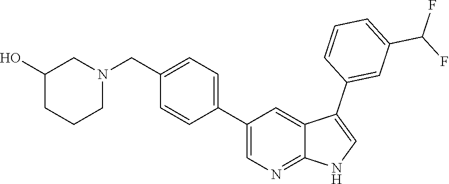 [SMILES: OC1CCCN(Cc2ccc(-c3cnc4[nH]cc(-c5cccc(C(F)F)c5)c4c3)cc2)C1] | E9 | 434.0 [M + H] | 12.09 (s, 1H), 8.56 (s, 1H), 8.43 (s, 1H), 8.01-7.93 (m, 3H), 7.70- 7.68 (m, 2H), 7.59 (t, J = 8.0 Hz, 1H), 7.46-7.38 (m, 3H), 7.10 (t, J = 6.4 Hz, 1H), 4.57 (d, J = 4.8 Hz, 1H), 3.55-3.31 (m, 3H), 2.82-2.80 (m, 1H), 2.68-2.66 (m, 1H), 1.89-1.81 (m, 4H), 1.43- 1.41 (m, 1H), 1.08-1.01 (m, 1H) |
| | 1-(4-(3-(3- | | | |
| | (difluoromethyl)phenyl)-1H- | | | |
| | pyrrolo[2,3-b]pyridin-5- | | | |
| | yl)benzyl)piperidin-3-ol | | | |
| 10 | 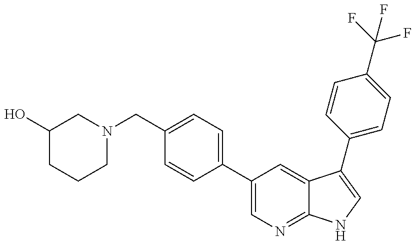 [SMILES: OC1CCCN(Cc2ccc(-c3cnc4[nH]cc(-c5ccc(C(F)(F)F)cc5)c4c3)cc2)C1] | E10 | 450.2 [M − H] | 12.19 (s, 1H), 8.57 (s, 1H), 8.48 (s, 1H), 8.10 (s, 1H), 8.02 (d, J = 8.4 Hz, 2H), 7.76-7.70 (m, 4H), 7.38 (d, J = 7.6 Hz, 2H), 4.57 (d, J = 4.8 Hz, 1H), 3.56-3.43 (m, 3H), 2.81 (d, J = 6.8 Hz, 1H), 2.65 (s, 1H), 1.89-1.59 (m, 4H), 1.43-1.40 (m, 1H), 1.06-1.03 (m, 1H) |
| | 1-(4-(3-(4- | | | |
| | (trifluoromethyl)phenyl)-1H- | | | |
| | pyrrolo[2,3-b]pyridin-5- | | | |
| | yl)benzyl)piperidin-3-ol | | | |
| 11 | 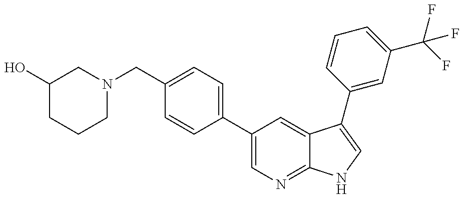 [SMILES: OC1CCCN(Cc2ccc(-c3cnc4[nH]cc(-c5cccc(C(F)(F)F)c5)c4c3)cc2)C1] | E11 | 452.3 [M + H] | 12.15 (s, 1H), 8.55 (s, 1H), 8.41 (s, 1H), 8.15-8.11 (m, 2H), 8.02 (s, 1H), 7.71-7.67 (m, 3H), 7.58 (d, J = 8 Hz, 1H), 7.39 (d, J = 8 Hz, 2H), 4.56 (d, J = 8 Hz, 1H), 3.53 (s, 1H), 3.46-3.41 (m, 2H), 2.81 (d, J = 8 Hz, 1H), 1.65-1.49 (m, 1H), 1.87-1.77 (m, 2H), 1.72-1.70 (m, 1H), 1.62-1.59 (m, 1H), 1.43-1.40 (m, 1H), 1.06- 1.04 (m, 1H) |
| | 1-(4-(3-(3- | | | |
| | (trifluoromethyl)phenyl)-1H- | | | |
| | pyrrolo[2,3-b]pyridin-5- | | | |
| | yl)benzyl)piperidin-3-ol | | | |
| 12 | 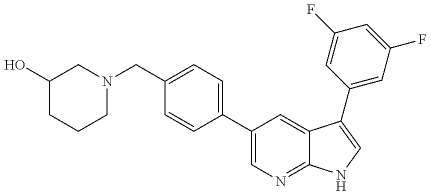 [SMILES: OC1CCCN(Cc2ccc(-c3cnc4[nH]cc(-c5cc(F)cc(F)c5)c4c3)cc2)C1] | E12 | 420.3 [M + H] | 12.23 (s, 1H), 8.63 (s, 1H), 8.53 (s, 1H), 8.17 (s, 1H), 7.80 (d, J = 7.6 Hz, 2H), 7.59 (d, J = 8.0 Hz, 2H), 7.46 (d, J = 7.6 Hz, 2H), 7.12 (t, J = 9.2 Hz, 1H), 4.61 (s, 1H), 3.64-3.43 (m, 3H), 2.89 (d, J = 9.6 Hz, 1H), 2.73 (s, 1H), 1.98-1.65 (m, 4H), 1.54-1.48 (m, 1H), 1.17-1.09 (m, 1H) |
| | 1-(4-(3-(3,5-difluorophenyl)- | | | |
| | 1H-pyrrolo[2,3-b]pyridin-5- | | | |
| | yl)benzyl)piperidin-3-ol | | | |
| 13 | 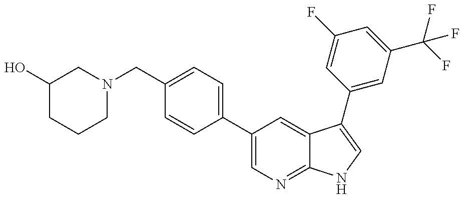 [SMILES: OC1CCCN(Cc2ccc(-c3cnc4[nH]cc(-c5cc(F)cc(C(F)(F)F)c5)c4c3)cc2)C1] | E13 | 470.3 [M + H] | 12.25 (s, 1H), 8.57 (s, 1H), 8.44 (s, 1H), 8.20 (s, 1H), 7.98 (d, J = 10.0 Hz, 1H), 7.91 (s, 1H), 7.71 (d, J = 8.0 Hz, 2H), 7.49 (d, J = 8.4 Hz, 1H), 7.39 (d, J = 8.0 Hz, 2H), 4.58 (d, J = 4.8 Hz, 1H), 3.56-3.41 (m, 3H), 2.81 (d, J = 7.2 Hz, 1H), 2.68 (s, 1H), 1.89- 1.59 (m, 4H), 1.43-1.40 (m, 1H), 1.06-1.04 (m, 1H) |
| | 1-(4-(3-(3-fluoro-5- | | | |
| | (trifluoromethyl)phenyl)-1H- | | | |
| | pyrrolo[2,3-b]pyridin-5- | | | |
| | yl)benzyl)piperidin-3-ol | | | |
| 14 | 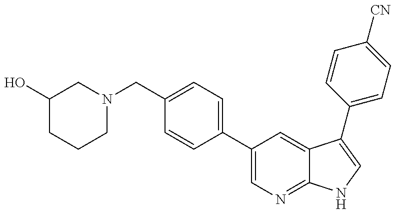 [SMILES: N#Cc1ccc(-c2c[nH]c3ncc(-c4ccc(CN5CCCC(O)C5)cc4)cc23)cc1] | E14 | 407.2 [M − H] | 12.01 (s, 1H), 8.57 (s, 1H), 8.49 (s, 1H), 8.17 (s, 1H), 8.02 (d, J = 8.0 Hz, 2H), 7.84 (d, J = 8.0 Hz, 2H), 7.71 (d, J = 8.0 Hz, 2H), 7.38 (d, J = 8.0 Hz, 2H), 4.57 (d, J = 3.6 Hz, 1H), 3.53 (s, 1H), 3.39-3.34 (m, 2H), 2.80-2.78 (m, 1H), 2.66-2.64 (m, 1H), 1.86- 1.85 (m, 2H), 1.69-1.65 (m, 1H), 1.60-1.57 (m, 1H), 1.43-1.40 (m, 1H), 1.06-1.03 (m, 1H) |
| | 4-(5-(4-((3-hydroxypiperidin-1- | | | |
| | yl)methyl)phenyl)-1H- | | | |
| | pyrrolo[2,3-b]pyridin-3- | | | |
| | yl)benzonitrile | | | |
| 15 | 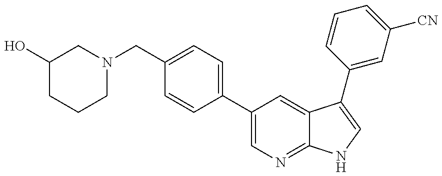 [SMILES: N#Cc1cccc(-c2c[nH]c3ncc(-c4ccc(CN5CCCC(O)C5)cc4)cc23)c1] | E15 | 407.2 [M − H] | 12.16 (s, 1H), 8.52 (d, J = 6.8 Hz, 2H), 8.23 (s, 1H), 8.16 (d, J = 7.6 Hz, 1H), 8.09 (s, 1H), 7.73-7.60 (m, 4H), 7.38 (d, J = 8.0 Hz, 2H), 4.57 (s, 1H), 3.56- 3.41 (m, 3H), 2.80 (d, J = 7.2 Hz, 1H), 2.68 (s, 1H), 1.88-1.39 (m, 6H) |
| | 3-(5-(4-((3-hydroxypiperidin-1- | | | |
| | yl)methyl)phenyl)-1H- | | | |
| | pyrrolo[2,3-b]pyridin-3- | | | |
| | yl)benzonitrile | | | |
| 16 | 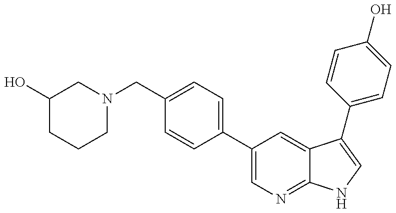 [SMILES: Oc1ccc(-c2c[nH]c3ncc(-c4ccc(CN5CCCC(O)C5)cc4)cc23)cc1] | E16 | 398.2 [M − H] | 11.79 (s, 1H), 9.32 (s, 1H), 8.53 (d, J = 2.0 Hz, 1H), 8.35 (d, J = 1.6 Hz, 1H), 7.70 (d, J = 11.2 Hz, 3H), 7.57 (t, J = 8.4 Hz, 2H), 7.39 (d, J = 8.4 Hz, 2H), 6.86 (d, J = 8.4 Hz, 2H), 4.54 (d, J = 4.8 Hz, 1H), 3.58-3.56 (m, 3H), 2.83 (d, J = 6.8 Hz, 1H), 2.70-2.67 (m, 1H), 1.91-1.61 (m, 4H), 1.45-1.42 (m, 1H), 1.09- 1.06 (m, 1H) |
| | 1-(4-(3-(4-hydroxyphenyl)-1H- | | | |
| | pyrrolo[2,3-b]pyridin-5- | | | |
| | yl)benzyl)piperidin-3-ol | | | |
| 17 | 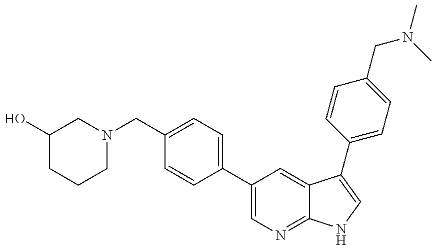 [SMILES: CN(C)Cc1ccc(-c2c[nH]c3ncc(-c4ccc(CN5CCCC(O)C5)cc4)cc23)cc1] | E17 | 441.4 [M + H] | 11.98 (s, 1H), 8.55 (s, 1H), 8.41 (s, 1H), 7.91 (s, 1H), 7.77-7.71 (m, 4H), 7.40-7.31 (m, 4H), 4.54-4.52 (m, 1H), 3.46-3.40 (m, 4H), 2.82-2.79 (m, 2H), 2.65- 2.61 (m, 1H), 2.35-2.31 (m, 8H), 1.88-1.61 (m, 2H), 1.60 (s, 2H) |
| | 1-(4-(3-(4- | | | |
| | ((dimethylamino)methyl)phenyl)- | | | |
| | 1H-pyrrolo[2,3-b]pyridin-5- | | | |
| | yl)benzyl)piperidin-3-ol | | | |
| 18 | 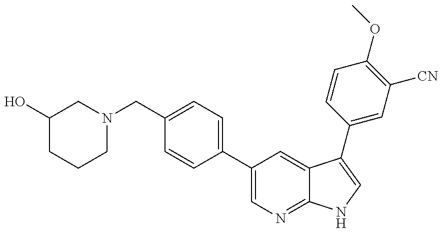 [SMILES: COc1ccc(-c2c[nH]c3ncc(-c4ccc(CN5CCCC(O)C5)cc4)cc23)cc1C#N] | E18 | 439.2 [M + H] | 12.01 (s, 1H), 8.54 (s, 1H), 8.41 (s, 1H), 8.09 (t, J = 2.4 Hz, 2H), 7.94 (m, 1H), 7.70 (d, J = 7.6 Hz, 2H), 7.39-7.29 (m, 3H), 4.54 (d, J = 4.8 Hz, 1H), 3.94 (s, 3H), 3.52-3.41 (m, 3H), 2.80 (d, J = 7.2 Hz, 1H), 2.30 (s, 1H), 1.88- 1.58 (m, 4H), 1.46-1.36 (m, 1H), 1.08-1.02 (m, 1H) |
| | 5-(5-(4-((3-hydroxypiperidin-1- | | | |
| | yl)methyl)phenyl)-1H- | | | |
| | pyrrolo[2,3-b]pyridin-3-yl)-2- | | | |
| | methoxybenzonitrile | | | |
| 19 | 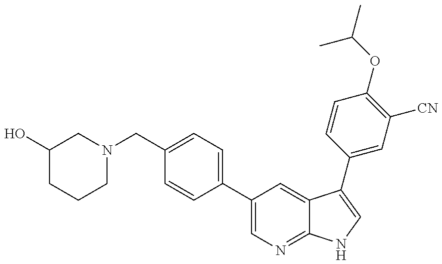 [SMILES: CC(C)Oc1ccc(-c2c[nH]c3ncc(-c4ccc(CN5CCCC(O)C5)cc4)cc23)cc1C#N] | E19 | 467.2 [M + H] | 12.02 (s, 1H), 8.56 (d, J = 2.0 Hz, 1H), 8.43 (d, J = 2.0 Hz, 1H), 8.09 (d, J = 2.4 Hz, 1H), 8.05 (d, J = 2.4 Hz, 1H), 7.96 (s, 1H), 7.72 (d, J = 8.0 Hz, 2H), 7.40 (d, J = 8.0 Hz, 2H), 7.34 (d, J = 8.8 Hz, 1H), 4.85-4.81 (m, 1H), 4.58-4.57 (m, 1H), 3.58- 3.55 (m, 1H), 3.47-3.42 (m, 2H), 2.84-2.82 (m, 1H), 2.68-2.67 (m, 1H), 1.92-1.87 (m, 2H), 1.75- 1.70 (m, 1H), 1.65-1.61 (m, 1H), 1.46-1.43 (m, 1H), 1.36 (m, 6H), 1.09-1.05 (m, 1H) |
| | 5-(5-(4-((3-hydroxypiperidin-1- | | | |
| | yl)methyl)phenyl)-1//- | | | |
| | pyrrolo[2,3-b]pyridin-3-yl)-2- | | | |
| | isopropoxybenzonitrile | | | |
| 20 | 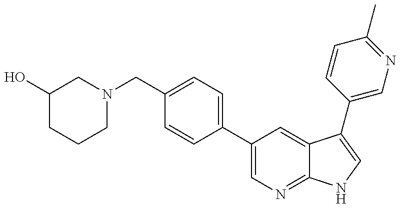 [SMILES: Cc1ccc(-c2c[nH]c3ncc(-c4ccc(CN5CCCC(O)C5)cc4)cc23)cn1] | E20 | 399.4 [M + H] | 12.05 (s, 1H), 8.86 (s, 1H), 8.55 (s, 1H), 8.39 (s, 1H), 8.06 (d, J = 6.0 Hz, 1H), 7.97 (s, 1H), 7.71- 7.68 (m, 2H), 7.38-7.36 (m, 2H), 7.30-7.28 (m, 1H), 4.55 (d, J = 4.4 Hz, 1H), 3.55-3.52 (m, 1H), 3.44-3.42 (m, 2H), 2.81-2.79 (m, 2H), 2.64-2.62 (m, 2H), 1.86- 1.81 (m, 3H), 1.69-1.66 (m, 2H), 1.48-1.44 (m, 2H) |
| | 1-(4-(3-(6-methylpyridin-3-yl)- | | | |
| | 1H-pyrrolo[2,3-b]pyridin-5- | | | |
| | yl)benzyl)piperidin-3-ol | | | |
| 21 | 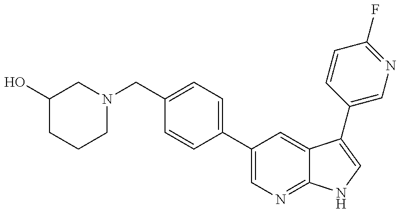 [SMILES: OC1CCCN(Cc2ccc(-c3cnc4[nH]cc(-c5ccc(F)nc5)c4c3)cc2)C1] | E21 | 403.1 [M + H] | 12.05 (s, 1H), 8.67 (s, 1H), 8.59 (s, 1H), 8.45-8.39 (m, 2H), 8.04 (s, 1H), 7.74 (d, J = 8 Hz, 2H), 7.40 (d, J = 8 Hz , 2H), 7.25 (d, J = 8 Hz, 1H), 4.56-4.55 (m, 1H), 3.58-3.55 (m, 1H), 3.47- 3.44 (m, 2H), 2.84-2.81 (m, 1H), 2.70-2.67 (m, 1H), 1.92-1.89 (m, 3H), 1.75-1.72 (m, 1H), 1.45- 1.42 (m, 1H), 1.09-1.06 (m, 1H) |
| | 1-(4-(3-(6-fluoropyridin-3-yl)- | | | |
| | 1H-pyrrolo[2,3-b]pyridin-5- | | | |
| | yl)benzyl)piperidin-3-ol | | | |
| 22 | 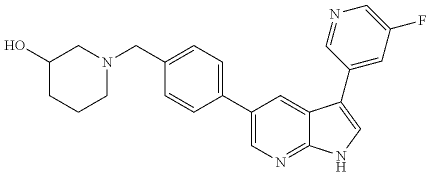 [SMILES: OC1CCCN(Cc2ccc(-c3cnc4[nH]cc(-c5cncc(F)c5)c4c3)cc2)C1] | E22 | 403.3 [M + H] | 12.24 (s, 1H), 8.93 (s, 1H), 8.58 (s, 1H), 8.51 (s, 1H), 8.43 (s, 1H), 8.16-8.14 (m, 2H), 7.75 (d, J = 7.2 Hz, 2H), 7.38 (d, J = 7.6 Hz, 2H), 4.56 (s, 1H), 3.56-3.31 (m, 3H), 2.81 (d, J = 8.0 Hz, 1H), 2.65 (s, 1H), 1.87-1.59 (m, 6H) |
| | 1-(4-(3-(5-fluoropyridin-3-yl)- | | | |
| | 1H-pyrrolo[2,3-b]pyridin-5- | | | |
| | yl)benzyl)piperidin-3-ol | | | |
| 23 | 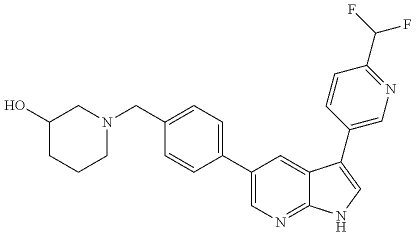 [SMILES: OC1CCCN(Cc2ccc(-c3cnc4[nH]cc(-c5ccc(C(F)F)nc5)c4c3)cc2)C1] | E23 | 435.0 [M + H] | 12.25 (s, 1H), 9.13 (d, J = 2.0 Hz, 1H), 8.62-8.61 (m, 1H), 8.53-8.51 (m, 1H), 8.44-8.41 (m, 1H), 8.19 (s, 1H), 7.77-7.74 (m, 3H), 7.41-7.39 (m, 2H), 6.99 (t, J = 5.8 Hz, 1H), 4.57 (d, J = 4.8 Hz, 1H), 3.58-3.56 (m, 1H), 3.50-3.48 (m, 2H), 3.44-3.42 (m, 1H), 2.84-2.82 (m, 1H), 2.70- 2.68 (m, 2H), 1.90-1.88 (m, 2H), 1.76-1.75 (m, 1H), 1.71-1.69 (m, 1H) |
| | 1-(4-(3-(6- | | | |
| | (difluoromethyl)pyridin-3-yl)- | | | |
| | 1H-pyrrolo[2,3-b]pyridin-5- | | | |
| | yl)benzyl)piperidin-3-ol | | | |
| 24 | 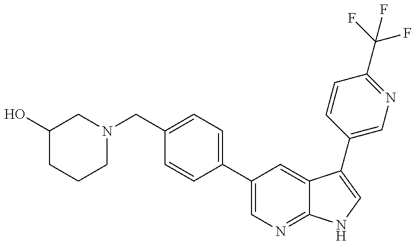 [SMILES: OC1CCCN(Cc2ccc(-c3cnc4[nH]cc(-c5ccc(C(F)(F)F)nc5)c4c3)cc2)C1] | E24 | 451.2 [M − H] | 12.34 (s, 1H), 9.23 (s, 1H), 8.61 (s, 1H), 8.55 (s, 1H), 8.48 (d, J = 8.5 Hz, 1H), 8.26 (d, J = 2.8 Hz, 1H), 7.90 (d, J = 8.3 Hz, 1H), 7.75 (d, J = 7.8 Hz, 2H), 7.39 (d, J = 7.7 Hz, 2H), 4.56 (d, J = 4.9 Hz, 1H), 3.55 (d, J = 13.2 Hz, 1H), 3.44 (d, J = 13.2 Hz, 2H), 2.81 (d, J = 10.0 Hz, 1H), 1.89- 1.84 (m, 4H), 1.59-1.54 (m, 1H), 1.42-1.40 (m, 1H), 1.05-0.99 (m, 1H) |
| | 1-(4-(3-(6- | | | |
| | (trifluoromethyl)pyridin-3-yl)- | | | |
| | 1H-pyrrolo[2,3-b]pyridin-5- | | | |
| | yl)benzyl)piperidin-3-ol | | | |
| 25 | 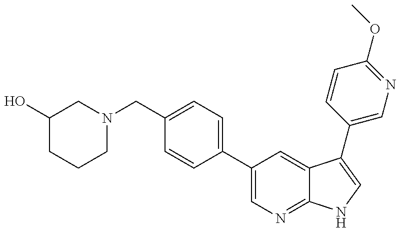 [SMILES: COc1ccc(-c2c[nH]c3ncc(-c4ccc(CN5CCCC(O)C5)cc4)cc23)cn1] | E25 | 415.3 [M + H] | 11.97 (s, 1H), 8.58-8.55 (m, 2H), 8.37 (s, 1H), 8.10-8.08 (m, 1H), 7.87 (s, 1H), 7.71-7.69 (m, 2H), 7.37-7.35 (m, 2H), 6.90- 6.88 (m, 1H), 4.52-4.50 (m, 1H), 3.88 (s, 3H), 3.52-3.50 (m, 1H), 3.44-3.42 (m, 2H), 2.81-2.79 (m, 1H), 2.64-2.62 (m, 1H), 1.86- 1.60 (m, 4H), 1.42-1.40 (m, 1H), 1.06-1.04 (m, 1H) |
| | 1-(4-(3-(6-methoxypyridin-3- | | | |
| | yl)-1H-pyrrolo[2,3-b]pyridin-5- | | | |
| | yl)benzyl)piperidin-3-ol | | | |
| | Racemate | | | |
| 26 | 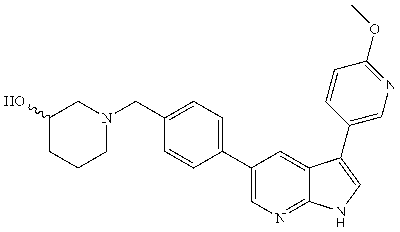 [SMILES: COc1ccc(-c2c[nH]c3ncc(-c4ccc(CN5CCCC(O)C5)cc4)cc23)cn1] | E25 | 415.3 [M + H] | 11.96 (s, 1H), 8.57-8.55 (m, 2H), 8.36 (s, 1H), 8.10-8.08 (m, 1H), 7.87 (s, 1H), 7.71-7.69 (m, 2H), 7.37-7.35 (m, 2H), 6.90- 6.88 (m, 1H), 4.53-4.50 (m, 1H), 3.87 (s, 3H), 3.55-3.51 (m, 1H), 3.44-3.42 (m, 2H), 2.81-2.79 (m, 1H), 2.67-2.65 (m, 1H), 1.89- 1.60 (m, 4H), 1.39-1.37 (m, 1H), 1.06-1.04 (m, 1H) |
| | 1-(4-(3-(6-methoxypyridin-3- | | | |
| | yl)-1H-pyrrolo[2,3-b]pyridin-5- | | | |
| | yl)benzyl)piperidin-3-ol | | | |
| | Enantiomer 1 | | | |
| 27 | 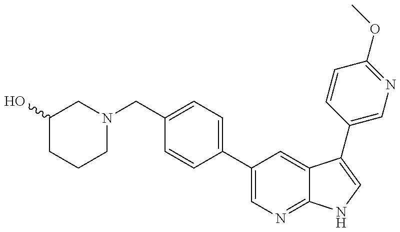 [SMILES: COc1ccc(-c2c[nH]c3ncc(-c4ccc(CN5CCCC(O)C5)cc4)cc23)cn1] | E25 | 415.3 [M + H] | 11.96 (s, 1H), 8.58-8.55 (m, 2H), 8.36 (s, 1H), 8.10-8.08 (m, 1H), 7.87 (s, 1H), 7.73-7.69 (m, 2H), 7.37-7.35 (m, 2H), 6.90- 6.88 (m, 1H), 4.53-4.50 (m, 1H), 3.87 (s, 3H), 3.55-3.51 (m, 1H), 3.44-3.42 (m, 2H), 2.81-2.79 (m, 1H), 2.67-2.65 (m, 1H), 1.89- 1.60 (m, 4H), 1.39-1.37 (m, 1H), 1.06-1.04 (m, 1H) |
| | 1-(4-(3-(6-methoxypyridin-3- | | | |
| | yl)-1H-pyrrolo[2,3-b]pyridin-5- | | | |
| | yl)benzyl)piperidin-3-ol | | | |
| | Enantiomer 2 | | | |
| 28 | 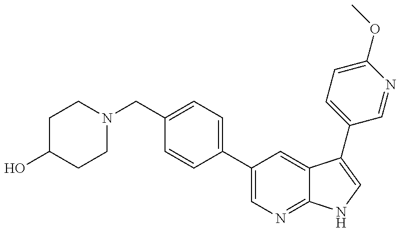 [SMILES: COc1ccc(-c2c[nH]c3ncc(-c4ccc(CN5CCC(O)CC5)cc4)cc23)cn1] | E26 | 415.2 [M + H] | 11.99 (s, 1H), 8.57 (dd, J = 14.3, 2.2 Hz, 2H), 8.37 (s, 1H), 8.10 (dd, J = 8.5, 2.5 Hz, 1H), 7.88 (d, J = 2.6 Hz, 1H), 7.71 (d, J = 7.9 Hz, 2H), 7.37 (d, J = 7.8 Hz, 2H), 6.89 (d, J = 8.5 Hz, 1H), 4.53 (d, J = 4.1 Hz, 1H), 3.88 (s, 3H), 3.45 (d, J = 8.1 Hz, 3H), 2.69 (s, 2H), 2.03 (t, J = 10.4 Hz, 2H), 1.68 (s, 2H), 1.37 (d, J = 11.3 Hz, 2H) |
| | 1-(4-(3-(6-methoxypyridin-3- | | | |
| | yl)-1H-pyrrolo[2,3-b]pyridin-5- | | | |
| | yl)benzyl)piperidin-4-ol | | | |
| 29 | 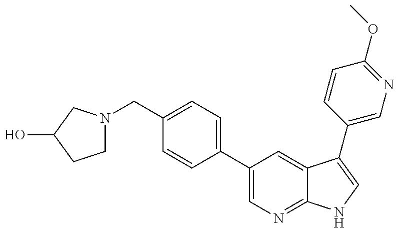 [SMILES: COc1ccc(-c2c[nH]c3ncc(-c4ccc(CN5CCC(O)C5)cc4)cc23)cn1] | E27 | 399.3 [M + H] | 11.95 (s, 1H), 8.55 (d, J = 12.4 Hz, 2H), 8.35 (s, 1H), 8.09 (d, J = 8.8 Hz, 1H), 7.86 (s, 1H), 7.70 (d, J = 7.6 Hz, 2H), 7.37 (d, J = 7.2 Hz, 2H), 6.89 (d, J = 8.4 Hz, 1H), 4.62 (d, J = 4.0 Hz, 1H), 4.18 (s, 1H), 3.87 (s, 3H), 3.57 (d, J = 10.8 Hz, 2H), 2.67-2.56 (m, 2H), 2.31 (s, 1H), 2.04-1.96 (m, 2H), 1.53 (s, 1H) |
| | 1-(4-(3-(6-methoxypyridin-3- | | | |
| | yl)-1H-pyrrolo[2,3-b]pyridin-5- | | | |
| | yl)benzyl)pyrrolidin-3-ol | | | |
| 30 | 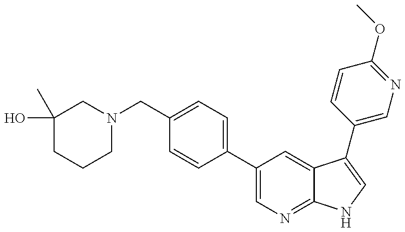 [SMILES: COc1ccc(-c2c[nH]c3ncc(-c4ccc(CN5CCCC(C)(O)C5)cc4)cc23)cn1] | E28 | 429.4 [M + H] | 11.96 (s, 1H), 8.59-8.56 (m, 2H), 8.37 (s, 1H), 8.12-8.09 (m, 1H), 7.88 (s, 1H), 7.71-7.70 (m, 2H), 7.40 (d, J = 8.0 Hz, 2H), 6.91-6.88 (m, 1H), 4.12 (s, 1H), 3.88 (s, 3H), 3.54-3.43 (m, 2H), 2.48 (s, 2H), 2.38-2.31 (m, 2H), 2.09-2.06 (m, 1H), 1.63 (s, 1H), 1.46-1.42 (m, 2H), 1.36 (s, 3H) |
| 31 | 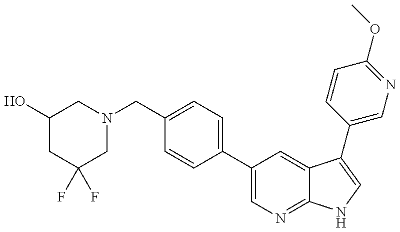 [SMILES: COc1ccc(-c2c[nH]c3ncc(-c4ccc(CN5CC(O)CC(F)(F)C5)cc4)cc23)cn1] | E29 | 451.2 [M + H] | 11.96 (s, 1H), 8.57 (d, J = 9.2 Hz, 2H), 8.37 (s, 1H), 8.10 (d, J = 8.8 Hz, 1H), 7.87 (s, 1H), 7.74 (d, J = 8.0 Hz, 2H), 7.38 (d, J = 8.0 Hz, 2H), 6.89 (d, J = 8.4 Hz, 1H), 5.01 (d, J = 4.8 Hz, 1H), 3.87 (s, 3H), 3.69-3.59 (m, 3H), 2.97-2.89 (m, 2H), 2.36-2.30 (m, 2H), 1.89 (t, J = 10.0 Hz, 1H), 1.61 (m, 1H) |
| | 5,5-difluoro-1-(4-(3-(6- | | | |
| | methoxypyridin-3-yl)-1H- | | | |
| | pyrrolo[2,3-b]pyridin-5- | | | |
| | yl)benzyl)piperidin-3-ol | | | |
| 32 | 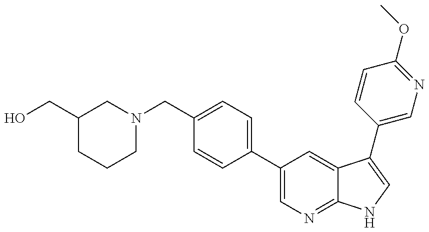 [SMILES: COc1ccc(-c2c[nH]c3ncc(-c4ccc(CN5CCCC(CO)C5)cc4)cc23)cn1] | E30 | 429 [M + H] | 11.97 (s, 1H), 8.59-8.57 (m, 2H), 8.38 (s, 1H), 8.11 (d, J = 8.8 Hz, 1H), 7.88 (s, 1H), 7.72- 7.70 (m, 2H), 7.38-7.36 (m, 2H), 6.90 (d, J = 8.8 Hz, 1H), 4.39- 4.37 (m, 1H), 3.88 (s, 3H), 3.51- 3.49 (m, 2H), 3.19-3.17 (m, 2H), 2.90-2.86 (m, 2H), 1.94-1.89 (m, 2H), 1.61-1.55 (m, 5H) |
| | (1-(4-(3-(6-methoxypyridin-3- | | | |
| | yl)-1H-pyrrolo[2,3-b]pyridin-5- | | | |
| | yl)benzyl)piperidin-3- | | | |
| | yl)methanol | | | |
| 33 | 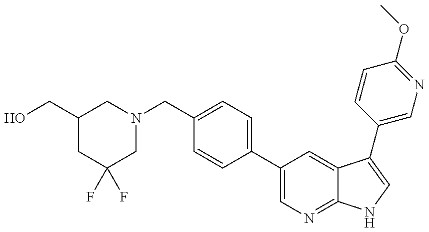 [SMILES: COc1ccc(-c2c[nH]c3ncc(-c4ccc(CN5CC(CO)CC(F)(F)C5)cc4)cc23)cn1] | E31 | 465.4 [M + H] | 11.97 (s, 1H), 8.57 (d, J = 7.2 Hz, 2H), 8.38 (s, 1H), 8.11 (d, J = 6.4 Hz, 1H), 7.87 (s, 1H), 7.74 (d, J = 7.6 Hz, 2H), 7.39 (d, J = 8.0 Hz, 2H), 6.90 (d, J = 8.8 Hz, 1H), 4.57 (s, 1H), 3.88 (s, 3H), 3.62 (s, 2H), 2.99 (s, 2H), 2.88 (d, J = 4.8 Hz, 2H), 2.31 (s, 1H), 2.05 (s, 2H), 1.88 (d, J = 6.8 Hz, 2H) |
| | (5,5-difluoro-1-(4-(3-(6- | | | |
| | methoxypyridin-3-yl)-1H- | | | |
| | pyrrolo[2,3-b]pyridin-5- | | | |
| | yl)benzyl)piperidin-3- | | | |
| | yl)methanol | | | |
| 34 | 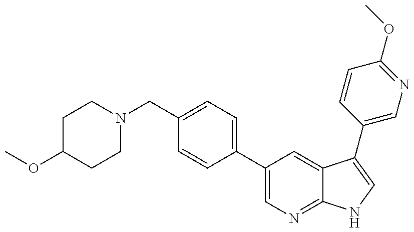 [SMILES: COc1ccc(-c2c[nH]c3ncc(-c4ccc(CN5CCC(OC)CC5)cc4)cc23)cn1] | E32 | 429.0 [M + H] | 11.98 (s, 1H), 8.58-8.55 (d, J = 12.4 Hz, 2H), 8.36 (s, 1H), 8.11- 8.09 (d, J = 8.8 Hz, 1H), 7.88 (s, 2H), 7.70 (s, 1H), 7.37 (s, 2H), 6.90-6.88 (d, J = 8.4 Hz, 1H), 3.87 (s, 3H), 3.47 (s, 2H), 3.20 (s, 4H), 2.71-2.61 (m, 2H), 2.21- 2.05 (m, 2H), 1.85-1.75 (m, 2H), 1.49-1.35 (m, 2H) |
| | 5-(4-((4-methoxypiperidin-1- | | | |
| | yl)methyl)phenyl)-3-(6- | | | |
| | methoxypyridin-3-yl)-1H- | | | |
| | pyrrolo[2,3-b]pyridine | | | |
| 35 | 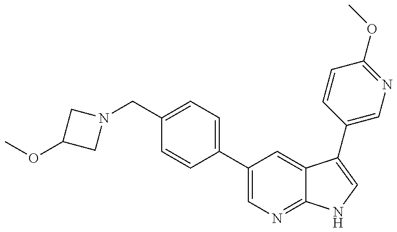 [SMILES: COc1ccc(-c2c[nH]c3ncc(-c4ccc(CN5CC(OC)C5)cc4)cc23)cn1] | E33 | 401.2 [M + H] | 11.98 (s, 1H), 8.56 (dd, J = 16.2, 2.3 Hz, 2H), 8.35 (s, 1H), 8.10 (dd, J = 8.6, 2.5 Hz, 1H), 7.87 (s, 1H), 7.70 (d, J = 7.8 Hz, 2H), 7.34 (d, J = 7.7 Hz, 2H), 6.89 (d, J = 8.6 Hz, 1H), 3.96 (d, J = 5.8 Hz, 1H), 3.87 (s, 3H), 3.59 (s, 2H), 3.13 (s, 3H), 2.84 (t, J = 6.5 Hz, 4H) |
| | 5-(4-((3-methoxyazetidin-1- | | | |
| | yl)methyl)phenyl)-3-(6- | | | |
| | methoxypyridin-3-yl)-1H- | | | |
| | pyrrolo[2,3-b]pyridine | | | |
| 36 | 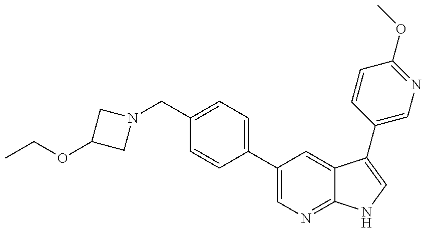 [SMILES: CCOC1CN(Cc2ccc(-c3cnc4[nH]cc(-c5ccc(OC)nc5)c4c3)cc2)C1] | E34 | 415.3 [M + H] | 12.00 (s, 1H), 8.57 (d, J = 12.0 Hz, 2H), 8.37 (s, 1H), 8.10 (d, J = 8.4 Hz, 1H), 7.89 (s, 1H), 7.60-7.58 (m, 2H), 7.42-7.40 (m, 2H), 6.89 (d, J = 8.4 Hz, 1H), 4.12-4.10 (m, 1H), 3.88 (s, 3H), 3.75-3.73 (m, 2H), 3.32-3.28 (m, 6H), 1.08 (t, J = 6.8 Hz, 3H) |
| | 5-(4-((3-ethoxyazetidin-1- | | | |
| | yl)methyl)phenyl)-3-(6- | | | |
| | methoxypyridin-3-yl)-1H- | | | |
| | pyrrolo[2,3-b]pyridine | | | |
| 37 | 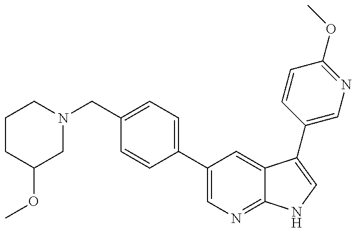 [SMILES: COc1ccc(-c2c[nH]c3ncc(-c4ccc(CN5CCCC(OC)C5)cc4)cc23)cn1] | E35 | 427.3 [M − H] | 11.99 (s, 1H), 8.59-8.58 (m, 2H), 8.55 (s, 1H), 8.12-8.09 (m, 1H), 7.89-7.87 (m, 1H), 7.73- 7.71 (m, 2H), 7.38-7.36 (m, 2H), 6.91-6.89 (m, 1H), 3.88 (s, 3H), 3.51 (s, 2H), 3.20 (s, 3H), 2.91- 2.89 (m, 1H), 2.65-2.63 (m, 2H), 1.91-1.85 (m, 3H), 1.65-1.63 (m, 1H), 1.44-1.42 (m, 1H), 1.11- 1.09 (m, 1H) |
| | 5-(4-((3-methoxypiperidin-1- | | | |
| | yl)methyl)phenyl)-3-(6- | | | |
| | methoxypyridin-3-yl)-1H- | | | |
| | pyrrolo[2,3-b]pyridine | | | |
| | Racemate | | | |
| 38 | 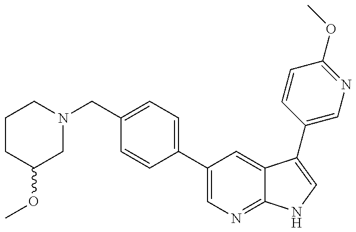 [SMILES: COc1ccc(-c2c[nH]c3ncc(-c4ccc(CN5CCCC(OC)C5)cc4)cc23)cn1] | E36 | 427.3 [M − H] | 11.99 (s, 1H), 8.57 (d, J = 12.0 Hz, 1H), 8.38 (s, 1H), 8.12-8.09 (m, 2H), 7.89 (s, 1H), 7.72 (d, J = 7.6 Hz, 2H), 7.37 (d, J = 7.6 Hz, 2H), 6.90 (d, J = 8.4 Hz, 1H), 3.88 (s, 3H), 3.51 (s, 2H), 3.20 (s, 3H), 2.90 (d, J = 9.2 Hz, 1H), 2.64 (d, J = 9.6 Hz, 1H), 1.94-1.85 (m, 3H), 1.62 (s, 1H), 1.41-1.06 (m, 3H) |
| | 5-(4-((3-methoxypiperidin-1- | | | |
| | yl)methyl)phenyl)-3-(6- | | | |
| | methoxypyridin-3-yl)-1H- | | | |
| | pyrrolo[2,3-b]pyridine | | | |
| | Enantiomer 1 | | | |
| 39 | 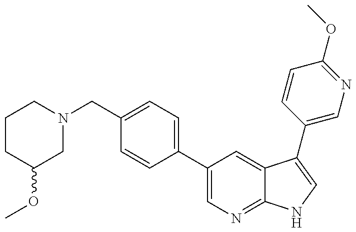 [SMILES: COc1ccc(-c2c[nH]c3ncc(-c4ccc(CN5CCCC(OC)C5)cc4)cc23)cn1] | E37 | 427.3 [M − H] | 11.99 (s, 1H), 8.57 (d, J = 12.8 Hz, 2H), 8.38 (s, 1H), 8.12-8.09 (m, 1H), 7.89 (s, 1H), 7.72 (d, J = 8.4 Hz, 2H), 7.37 (d, J = 8.4 Hz, 2H), 6.89 (d, J = 8.8 Hz, 1H), 3.88 (s, 3H), 3.51 (s, 2H), 3.20 (s, 3H), 2.90 (d, J = 8.8 Hz, 1H), 2.65-2.02 (m, 2H), 1.93- 1.85 (m, 3H), 1.87-1.62 (m, 3H) |
| | 5-(4-((3-methoxypiperidin-1- | | | |
| | yl)methyl)phenyl)-3-(6- | | | |
| | methoxypyridin-3-yl)-1H- | | | |
| | pyrrolo[2,3-b]pyridine | | | |
| | Enantiomer 2 | | | |
| 40 | 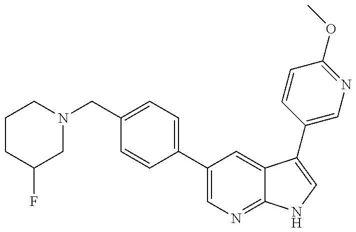 [SMILES: COc1ccc(-c2c[nH]c3ncc(-c4ccc(CN5CCCC(F)C5)cc4)cc23)cn1] | E38 | 417.2 [M + H] | 12.00 (s, 1H), 8.59 (d, J = 12 Hz, 2H), 8.39 (s, 1H), 8.13-8.11 (m, 1H), 7.90 (s, 1H), 7.74-7.73 (m, 2H), 7.39-7.38 (m, 2H), 6.92-6.90 (m, 1H), 4.57 (bs, 1H), 3.89 (s, 3H), 3.55 (s, 2H), 2.71-2.66 (m, 1H), 2.26 (s, 3H), 1.71 (m, 2H), 1.48 (s, 2H) |
| | 5-(4-((3-fluoropiperidin-1- | | | |
| | yl)methyl)phenyl)-3-(6- | | | |
| | methoxypyridin-3-yl)-1H- | | | |
| | pyrrolo[2,3-b]pyridine | | | |
| 41 | 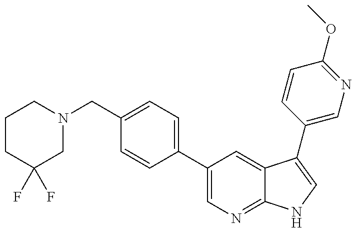 [SMILES: COc1ccc(-c2c[nH]c3ncc(-c4ccc(CN5CCCC(F)(F)C5)cc4)cc23)cn1] | E39 | 435.4 [M + H] | 11.99 (s, 1H), 8.59 (d, J = 11.6 Hz, 2H), 8.39 (s, 1H), 8.12 (d, J = 8.8 Hz, 1H), 7.89 (d, J = 1.2 Hz, 1H), 7.75 (d, J = 7.6 Hz, 2H), 7.40-7.38 (m, 2H), 6.91- 6.89 (m, 1H), 3.88 (s, 3H), 3.61 (s, 2H), 2.66-2.61 (m, 2H), 2.31 (s, 3H), 1.91-1.84 (m, 1H), 1.65 (s, 2H) |
| | 5-(4-((3,3-difluoropiperidin-1- | | | |
| | yl)methyl)phenyl)-3-(6- | | | |
| | methoxypyridin-3-yl)-1H- | | | |
| | pyrrolo[2,3-b]pyridine | | | |
| 42 | 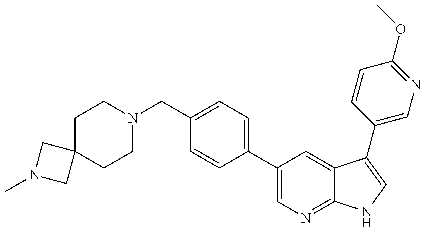 [SMILES: COc1ccc(-c2c[nH]c3ncc(-c4ccc(CN5CCC6(CC5)CN(C)C6)cc4)cc23)cn1] | E40 | 454.3 [M + H] | 11.99 (s, 1H), 8.59-8.57 (m, 2H), 8.37 (s, 1H), 8.12-8.10 (m, 1H), 7.89 (s, 1H), 7.72-7.70 (m, 2H), 7.38-7.36 (m, 2H), 6.91- 6.89 (m, 1H), 3.88 (s, 3H), 3.43- 3.41 (m, 2H), 2.92-2.90 (m, 4H), 2.31-2.27 (m, 4H), 2.22 (s, 3H), 1.64-1.62 (m, 4H) |
| | 7-(4-(3-(6-methoxypyridin-3- | | | |
| | yl)-1H-pyrrolo[2,3-b]pyridin-5- | | | |
| | yl)benzyl)-2-methyl-2,7- | | | |
| | diazaspiro[3.5]nonane | | | |
| 43 | 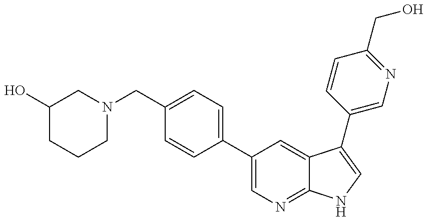 [SMILES: OCc1ccc(-c2c[nH]c3ncc(-c4ccc(CN5CCCC(O)C5)cc4)cc23)cn1] | E41 | 415.4 [M + H] | 12.08 (s, 1H), 8.90 (s, 1H), 8.56 (s, 1H), 8.42 (s, 1H), 8.19 (d, J = 7.6 Hz, 1H), 8.10 (s, 1H), 7.71 (d, J = 6.4 Hz, 2H), 7.51 (d, J = 7.6 Hz, 1H), 7.37 (d, J = 6.4 Hz, 2H), 5.39-5.37 (m, 1H), 4.57 (s, 2H), 3.52-3.41 (s, 4H), 2.79 (s, 1H), 2.65 (s, 1H), 1.86-1.69 (m, 4H), 1.42 (s, 1H), 1.06 (s, 1H) |
| | 1-(4-(3-(6- | | | |
| | (hydroxymethyl)pyridin-3-yl)- | | | |
| | 1H-pyrrolo[2,3-b]pyridin-5- | | | |
| | yl)benzyl)piperidin-3-ol | | | |
| 44 | 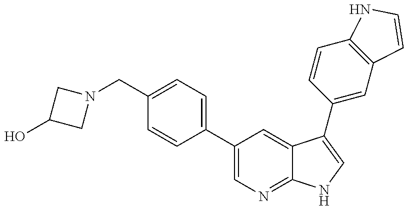 [SMILES: OC1CN(Cc2ccc(-c3cnc4[nH]cc(-c5ccc6[nH]ccc6c5)c4c3)cc2)C1] | E42 | 395.3 [M + H] | 11.79 (s, 1H), 11.05 (s, 1H), 8.50 (s, 1H), 8.37 (s, 1H), 7.87 (s, 1H), 7.74 (s, 1H), 7.65 (d, J = 7.8 Hz, 2H), 7.45 (s, 2H), 7.34 (d, J = 8.2 Hz, 3H), 6.46 (s, 1H), 5.29 (d, J = 6.4 Hz, 1H), 4.18 (d, J = 6.4 Hz, 1H), 3.57 (s, 2H), 3.49 (s, 2H), 2.77 (d, J = 6.7 Hz, 2H) |
| | 1-(4-(3-(1H-indol-5-yl)-1H- | | | |
| | pyrrolo[2,3-b]pyridin-5- | | | |
| | yl)benzyl)azetidin-3-ol | | | |
| 45 | 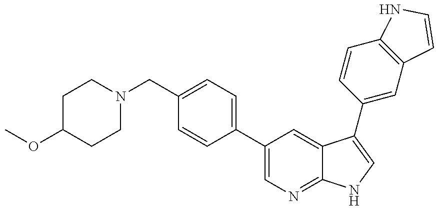 [SMILES: COC1CCN(Cc2ccc(-c3cnc4[nH]cc(-c5ccc6[nH]ccc6c5)c4c3)cc2)CC1] | E43 | 437.3 [M + H] | 11.77 (s, 1H), 11.03 (s, 1H), 7.88 (s, 2H), 7.73 (d, J = 2.5 Hz, 2H), 7.67 (d, J = 7.8 Hz, 2H), 7.46 (s, 2H), 7.41-7.30 (m, 4H), 3.48 (s, 2H), 3.31-3.29 (m, 6H), 3.20-3.18 (m, 2H), 2.65 (s, 1H), 2.11 (d, J = 11.2 Hz, 1H), 1.81 (d, J = 12.2 Hz, 1H), 1.42 (d, J = 10.8 Hz, 1H) |
| | 3-(1H-indol-5-yl)-5-(4-((4- | | | |
| | methoxypiperidin-1- | | | |
| | yl)methyl)phenyl)-1H- | | | |
| | pyrrolo[2,3-b]pyridine | | | |
| 46 | 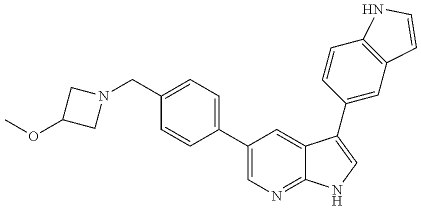 [SMILES: COC1CN(Cc2ccc(-c3cnc4[nH]cc(-c5ccc6[nH]ccc6c5)c4c3)cc2)C1] | E44 | 409.3 [M + H] | 11.77 (d, J = 2.8 Hz, 1H), 11.03 (s, 1H), 8.51 (s, 1H), 8.38 (s, 1H), 7.88 (s, 1H), 7.73 (d, J = 2.6 Hz, 1H), 7.66 (d, J = 7.8 Hz, 2H), 7.46 (s, 2H), 7.39-7.30 (m, 3H), 6.47 (s, 1H), 3.97 (d, J = 5.8 Hz, 1H), 3.60 (s, 2H), 3.48 (d, J = 6.7 Hz, 2H), 3.14 (s, 3H), 2.85 (t, J = 6.5 Hz, 2H) |
| | 3-(1H-indol-5-yl)-5-(4-((3- | | | |
| | methoxyazetidin-1- | | | |
| | yl)methyl)phenyl)-1H- | | | |
| | pyrrolo[2,3-b]pyridine | | | |
| 47 | 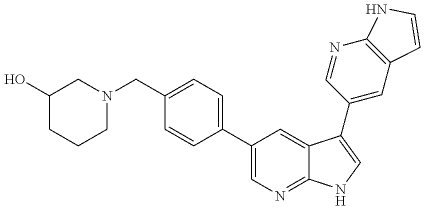 [SMILES: OC1CCCN(Cc2ccc(-c3cnc4[nH]cc(-c5cnc6[nH]ccc6c5)c4c3)cc2)C1] | E45 | 422.3 [M − H] | 11.94 (s, 1H), 11.61 (s, 1H), 8.59 (d, J = 1.6 Hz, 2H), 8.40 (s, 1H), 8.29 (s, 1H), 7.87 (s, 1H), 7.70 (d, J = 7.6 Hz, 2H), 7.46 (d, J = 3.2 Hz, 1H), 7.37 (d, J = 8 Hz, 2H), 6.48 (d, J = 3.6 Hz, 1H), 4.62-4.61 (m, 1H), 3.55- 3.44 (m, 3H), 2.80 (s, 1H), 2.68 (s, 1H), 1.86-1.02 (m, 6H) |
| | 1-(4-(1H,1′H-[3,5′- | | | |
| | bipyrrolo[2,3-b]pyridin]-5- | | | |
| | yl)benzyl)piperidin-3-ol | | | |
| 48 | 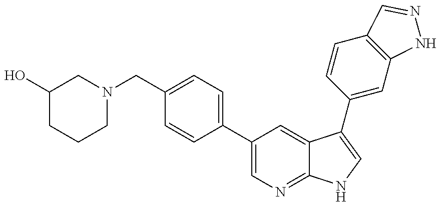 [SMILES: OC1CCCN(Cc2ccc(-c3cnc4[nH]cc(-c5ccc6cn[nH]c6c5)c4c3)cc2)C1] | E46 | 424.3 [M + H] | 12.91 (s, 2H), 8.56 (s, 1H), 8.45 (s, 1H), 8.04-7.98 (m, 2H), 7.84- 7.79 (m, 2H), 7.69 (d, J = 8 Hz, 2H), 7.54 (d, J = 8 Hz , 1H), 7.39 (d, J = 8 Hz, 2H), 4.57 (d, 1H), 3.54 (d, J = 16 Hz, 2H), 3.43 (t, J = 8 Hz, 2H), 2.80 (d, J = 8 Hz, 1H), 2.68-2.65 (m, 1H), 1.87- 1.78 (m, 3H), 1.67-1.65 (m, 1H), 1.59-1.57 (m, 1H) |
| | 1-(4-(3-(1H-indazol-6-yl)-1H- | | | |
| | pyrrolo[2,3-b]pyridin-5- | | | |
| | yl)benzyl)piperidin-3-ol | | | |
| 49 | 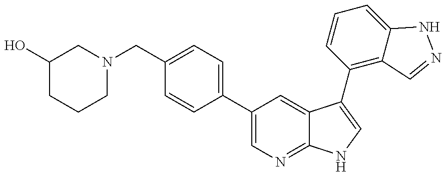 [SMILES: OC1CCCN(Cc2ccc(-c3cnc4[nH]cc(-c5cccc6[nH]ncc56)c4c3)cc2)C1] | E47 | 422.3 [M − H] | 13.21 (s, 1H), 12.12 (s, 1H), 8.60 (d, J = 2.0 Hz, 1H), 8.35 (d, J = 1.6 Hz, 1H), 8.25 (s, 1H), 8.02 (d, J = 2.8 Hz, 1H), 7.68 (d, J = 8.0 Hz, 2H), 7.46 (s, 3H), 7.39 (d, J = 8.0 Hz, 2H), 4.56 (d, J = 4.8 Hz, 1H), 3.57-3.38 (m, 3H), 2.82 (d, J = 6.8 Hz, 1H), 2.67 (s, 1H), 1.89-1.60 (m, 4H), 1.45 (m, 1H), 1.09 (m, 1H) |
| | 1-(4-(3-(1H-indazol-4-yl)-1H- | | | |
| | pyrrolo[2,3-b]pyridin-5- | | | |
| | yl)benzyl)piperidin-3-ol | | | |
| 50 | 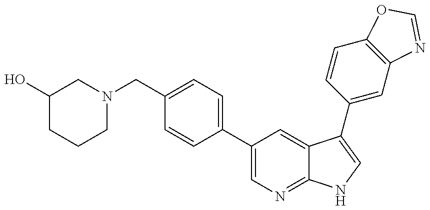 [SMILES: OC1CCCN(Cc2ccc(-c3cnc4[nH]cc(-c5ccc6ocnc6c5)c4c3)cc2)C1] | E48 | 425.2 [M + H] | 12.01 (bs, 1H), 8.76 (s 1H), 8.57 (s, 1H), 8.44 (s, 1H), 8.15 (s, 1H), 7.97 (s, 1H), 7.88-7.82 (m, 2H), 7.72 (d, J = 6.8 Hz, 2H), 7.39 (d, J = 6.8 Hz, 2H), 4.57- 4.56 (m, 1H), 3.57-3.54 (m, 2H), 3.46-3.42 (m, 2H), 2.83-2.81 (m, 2H), 2.66 (m, 1H), 1.90-1.41 (m, 4H) |
| | 1-(4-(3-(benzo[d]oxazol-5-yl)- | | | |
| | 1H-pyrrolo[2,3-b]pyridin-5- | | | |
| | yl)benzyl)piperidin-3-ol | | | |
| 51 | 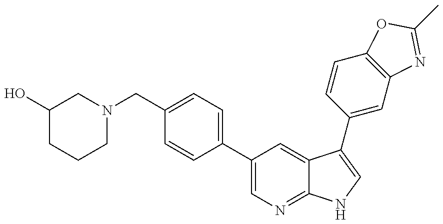 [SMILES: Cc1nc2cc(-c3c[nH]c4ncc(-c5ccc(CN6CCCC(O)C6)cc5)cc34)ccc2o1] | E49 | 437.3 [M − H] | 11.98 (s, 1H), 8.57 (d, J = 1.6 Hz, 1H), 8.42 (s, 1H), 8.01 (d, J = 2.4 Hz, 2H), 7.94 (d, J = 2.6 Hz, 1H), 7.77-7.70 (m, 4H), 7.40 (d, J = 8.0 Hz, 1H), 4.55 (d, J = 4.8 Hz, 1H), 3.57-3.55 (m, 1H), 3.46 (d, J = 13.2 Hz, 2H), 2.84 (d, J = 10.3 Hz, 1H), 2.68-2.67 (m, 2H), 2.64 (s, 2H), 2.46 (s, 1H), 1.95-1.78 (m, 3H), 1.73- 1.71 (m, 1H), 1.61-1.59 (m, 1H) |
| | 1-(4-(3-(2- | | | |
| | methylbenzo[d]oxazol-5-yl)- | | | |
| | 1H-pyrrolo[2,3-b]pyridin-5- | | | |
| | yl)benzyl)piperidin-3-ol | | | |
| 52 | 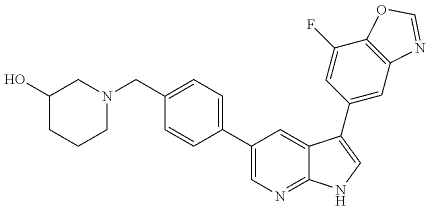 [SMILES: OC1CCCN(Cc2ccc(-c3cnc4[nH]cc(-c5cc(F)c6ocnc6c5)c4c3)cc2)C1] | E50 | 441.3 [M − H] | 12.03 (s, 1H), 8.79 (s, 1H), 8.51- 8.50 (m, 1H), 8.42-8.41 (m, 1H), 7.98-7.96 (m, 2H), 7.76-7.74 (m, 1H), 7.68 (d, J = 7.6 Hz, 2H), 7.34 (d, J = 8.0 Hz, 2H), 4.55 (d, J = 4.8 Hz, 1H), 3.51-3.48 (m, 2H), 3.44-3.42 (m, 1H), 2.77- 2.75 (m, 1H), 2.63-2.61 (m, 1H), 1.82-1.61 (m, 4H), 1.45-1.43 (m, 1H), 1.19-1.15 (m, 1H) |
| | 1-(4-(3-(7- | | | |
| | fluorobenzo[d]oxazol-5-yl)-1H- | | | |
| | pyrrolo[2,3-b]pyridin-5- | | | |
| | yl)benzyl)piperidin-3-ol | | | |
| 53 | 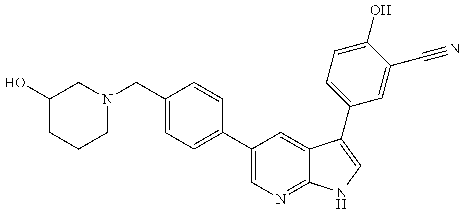 [SMILES: N#Cc1cc(-c2c[nH]c3ncc(-c4ccc(CN5CCCC(O)C5)cc4)cc23)ccc1O] | E52 | 425.3 [M + H] | 11.57 (s, 1H), 8.46 (s, 1H), 8.22 (s, 1H), 7.67-7.65 (m, 2H), 7.49- 7.47 (m, 2H), 7.51-7.49 (m, 2H), 7.38-7.36 (d, J = 7.6 Hz, 2H), 6.32 (s, 1H), 4.56 (s, 2H), 3.55 (d, J = 13.2 Hz , 1H), 3.44 (d, J = 13.2 Hz, 1H), 2.82-2.80 (m, 1H), 2.69-2.66 (m, 2H), 1.86 (s, 1H), 1.80-1.78 (d, J = 8.4 Hz, 1H), 1.72 (s, 1H), 1.69 (s, 1H), 1.44 (s, 1H) |
| | 2-hydroxy-5-(5-(4-((3- | | | |
| | hydroxypiperidin-1- | | | |
| | yl)methyl)phenyl)-1H- | | | |
| | pyrrolo[2,3-b]pyridin-3- | | | |
| | yl)benzonitrile | | | |
| 54 | 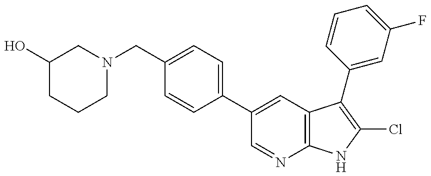 [SMILES: OC1CCCN(Cc2ccc(-c3cnc4[nH]c(Cl)c(-c5cccc(F)c5)c4c3)cc2)C1] | E53 | 434.2 [M − H] | 12.96 (s, 1H), 8.56 (s, 1H), 8.15 (s, 1H), 7.68 (d, J = 8.0 Hz, 2H), 7.59-7.54 (m, 2H), 7.53-7.50 (m, 1H), 7.38 (d, J = 8.4 Hz, 2H), 7.21-7.17 (m, 1H), 4.55-4.54 (m, 1H), 3.57-3.53 (m, 1H), 3.47 (s, 2H), 2.19-2.17 (m, 1H), 2.77- 2.75 (m, 1H), 1.89-1.81 (m, 2H), 1.74-1.69 (m, 2H), 1.60-1.52 (m, 2H) |
| | 1-(4-(2-chloro-3-(3- | | | |
| | fluorophenyl)-1H-pyrrolo[2,3- | | | |
| | b]pyridin-5- | | | |
| | yl)benzyl)piperidin-3-ol | | | |
| 55 | 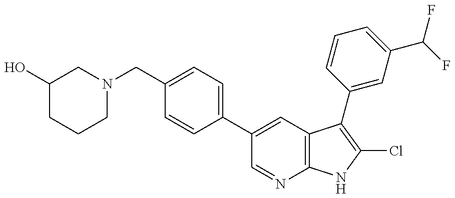 [SMILES: OC1CCCN(Cc2ccc(-c3cnc4[nH]c(Cl)c(-c5cccc(C(F)F)c5)c4c3)cc2)C1] | E54 | 466.1 [M − H] | 12.85 (s, 1H), 8.51 (d, J = 4.0 Hz, 1H), 8.07 (d, J = 2.0 Hz, 1H), 7.79 (d, J = 8.0 Hz, 2H), 7.60 (t, J = 8.0 Hz, 3H), 7.51 (d, J = 8.0 Hz, 1H), 7.31 (d, J = 8.0 Hz, 2H), 7.21-6.93 (m, 1H), 4.48 (d, J = 4.0 Hz, 1H), 3.47 (d, J = 12.0 Hz, 1H), 3.41-3.35 (m, 2H), 2.73 (d, J = 4.0 Hz, 1H), 2.60- 2.58 (m, 1H), 1.83-1.78 (m, 2H), 1.65 (t, J = 2.4 Hz, 1H), 1.53 (d, J = 12.0 Hz, 1H), 1.40-1.30 (m, 1H), 1.01-0.95 (m, 1H) |
| | 1-(4-(2-chloro-3-(3- | | | |
| | (difluoromethyl)phenyl)-1H- | | | |
| | pyrrolo[2,3-b]pyridin-5- | | | |
| | yl)benzyl)piperidin-3-ol | | | |
| 56 | 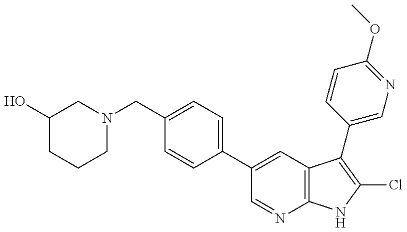 [SMILES: COc1ccc(-c2c(Cl)[nH]c3ncc(-c4ccc(CN5CCCC(O)C5)cc4)cc23)cn1] | E55 | 449.2 [M + H] | 11.96 (s, 1H), 8.55-8.54 (m, 1H), 8.44-8.43 (m, 1H), 8.08- 8.07 (m, 1H), 7.99-7.97 (m, 1H), 7.66 (d, J = 8.0 Hz, 2H), 7.35 (d, J = 7.6 Hz, 2H), 6.93 (d, J = 8.8 Hz, 1H), 4.51 (d, J = 4.8 Hz, 1H), 3.90 (s, 3H), 3.54-3.52 (m, 1H), 3.47 (s, 2H), 2.79-2.77 (m, 1H), 2.65-2.63 (m, 1H), 1.88- 1.70 (m, 3H), 1.61-1.59 (m, 1H), 1.49-1.47 (m, 1H), 1.09-1.07 (m, 1H) |
| | 1-(4-(2-chloro-3-(6- | | | |
| | methoxypyridin-3-yl)-1H- | | | |
| | pyrrolo[2,3-b]pyridin-5- | | | |
| | yl)benzyl)piperidin-3-ol | | | |
| 57 | 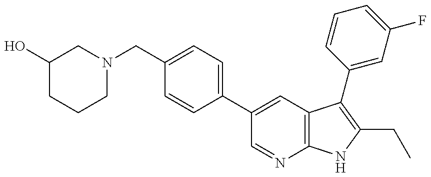 [SMILES: CCc1[nH]c2ncc(-c3ccc(CN4CCCC(O)C4)cc3)cc2c1-c1cccc(F)c1] | E56 | 428.2 [M − H] | 11.88 (s, 1H), 8.48 (d, J = 2.0 Hz, 1H), 8.04 (d, J = 2.0 Hz, 1H), 7.65 (d, J = 8.0 Hz, 2H), 7.54-7.50 (m, 1H), 7.38-7.29 (s, 4H), 7.17-7.13 (s, 1H), 4.54 (s, 1H), 3.56-3.47 (m, 3H), 2.91- 2.89 (m, 3H), 1.88-1.71 (m, 6H), 1.34-1.30 (m, 4H) |
| | 1-(4-(2-ethyl-3-(3- | | | |
| | fluorophenyl)-1H-pyrrolo[2,3- | | | |
| | b]pyridin-5- | | | |
| | yl)benzyl)piperidin-3-ol | | | |
| 58 | 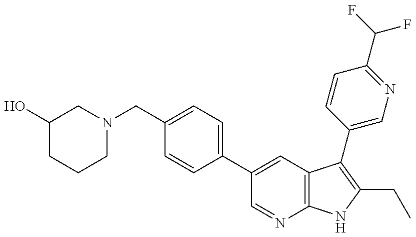 [SMILES: CCc1[nH]c2ncc(-c3ccc(CN4CCCC(O)C4)cc3)cc2c1-c1ccc(C(F)F)nc1] | E57 | 461.2 [M − H] | 9.89 (s, 1H), 8.84 (s, J = 4 Hz, 1H), 8.55 (d, J = 4 Hz, 1H), 8.07 (s, 1H), 7.99-7.97 (m, 1H), 7.78 (d, J = 8 Hz, 1H), 7.59 (d, J = 8 Hz, 2H), 7.48 (d, J = 8 Hz, 2H), 7.02-6.60 (m, 1H), 3.96 (s, 1H), 3.73 (s, 2H), 3.05-2.99 (m, 2H), 2.85-2.69 (m, 4H), 2.46 (s, 1H), 2.02-1.97 (m, 2H), 1.64 (s, 2H), 1.47 (t, J = 8 Hz, 3H) |
| | 1-(4-(3-(6- | | | |
| | (difluoromethyl)pyridin-3-yl)- | | | |
| | 2-ethyl-1H-pyrrolo[2,3- | | | |
| | b]pyridin-5- | | | |
| | yl)benzyl)piperidin-3-ol | | | |
| 59 | 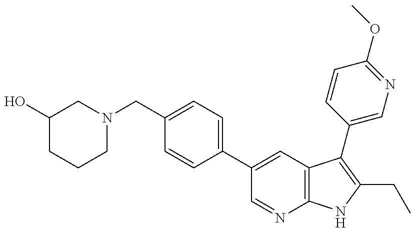 [SMILES: CCc1[nH]c2ncc(-c3ccc(CN4CCCC(O)C4)cc3)cc2c1-c1ccc(OC)nc1] | E58 | 441.3 [M − H] | 11.83 (s, 1H), 8.46 (d, J = 1.6 Hz, 1H), 8.26 (d, J = 2.4 Hz, 1H), 7.95 (s, 1H), 7.85-7.82 (m, 1H), 7.61 (d, J = 7.6 Hz, 2H), 7.34 (d, J = 7.2 Hz, 2H), 6.91 (d, J = 8.4 Hz, 1H), 4.58 (s, 1H), 3.89 (s, 3H), 3.47-3.45 (m, 1H), 3.43-3.41 (m, 2H), 2.86-2.84 (m, 2H), 2.65-2.63 (m, 2H), 1.89- 1.81 (m, 4H), 1.42-1.39 (m, 1H), 1.27 (t, J = 7.2 Hz, 3H), 1.05- 1.03 (m, 1H). |
| | 1-(4-(2-ethyl-3-(6- | | | |
| | methoxypyridin-3-yl)-1H- | | | |
| | pyrrolo[2,3-b]pyridin-5- | | | |
| | yl)benzyl)piperidin-3-ol | | | |
| 60 | 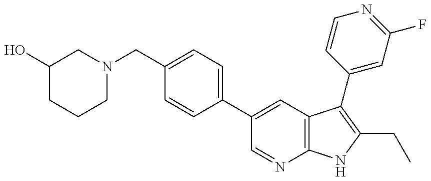 [SMILES: CCc1[nH]c2ncc(-c3ccc(CN4CCCC(O)C4)cc3)cc2c1-c1ccnc(F)c1] 1-(4-(2-ethyl-3-(2- fluoropyridin-4-yl)-1H- | E59 | 431.2 [M + H] | 12.08 (s, 1H), 8.45 (d, J = 2.0 Hz, 1H), 8.22 (d, J = 5.2 Hz, 1H), 8.11 (d, J = 1.6 Hz, 1H), 7.61 (d, J = 8.0 Hz, 2H), 7.45 (d, J = 5.2 Hz, 1H), 7.31 (d, J = 7.6 Hz, 2H), 7.20 (s, 1H), 4.47 (d, J = 4.4 Hz, 1H), 3.49-3.47 (m, 1H), 3.38-3.35 (m, 2H), 2.90- 2.85 (m, 2H), 2.75-2.73 (m, 1H), 2.60-2.58 (m, 1H), 1.84-1.79 (m, 3H), 1.67-1.64 (m, 1H), 1.37- 1.35 (m, 1H), 1.26 (t, J = 7.6 Hz, 3H), 1.04-1.01 (m, 1H) |
| | pyrrolo[2,3-b]pyridin-5- | | | |
| | yl)benzyl)piperidin-3-ol | | | |
| 61 | 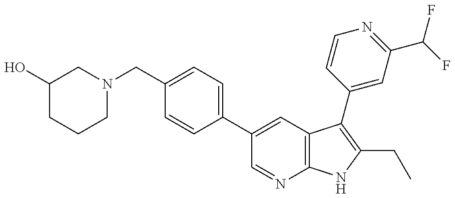 [SMILES: CCc1[nH]c2ncc(-c3ccc(CN4CCCC(O)C4)cc3)cc2c1-c1ccnc(C(F)F)c1] 1-(4-(3-(2- | E60 | 461.2 [M − H] | 12.16 (s, 1H), 8.73 (d, J = 4.0 Hz, 1H), 8.53 (s, 1H), 8.17 (s, 1H), 7.76 (s, 2H), 7.67 (d, J = 8.0 Hz, 2H), 7.38 (d, J = 4.0 Hz, 2H), 7.17-6.89 (m, 1H), 4.54 (d, J = 4.0 Hz , 1H), 3.55 (d, J = 16.0 Hz, 1H), 3.44 ( d, J = 16.0 Hz, 2H), 2.96 (d, J = 4.0 Hz, 1H), 2.93-2.91 (m, 2H), 2.82 (d, J = 8.0 Hz ,1H), 1.90 (t, J = 8.0 Hz, 1H) 1.73 (t, J = 8.0 Hz, 2H), 1.62 (d, J = 16.0 Hz, 1H), 1.48- 1.41 (m, 1H), 1.36 (t, J = 8.0 Hz, 3H), 1.11-1.06 (m, 1H) |
| | (difluoromethyl)pyridin-4-yl)- | | | |
| | 2-ethyl-1H-pyrrolo[2,3- | | | |
| | b]pyridin-5- | | | |
| | yl)benzyl)piperidin-3-ol | | | |
| 62 | 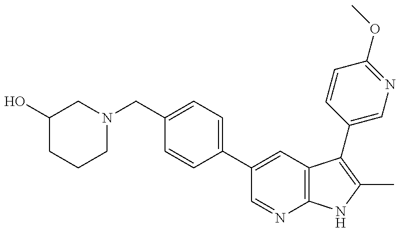 [SMILES: COc1ccc(-c2c(C)[nH]c3ncc(-c4ccc(CN5CCCC(O)C5)cc4)cc23)cn1] | E61 | 429.2 [M + H] | 11.81 (s, 1H), 8.44 (d, J = 1.6 Hz, 1H), 8.32 (d, J = 2.4 Hz, 1H), 7.99 (s, 1H), 7.90-7.88 (m, 1H), 7.65-7.63 (m, 2H), 7.38- 7.33 (m, 2H), 6.92 (d, J = 8.4 Hz, 1H), 4.55 (d, J = 4.8 Hz, 1H), 3.89 (s, 3H), 3.53-3.51 (m, 1H), 3.47-3.45 (m, 2H), 2.80- 2.78 (m, 1H), 2.65-2.63 (m, 1H), 2.50 (s, 3H), 1.87-1.83 (m, 2H), 1.61-1.59 (m, 2H), 1.49-1.47 (m, 1H), 1.08-1.06 (m, 1H) |
| | 1-(4-(3-(6-methoxypyridin-3- | | | |
| | yl)-2-methyl-1H-pyrrolo[2,3- | | | |
| | b]112yridine-5- | | | |
| | yl)benzyl)piperidin-3-ol | | | |
| 63 | 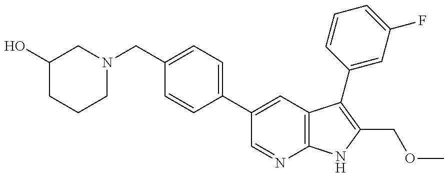 [SMILES: COCc1[nH]c2ncc(-c3ccc(CN4CCCC(O)C4)cc3)cc2c1-c1cccc(F)c1] 1-(4-(3-(3-fluorophenyl)-2- (methoxymethyl)-1H- | E62 | 444.2 [M − H] | 12.26 (s, 1H), 8.58 (d, J = 2.0 Hz, 1H), 8.19 (d, J = 1.6 Hz, 1H), 7.68 (d, J = 8.0 Hz, 2H), 7.56-7.52 (m, 1H), 7.47-7.46 (m, 1H), 7.42-7.37 (m, 3H), 7.18- 7.17 (m, 1H), 4.59 (s, 2H), 4.54 (d, J = 4.0 Hz, 1H), 3.57-3.54 (m, 1H), 3.46-3.42 (m, 2H), 3.35 (s, 3H), 2.83-2.81 (m, 1H), 2.68- 2.66 (m, 1H), 1.91-1.86 (m, 1H), 1.81-1.79 (m, 1H), 1.74-1.70 (m, 1H), 1.64-1.60 (m, 1H), 1.48- 1.46 (m, 1H), 1.22-1.20 (m, 1H) |
| | pyrrolo[2,3-b]pyridin-5- | | | |
| | yl)benzyl)piperidin-3-ol | | | |
| 64 | 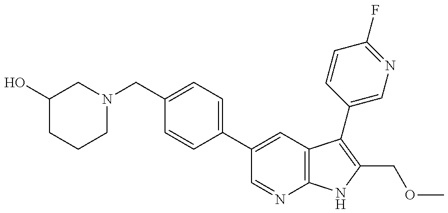 [SMILES: COCc1[nH]c2ncc(-c3ccc(CN4CCCC(O)C4)cc3)cc2c1-c1ccc(F)nc1] | E63 | 445.3 [M − H] | 12.24 (s, 1H), 8.57 (d, J = 2.4 Hz, 2H), 8.41 (d, J = 2.0 Hz, 1H), 8.20-8.15 (m, 2H), 7.67 (d, J = 8.0 Hz, 2H), 7.36-7.28 (m, 2H), 4.54-4.52 (m, 3H), 3.53- 3.39 (m, 3H), 2.78 (d, J = 7.2 Hz, 1H), 1.84-1.57 (m, 6H), 1.44-1.38 (m, 3H), 1.07-1.01 (m, 1H) |
| | 1-(4-(3-(6-fluoropyridin-3-yl)- | | | |
| | 2-(methoxymethyl)-1H- | | | |
| | pyrrolo[2,3-b]pyridin-5- | | | |
| | yl)benzyl)piperidin-3-ol | | | |
| 65 | 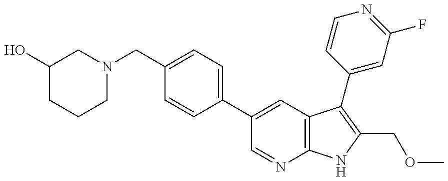 [SMILES: COCc1[nH]c2ncc(-c3ccc(CN4CCCC(O)C4)cc3)cc2c1-c1ccnc(F)c1] | E64 | 445.3 [M − H] | 12.47 (s, 1H), 8.60 (d, J = 1.6 Hz, 1H), 8.31-8.28 (m, 2H), 7.70 (d, J = 8.0 Hz, 2H), 7.61 (d, J = 4.8 Hz, 1H), 7.38-7.35 (m, 3H), 4.63 (s, 2H), 4.57 (d, J = 4.8 Hz, 1H), 3.55-3.41 (m, 6H), 2.80 (d, J = 8.0 Hz, 1H), 2.65 (s, 1H), 1.88-1.61 (m, 4H), 1.58-1.39 (m, 1H), 1.08-1.02 (m, 1H) |
| | 1-(4-(3-(2-fluoropyridin-4-yl)- | | | |
| | 2-(methoxymethyl)-1H- | | | |
| | pyrrolo[2,3-b]pyridin-5- | | | |
| | yl)benzyl)piperidin-3-ol | | | |
| 66 | 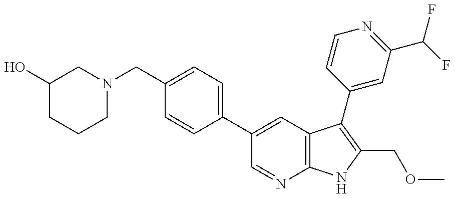 [SMILES: COCc1[nH]c2ncc(-c3ccc(CN4CCCC(O)C4)cc3)cc2c1-c1ccnc(C(F)F)c1] | E65 | 477.3 [M − H] | 12.48 (s, 1H), 8.71 (d, J = 4.8 Hz, 1H), 8.60 (d, J = 2.0 Hz, 1H), 8.29 (d, J = 2.0 Hz, 1H), 7.86-7.82 (m, 2H), 7.68 (d, J = 7.6 Hz, 2H), 7.37 (d, J = 8.4 Hz, 2H), 7.01 (t, J = 6.0 Hz, 1H), 4.59 (s, 2H), 4.55 (d, J = 4.8 Hz, 1H), 3.51-3.36 (m, 5H), 2.64 (d, J = 1.6 Hz, 1H), 2.48-2.37 (m, 4H), 1.45-1.01 (m, 4H) |
| | 1-(4-(3-(2- | | | |
| | (difluoromethyl)pyridin-4-yl)- | | | |
| | 2-(methoxymethyl)-1H- | | | |
| | pyrrolo[2,3-b]pyridin-5- | | | |
| | yl)benzyl)piperidin-3-ol | | | |
| 67 | 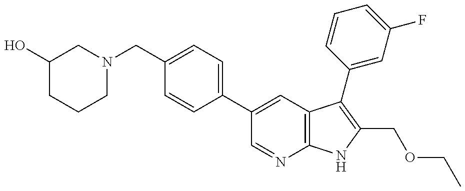 [SMILES: CCOCc1[nH]c2ncc(-c3ccc(CN4CCCC(O)C4)cc3)cc2c1-c1cccc(F)c1] 1-(4-(2-(ethoxymethyl)-3-(3- | E66 | 460.3 [M + H] | 12.15 (s, 1H), 8.57 (d, J = 2.0 Hz, 1H), 8.18 (d, J = 2.0 Hz, 1H), 7.68 (d, J = 8.4 Hz, 1H), 7.57-7.53 (m, 2H), 7.49-7.37 (m, 4H), 7.19-7.14 (m, 1H), 4.62 (s, 2H), 4.54 (brs, 1H), 3.59-3.53 (m, 3H), 3.49-3.42 (m, 2H), 3.19-3.14 (m, 1H), 2.83-2.81 (m, 1H), 1.91-1.89 (m, 1H), 1.88- 1.86 (m, 1H), 1.79-1.77 (m, 1H), 1.69-1.67 (m, 1H), 1.48-1.46 (m, 1H), 1.18 (t, J = 6.8 Hz, 3H), 1.09-1.05 (m, 1H) |
| | fluorophenyl)-1H-pyrrolo[2,3- | | | |
| | b]pyridin-5- | | | |
| | yl)benzyl)piperidin-3-ol | | | |
| 68 | 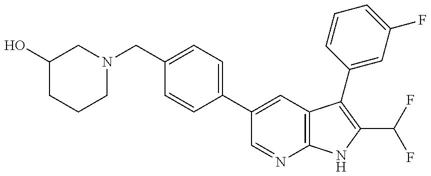 [SMILES: OC1CCCN(Cc2ccc(-c3cnc4[nH]c(C(F)F)c(-c5cccc(F)c5)c4c3)cc2)C1] | E67 | 452.2 [M + H] | 12.86 (s, 1H), 8.69 (s, 1H), 8.19 (s, 1H), 7.68 (d, J = 8.0 Hz, 2H), 7.58-7.54 (m, 2H), 7.41-7.37 (m, 4H), 7.24- 7.21 (m, 1H), 4.54 (d, J = 4.8 Hz, 1H), 3.57-3.53 (m, 1H), 3.48 (s, 2H), 3.25-3.23 (m, 2H), 1.91-1.80 (m, 2H), 1.57- 1.51 (m, 2H), 1.34-1.30 (m, 2H) |
| | 1-(4-(2-(difluoromethyl)-3-(3- | | | |
| | fluorophenyl)-1H-pyrrolo[2,3- | | | |
| | b]pyridin-5- | | | |
| | yl)benzyl)piperidin-3-ol | | | |
| 69 | 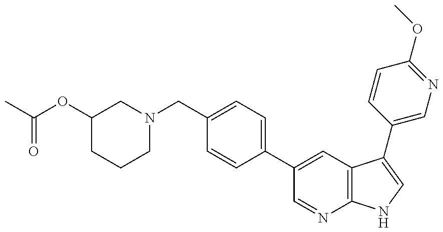 [SMILES: COc1ccc(-c2c[nH]c3ncc(-c4ccc(CN5CCCC(OC(C)=O)C5)cc4)cc23)cn1] | E69 | 457.3 [M + H] | 11.99 (s, 1H), 8.59-8.56 (m, 2H), 8.37 (s, 1H), 8.11 (d, J = 8.8 Hz, 1H), 7.88 (s, 1H), 7.73- 7.71 (m, 2H), 7.37 (d, J = 7.2 Hz, 2H), 6.90 (d, J = 8.4 Hz, 1H), 4.67 (s, 1H), 3.88 (s, 3H), 3.57-3.55 (m, 2H), 2.79-2.77 (m, 1H), 2.65-2.63 (m, 1H), 2.08- 2.06 (m, 2H), 1.96 (s, 3H), 1.83- 1.81 (m, 1H), 1.50-1.48 (m, 1H), 1.33-1.31 (m, 1H), 1.21-1.19 (m, 1H) |
| | 1-(4-(3-(6-methoxypyridin-3- | | | |
| | yl)-1H-pyrrolo[2,3-b]pyridin-5- | | | |
| | yl)benzyl)piperidin-3-yl acetate | | | |
| 70 | 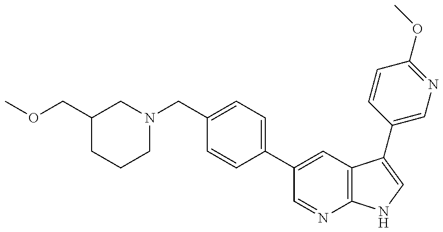 [SMILES: COCC1CCCN(Cc2ccc(-c3cnc4[nH]cc(-c5ccc(OC)nc5)c4c3)cc2)C1] | E70 | 443.3 [M + H] | 11.97 (s, 1H), 8.56 (d, J = 10.0 Hz, 2H), 8.36 (s, 1H), 8.10-8.08 (m, 1H), 7.86 (s, 1H), 7.70 (d, J = 8.0 Hz, 2H), 7.36 (d, J = 8.0 Hz, 2H), 6.89-6.87 (s, 1H), 3.90 (s, 3H), 3.48-3.45 (m, 4H), 3.15 (s, 3H), 3.10-3.08 (m, 1H), 2.84- 2.80 (m, 2H), 1.93-1.91 (m, 1H), 1.79-1.75 (m, 2H), 1.68-1.65 (m, 2H), 1.48-1.45 (m, 1H) |
| | 5-(4-((3- | | | |
| | (methoxymethyl)piperidin-1- | | | |
| | yl)methyl)phenyl)-3-(6- | | | |
| | methoxypyridin-3-yl)-1H- | | | |
| | pyrrolo[2,3-b]pyridine | | | |
| 71 | 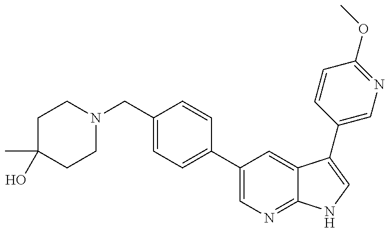 [SMILES: COc1ccc(-c2c[nH]c3ncc(-c4ccc(CN5CCC(C)(O)CC5)cc4)cc23)cn1] | E71 | 429.4 [M + H] | 11.95 (s, 1H), 8.56 (dd, J = 12.7, 2.3 Hz, 2H), 8.36 (s, 1H), 8.10 (dd, J = 8.6, 2.6 Hz, 1H), 7.87 (s, 1H), 7.69 (d, J = 8.0 Hz, 2H), 7.37 (d, J = 8.0 Hz, 2H), 6.89 (d, J = 8.6 Hz, 1H), 4.05 (s, 1H), 3.87 (s, 3H), 3.47 (s, 2H), 2.37- 2.33 (m, 4H), 1.45-1.41 (m, 4H), 1.08 (s, 3H) |
| | 1-(4-(3-(6-methoxypyridin-3- | | | |
| | yl)-1H-pyrrolo[2,3-b]pyridin-5- | | | |
| | yl)benzyl)-4-methylpiperidin-4- | | | |
| | ol | | | |
| 72 | 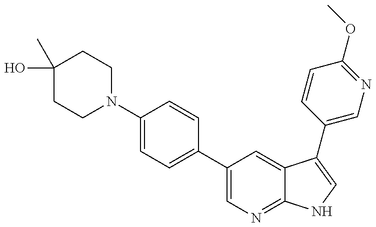 [SMILES: COc1ccc(-c2c[nH]c3ncc(-c4ccc(N5CCC(C)(O)CC5)cc4)cc23)cn1] | E72 | 415.4 [M + H] | 11.87 (s, 1H), 8.55 (s, 1H), 8.48 (s, 1H), 8.26 (s, 1H), 8.08 (d, J = 8.4 Hz, 1H), 7.83 (d, J = 8.0 Hz, 1H), 7.58 (d, J = 8.0 Hz, 2H), 7.01 (d, J = 8.0 Hz, 2H), 6.89 (d, J = 8.8 Hz, 1H), 4.26 (s, 1H), 3.87 (s, 3H), 3.35 (s, 2H), 3.16 (s, 2H), 1.55-1.41 (m, 4H), 1.14 (s, 3H) |
| | 1-(4-(3-(6-methoxypyridin-3- | | | |
| | yl)-1H-pyrrolo[2,3-b]pyridin-5- | | | |
| | yl)phenyl)-4-methylpiperidin-4- | | | |
| | ol | | | |
| 73 | 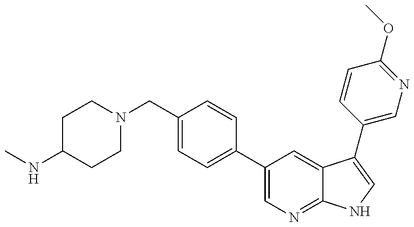 [SMILES: CNC1CCN(Cc2ccc(-c3cnc4[nH]cc(-c5ccc(OC)nc5)c4c3)cc2)CC1] | E73 | 428.3 [M + H] | 12.04 (s, 1H), 9.96 (bs, 1H), 8.83-8.81 (m, 1H), 8.60-8.58 (m, 1H), 8.42 (s, 1H), 8.09 (d, J = 8.4 Hz, 1H), 7.91 (d, J = 6.4 Hz, 2H), 7.58 (d, J = 8.0 Hz, 2H), 6.90 (d, J = 8.0 Hz, 2H), 4.34 (s, 2H), 3.80 ( s, 3H), 3.53-3.50 (m, 2H), 3.20-3.18 (m, 1H), 3.00- 2.98 (m, 2H), 2.72-2.70 (m, 1H), 2.56 (s, 3H), 2.30-2.28 (m, 2H), 1.74-1.72 (m, 1H) |
| | 1-(4-(3-(6-methoxypyridin-3- | | | |
| | yl)-1H-pyrrolo[2,3-b]pyridin-5- | | | |
| | yl)phenyl)-N-methylpiperidin- | | | |
| | 4-amine | | | |
| 74 | 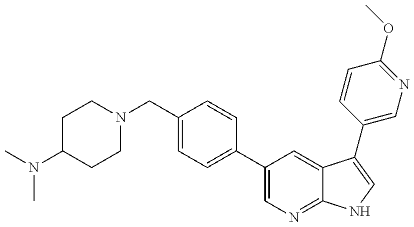 [SMILES: COc1ccc(-c2c[nH]c3ncc(-c4ccc(CN5CCC(N(C)C)CC5)cc4)cc23)cn1] | E74 | 440.4 [M − H] | 11.95 (s, 1H), 8.57-8.54 (m, 2H), 8.36 (s, 2H), 8.11-8.08 (m, 3H), 7.87-7.86 (m, 1H), 7.37- 7.35 (m, 1H), 6.90 (d, J = 8.4 Hz, 1H), 3.88 (s, 3H), 3.46 (s, 2H), 2.85-2.82 (m, 2H), 2.13 (s, 6H), 2.04-1.98 (m, 1H), 1.94- 1.89 (m, 2H), 1.71 (d, J = 12.0 Hz, 2H), 1.4-1.32 (m, 2H) |
| | 1-(4-(3-(6-methoxypyridin-3- | | | |
| | yl)-1H-pyrrolo[2,3-b]pyridin-5- | | | |
| | yl)benzyl)-N,N- | | | |
| | dimethylpiperidin-4-amine | | | |
| 75 | 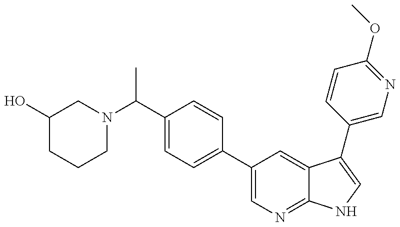 [SMILES: COc1ccc(-c2c[nH]c3ncc(-c4ccc(C(C)N5CCCC(O)C5)cc4)cc23)cn1] | E75 | 427.3 [M + H] | 11.98 (s, 1H), 8.58-8.56 (m, 2H), 8.37 (s, 1H), 8.10-8.08 (m, 1H), 7.88 (s, 1H), 7.72-7.69 (m, 2H), 7.38-7.36 (m, 2H), 6.89 (d, J = 8.0 Hz, 1H), 4.54-4.49 (m, 1H), 3.88 (s, 3H), 3.54-3.48 (m, 2H), 2.81-2.78 (m, 1H), 1.83- 1.73 (m, 3H), 1.62-1.54 (m, 2H), 1.42-1.36 (m, 5H) |
| | 1-(1-(4-(3-(6-methoxypyridin- | | | |
| | 3-yl)-1H-pyrrolo[2,3-b]pyridin- | | | |
| | 5-yl)phenyl)ethyl)piperidin-3-ol | | | |
| 76 | 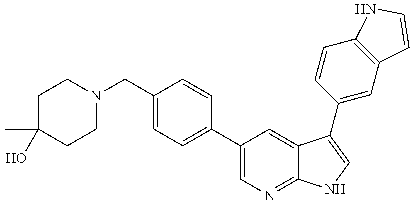 [SMILES: CC1(O)CCN(Cc2ccc(-c3cnc4[nH]cc(-c5ccc6[nH]ccc6c5)c4c3)cc2)CC1] | E76 | 437.3 [M + H] | 11.76 (s, 1H), 11.03 (s, 1H), 8.51 (s, 1H), 8.38 (s, 1H), 7.87 (s, 1H), 7.73 (s, 1H), 7.67-7.65 (m, 2H), 7.46 (s, 2H), 7.38-7.37 (m, 2H), 7.32 (s, 1H), 6.46 (s, 1H), 4.04 (s, 1H), 3.48 (s, 2H), 2.38-2.33 (m, 4H), 1.45-1.43 (m, 4H), 1.09 (s, 3H) |
| | 1-(4-(3-(1H-indol-5-yl)-1H- | | | |
| | pyrrolo[2,3-b]pyridin-5- | | | |
| | yl)benzyl)-4-methylpiperidin-4-ol | | | |
| 77 | 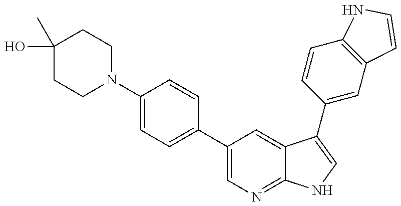 [SMILES: CC1(O)CCN(c2ccc(-c3cnc4[nH]cc(-c5ccc6[nH]ccc6c5)c4c3)cc2)CC1] | E77 | 423.4 [M + H] | 11.70 (s, 1H), 11.04 (s, 1H), 8.45 (s, 1H), 8.29 (s, 1H), 7.85 (s, 1H), 7.69 (s, 1H), 7.54 (d, J = 8.4 Hz, 2H), 7.44 (s, 2H), 7.32 (s, 1H), 7.02 (d, J = 8.4 Hz, 2H), 6.45 (s, 1H), 4.31 (s, 1H), 3.24 (bs, 5H), 1.55 (s, 3H), 1.13 (s, 3H) |
| | 1-(4-(3-(1H-indol-5-yl)-1H- | | | |
| | pyrrolo[2,3-b]pyridin-5- | | | |
| | yl)phenyl)-4-methylpiperidin-4-ol | | | |
| 78 | 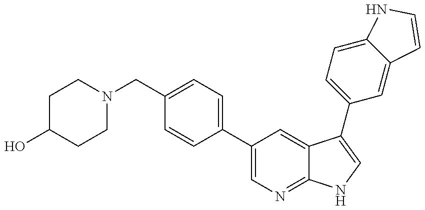 [SMILES: OC1CCN(Cc2ccc(-c3cnc4[nH]cc(-c5ccc6[nH]ccc6c5)c4c3)cc2)CC1] | E78 | 423.2 [M + H] | 11.76 (s, 1H), 11.03 (s, 1H), 8.51 (s, 1H), 8.38 (s, 1H), 7.87- 7.82 (m, 1H), 7.73-7.31 (m, 1H), 7.67-7.65 (m, 2H), 7.46 (s, 2H), 7.38-7.36 (m, 2H), 7.32 (s, 1H), 6.46 (s, 1H), 4.49 (d, J = 3.6 Hz, 1H), 3.46-3.44 (m, 3H), 2.69- 2.66 (m, 2H), 2.04 (t, J = 8.0 Hz, 2H), 1.71-1.68 (m, 2H), 1.42- 1.37 (m, 2H) |
| | 1-(4-(3-(1H-indol-5-yl)-1H- | | | |
| | pyrrolo[2,3-b]pyridin-5- | | | |
| | yl)benzyl)piperidin-4-ol | | | |
| 79 | 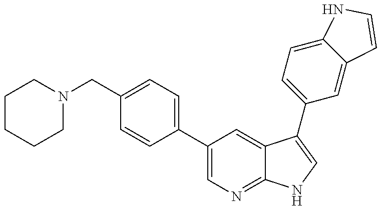 [SMILES: c1cc2cc(-c3c[nH]c4ncc(-c5ccc(CN6CCCCC6)cc5)cc34)ccc2[nH]1] | E79 | 407.2 [M + H] | 11.80 (bs, 1H), 11.06 (bs, 1H), 8.52 (s, 1H), 8.39 (s, 1H), 7.88 (s, 1H), 7.74 (s, 1H), 7.67 (d, J = 7.6 Hz, 4H), 7.46 (s, 1H), 7.38- 7.33 (m, 2H), 6.46 (s, 1H), 3.45 (s, 2H), 2.31-2.29 (m, 4H), 1.49- 1.20 (m, 6H) |
| | 3-(1H-indol-5-yl)-5-(4- | | | |
| | (piperidin-1-ylmethyl)phenyl)- | | | |
| | 1H-pyrrolo[2,3-b]pyridine | | | |
| 80 | 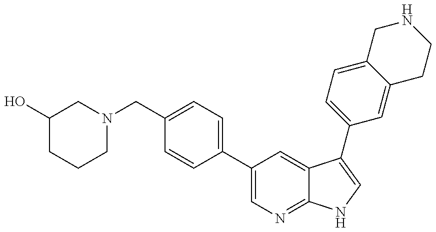 [SMILES: OC1CCCN(Cc2ccc(-c3cnc4[nH]cc(-c5ccc6c(c5)CCNC6)c4c3)cc2)C1] | E80 | 437.3 [M − H] | 11.86 (s, 1H), 8.52 (s, 1H), 8.35 (s, 1H), 7.79 (s, 1H), 7.67-7.66 (m, 2H), 7.48-7.46 (m, 1H), 7.41-7.36 (m, 3H), 7.07-7.05 (m, 1H), 4.54 (d, J = 3.2 Hz, 1H), 3.84 (s, 2H), 3.55-3.53 (m, 1H), 3.44-3.42 (m, 2H), 2.95-2.93 (m, 2H), 2.82-2.75 (m, 3H), 2.65- 2.63 (m, 2H), 1.87-1.85 (m, 1H), 1.79-1.77 (m, 1H), 1.73-1.71 (m, 1H), 1.62-1.61 (m, 1H), 1.43- 1.41 (m, 1H), 1.06-1.04 (m, 1H) |
| | 1-(4-(3-(1,2,3,4- | | | |
| | tetrahydroisoquinolin-6-yl)-1H- | | | |
| | pyrrolo[2,3-b]pyridin-5- | | | |
| | yl)benzyl)piperidin-3-ol | | | |
| 81 | 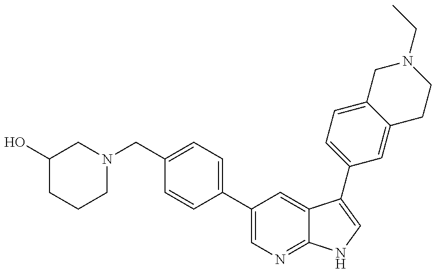 [SMILES: CCN1CCc2cc(-c3c[nH]c4ncc(-c5ccc(CN6CCCC(O)C6)cc5)cc34)ccc2C1] | E81 | 467.3 [M + H] | 11.88 (s, 1H), 8.53 (s, 1H), 8.37 (s, 1H), 7.81 (s, 1H), 7.69-7.67 (m, 2H), 7.52-7.50 (m, 2H), 7.39-7.37 (m, 2H), 7.11 (d, J = 7.6 Hz, 1H), 4.55 (d, J = 3.6 Hz, 1H), 3.56-3.51 (m, 3H), 3.49- 3.47 (m, 2H), 2.89-2.87 (m, 1H), 2.80-2.79 (m, 1H), 2.66-2.64 (m, 2H), 1.91-1.80 (m, 2H), 1.72- 1.70 (m, 1H), 1.44-1.41 (m, 1H), 1.12-1.08 (m, 3H) |
| | 1-(4-(3-(2-ethyl-1,2,3,4- | | | |
| | tetrahydroisoquinolin-6-yl)-1H- | | | |
| | pyrrolo[2,3-b]pyridin-5- | | | |
| | yl)benzyl)piperidin-3-ol | | | |
| 82 | 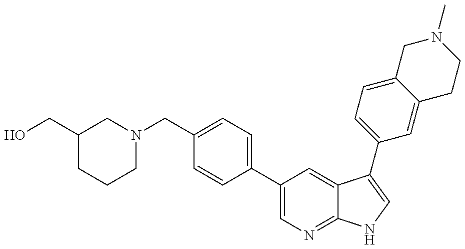 [SMILES: CN1CCc2cc(-c3c[nH]c4ncc(-c5ccc(CN6CCCC(CO)C6)cc5)cc34)ccc2C1] | E82 | 467.4 [M + H] | 11.91 (s, 1H), 8.53 (s, 1H), 8.37 (s, 1H), 7.82 (s, 1H), 7.68 (d, J = 7.6 Hz, 2H), 7.52-7.47 (m, 2H), 7.38 (d, J = 7.6 Hz, 2H), 7.11- 7.09 (m, 1H), 4.38 (s, 1H), 3.49- 3.45 (m, 4H), 3.18-3.16 (m, 2H), 2.89 (s, 2H), 2.75-2.73 (m, 1H), 2.65-2.63 (m, 2H), 2.34 (s, 3H), 1.90-1.88 (m, 1H), 1.62-1.60 (m, 4H), 1.46-1.44 (m, 1H) 1.26- 1.24 (m, 1H), 0.88-0.86 (m, 1H) |
| | (1-(4-(3-(2-methyl-1,2,3,4- | | | |
| | tetrahydroisoquinolin-6-yl)-1H- | | | |
| | pyrrolo[2,3-b]pyridin-5- | | | |
| | yl)benzyl)piperidin-3- | | | |
| | yl)methanol | | | |
| 83 | 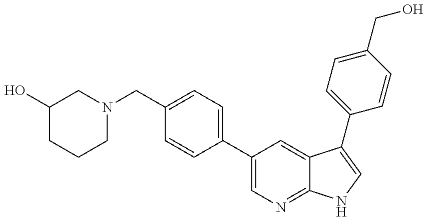 [SMILES: OCc1ccc(-c2c[nH]c3ncc(-c4ccc(CN5CCCC(O)C5)cc4)cc23)cc1] | E83 | 414.2 [M + H] | 11.94 (bs, 1H), 8.56 (d, J = 1.6 Hz, 1H), 8.41 (d, J = 1.6 Hz, 1H), 7.87 (d, J = 2.4 Hz, 1H), 7.75-7.70 (m, 4H), 7.40 (d, J = 8.0 Hz, 4H), 5.15 (d, J = 5.6 Hz, 1H), 4.57 (bs, 1H), 3.53 (d, J = 5.6 Hz, 2H), 3.58-3.43 (m, 3H), 2.84 (d, J = 7.6 Hz, 1H), 2.67 (d, J = 1.6 Hz, 1H), 1.91-1.62 (m, 4H), 1.46-1.07 (m, 2H) |
| | 1-(4-(3-(4- | | | |
| | (hydroxymethyl)phenyl)-1H- | | | |
| | pyrrolo[2,3-b]pyridin-5- | | | |
| | yl)benzyl)piperidin-3-ol | | | |
| 84 | 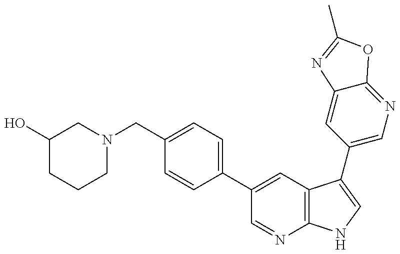 [SMILES: Cc1nc2cc(-c3c[nH]c4ncc(-c5ccc(CN6CCCC(O)C6)cc5)cc34)cnc2o1] | E84 | 440.3 [M + H] | 12.05 (s, 1H), 8.65 (d, J = 2.0 Hz, 1H), 8.52 (d, J = 2.0 Hz, 1H), 8.41 (dd, J = 13.3, 2.1 Hz, 2H), 7.98 (s, 1H), 7.68 (d, J = 8.0 Hz, 2H), 7.32 (d, J = 8.0 Hz, 2H), 4.47-4.45 (m, 1H), 3.49 (d, J = 13.2 Hz, 1H), 3.38 (d, J = 13.3 Hz, 2H), 2.76 (dd, J = 10.8, 3.8 Hz, 1H), 2.61 (s, 4H), 1.84- 1.79 (m, 3H), 1.74-1.61 (m, 1H), 1.57-1.55 (m, 1H), 1.38-1.36 (m, 1H). |
| | 1-(4-(3-(2-methyloxazolo[5,4- | | | |
| | b]pyridin-6-yl)-1H-pyrrolo[2,3- | | | |
| | b]pyridin-5- | | | |
| | yl)benzyl)piperidin-3-ol | | | |

Examples 26 and 27 were obtained by subjecting Example 25 to chiral preparative HPLC.

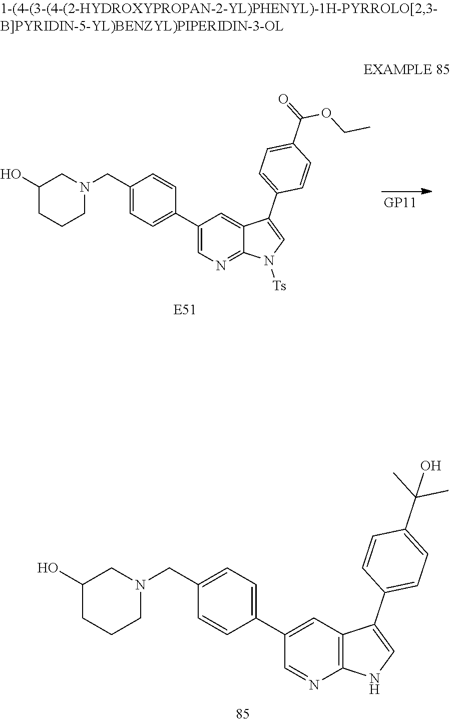 [SMILES: CCOC(=O)c1ccc(-c2cn(S(=O)(=O)c3ccc(C)cc3)c3ncc(-c4ccc(CN5CCCC(O)C5)cc4)cc23)cc1 ; CC(C)(O)c1ccc(-c2c[nH]c3ncc(-c4ccc(CN5CCCC(O)C5)cc4)cc23)cc1]

The title compound was synthesized by following General Procedure GP 11, starting from ethyl 4-(5-(4-((3-hydroxypiperidin-1-yl)methyl)phenyl)-1-tosyl-1H-pyrrolo[2,3-b]pyridin-3-yl)benzoate (E51, 0.150 g, 0.246 mmol), and was obtained as a gum (0.020 g, 18% yield). ¹H NMR (400 MHz, DMSO-d₆) δ=11.92 (bs, 1H), 8.55 (d, J=2.0 Hz, 1H), 8.40 (d, J=2.0 Hz, 1H), 7.84 (s, 1H), 7.72-7.69 (m, 4H), 7.54 (d, J=8.4 Hz, 2H), 7.40 (d, J=8.0 Hz, 2H), 4.99 (bs, 1H), 4.57 (bs, 1H), 3.58-3.36 (m, 3H), 2.83 (d, J=6.8 Hz, 1H), 2.69 (t, J=8.0 Hz, 1H), 1.92-1.61 (m, 4H), 1.47 (s, 6H), 1.42 (s, 1H), 1.12-1.04 (m, 1H). LC/MS: 440.1 [M−H].

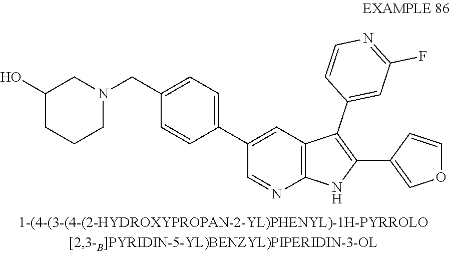 [SMILES: OC1CCCN(Cc2ccc(-c3cnc4[nH]c(-c5ccoc5)c(-c5ccnc(F)c5)c4c3)cc2)C1]

The title compound was synthesized by following General Procedure GP6, starting from tert-butyl 3-(2-fluoropyridin-4-yl)-2-(furan-3-yl)-5-(4-((3-hydroxypiperidin-1-yl)methyl)phenyl)-1H-pyrrolo[2,3-b]pyridine-1-carboxylate (E68, 0.150 g, 0.264 mmol), and was obtained as an off-white solid (0.05 g, 40% yield). ¹H NMR (400 MHz, DMSO-d₆) δ=12.43 (s, 1H), 8.60 (d, J=2.0 Hz, 1H), 8.28 (d, J=5.2 Hz, 1H), 8.14-8.09 (m, 2H), 7.81-7.80 (m, 1H), 7.71-7.69 (m, 2H), 7.48 (d, J=5.2 Hz, 1H), 7.38 (d, J=8.0 Hz, 2H), 7.29 (s, 1H), 6.64-6.63 (m, 1H), 4.55 (d, J=4.8 Hz, 1H), 3.57-3.54 (m, 1H), 3.52 (s, 2H), 2.82-2.80 (m, 1H), 2.68-2.66 (m, 1H), 2.07-2.04 (m, 2H), 1.79-1.75 (m, 1H), 1.64-1.59 (m, 1H), 1.48-1.44 (m, 1H), 1.12-1.09 (m, 1H). LC/MS: 469.2 [M+H].

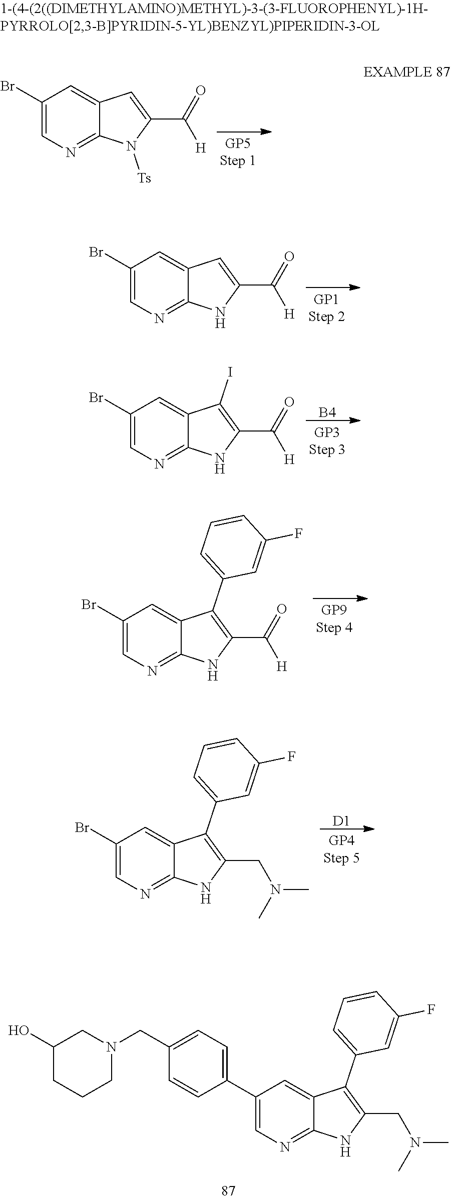

### Step 1: Synthesis of 5-bromo-1H-pyrrolo[2,3-b]pyridine-2-carbaldehyde

The title compound was synthesized by following General Procedure GP5 (with TBAF), starting from 5-bromo-1-tosyl-1H-pyrrolo[2,3-b]pyridine-2-carbaldehyde (step 1 of Intermediate A6, 3.5 g, 9.23 mmol), and was obtained as an off-white solid (1.8 g, crude) which was used without further purification. LC/MS: 226.0 [M+H].

### Step 2: Synthesis of 5-bromo-3-iodo-1H-pyrrolo[2,3-b]pyridine-2-carbaldehyde

The title compound was synthesized by following General Procedure GP1, starting from 5-bromo-1H-pyrrolo[2,3-b]pyridine-2-carbaldehyde (1.80 g, 8.00 mmol), and was obtained as a brown solid (1.0 g, crude) which was used without further purification. LC/MS: 352.9 [M+H].

### Step 3: Synthesis of 5-bromo-3-(3-fluorophenyl)-1H-pyrrolo[2,3-b]pyridine-2-carbaldehyde

The title compound was synthesized by following General Procedure GP3, starting from 5-bromo-3-iodo-1H-pyrrolo[2,3-b]pyridine-2-carbaldehyde (1.000 g, 2.80 mmol) and (3-fluorophenyl)boronic acid (B4, 0.399 g, 2.85 mmol), and was obtained as a light brown solid (0.5 g, 55% yield). LC/MS: 321.0 [M+H].

### Step 4: Synthesis of 1-(5-bromo-3-(3-fluorophenyl)-1H-pyrrolo[2,3-b]pyridin-2-yl)-N,N-dimethylmethanamine

The title compound was synthesized by following General Procedure GP9, starting from 5-bromo-3-(3-fluorophenyl)-1H-pyrrolo[2,3-b]pyridine-2-carbaldehyde (0.150 g, 0.47 mmol) and dimethylamine hydrochloride (0.042 g, 0.51 mmol), and was obtained as a pale brown solid (0.06 g, 37% yield). LC/MS: 350.1 [M+H].

### Step 5: Synthesis of 1-(4-(2-((dimethylamino)methyl)-3-(3-fluorophenyl)-1H-pyrrolo[2,3-b]pyridin-5-yl)benzyl)piperidin-3-ol

The title compound was synthesized by following General Procedure GP4, starting from 1-(5-bromo-3-(3-fluorophenyl)-1H-pyrrolo[2,3-b]pyridin-2-yl)-N,N-dimethylmethanamine (0.060 g, 0.20 mmol) and 1-(4-(4,4,5,5-tetramethyl-1,3,2-dioxaborolan-2-yl)benzyl)piperidin-3-ol (D1, 0.063 g, 0.20 mmol), and was obtained as an off-white solid (0.003 g, 3.3% yield). ¹H NMR (400 MHz, DMSO-d₆) δ=11.92 (s, 1H), 8.46 (d, J=2.0 Hz, 1H), 8.06 (d, J=2.0 Hz, 1H), 7.59 (d, J=8.0 Hz, 2H), 7.48-7.43 (m, 3H), 7.30 (d, J=8.0 Hz, 2H), 7.09-7.05 (m, 1H), 4.49 (d, J=4.8 Hz, 1H), 3.49-3.27 (m, 5H), 2.74 (d, J=6.8 Hz, 1H), 2.13 (s, 6H), 1.83-1.72 (m, 4H), 1.64-1.53 (m, 1H), 1.37-1.34 (m, 1H), 1.01-0.97 (m, 1H). LC/MS: 459.2 [M+H].

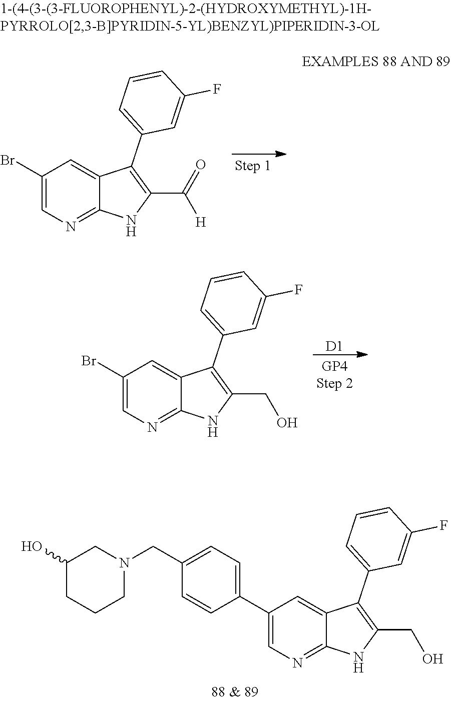 [SMILES: O=Cc1[nH]c2ncc(Br)cc2c1-c1cccc(F)c1 ; OCc1[nH]c2ncc(Br)cc2c1-c1cccc(F)c1 ; "D1" ; OCc1[nH]c2ncc(-c3ccc(CN4CCCC(O)C4)cc3)cc2c1-c1cccc(F)c1]

### Step 1: Synthesis of (5-bromo-3-(3-fluorophenyl)-1H-pyrrolo[2,3-b]pyridin-2-yl)methanol

NaBH₄ (0.071 g, 1.88 mmol) was added to a solution of 5-bromo-3-(3-fluorophenyl)-1H-pyrrolo[2,3-b]pyridine-2-carbaldehyde (step 3 of Example 87, 0.500 g, 1.57 mmol) in methanol (5 mL) at 0° C. and the resulting mixture was allowed to warm to 25° C. and was stirred for 1 h. Following completion of the reaction, the mixture was poured into a saturated NH₄Cl solution (20 mL) and extracted with EtOAc (2×50 mL). The combined organic phases were washed with brine, dried over anhydrous Na₂SO, filtered, and concentrated under reduced pressure to afford the title compound as an off-white solid (0.11 g, 22% yield) which was used without further purification. LC/MS: 323.0 [M+H].

### Step 2: Synthesis of 1-(4-(3-(3-fluorophenyl)-2-(hydroxymethyl)-1H-pyrrolo[2,3-b]pyridin-5-yl)benzyl)piperidin-3-ol

The title compound was synthesized by following General Procedure GP4, starting from (5-bromo-3-(3-fluorophenyl)-1H-pyrrolo[2,3-b]pyridin-2-yl)methanol (0.110 g, 0.343 mmol) and 1-(4-(4,4,5,5-tetramethyl-1,3,2-dioxaborolan-2-yl)benzyl)piperidin-3-ol (D1, 0.109 g, 0.343 mmol), and was obtained as a mixture of enantiomers which were resolved by chiral preparative HPLC (using a CHIRALPAK IA 250×30 mm, 5 μm column and eluting with 0.1% diethylamine in EtOH).

Enantiomer 1: off-white solid (0.010 g, 6.7% yield). ¹H NMR (400 MHz, DMSO-d₆) δ=12.00 (s, 1H), 8.55 (d, J=2.0 Hz, 1H), 8.16 (d, J=2.0 Hz, 1H), 7.67 (d, J=8.0 Hz, 2H), 7.56-7.44 (m, 3H), 7.38 (d, J=8.0 Hz, 2H), 7.15 (t, J=9.2 Hz, 1H), 5.46 (t, J=5.2 Hz, 1H), 4.68 (d, J=5.2 Hz, 2H), 4.59 (d, J=5.2 Hz, 1H), 3.56-3.53 (m, 1H), 3.52-3.50 (m, 2H), 2.82-2.80 (m, 2H), 1.90-1.72 (m, 3H), 1.63-1.60 (m, 1H), 1.44-1.41 (m, 1H), 1.23-1.21 (m, 1H), 1.08-1.06 (m, 1H). LC/MS: 430.2 [M−H]

Enantiomer 2: off-white solid (0.011 g, 7.4% yield). ¹H NMR (400 MHz, DMSO-d₆) δ=12.00 (s, 1H), 8.54 (d, J=4 Hz, 1H), 8.16 (d, J=4 Hz, 1H), 7.67 (d, J=8 Hz, 2H), 7.54-7.45 (m, 3H), 7.38 (d, J=8 Hz, 2H), 7.16 (t, J=8 Hz, 1H), 5.46 (t, J=4 Hz, 1H), 4.67 (d, J=4 Hz, 2H), 4.58 (d, J=4 Hz, 1H), 2.81 (d, J=8 Hz, 2H), 1.91-1.70 (m, 4H), 1.61 (d, J=12 Hz, 1H), 1.44-1.41 (m, 1H), 1.26-1.23 (m, 1H) 1.10-1.03 (m, 2H). LC/MS: 430.2 [M−H].

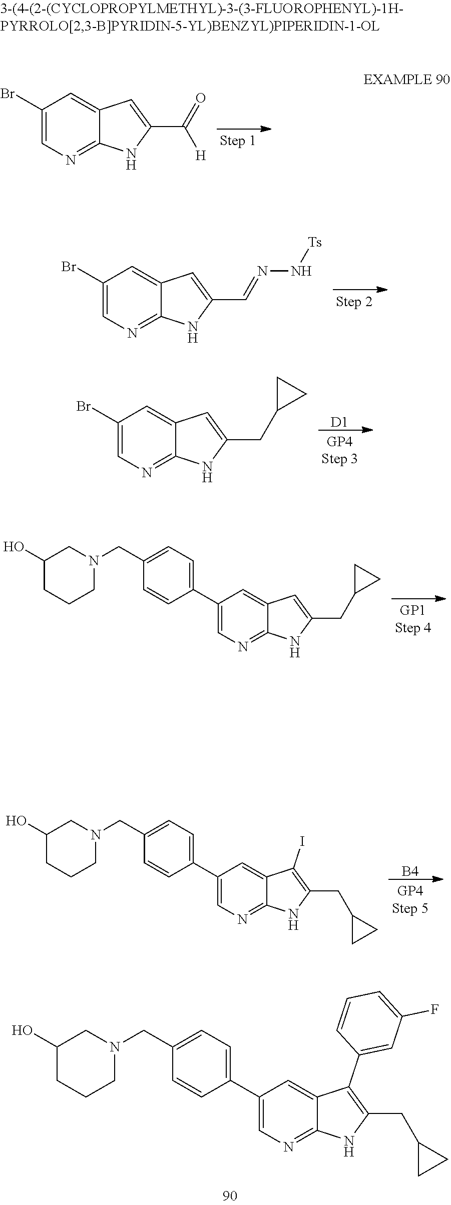

### Step 1: Synthesis of N-((5-bromo-1H-pyrrolo[2,3-b]pyridin-2-yl)methylene)-4-methylbenzenesulfonohydrazide

4-Methylbenzenesulfonohydrazide (1.74 g, 9.33 mmol) was added to a solution of 5-bromo-1H-pyrrolo[2,3-b]pyridine-2-carbaldehyde (step 1 of Example 87, 2.10 g, 9.33 mmol) in methanol (30 mL) and the resulting mixture was heated to reflux temperature for 12 h. Following completion of the reaction, the mixture was diluted with water (100 mL) and the resulting precipitate was collected by filtration and dried under reduced pressure to give the title compound (2.90 g, 79% yield) as an off-white solid. LC/MS: 393.1 [M+H].

### Step 2: Synthesis of 5-bromo-2-(cyclopropylmethyl)-1H-pyrrolo[2,3-b]pyridine

Cs₂CO₃ (1.240 g, 3.81 mmol) and cyclopropylboronic acid (0.218 g, 2.54 mmol) were added to a solution of N-((5-bromo-1H-pyrrolo[2,3-b]pyridin-2-yl)methylene)-4-methylbenzenesulfonohydrazide (1.000 g, 2.54 mmol) in 1,4-dioxane (10 mL) and the resulting mixture was stirred at 110° C. for 12 h. Following completion of the reaction, the mixture was diluted with water (50 mL) and extracted with EtOAc (2×100 mL). The combined organic phases were washed with brine (50 mL), dried over anhydrous Na₂SO₄, filtered, and concentrated under reduced pressure to give crude material which was purified by flash chromatography (eluting with 10 to 50% EtOAc in n-hexane), affording the title compound as an off-white solid (0.290 g, 45% yield). LC/MS: 253.1 [M+H].

### Step 3: Synthesis of 1-(4-(2-(cyclopropylmethyl)-1H-pyrrolo[2,3-b]pyridin-5-yl)benzyl)piperidin-3-ol

The title compound was synthesized by following General Procedure GP4, starting from 5-bromo-2-(cyclopropylmethyl)-1H-pyrrolo[2,3-b]pyridine (0.290 g, 1.155 mmol) and 1-(4-(4,4,5,5-tetramethyl-1,3,2-dioxaborolan-2-yl)benzyl)piperidin-3-ol (D1, 0.366 g, 1.150 mmol), and was obtained as an off-white solid (0.230 g, 55% yield). LC/MS: 362.3 [M+H].

### Step 4: Synthesis of 1-(4-(2-(cyclopropylmethyl)-3-iodo-1H-pyrrolo[2,3-b]pyridin-5-yl)benzyl)piperidin-3-ol

The title compound was synthesized by following General Procedure GP1, starting from 1-(4-(2-(cyclopropylmethyl)-1H-pyrrolo[2,3-b]pyridin-5-yl)benzyl)piperidin-3-ol (0.230 g, 0.636 mmol), and was obtained as an off-white solid (0.230 g, 74% yield) which was used without further purification. LC/MS: 488.1 [M+H].

### Step 5: Synthesis of 1-(4-(2-(cyclopropylmethyl)-3-(3-fluorophenyl)-1H-pyrrolo[2,3-b]pyridin-5-yl)benzyl)piperidin-3-ol

The title compound was synthesized by following General Procedure GP4, starting from 1-(4-(2-(cyclopropylmethyl)-3-iodo-1H-pyrrolo[2,3-b]pyridin-5-yl)benzyl)piperidin-3-ol (0.200 g, 0.410 mmol) and (3-fluorophenyl)boronic acid (B4, 0.057 g, 0.41 mmol), and was obtained as an off-white solid (0.035 g, 19% yield). ¹H NMR (400 MHz, DMSO-d₆) δ=11.86 (s, 1H), 8.50 (d, J=2.0 Hz, 1H), 8.03 (d, J=1.6 Hz, 1H), 7.65 (d, J=8.0 Hz, 2H), 7.55-7.50 (m, 1H), 7.38-7.33 (m, 4H), 7.13 (t, J=6.8 Hz, 1H), 4.54 (d, J=4.8 Hz, 1H), 3.56-3.42 (m, 3H), 2.96-2.78 (m, 3H), 2.67 (s, 1H), 1.90-1.60 (m, 4H), 1.47-1.68 (m, 1H), 1.17-1.02 (m, 2H), 0.48-0.43 (m, 2H), 0.27-0.21 (m, 2H). LC/MS: 456.3 [M+H].

### Biological Example 1

### TYRO3 Enzymatic Assay

Poly(Glu, Tyr) sodium salt (Glu:Tyr (4:1), quality level 200, molecular weight 5,000-20,000, obtained from Sigma Aldrich, catalogue #P7244, assay concentration 0.20 mg/mL) and recombinant human TYRO3 (obtained from ThermoFisher Scientific, catalogue #PV3828, assay concentration 2 nM) were mixed in assay buffer (20 mM Hepes pH 7.5, 10 mM MgCl₂, 1 mM EGTA, 0.02% Brij35, 0.02 mg/ml BSA, 0.1 mM Na₃VO₄, 2 mM DTT, 1% DMSO, with added MnCl₂ at a final concentration of 2 mM). Compounds of interest (in DMSO, serial 3-fold dilution from 10 μM to 0.5 nM) or control (1% DMSO) were dispensed into the kinase reaction mixture by Acoustic technology (Echo550; nanoliter range). After incubation at room temperature for 20 minutes, the kinase reaction was initiated by addition of [³²P]-ATP (Specific activity 10 μCi/μl) and the mixture was incubated at room temperature for 2 hours. The reaction was then stopped by spotting the reaction mixture on strips of phosphocellulose P81 paper. Following washing, the radioactivity of the P81 paper was measured and kinase activity data were expressed as the percent remaining kinase activity in test samples compared to vehicle (dimethyl sulfoxide) reactions. IC₅₀ values and curve fits were obtained using Prism (GraphPad Software).

TABLE 2
Activity of Representative Compounds of Structure (I)

| Compound No. | TYRO3 IC₅₀ (nM) |
|---|---|
| 1 | *** |
| 2 | **** |
| 3 | *** |
| 4 | **** |
| 5 | *** |
| 6 | *** |
| 7 | *** |
| 8 | *** |
| 9 | **** |
| 10 | ** |
| 11 | *** |
| 12 | **** |
| 13 | * |
| 14 | **** |
| 15 | **** |
| 16 | **** |
| 17 | **** |
| 18 | **** |
| 19 | *** |
| 20 | *** |
| 21 | *** |
| 22 | *** |
| 23 | **** |
| 24 | *** |
| 25 | **** |
| 26 | **** |
| 27 | *** |
| 28 | ** |
| 29 | *** |
| 30 | *** |
| 31 | * |
| 32 | *** |
| 33 | * |
| 34 | *** |
| 35 | *** |
| 36 | ** |
| 37 | ** |
| 38 | ** |
| 39 | ** |
| 40 | ** |
| 41 | * |
| 42 | ** |
| 43 | *** |
| 44 | **** |
| 45 | **** |
| 46 | **** |
| 47 | **** |
| 48 | **** |
| 49 | **** |
| 50 | **** |
| 51 | **** |
| 52 | **** |
| 53 | **** |
| 54 | *** |
| 55 | ** |
| 56 | **** |
| 57 | **** |
| 58 | **** |
| 59 | **** |
| 60 | **** |
| 61 | **** |
| 62 | *** |
| 63 | **** |
| 64 | *** |
| 65 | **** |
| 66 | **** |
| 67 | **** |
| 68 | *** |
| 69 | ** |
| 70 | *** |
| 71 | *** |
| 72 | * |
| 73 | ** |
| 74 | ** |
| 75 | **** |
| 76 | **** |
| 77 | ** |
| 78 | **** |
| 79 | **** |
| 80 | **** |
| 81 | *** |
| 82 | *** |
| 83 | **** |
| 84 | **** |
| 85 | **** |
| 86 | **** |
| 87 | * |
| 88 | ** |
| 89 | *** |
| 90 | **** |

For TYRO3 IC₅₀ activity in Table 2:
* IC₅₀ greater than 501 nM
** IC₅₀ range from 151-500 nM
*** IC₅₀ range from 51-150 nM
**** IC₅₀ less than 51 nM

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Application No. 63/254,275 filed on Oct. 11, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having the following Structure (I): 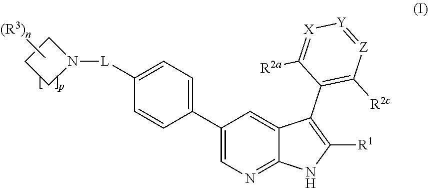
or a pharmaceutically acceptable salt, stereoisomer, tautomer, or prodrug thereof, wherein:
X is N or CR^{2d};
Y is N or CR^{2b};
Z is N or CR^{2e};
R¹ is hydrogen, C₁-C₆ alkyl, C₁-C₆ hydroxyalkyl, halo, C₁-C₆ haloalkyl, C₂-C₆ alkoxyalkyl, C₁-C₆ aminylalkyl, optionally substituted cycloalkylalkyl, or optionally substituted heteroaryl;
R^{2a}, R^{2b}, R^{2c}, R^{2d}, and R^{2e} are each, independently, hydrogen, C₁-C₆ alkyl, halo, cyano, C₁-C₆ haloalkyl, —R⁵OR⁶, or —R⁵N(R⁶)₂;
or R^{2b} and R^{2e}, with the carbons to which they are attached, form a heterocyclyl optionally substituted with one or more alkyl groups;
or R^{2b} and R^{2e}, with the adjacent carbons to which they are attached, form a heteroaryl that is optionally substituted with one or more alkyl groups;
R³ is, at each occurrence, independently alkyl, halo, —R⁵OR⁶, —R⁵OC(═O)R⁶, or —R⁵N(R⁶)₂;
or two R³'s, when attached to different carbons, join to form —CH₂O—;
or two R³'s, with the carbon to which they are both attached, form an optionally substituted heterocyclyl;
L is a direct bond or —C(R⁴)₂—;
R⁴ is, at each occurrence, independently hydrogen or alkyl;
R⁵ is, at each occurrence, independently a direct bond or an alkylene chain optionally substituted with one or more alkyl groups;
R⁶ is, at each occurrence, independently hydrogen, alkyl, or haloalkyl;
n is 0, 1, 2, or 3; and
p is 1, 2, or 3
provided that:
when one or more occurrences of R³ are fluoro, then at least one of X and Y is N; and
when p is 2 and one occurrence of R³ is —OH, then at least one of X and Y is N.

2. The compound of claim 1, wherein R¹ is hydrogen, C₁-C₃ alkyl, C₁-C₃ hydroxyalkyl, halo, C₁-C₂ haloalkyl, C₂-C₆ alkoxyalkyl, C₁-C₄ aminylalkyl, optionally substituted cyclopropylalkyl, or optionally substituted 5-membered heteroaryl.

3. The compound of any one of claims 1-2, wherein R¹ is methyl, ethyl, isopropyl, difluoromethyl, trifluoromethyl, methoxymethyl, ethoxymethyl, fluoro, chloro, bromo, or has one of the following structures:

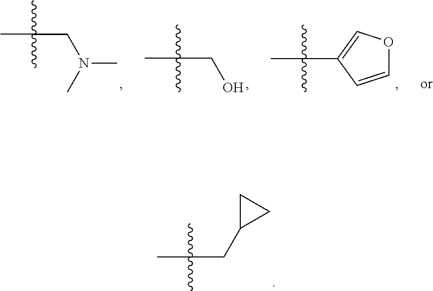

4. The compound of any one of claims 1-2, wherein R¹ is methyl, ethyl, isopropyl, difluoromethyl, trifluoromethyl, methoxymethyl, fluoro, chloro, or bromo.

5. The compound of any one of claims 1-2, wherein R¹ is methyl, ethyl, difluoromethyl, methoxymethyl, ethoxymethyl, or chloro.

6. The compound of any one of claims 1-2, wherein R¹ has one of the following structures:

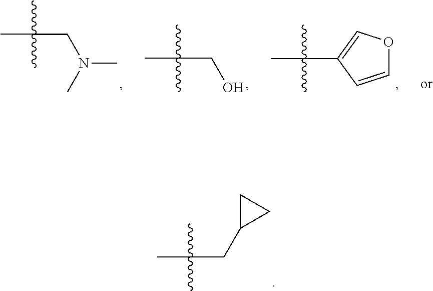

7. The compound of any one of claims 1-2, wherein R¹ is hydrogen.

8. The compound of any one of claims 1-7, wherein X is N.

9. The compound of any one of claims 1-8, wherein Y is N.

10. The compound of any one of claims 1-9, wherein Z is N.

11. The compound of any one of claims 1-7 or 9-10, wherein X is CR^{2d}.

12. The compound of any one of claims 1-8 or 10-11, wherein Y is CR^{2b}.

13. The compound of any one of claims 1-9 or 11-12, wherein Z is CR².

14. The compound of any one of claims 1-13, wherein the compound has one of the following structures (Ia), (Ib), (Ic), (Id), (Ie), (If), (Ig), (Ih), (Ii), or (Ij): 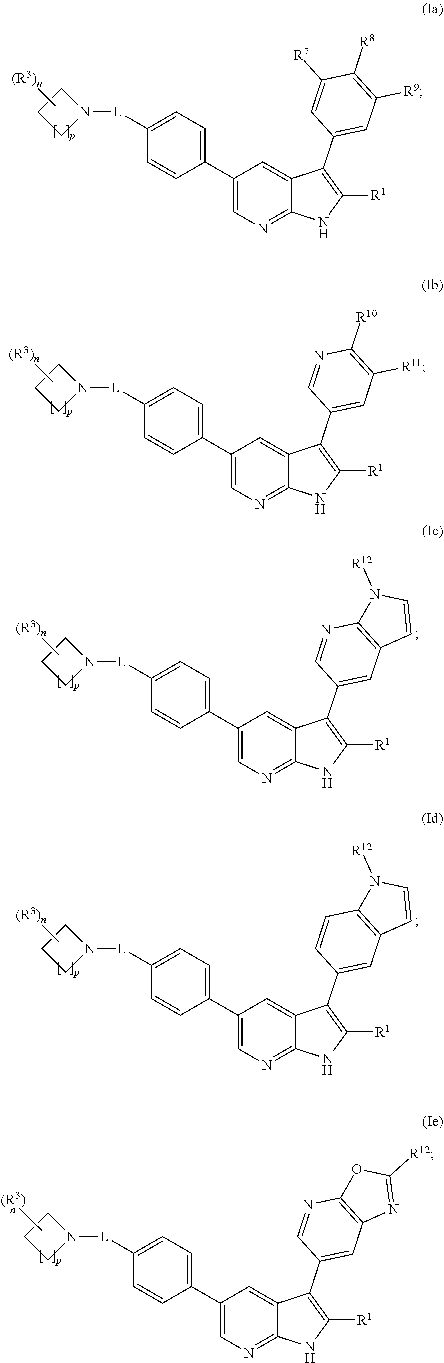 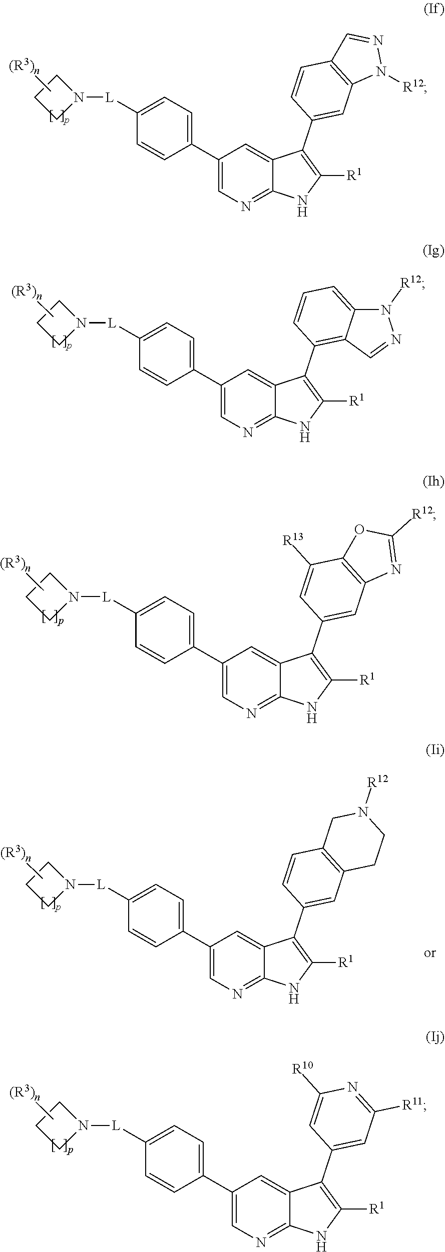
or a pharmaceutically acceptable salt, stereoisomer, tautomer, or prodrug thereof, wherein:
R⁷, R¹, R⁹, R¹⁰, and R¹¹ are each, independently, C₁-C₆ alkyl, halo, cyano, C₁-C₆ haloalkyl, —R⁵OR⁶, or —R⁵N(R⁶)₂;
R¹² is hydrogen or C₁-C₆ alkyl; and
R¹³ is hydrogen or halo.

15. The compound of any one of claims 1-13, wherein at least one of R^{2a}, R^{2b}, R^{2c}, R^{2d}, and R^{2e} is selected from the group consisting of C₁-C₃ alkyl, halo, cyano, C₁-C₂ haloalkyl, —R⁵OR⁶, or —R⁵N(R⁶)₂.

16. The compound of any one of claims 1-13, wherein at least one of R^{2a}, R^{2b}R^{2c}, R^{2d}, and R^{2e} is selected from the group consisting of methyl, hydroxymethyl, hydroxyisopropyl, hydroxyl, cyano, fluoro, chloro, trifloromethyl, difluoromethyl, methoxy, isopropoxy, and dimethylaminomethyl.

17. The compound of any one of claims 1-16, wherein L is a direct bond.

18. The compound of any one of claims 1-16, wherein L is —C(R⁴)₂—.

19. The compound of claim 18, wherein L is —CH₂— or —CH(CH₃)—.

20. The compound of any one of claims 1-19, wherein p is 1.

21. The compound of any one of claims 1-19, wherein p is 2.

22. The compound of any one of claims 1-19, wherein p is 3.

23. The compound of any one of claims 1-22, wherein n is 1, 2, or 3.

24. The compound of any one of claims 1-23, wherein at least one occurrence of R³ is methyl, hydroxyl, methoxy, ethoxy, methylamino, dimethylamino, fluoro, methoxymethyl, hydroxymethyl, or —OC(═O)CH₃.

25. The compound of any one of claims 1-22, wherein n is 0.

26. The compound of any one of claims 1-25, wherein 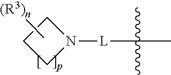
has one of the following structures:

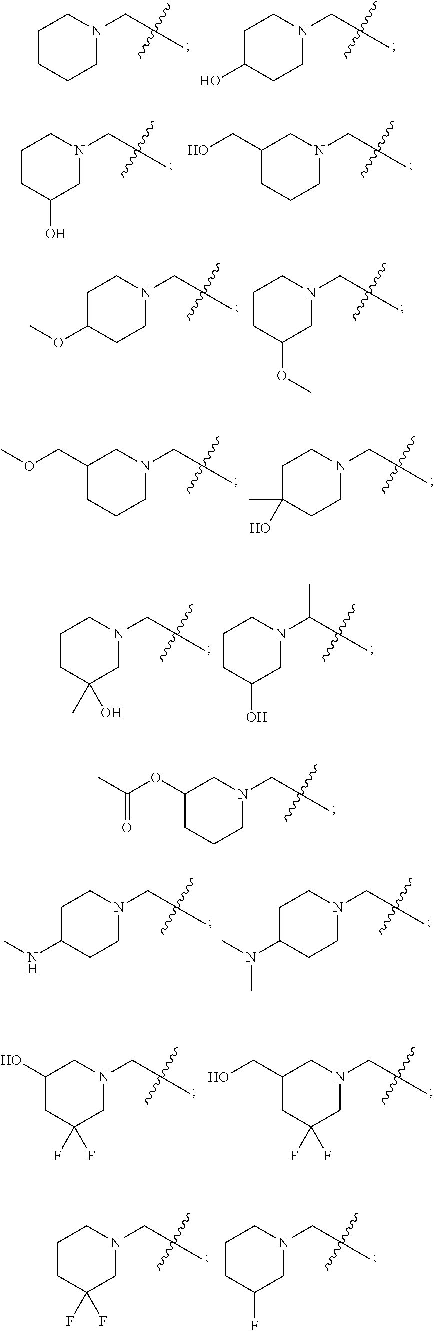

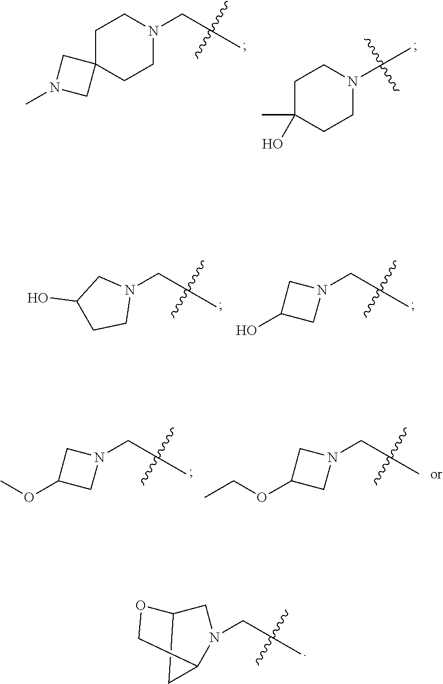

27. The compound of any one of claims 1-25, wherein 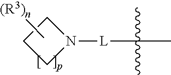
has one of the following structures:

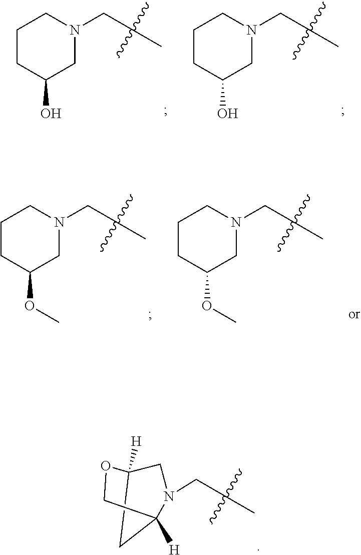

28. A compound having one of the structures listed in Table 1 or a pharmaceutically acceptable salt, stereoisomer, tautomer, or prodrug thereof.

29. The compound of any one of claims 1-28, wherein the compound is an inhibitor of TYRO3.

30. A pharmaceutical composition comprising the compound of any one of claims 1-29 or a pharmaceutically acceptable salt, stereoisomer, tautomer, or prodrug thereof, and a pharmaceutically acceptable carrier, diluent, or excipient.

31. A method of treating a disease or disorder, comprising administering a therapeutically effective amount of a compound of any one of claims 1-29 or a pharmaceutically acceptable salt, stereoisomer, tautomer, or prodrug thereof, or the pharmaceutical composition of claim 30, to a subject in need thereof.

32. The method of claim 31, wherein the disease or disorder is a TYRO3-mediated disease or disorder.

33. The method of claim 32, wherein the disease or disorder is cancer.

34. The method of any one of claims 31-33, wherein the disease or disorder comprises a solid tumor.

35. The method of any one of claims 31-34, wherein the disease or disorder is leukemia, lymphoma, brain cancer, genitourinary tract cancer, endocrine system cancer, gastrointestinal tract cancer, colon cancer, rectal cancer, breast cancer, kidney cancer, lymphatic system cancer, stomach cancer, lung cancer, pancreatic cancer, skin cancer, or combinations thereof.

36. The method of any one of claims 31-35, wherein the disease or disorder is bladder tumor, diffuse large B-Cell lymphoma, adenoid cystic carcinoma of salivary gland, Burkitt lymphoma, multiple myeloma, pancreatic ductal adenocarcinoma, hairy cell leukemia, metastatic prostate cancer, melanoma, colorectal cancer, or a combination thereof.

37. The method of any one of claims 31-35, wherein the cancer is glioma.

38. The method of claim 37, wherein the glioma is glioblastoma multiforme, an ependymoma, astrocytoma, oligodendroglioma, oligoastrocytoma, a juvenile pilocytic astrocytoma, subependymal giant cell astrocytoma, ganglioglioma, subependymoma, xanthoastrocytoma, anaplastic, brainstem glioma, cerebellar astrocytoma, childhood desmoplastic astrocytoma, subependymal giant cell astrocytoma, diffuse astrocytoma, mixed glioma, optic glioma, cerebral gliomatosis, paraganglioma, ganglioglioma cells, or a combination thereof.

39. A method for inhibiting a TYRO3-mediated disease or disorder, the method comprising administering a therapeutically effective amount of a compound having the following Structure (II): 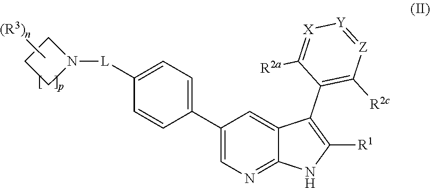
or a pharmaceutically acceptable salt, stereoisomer, tautomer, or prodrug thereof, wherein:
X is N or CR^{2d};
Y is N or CR^{2b};
Z is N or CR^{2e};
R¹ is hydrogen, C₁-C₆ alkyl, C₁-C₆ hydroxyalkyl, halo, C₁-C₆ haloalkyl, C₂-C₆ alkoxyalkyl, C₁-C₆ aminylalkyl, optionally substituted cycloalkylalkyl, or optionally substituted heteroaryl;
R^{2a}, R^{2b}, R^{2c}, R^{2d}, and R^{2e} are each, independently, hydrogen, C₁-C₆ alkyl, halo, cyano, C₁-C₆ haloalkyl, —R⁵OR⁶, or —R⁵N(R⁶)₂;
or R^{2b} and R^{2e}, with the carbons to which they are attached, form a heterocyclyl optionally substituted with one or more alkyl groups;
or R^{2b} and R^{2e}, with the adjacent carbons to which they are attached, form a heteroaryl that is optionally substituted with one or more alkyl groups;
R³ is, at each occurrence, independently alkyl, halo, —R⁵OR⁶, —R⁵OC(═O)R⁶, or —R⁵N(R⁶)₂;
or two R³'s, when attached to different carbons, join to form —CH₂O—;
or two R³'s, with the carbon to which they are both attached, form an optionally substituted heterocyclyl;
L is a direct bond or —C(R⁴)²—
R⁴ is, at each occurrence, independently hydrogen or alkyl;
R⁵ is, at each occurrence, independently a direct bond or an alkylene chain optionally substituted with one or more alkyl groups;
R⁶ is, at each occurrence, independently hydrogen, alkyl, or haloalkyl;
n is 0, 1, 2, or 3; and
p is 1, 2, or 3.

40. The method of claim 39, wherein the disease or disorder is cancer.

41. The method of claim 40, wherein the disease or disorder comprises a solid tumor.

42. The method of any one of claims 39-41, wherein the disease or disorder is leukemia, lymphoma, brain cancer, genitourinary tract cancer, endocrine system cancer, gastrointestinal tract cancer, colon cancer, rectal cancer, breast cancer, kidney cancer, lymphatic system cancer, stomach cancer, lung cancer, pancreatic cancer, skin cancer, or combinations thereof.

43. The method of any one of claims 39-41, wherein the disease or disorder is bladder tumor, diffuse large B-Cell lymphoma, adenoid cystic carcinoma of salivary gland, Burkitt lymphoma, multiple myeloma, pancreatic ductal adenocarcinoma, hairy cell leukemia, metastatic prostate cancer, melanoma, colorectal cancer, or a combination thereof.

44. The method of any one of claims 39-41, wherein the cancer is glioma.

45. The method of claim 44, wherein the glioma is glioblastoma multiforme, an ependymoma, astrocytoma, oligodendroglioma, oligoastrocytoma, a juvenile pilocytic astrocytoma, subependymal giant cell astrocytoma, ganglioglioma, subependymoma, xanthoastrocytoma, anaplastic, brainstem glioma, cerebellar astrocytoma, childhood desmoplastic astrocytoma, subependymal giant cell astrocytoma, diffuse astrocytoma, mixed glioma, optic glioma, cerebral gliomatosis, paraganglioma, ganglioglioma cells, or a combination thereof.
